(19) United States
(12) Patent Application Publication
(10) Pub. No.: US 2023/0050369 A1
(43) Pub. Date: Feb. 16, 2023

(54) ORGANIC ELECTROLUMINESCENT ELEMENT, COMPOUND AND ELECTRONIC DEVICE

(71) Applicant: IDEMITSU KOSAN CO.,LTD., Chiyoda-ku, Tokyo (JP)

(72) Inventors: Takushi SHIOMI, Chiyoda-ku, Tokyo (JP); Hiromi NAKANO, Chiyoda-ku, Tokyo (JP); Toshinari OGIWARA, Chiyoda-ku, Tokyo (JP); Hisato MATSUMOTO, Chiyoda-ku, Tokyo (JP)

(73) Assignee: IDEMITSU KOSAN CO.,LTD., Chiyoda-ku, Tokyo (JP)

(21) Appl. No.: 17/784,426

(22) PCT Filed: Dec. 8, 2020
(86) PCT No.: PCT/JP2020/045640

§ 371 Date: Jun. 10, 2022

(30) Foreign Application Priority Data
Dec. 10, 2019 (JP) 2019-222841

(51) Int. Cl. H01L 51/00; H01L 51/50

(52) U.S. Cl. CPC H01L 51/0073; H01L 51/0054; H01L 51/0052; H01L 51/0071; H01L 51/0064; H01L 51/0072; H01L 51/0059; H01L 51/0067; H01L 51/5016

(57) ABSTRACT

An organic electroluminescence device includes: an anode; a cathode; and an emitting layer provided between the anode and the cathode, in which the emitting layer contains a delayed fluorescent compound M2 and a compound M3 represented by a formula (3) and a singlet energy S₁(M2) of the compound M2 and a singlet energy S₁(M3) of the compound M3 satisfy a relationship of a numerical formula (Numerical Formula 1) below, S₁(M3)>S₁(M2) . . . (Numerical Formula 1). In the formula (3), A3 is a group represented by a formula (3a) or the like.

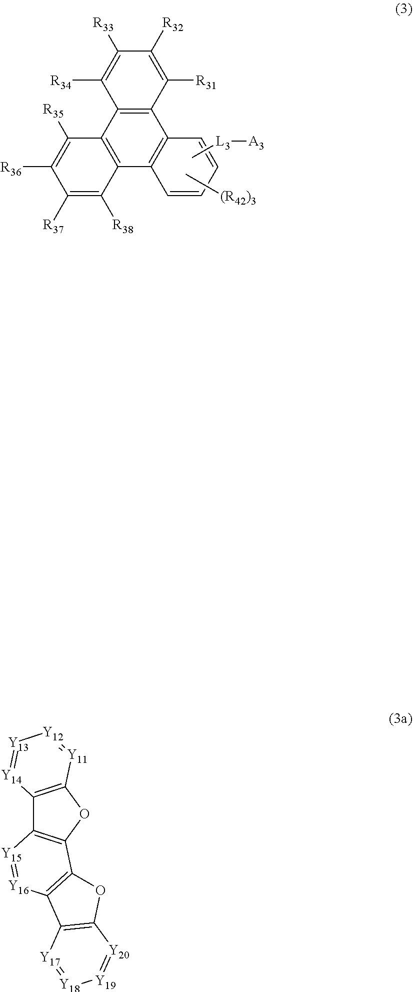

### TECHNICAL FIELD

The present invention relates to an organic electroluminescence device, a compound, and an electronic device.

### BACKGROUND ART

When a voltage is applied to an organic electroluminescence device (hereinafter, occasionally referred to as an organic EL device), holes are injected from an anode and electrons are injected from a cathode into an emitting layer. The injected holes and electrons are recombined in the emitting layer to form excitons. Specifically, according to the electron spin statistics theory, singlet excitons and triplet excitons are generated at a ratio of 25%:75%.

A fluorescent organic EL device using light emission from singlet excitons has been applied to a full-color display such as a mobile phone and a television set, but an internal quantum efficiency is said to be at a limit of 25%. Accordingly, studies have been made to improve a performance of the organic EL device.

For instance, the organic EL device is expected to emit light more efficiently using triplet excitons in addition to singlet excitons. In view of the above, a highly efficient fluorescent organic EL device using thermally activated delayed fluorescence (hereinafter, sometimes simply referred to as “delayed fluorescence”) has been proposed and studied.

A TADF (Thermally Activated Delayed Fluorescence) mechanism uses such a phenomenon that inverse intersystem crossing from triplet excitons to singlet excitons thermally occurs when a material having a small energy difference (ΔST) between singlet energy level and triplet energy level is used. Thermally activated delayed fluorescence is explained in “Yuki Hando-tai no Debaisu Bussei (Device Physics of Organic Semiconductors)” (edited by ADACHI, Chihaya, published by Kodansha, issued on Apr. 1, 2012, on pages 261-268).

Organic EL devices using such a TADF mechanism are disclosed in Patent Literatures 1 to 3.

Patent Literatures 4 to 6 each disclose a compound having a fused dibenzothienyl group and a triphenylenyl group as a compound usable for an organic EL device.

### CITATION LIST

### Patent Literature(s)

Patent Literature 1: JP 2015-109428 A

Patent Literature 2: International Publication No. WO2015/060352 A

Patent Literature 3: International Publication No. WO2015/098975 A

Patent Literature 4: Korean Patent Publication No. 2012-0120886 A

Patent Literature 5: Korean Patent Publication No. 2013-0067284 A

Patent Literature 6: Korean Patent Publication No. 2014-0116043 A

### SUMMARY OF THE INVENTION

### Problems to be Solved by the Invention

In order to improve performance of an electronic device such as a display, an organic EL device has been required to be further improved in performance.

An object of the invention is to provide a compound capable of achieving higher performance, especially a longer lifetime of an organic electroluminescence device, an organic electroluminescence device with higher performance, especially a longer lifetime, and an electronic device including the organic electroluminescence device.

### Means for Solving the Problem(s)

According to an aspect of the invention, there is provided an organic electroluminescence device including: an anode; a cathode; an emitting layer provided between the anode and the cathode, in which the emitting layer contains a delayed fluorescent compound M2 and a compound M3 represented by a formula (3) below, and a singlet energy S₁(M2) of the compound M2 and a singlet energy S₁(M3) of the compound M3 satisfy a relationship of a numerical formula (Numerical Formula 1) below.

S₁(M3)>S₁(M2) (Numerical Formula 1)

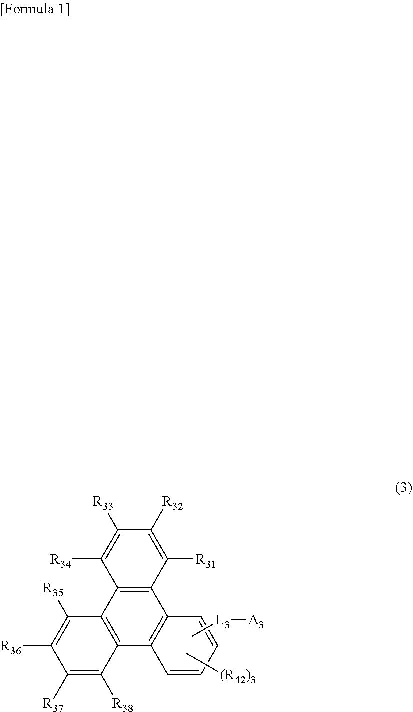

In the formula (3):

A₃ is a group represented by a formula (3a), (3b), (3c), (3d), (3e) or (3f) below;

L₃ is a single bond or a linking group, and L₃ as a linking group is a substituted or unsubstituted arylene group having 6 to 40 ring carbon atoms, a substituted or unsubstituted divalent heterocyclic group having 5 to 30 ring atoms, a group represented by -L₃₁-L₃₂-, or a group represented by -L₃₁-L₃₂-L₃₃-;

L₃₁, L₃₂ and L₃₃ are each independently a substituted or unsubstituted arylene group having 6 to 40 ring carbon atoms, or a substituted or unsubstituted divalent heterocyclic group having 5 to 30 ring atoms;

L₃₁, L₃₂ and L₃₃ are mutually the same or different;

R₃₁ to R₃₈ and R₄₂ are each independently a hydrogen atom or a substituent;

a substituent, if present, for L₃, and R₃₁ to R₃₈ and R₄₂ as a substituent are each independently a halogen atom, a cyano group, a substituted or unsubstituted aryl group having 6 to 30 ring carbon atoms, a substituted or unsubstituted heterocyclic group having 5 to 30 ring atoms, a substituted or unsubstituted alkyl group having 1 to 30 carbon atoms, a substituted or unsubstituted cycloalkyl group having 3 to 30 ring carbon atoms, a substituted or unsubstituted alkyl halide group having 1 to 30 carbon atoms, a substituted or unsubstituted alkenyl group having 2 to 30 carbon atoms, a substituted or unsubstituted alkynyl group having 2 to 30 carbon atoms, a substituted or unsubstituted alkylsilyl group having 3 to 30 carbon atoms, a substituted or unsubstituted arylsilyl group having 6 to 60 ring carbon atoms, a substituted or unsubstituted arylphosphoryl group having 6 to 60 ring carbon atoms, a hydroxy group, a substituted or unsubstituted alkoxy group having 1 to 30 carbon atoms, a substituted or unsubstituted aryloxy group having 6 to 30 ring carbon atoms, an amino group, a substituted or unsubstituted alkylamino group having 2 to 30 carbon atoms, a substituted or unsubstituted arylamino group having 6 to 60 ring carbon atoms, a thiol group, a substituted or unsubstituted alkylthio group having 1 to 30 carbon atoms, or a substituted or unsubstituted arylthio group having 6 to 30 ring carbon atoms; and

a plurality of R₄₂ are mutually the same or different.

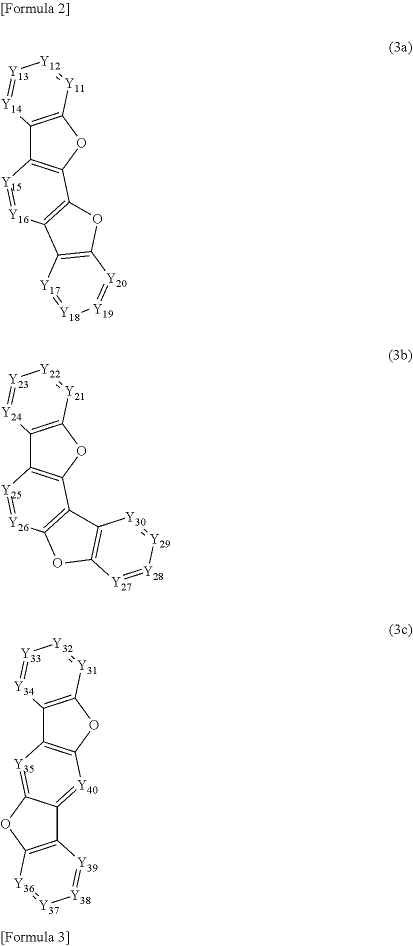

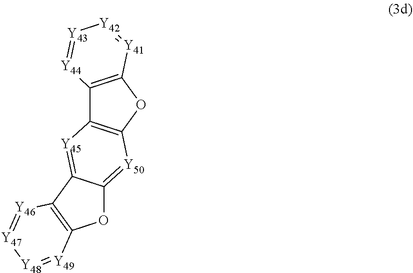

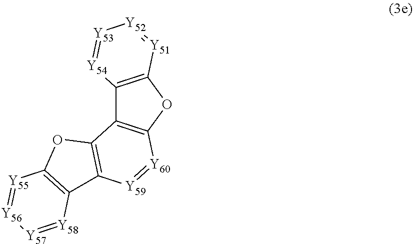

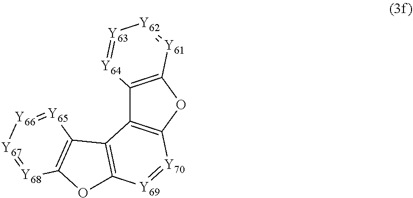

In the formula (3a):

Y₁₁ to Y₂₀ are each independently a nitrogen atom, CR₃₁₀, or a carbon atom bonded to L₃;

at least one of Y₁₁ to Y₂₀ is a carbon atom bonded to L₃;

in the formula (3b):

Y₂₁ to Y₃₀ are each independently a nitrogen atom, CR₃₁₀, or a carbon atom bonded to L₃;

at least one of Y₂₁ to Y₃₀ is a carbon atom bonded to L₃;

in the formula (3c):

Y₃₁ to Y₄₀ are each independently a nitrogen atom, CR₃₁₀, or a carbon atom bonded to L₃;

at least one of Y₃₁ to Y₄₀ is a carbon atom bonded to L₃;

in the formula (3d):

Y₄₁ to Y₅₀ are each independently a nitrogen atom, CR₃₁₀, or a carbon atom bonded to L₃;

at least one of Y₄₁ to Y₅₀ is a carbon atom bonded to L₃;

in the formula (3e):

Y₅₁ to Y₆₀ are each independently a nitrogen atom, CR₃₁₀, or a carbon atom bonded to L₃;

at least one of Y₅₁ to Y₆₀ is a carbon atom bonded to L₃;

in the formula (3f):

Y₆₁ to Y₇₀ are each independently a nitrogen atom, CR₃₁₀, or a carbon atom bonded to L₃;

at least one of Y₆₁ to Y₇₀ is a carbon atom bonded to L₃;

in the formulae (3a), (3b), (3c), (3d), (3e) and (3f):

R₃₁₀ is each independently a hydrogen atom or a substituent;

R₃₁₀ as a substituent is each independently a halogen atom, a cyano group, a substituted or unsubstituted aryl group having 6 to 30 ring carbon atoms, a substituted or unsubstituted heterocyclic group having 5 to 30 ring atoms, a substituted or unsubstituted alkyl group having 1 to 30 carbon atoms, a substituted or unsubstituted cycloalkyl group having 3 to 30 ring carbon atoms, a substituted or unsubstituted alkyl halide group having 1 to 30 carbon atoms, a substituted or unsubstituted alkenyl group having 2 to 30 carbon atoms, a substituted or unsubstituted alkynyl group having 2 to 30 carbon atoms, a substituted or unsubstituted alkylsilyl group having 3 to 30 carbon atoms, a substituted or unsubstituted arylsilyl group having 6 to 60 ring carbon atoms, a substituted or unsubstituted arylphosphoryl group having 6 to 60 ring carbon atoms, a hydroxy group, a substituted or unsubstituted alkoxy group having 1 to 30 carbon atoms, a substituted or unsubstituted aryloxy group having 6 to 30 ring carbon atoms, an amino group, a substituted or unsubstituted alkylamino group having 2 to 30 carbon atoms, a substituted or unsubstituted arylamino group having 6 to 60 ring carbon atoms, a thiol group, a substituted or unsubstituted alkylthio group having 1 to 30 carbon atoms, or a substituted or unsubstituted arylthio group having 6 to 30 ring carbon atoms; and

a plurality of R₃₁₀ are mutually the same or different.

According to another aspect of the invention, a compound represented by a formula (300) below is provided.

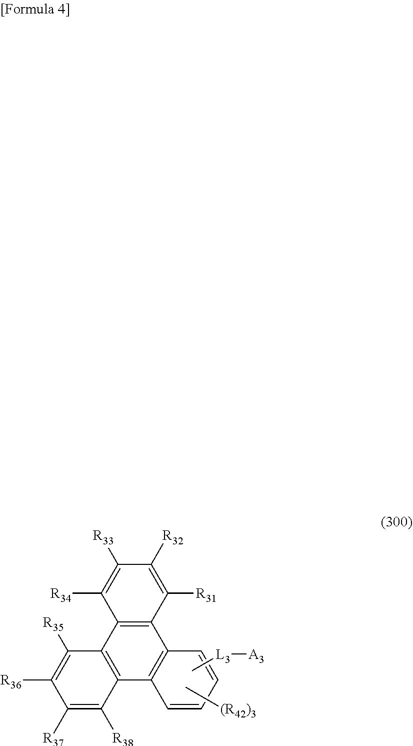

In the formula (300):

A₃ is a group represented by a formula (301a), (301b), (301c), (301d), (301e), (301f), (301g), (301h), (301i), (301j), (301k) or (301m);

L₃ is a single bond or a linking group, and L₃ as a linking group is a substituted or unsubstituted arylene group having 6 to 40 ring carbon atoms, a group represented by -L₃₁-L₃₂-, or a group represented by -L₃₁-L₃₂-L₃₃-;

L₃₁, L₃₂ and L₃₃ are each independently a substituted or unsubstituted arylene group having 6 to 40 ring carbon atoms;

L₃₁, L₃₂ and L₃₃ are mutually the same or different;

L₃, L₃₁, L₃₂ and L₃₃ are each not a substituted or unsubstituted anthracenylene group;

R₃₁ to R₃₈ and R₄₂ are each independently a hydrogen atom or a substituent;

a substituent, if present, for L₃, and R₃₁ to R₃₈ and R₄₂ as a substituent are each independently a halogen atom, a cyano group, a substituted or unsubstituted aryl group having 6 to 30 ring carbon atoms, a substituted or unsubstituted heterocyclic group having 5 to 30 ring atoms, a substituted or unsubstituted alkyl group having 1 to 30 carbon atoms, a substituted or unsubstituted cycloalkyl group having 3 to 30 ring carbon atoms, a substituted or unsubstituted alkyl halide group having 1 to 30 carbon atoms, a substituted or unsubstituted alkenyl group having 2 to 30 carbon atoms, a substituted or unsubstituted alkynyl group having 2 to 30 carbon atoms, a substituted or unsubstituted alkylsilyl group having 3 to 30 carbon atoms, a substituted or unsubstituted arylsilyl group having 6 to 60 ring carbon atoms, a substituted or unsubstituted arylphosphoryl group having 6 to 60 ring carbon atoms, a hydroxy group, a substituted or unsubstituted alkoxy group having 1 to 30 carbon atoms, a substituted or unsubstituted aryloxy group having 6 to 30 ring carbon atoms, an amino group, a substituted or unsubstituted alkylamino group having 2 to 30 carbon atoms, a substituted or unsubstituted arylamino group having 6 to 60 ring carbon atoms, a thiol group, a substituted or unsubstituted alkylthio group having 1 to 30 carbon atoms, or a substituted or unsubstituted arylthio group having 6 to 30 ring carbon atoms; and

a plurality of R₄₂ are mutually the same or different.

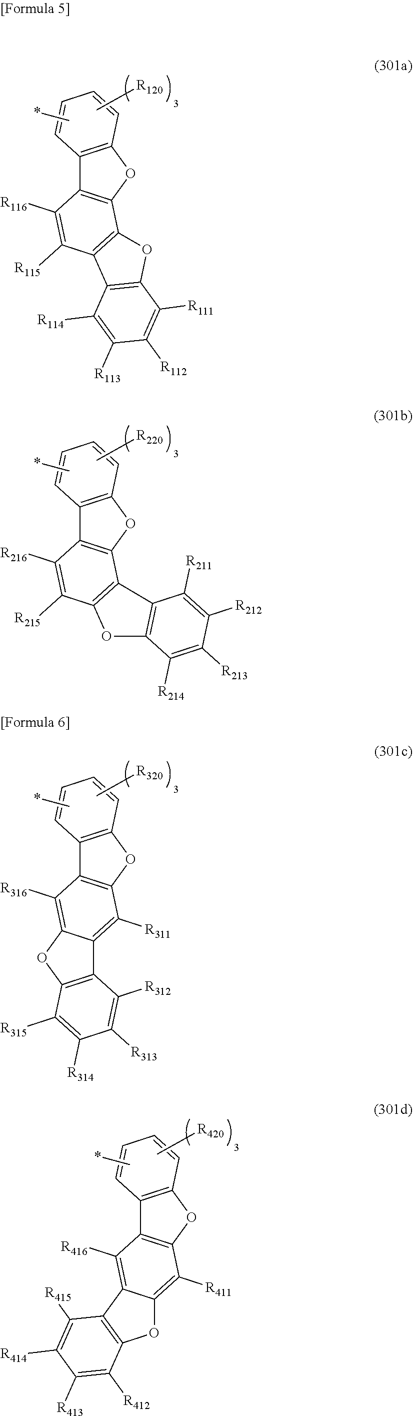

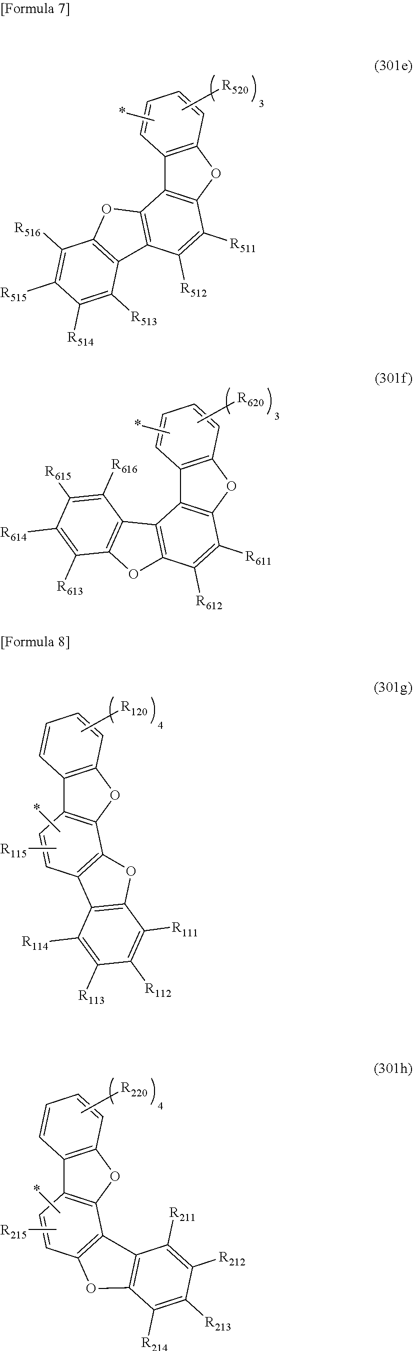

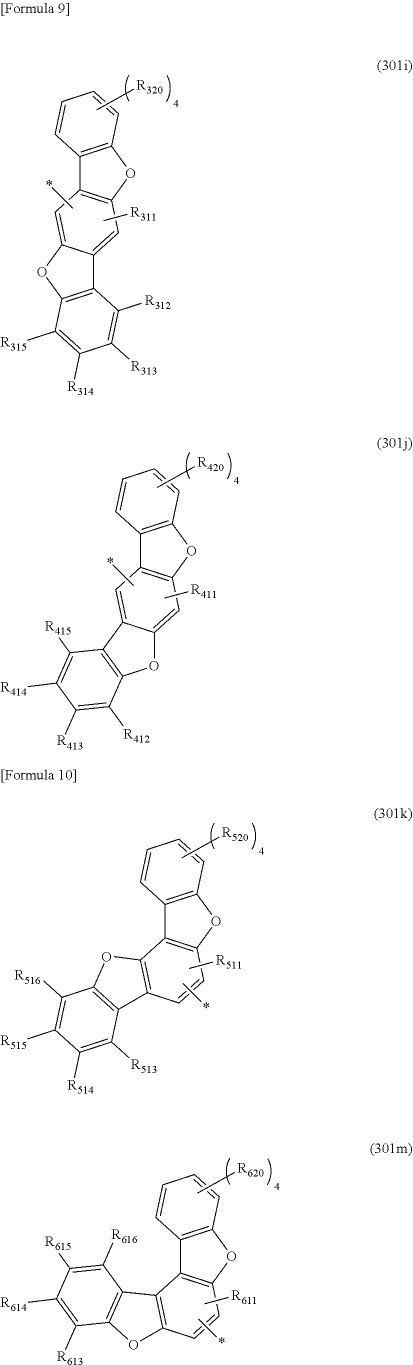

In the formulae (301a), (301b), (301c), (301d), (301e), (301f), (301g), (301h), (301i), (301j), (301k) and (301m):

R₁₁₁ to R₁₁₆, R₂₁₁ to R₂₁₆, R₃₁₁ to R₃₁₆, R₄₁₁ to R₄₁₆, R₅₁₁ to R₅₁₆, R₆₁₁ to R₆₁₆, R₁₂₀, R₂₂₀, R₃₂₀, R₄₂₀, R₅₂₀ and R₆₂₀ are each independently a hydrogen atom or a substituent;

R₁₁₁ to R₁₁₆, R₂₁₁ to R₂₁₆, R₃₁₁ to R₃₁₆, R₄₁₁ to R₄₁₆, R₅₁₁ to R₅₁₆, R₆₁₁ to R₆₁₆, R₁₂₀, R₂₂₀, R₃₂₀, R₄₂₀, R₅₂₀ and R₆₂₀ as a substituent are each independently a halogen atom, a cyano group, a substituted or unsubstituted aryl group having 6 to 30 ring carbon atoms, a substituted or unsubstituted heterocyclic group having 5 to 30 ring atoms, a substituted or unsubstituted alkyl group having 1 to 30 carbon atoms, a substituted or unsubstituted cycloalkyl group having 3 to 30 ring carbon atoms, a substituted or unsubstituted alkyl halide group having 1 to 30 carbon atoms, a substituted or unsubstituted alkenyl group having 2 to 30 carbon atoms, a substituted or unsubstituted alkynyl group having 2 to 30 carbon atoms, a substituted or unsubstituted alkylsilyl group having 3 to 30 carbon atoms, a substituted or unsubstituted arylsilyl group having 6 to 60 ring carbon atoms, a substituted or unsubstituted arylphosphoryl group having 6 to 60 ring carbon atoms, a hydroxy group, a substituted or unsubstituted alkoxy group having 1 to 30 carbon atoms, a substituted or unsubstituted aryloxy group having 6 to 30 ring carbon atoms, an amino group, a substituted or unsubstituted alkylamino group having 2 to 30 carbon atoms, a substituted or unsubstituted arylamino group having 6 to 60 ring carbon atoms, a thiol group, a substituted or unsubstituted alkylthio group having 1 to 30 carbon atoms, or a substituted or unsubstituted arylthio group having 6 to 30 ring carbon atoms;

R₁₁₁ to R₁₁₄, R₂₁₁ to R₂₁₄, R₃₁₂ to R₃₁₅, R₄₁₂ to R₄₁₅, R₅₁₃ to R₅₁₆, R₆₁₃ to R₆₁₆, R₁₂₀, R₂₂₀, R₃₂₀, R₄₂₀, R₅₂₀ and R₆₂₀ are each not an unsubstituted triphenylenyl group; a plurality of R₁₂₀ are mutually the same or different;

a plurality of R₂₂₀ are mutually the same or different;

a plurality of R₃₂₀ are mutually the same or different;

a plurality of R₄₂₀ are mutually the same or different;

a plurality of R₅₂₀ are mutually the same or different;

a plurality of R₆₂₀ are mutually the same or different; and

* represents a bonding position to L₃.

According to still another aspect of the invention, there is provided an organic electroluminescence device including: an anode; a cathode; and an emitting layer provided between the anode and the cathode, in which the emitting layer contains the delayed fluorescent compound M2 and the compound according to the above aspect of the invention.

According to a further aspect of the invention, an electronic device including the organic electroluminescence device according to any one of the above aspects of the invention is provided.

According to the above aspects of the invention, a compound capable of achieving higher performance, especially a longer lifetime, of an organic electroluminescence device, an organic electroluminescence device with higher performance, especially a longer lifetime, and an electronic device including the organic electroluminescence device can be provided.

### BRIEF DESCRIPTION OF DRAWING(S)

FIG. 1 schematically shows an exemplary arrangement of an organic electroluminescence device according to a first exemplary embodiment of the invention.

FIG. 2 schematically shows a device of measuring transient PL.

FIG. 3 shows an example of decay curves of the transient PL.

FIG. 4 schematically shows a relationship in energy level and energy transfer between a compound M3 and a compound M2 in an emitting layer of an exemplary organic electroluminescence device according to the first exemplary embodiment of the invention.

FIG. 5 schematically shows a relationship in energy level and energy transfer between the compound M3, the compound M2 and a compound M1 in an emitting layer of an exemplary organic electroluminescence device according to a second exemplary embodiment of the invention.

### DESCRIPTION OF EMBODIMENT(S)

### First Exemplary Embodiment

An arrangement of an organic EL device according to a first exemplary embodiment of the invention will be described below.

The organic EL device includes an organic layer between both electrodes of an anode and a cathode. The organic layer includes at least one layer formed of an organic compound. Alternatively, the organic layer is provided by laminating a plurality of layers each formed of an organic compound. The organic layer may further contain an inorganic compound. In the organic EL device according to the exemplary embodiment, at least one layer of the organic layer is an emitting layer. Therefore, for instance, the organic layer may consist of a single emitting layer or, alternatively, may further include at least one layer usable for an organic EL device. Examples of the layer usable in the organic EL device, which are not particularly limited, include at least one layer selected from the group consisting of a hole injecting layer, hole transporting layer, electron injecting layer, electron transporting layer, and blocking layer.

The organic EL device according to the exemplary embodiment includes an emitting layer between the anode and the cathode.

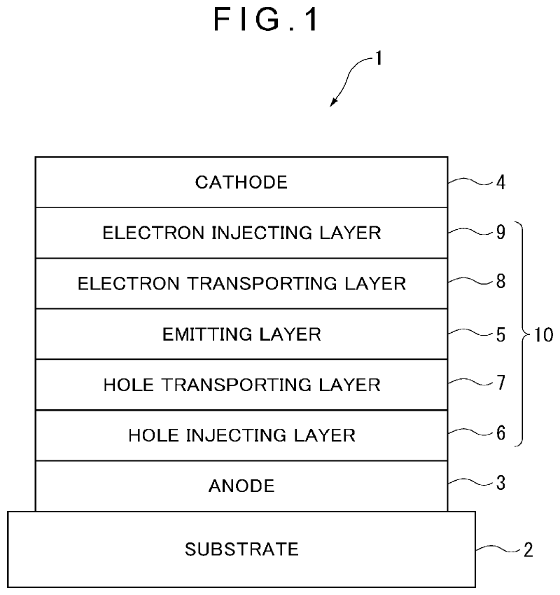

FIG. 1 schematically shows an exemplary arrangement of an organic EL device according to the first exemplary embodiment.

An organic EL device 1 includes a light-transmissive substrate 2, an anode 3, a cathode 4, and an organic layer 10 provided between the anode 3 and the cathode 4. The organic layer 10 includes a hole injecting layer 6, a hole transporting layer 7, an emitting layer 5, an electron transporting layer 8, and an electron injecting layer 9, which are sequentially laminated on the anode 3.

In an exemplary arrangement of the exemplary embodiment, the emitting layer may contain a metal complex.

In an exemplary arrangement of the exemplary embodiment, the emitting layer preferably does not contain a phosphorescent material (dopant material).

In an exemplary arrangement of the exemplary embodiment, the emitting layer preferably does not contain a heavy-metal complex and a phosphorescent rare earth metal complex. Examples of the heavy-metal complex herein include iridium complex, osmium complex, and platinum complex.

In an exemplary arrangement of the exemplary embodiment, the emitting layer also preferably does not contain a metal complex.

In the organic EL device according to the exemplary embodiment, the emitting layer contains a delayed fluorescent compound M2 and a compound M3 represented by a formula (3) below.

In this arrangement, the compound M2 is preferably a dopant material (also referred to as a guest material, emitter or luminescent material), and the compound M3 is preferably a host material (also referred to as a matrix material).

The compound M3 represented by the formula (3) has both a triphenylene structure for appropriately injecting and/or transporting holes and A₃ having a five-ring fused ring structure with high thermal stability (benzofuranodibenzofuran structure).

The organic EL device according to the exemplary embodiment contains the compound M3 as well as the delayed fluorescent compound M2 in the emitting layer. This presumably makes both charges, i.e., holes and electrons, well-balanced in the emitting layer and the emitting layer made from an amorphous thin film that is unlikely to be crystallized. Consequently, the device is considered to be improved in performance (e.g., a lifetime or luminous efficiency).

### Emitting Layer

### Compound M3

The emitting layer of the exemplary embodiment contains the compound M3 represented by the formula (3).

The compound M3 of the exemplary embodiment may be a thermally activated delayed fluorescent compound or a compound exhibiting no thermally activated delayed fluorescence. However, the compound M3 is preferably a compound exhibiting no thermally activated delayed fluorescence.

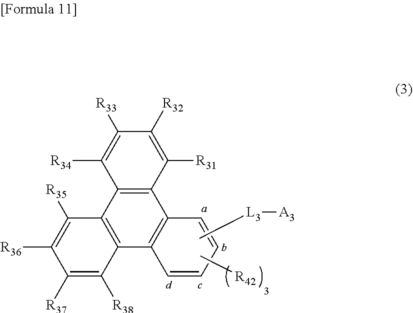

In the formula (3):

A₃ is a group represented by a formula (3a), (3b), (3c), (3d), (3e) or (3f) below;

L₃ is a single bond or a linking group, and L₃ as a linking group is a substituted or unsubstituted arylene group having 6 to 40 ring carbon atoms, a substituted or unsubstituted divalent heterocyclic group having 5 to 30 ring atoms, a group represented by -L₃₁-L₃₂-, or a group represented by -L₃₁-L₃₂-L₃₃-;

L₃₁, L₃₂ and L₃₃ are each independently a substituted or unsubstituted arylene group having 6 to 40 ring carbon atoms, or a substituted or unsubstituted divalent heterocyclic group having 5 to 30 ring atoms;

L₃₁, L₃₂ and L₃₃ are mutually the same or different;

R₃₁ to R₃₈ and R₄₂ are each independently a hydrogen atom or a substituent;

a substituent, if present, for L₃, and R₃₁ to R₃₈ and R₄₂ as a substituent are each independently a halogen atom, a cyano group, a substituted or unsubstituted aryl group having 6 to 30 ring carbon atoms, a substituted or unsubstituted heterocyclic group having 5 to 30 ring atoms, a substituted or unsubstituted alkyl group having 1 to 30 carbon atoms, a substituted or unsubstituted cycloalkyl group having 3 to 30 ring carbon atoms, a substituted or unsubstituted alkyl halide group having 1 to 30 carbon atoms, a substituted or unsubstituted alkenyl group having 2 to 30 carbon atoms, a substituted or unsubstituted alkynyl group having 2 to 30 carbon atoms, a substituted or unsubstituted alkylsilyl group having 3 to 30 carbon atoms, a substituted or unsubstituted arylsilyl group having 6 to 60 ring carbon atoms, a substituted or unsubstituted arylphosphoryl group having 6 to 60 ring carbon atoms, a hydroxy group, a substituted or unsubstituted alkoxy group having 1 to 30 carbon atoms, a substituted or unsubstituted aryloxy group having 6 to 30 ring carbon atoms, an amino group, a substituted or unsubstituted alkylamino group having 2 to 30 carbon atoms, a substituted or unsubstituted arylamino group having 6 to 60 ring carbon atoms, a thiol group, a substituted or unsubstituted alkylthio group having 1 to 30 carbon atoms, or a substituted or unsubstituted arylthio group having 6 to 30 ring carbon atoms; and

a plurality of R₄₂ are mutually the same or different.

In the formula (3), L₃ is bonded to a carbon atom at one of positions a, b, c and d shown in the formula (3) and three R₄₂ are bonded to respective ones of carbon atoms at remaining three of the positions a, b, c and d not bonded to L₃.

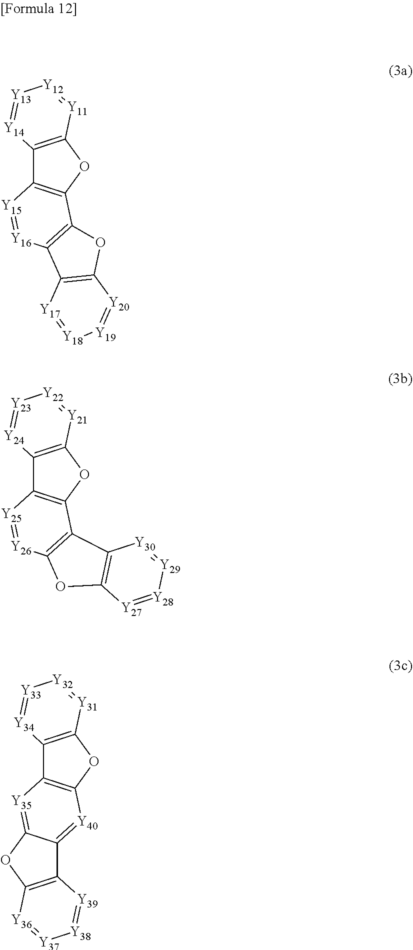

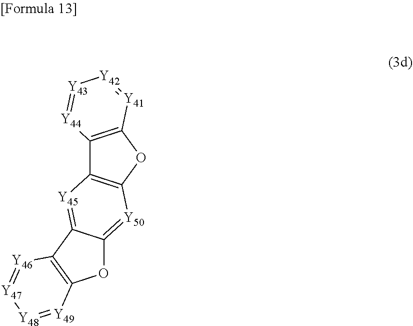

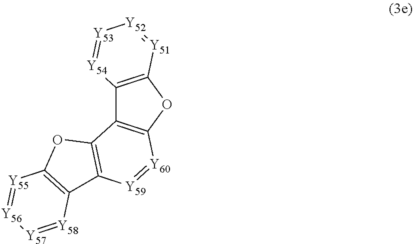

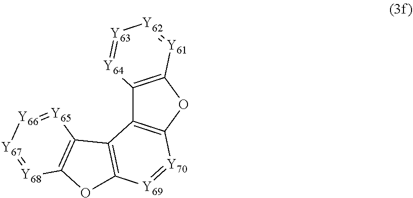

In the formula (3a), Y₁₁ to Y₂₀ are each independently a nitrogen atom, CR₃₁₀, or a carbon atom bonded to L₃, and at least one of Y₁₁ to Y₂₀ is a carbon atom bonded to L₃;

in the formula (3b), Y₂₁ to Y₃₀ are each independently a nitrogen atom, CR₃₁₀, or a carbon atom bonded to L₃, and at least one of Y₂₁ to Y₃₀ is a carbon atom bonded to L₃;

in the formula (3c), Y₃₁ to Y₄₀ are each independently a nitrogen atom, CR₃₁₀, or a carbon atom bonded to L₃, and at least one of Y₃₁ to Y₄₀ is a carbon atom bonded to L₃;

in the formula (3d), Y₄₁ to Y₅₀ are each independently a nitrogen atom, CR₃₁₀, or a carbon atom bonded to L₃, and at least one of Y₄₁ to Y₅₀ is a carbon atom bonded to L₃;

in the formula (3e), Y₅₁ to Y₆₀ are each independently a nitrogen atom, CR₃₁₀, or a carbon atom bonded to L₃, and at least one of Y₅₁ to Y₆₀ is a carbon atom bonded to L₃;

in the formula (3f), Y₆₁ to Y₇₀ are each independently a nitrogen atom, CR₃₁₀, or a carbon atom bonded to L₃, and at least one of Y₆₁ to Y₇₀ is a carbon atom bonded to L₃;

in the formulae (3a) to (3f):

R₃₁₀ is each independently a hydrogen atom or a substituent;

R₃₁₀ as a substituent is each independently a halogen atom, a cyano group, a substituted or unsubstituted aryl group having 6 to 30 ring carbon atoms, a substituted or unsubstituted heterocyclic group having 5 to 30 ring atoms, a substituted or unsubstituted alkyl group having 1 to 30 carbon atoms, a substituted or unsubstituted cycloalkyl group having 3 to 30 ring carbon atoms, a substituted or unsubstituted alkyl halide group having 1 to 30 carbon atoms, a substituted or unsubstituted alkenyl group having 2 to 30 carbon atoms, a substituted or unsubstituted alkynyl group having 2 to 30 carbon atoms, a substituted or unsubstituted alkylsilyl group having 3 to 30 carbon atoms, a substituted or unsubstituted arylsilyl group having 6 to 60 ring carbon atoms, a substituted or unsubstituted arylphosphoryl group having 6 to 60 ring carbon atoms, a hydroxy group, a substituted or unsubstituted alkoxy group having 1 to 30 carbon atoms, a substituted or unsubstituted aryloxy group having 6 to 30 ring carbon atoms, an amino group, a substituted or unsubstituted alkylamino group having 2 to 30 carbon atoms, a substituted or unsubstituted arylamino group having 6 to 60 ring carbon atoms, a thiol group, a substituted or unsubstituted alkylthio group having 1 to 30 carbon atoms, or a substituted or unsubstituted arylthio group having 6 to 30 ring carbon atoms; and

a plurality of R₃₁₀ are mutually the same or different.

In the compound M3, it is preferable that L₃ is bonded to a carbon atom at the position b or c shown in the formula (3).

In the compound M3, it is also preferable that L₃ is bonded to a carbon atom at the position a or d shown in the formula (3).

In the compound M3, when Y₁₁ to Y₁₄ and Y₁₇ to Y₂₀ in the formula (3a) are each CR₃₁₀, R₃₁₀ is preferably not a substituted or unsubstituted triphenylenyl group, more preferably not an unsubstituted triphenylenyl group.

In the compound M3, when Y₂₁ to Y₂₄ and Y₂₇ to Y₃₀ in the formula (3b) are each CR₃₁₀, R₃₁₀ is preferably not a substituted or unsubstituted triphenylenyl group, more preferably not an unsubstituted triphenylenyl group.

In the compound M3, when Y₃₁ to Y₃₄ and Y₃₆ to Y₃₉ in the formula (3c) are each CR₃₁₀, R₃₁₀ is preferably not a substituted or unsubstituted triphenylenyl group, more preferably not an unsubstituted triphenylenyl group.

In the compound M3, when Y₄₁ to Y₄₄ and Y₄₆ to Y₄₉ in the formula (3d) are each CR₃₁₀, R₃₁₀ is preferably not a substituted or unsubstituted triphenylenyl group, more preferably not an unsubstituted triphenylenyl group.

In the compound M3, when Y₅₁ to Y₅₄ and Y₅₅ to Y₅₈ in the formula (3e) are each CR₃₁₀, R₃₁₀ is preferably not a substituted or unsubstituted triphenylenyl group, more preferably not an unsubstituted triphenylenyl group.

In the compound M3, when Y₆₁ to Y₆₄ and Y₆₅ to Y₆₈ in the formula (3f) are each CR₃₁₀, R₃₁₀ is preferably not a substituted or unsubstituted triphenylenyl group, more preferably not an unsubstituted triphenylenyl group.

In the compound M3, R₃₁ to R₃₈, R₄₂ and R₃₁₀ are preferably each independently a hydrogen atom or a substituted or unsubstituted aryl group having 6 to 30 ring carbon atoms, more preferably a hydrogen atom or a substituted or unsubstituted phenyl group, further preferably a hydrogen atom.

In the compound M3, A₃ is preferably a group represented by a formula (30a), (30b), (30c), (30d), (30e) or (30f) below.

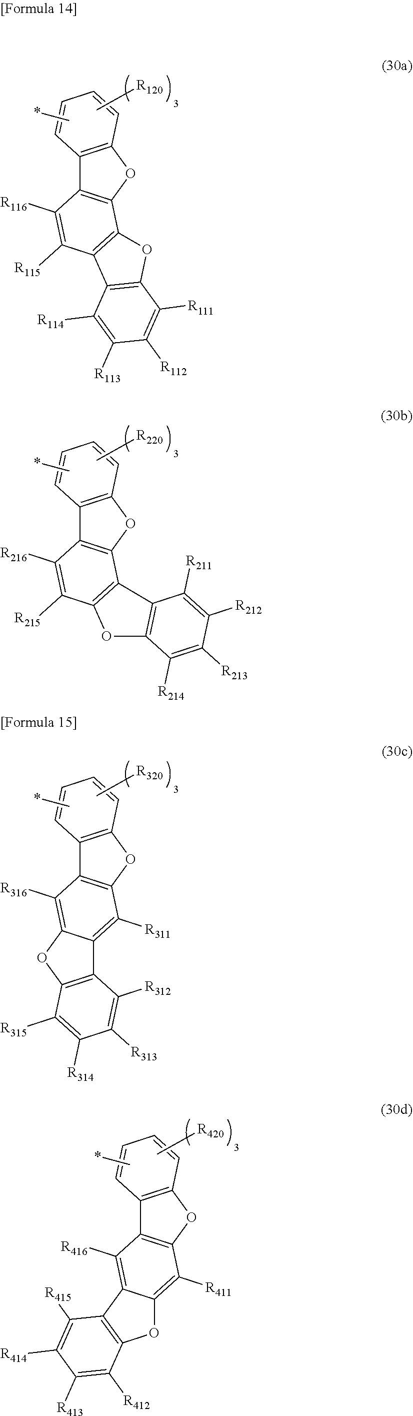

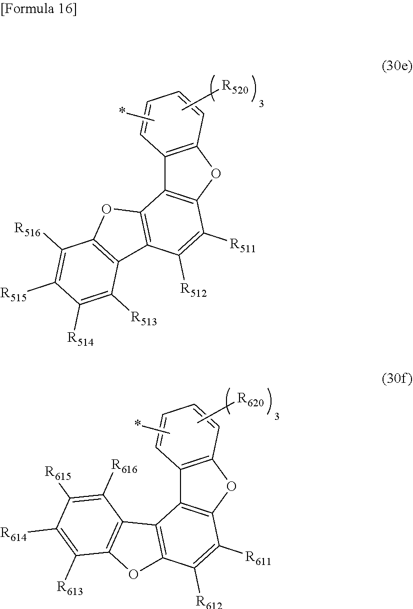

In the formulae (30a) to (30f): R₁₁₁ to R₁₁₆, R₂₁₁ to R₂₁₆, R₃₁₁ to R₃₁₆, R₄₁₁ to R₄₁₆, R₅₁₁ to R₅₁₆, R₆₁₁ to R₆₁₆, R₁₂₀, R₂₂₀, R₃₂₀, R₄₂₀, R₅₂₀ and R₆₂₀ each independently represent the same as R₃₁₀ in the formulae (3a), (3b), (3c), (3d), (3e) and (3f); a plurality of R₁₂₀ are mutually the same or different; a plurality of R₂₂₀ are mutually the same or different; a plurality of R₃₂₀ are mutually the same or different; a plurality of R₄₂₀ are mutually the same or different; a plurality of R₅₂₀ are mutually the same or different; a plurality of R₆₂₀ are mutually the same or different; and * represents a bonding position to L₃.

In the formulae (30a) to (30f), R₁₁₁ to R₁₁₆, R₂₁₁ to R₂₁₆, R₃₁₁ to R₃₁₆, R₄₁₁ to R₄₁₆, R₅₁₁ to R₅₁₆, R₆₁₁ to R₆₁₆, R₁₂₀, R₂₂₀, R₃₂₀, R₄₂₀, R₅₂₀ and R₆₂₀ are preferably each independently a hydrogen atom or a substituted or unsubstituted aryl group having 6 to 30 ring carbon atoms, more preferably a hydrogen atom or a substituted or unsubstituted phenyl group, further preferably a hydrogen atom.

In the compound M3, A₃ is also preferably a group represented by a formula (30g), (30h), (30i), (30j), (30k) or (30m) below.

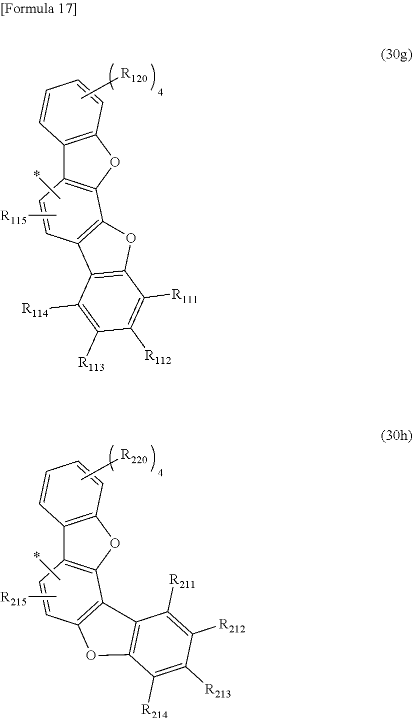

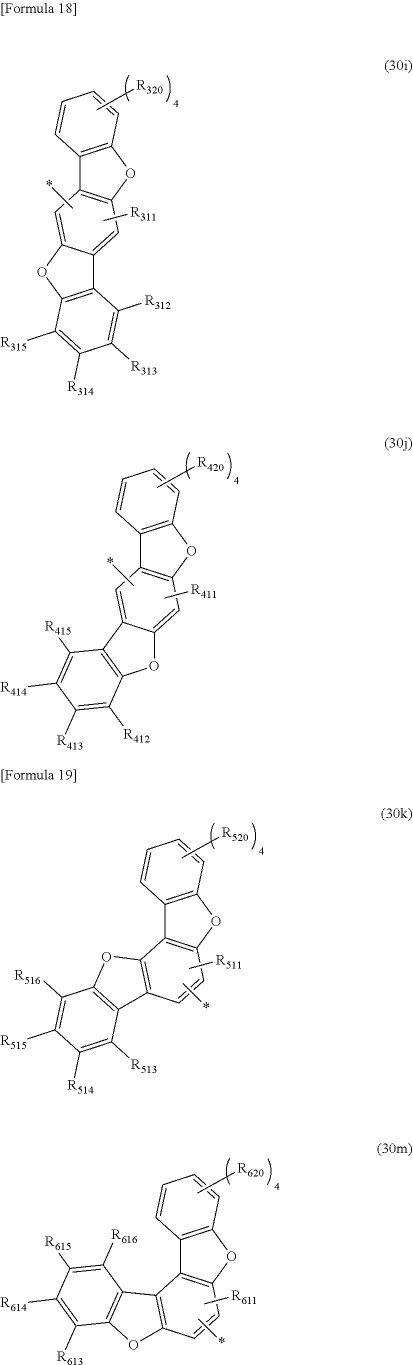

In the formulae (30g) to (30k) and (30m): R₁₁₁ to R₁₁₅, R₂₁₁ to R₂₁₅, R₃₁₁ to R₃₁₅, R₄₁₁ to R₄₁₅, R₅₁₁, R₅₁₃ to R₅₁₆, R₆₁₁, R₆₁₃ to R₆₁₆, R₁₂₀, R₂₂₀, R₃₂₀, R₄₂₀, R₅₂₀ and R₆₂₀ each independently represent the same as R₃₁₀ in the formulae (3a) to (3f); a plurality of R₁₂₀ are mutually the same or different; a plurality of R₂₂₀ are mutually the same or different; a plurality of R₃₂₀ are mutually the same or different; a plurality of R₄₂₀ are mutually the same or different; a plurality of R₅₂₀ are mutually the same or different; a plurality of R₆₂₀ are mutually the same or different; and * represents a bonding position to L₃.

In the formulae (30g) to (30k) and (30m), R₁₁₁ to R₁₁₅, R₂₁₁ to R₂₁₅, R₃₁₁ to R₃₁₅, R₄₁₁ to R₄₁₅, R₅₁₁, R₅₁₃ to R₅₁₆, R₆₁₁, R₆₁₃ to R₆₁₆, R₁₂₀, R₂₂₀, R₃₂₀, R₄₂₀, R₅₂₀ and R₆₂₀ are preferably each independently a hydrogen atom or a substituted or unsubstituted aryl group having 6 to 30 ring carbon atoms, more preferably a hydrogen atom or a substituted or unsubstituted phenyl group, further preferably a hydrogen atom.

In the compound M3, A₃ is preferably a group represented by a formula (31a), (31b), (31c), (31d), (31e) or (31f) below.

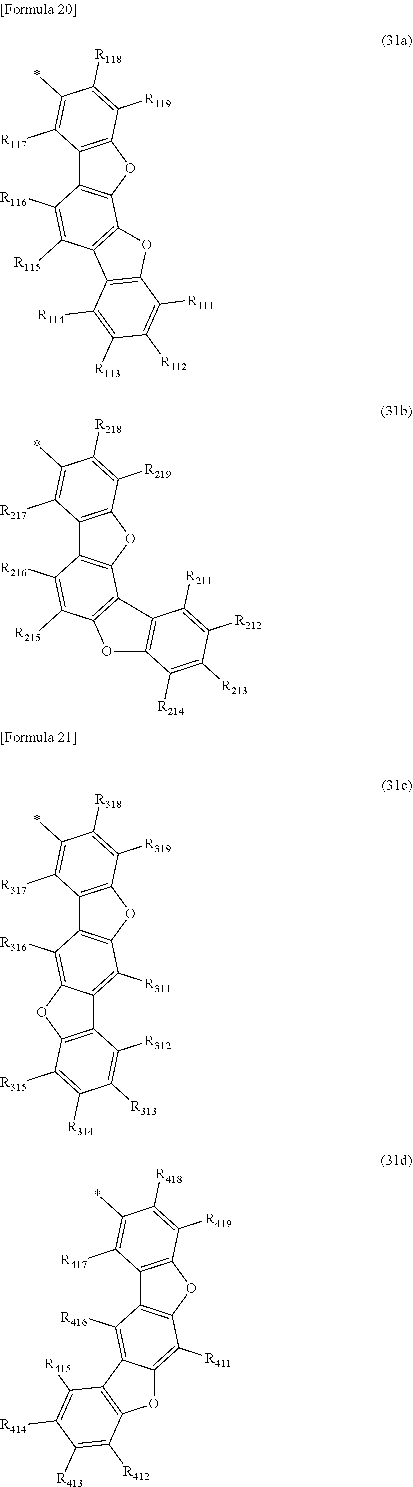

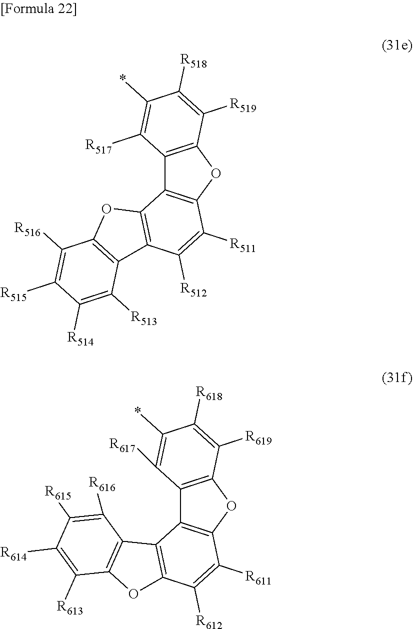

In the formulae (31a) to (31f): R₁₁₁ to R₁₁₉, R₂₁₁ to R₂₁₉, R₃₁₁ to R₃₁₉, R₄₁₁ to R₄₁₉, R₅₁₁ to R₅₁₉ and R₆₁₁ to R₆₁₉ each independently represent the same as R₃₁₀ in the formulae (3a) to (3f); and * represents a bonding position to L₃.

In the formulae (31a) to (31f), R₁₁₁ to R₁₁₉, R₂₁₁ to R₂₁₉, R₃₁₁ to R₃₁₉, R₄₁₁ to R₄₁₉, R₅₁₁ to R₅₁₉ and R₆₁₁ to R₆₁₉ are preferably each independently a hydrogen atom or a substituted or unsubstituted aryl group having 6 to 30 ring carbon atoms, more preferably a hydrogen atom or a substituted or unsubstituted phenyl group, further preferably a hydrogen atom.

In the compound M3, A₃ is also preferably a group represented by one of formulae (32a) to (35a), (32b) to (35b), (32c) to (35c), (32d) to (36d), (32e) to (35e) and (32f) to (35f) below.

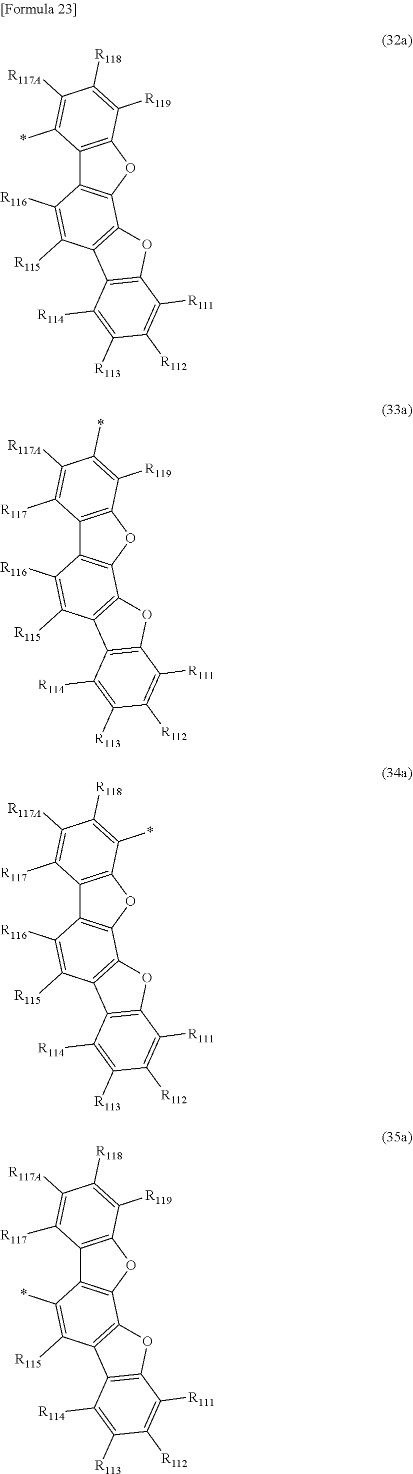

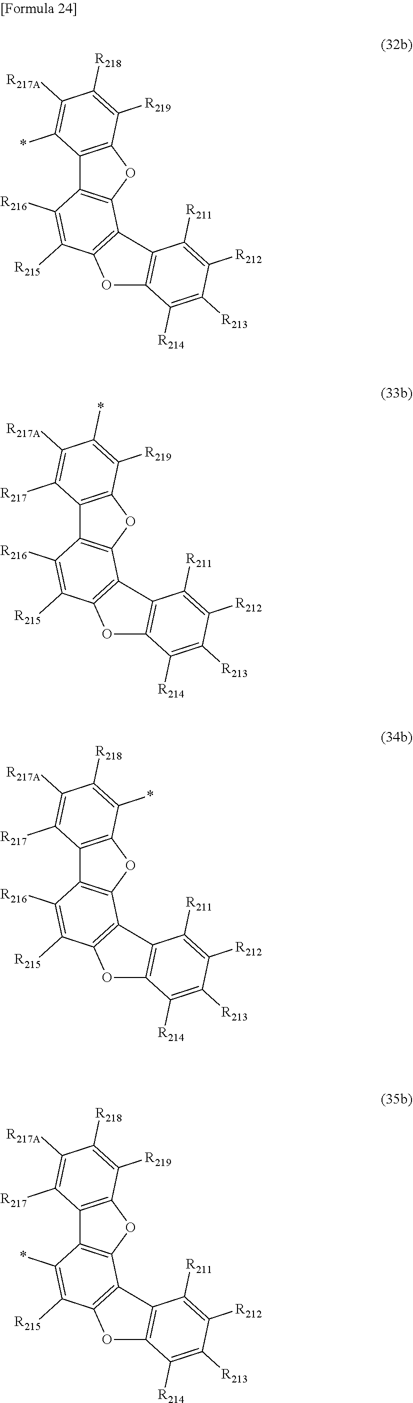

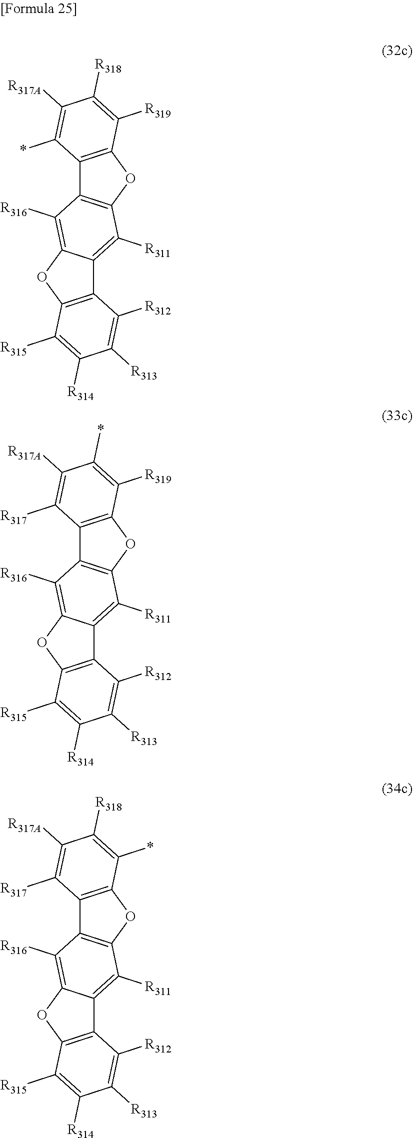

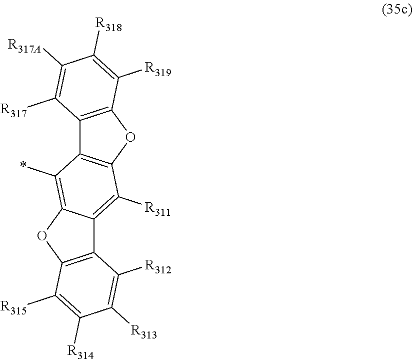

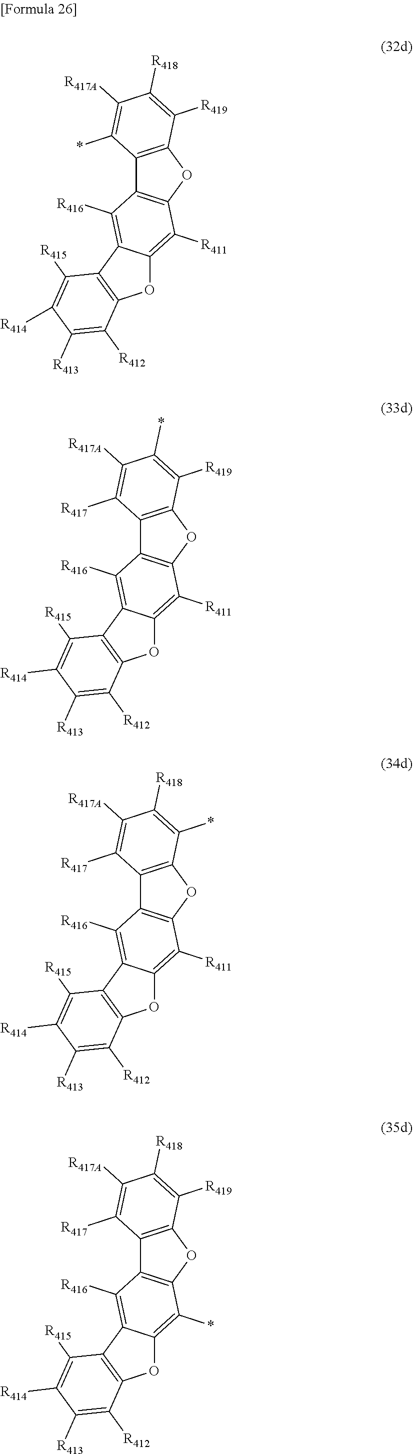

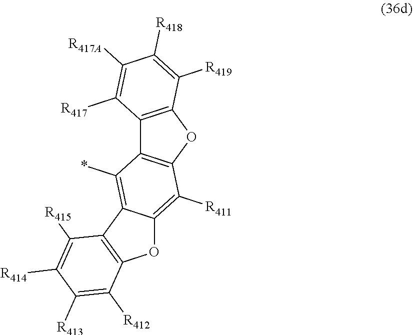

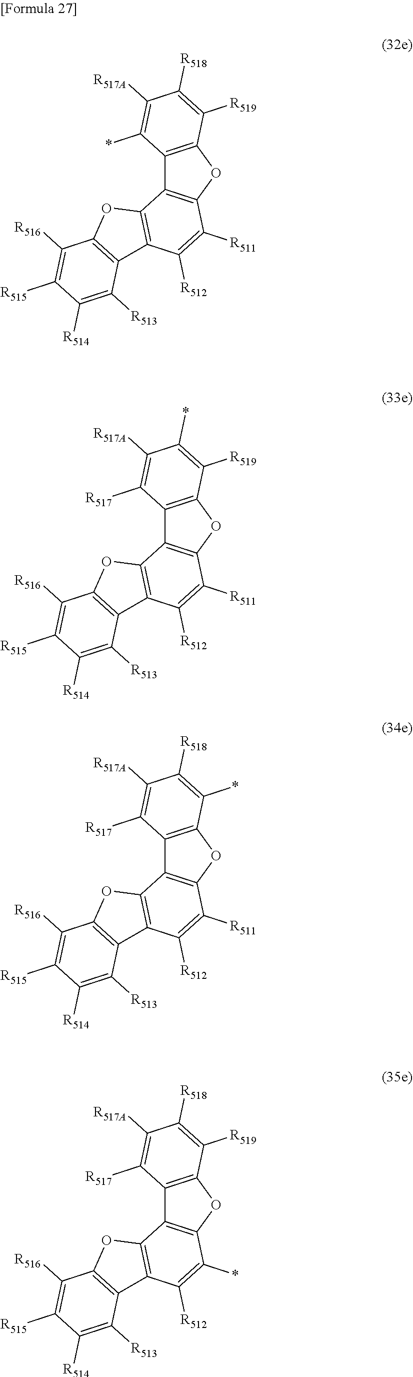

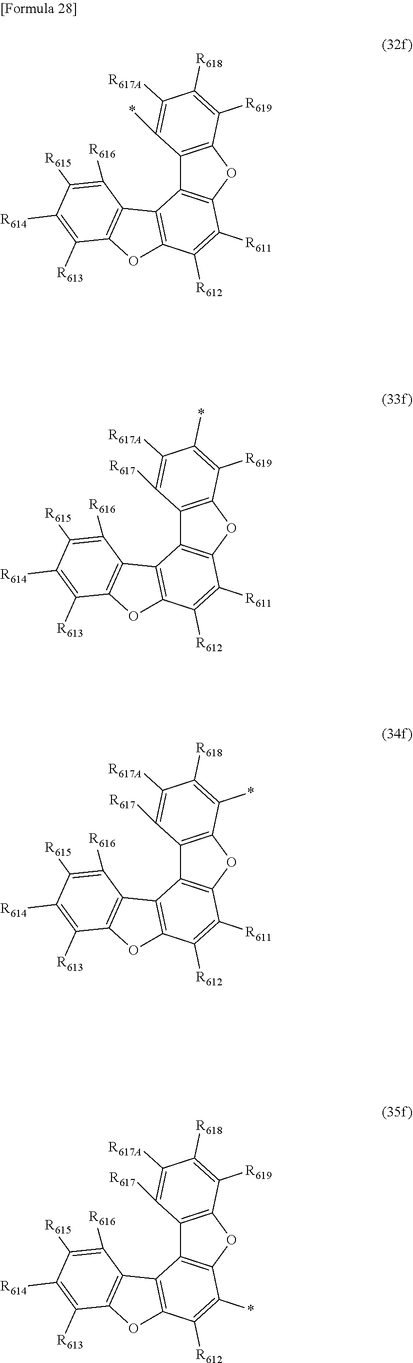

In the formulae (32a) to (35a), (32b) to (35b), (32c) to (35c), (32d) to (36d), (32e) to (35e) and (32f) to (35f): R₁₁₁ to R₁₁₉, R_{117A}, R₂₁₁ to R₂₁₉, R_{217A}, R₃₁₁ to R₃₁₉, R_{317A}, R₄₁₁ to R₄₁₉, R_{417A}, R₅₁₁ to R₅₁₉, R_{517A}, R₆₁₁ to R₆₁₉ and R_{617A} each independently represent the same as R₃₁₀ in the formulae (3a) to (3f), and * represents a bonding position to L₃.

In the formulae (32a) to (35a), (32b) to (35b), (32c) to (35c), (32d) to (36d), (32e) to (35e) and (32f) to (35f), R₁₁₁ to R₁₁₉, R_{117A}, R₂₁₁ to R₂₁₉, R_{217A}, R₃₁₁ to R₃₁₉, R_{317A}, R₄₁₁ to R₄₁₉, R_{417A}, R₅₁₁ to R₅₁₉, R_{517A}, R₆₁₁ to R₆₁₉ and R_{617A} are preferably each independently a hydrogen atom or a substituted or unsubstituted aryl group having 6 to 30 ring carbon atoms, more preferably a hydrogen atom or a substituted or unsubstituted phenyl group, further preferably a hydrogen atom.

The compound M3 is preferably represented by a formula (301), (302), (303), (304), (305) or (306) below.

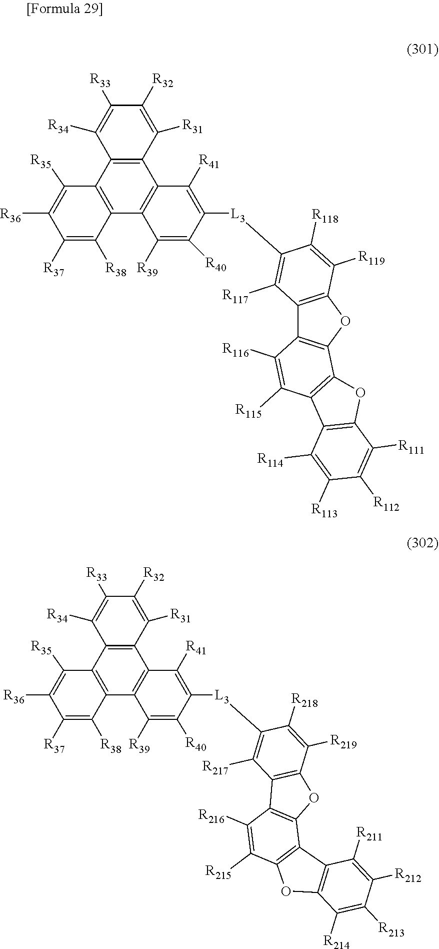

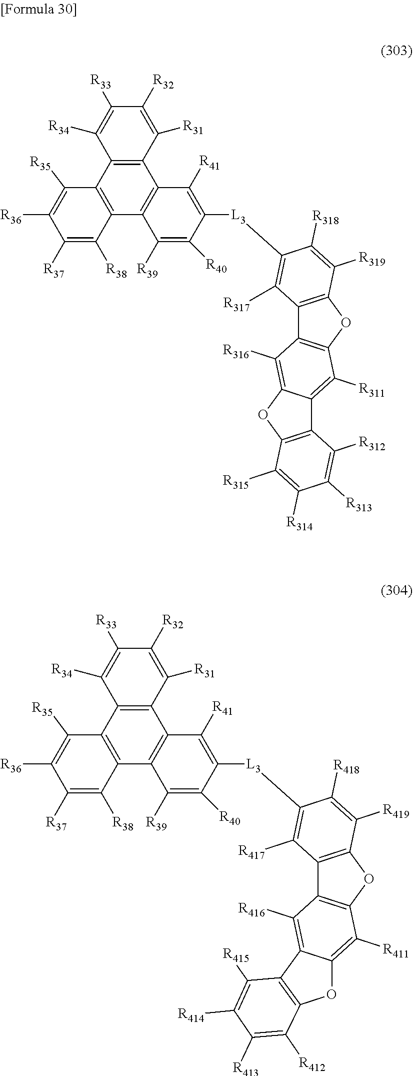

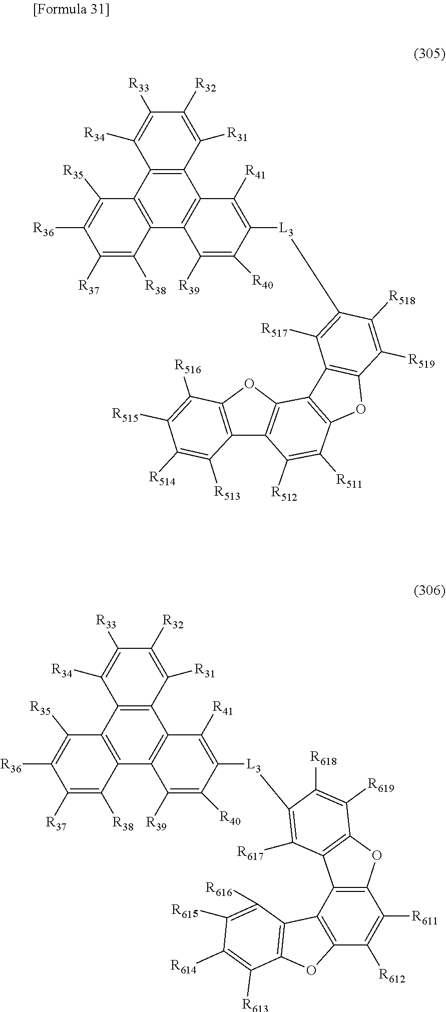

In the formulae (301) to (306): R₁₁₁ to R₁₁₉, R₂₁₁ to R₂₁₉, R₃₁₁ to R₃₁₉, R₄₁₁ to R₄₁₉, R₅₁₁ to R₅₁₉ and R₆₁₁ to R₆₁₉ each independently represent the same as R₃₁₀ in the formulae (3a) to (3f); R₃₁ to R₄₁ each represent the same as R₄₂ in the formula (3); and L₃ represents the same as L₃ in the formula (3).

In the formulae (301) to (306), R₁₁₁ to R₁₁₉, R₂₁₁ to R₂₁₉, R₃₁₁ to R₃₁₉, R₄₁₁ to R₄₁₉, R₅₁₁ to R₅₁₉, R₆₁₁ to R₆₁₉ and R₃₁ to R₄₁ are preferably each independently a hydrogen atom or a substituted or unsubstituted aryl group having 6 to 30 ring carbon atoms, more preferably a hydrogen atom or a substituted or unsubstituted phenyl group, further preferably a hydrogen atom.

The compound M3 is also preferably represented by one of formulae (307) to (331) below.

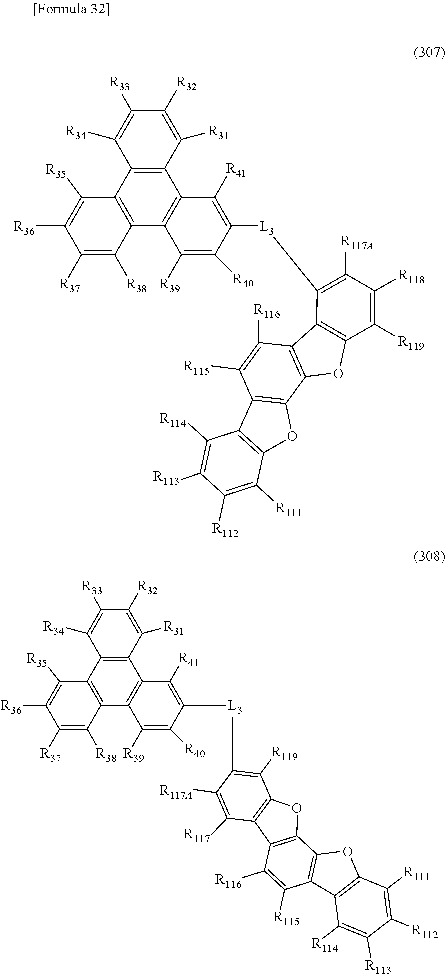

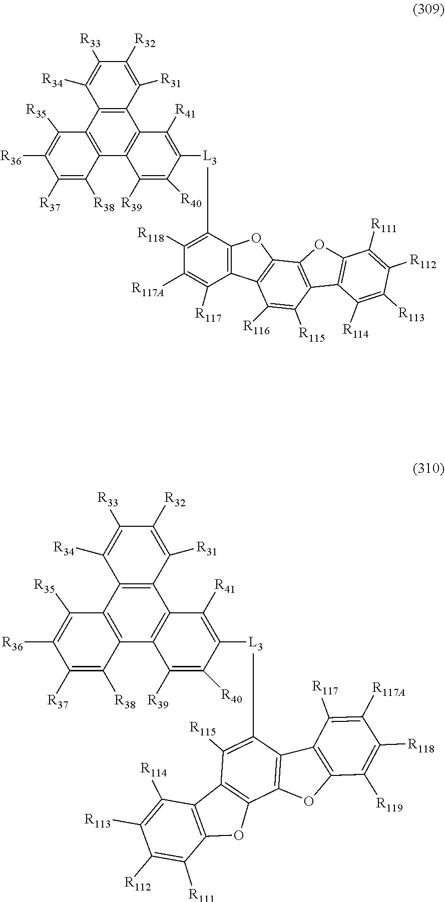

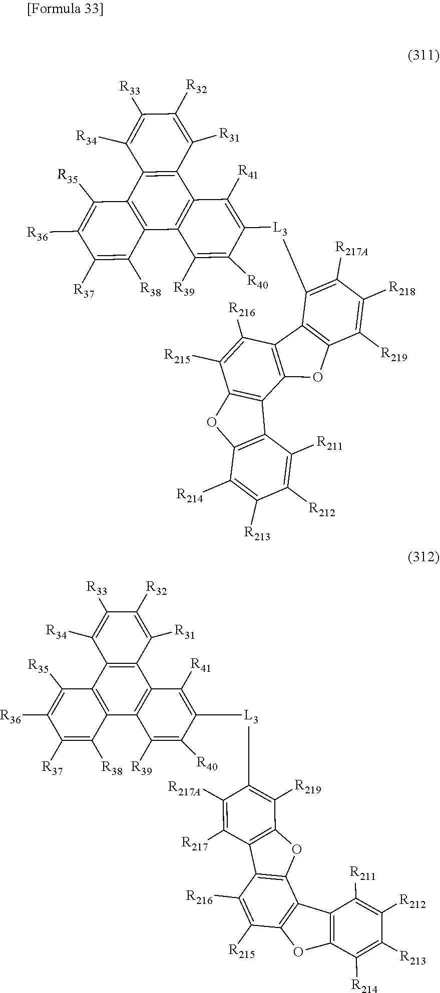

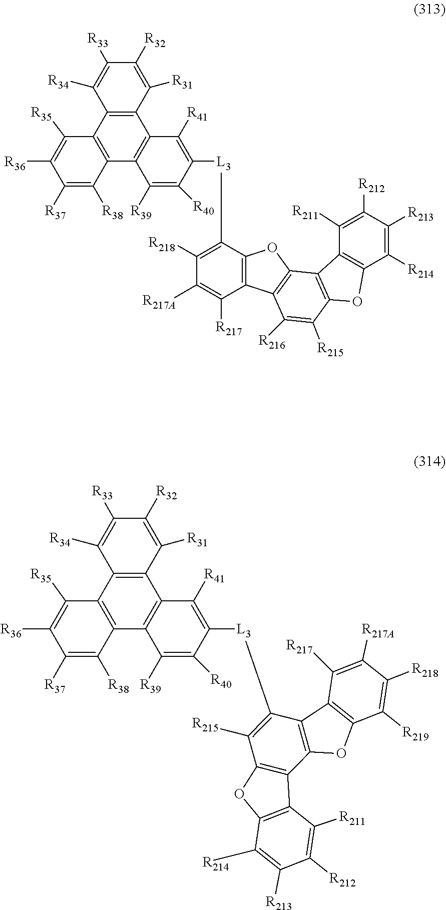

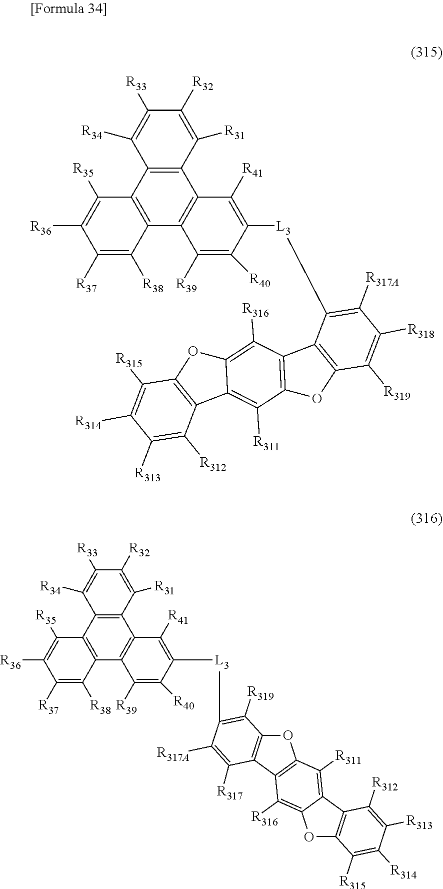

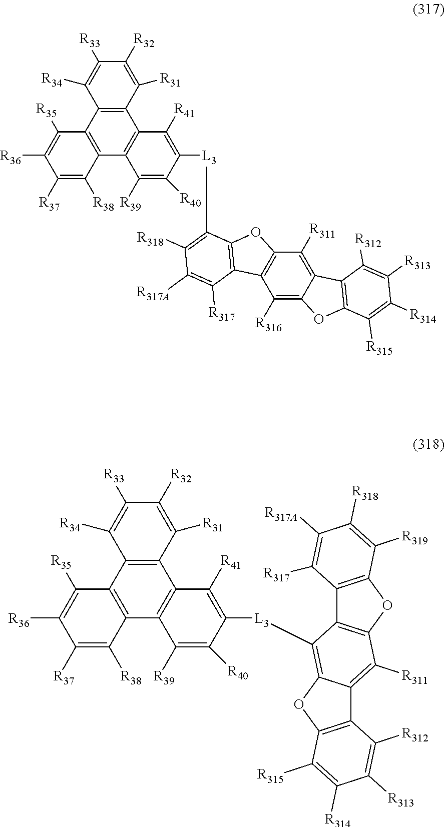

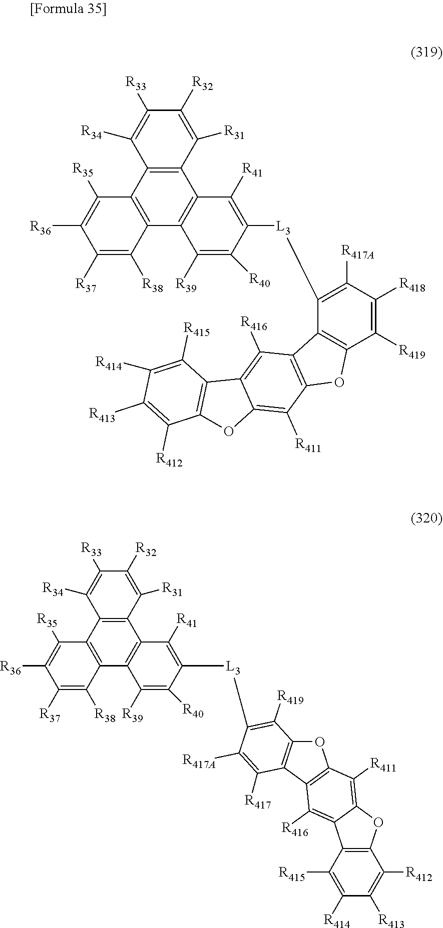

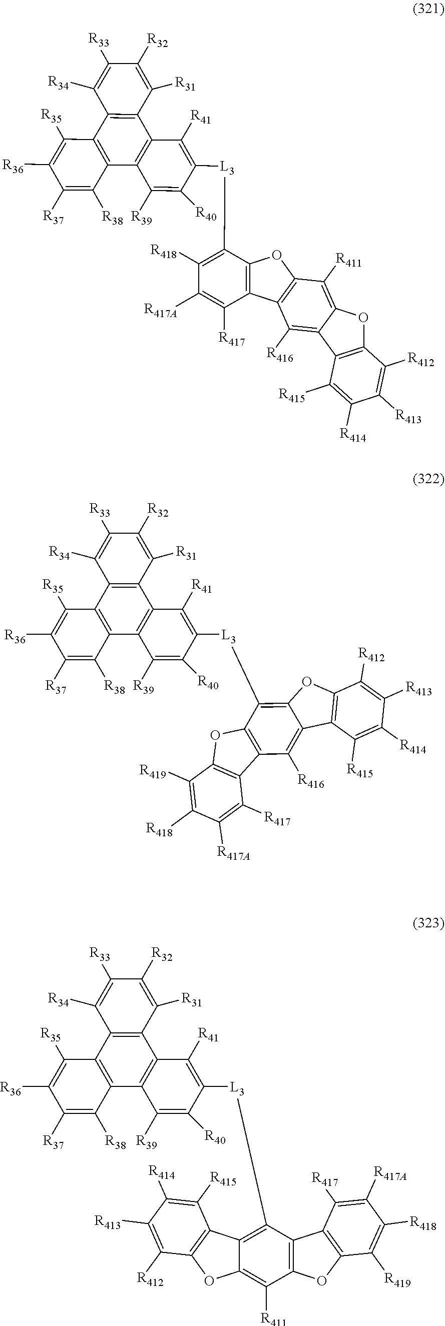

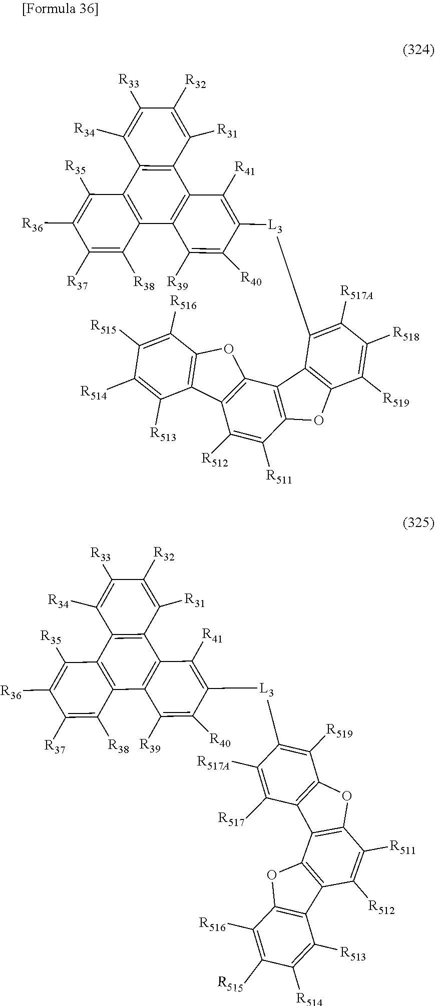

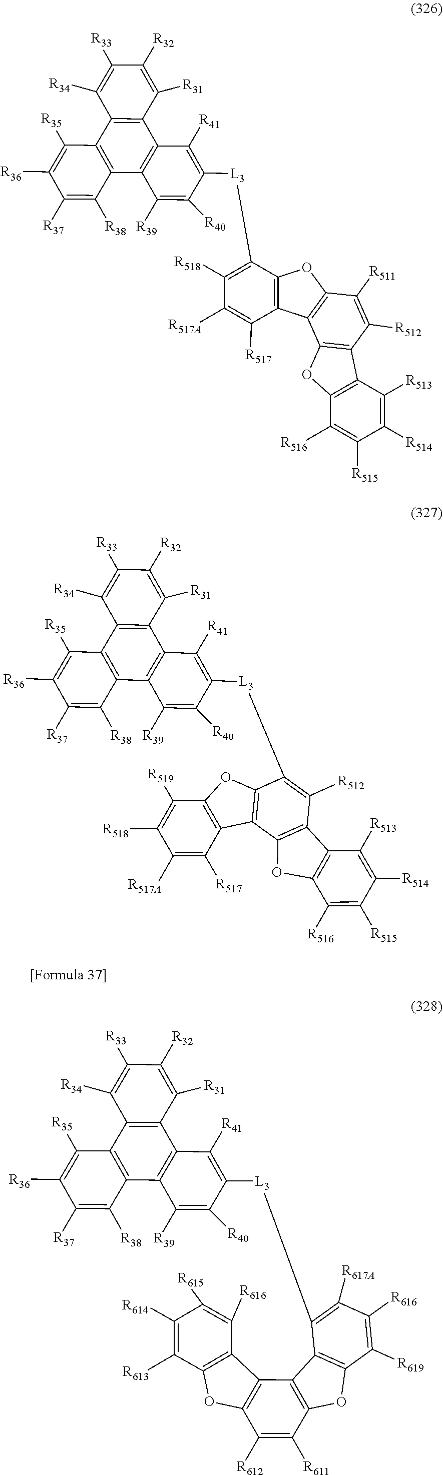

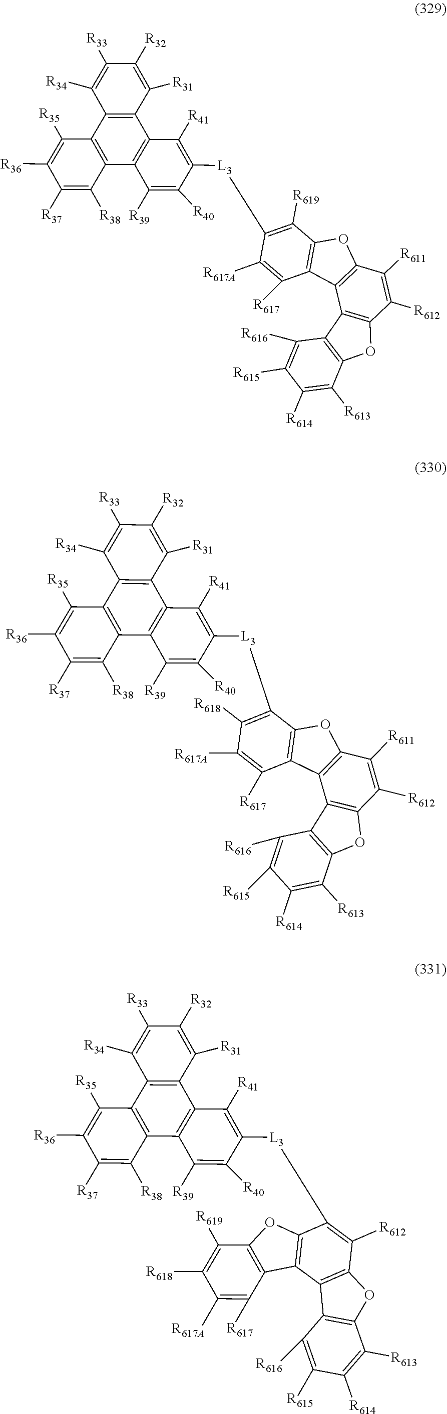

In the formulae (307) to (331): R₁₁₁ to R₁₁₉, R_{117A}, R₂₁₁ to R₂₁₉, R_{217A}, R₃₁₁ to R₃₁₉, R_{317A}, R₄₁₁ to R₄₁₉, R_{417A}, R₅₁₁ to R₅₁₉, R_{517A}, R₆₁₁ to R₆₁₉ and R_{617A} each independently represent the same as R₃₁₀ in the formulae (3a) to (3f); R₃₁ to R₄₁ each represent the same as R₄₂ in the formula (3); and L₃ represents the same as L₃ in the formula (3).

In the formulae (307) to (331), R₁₁₁ to R₁₁₉, R_{117A}, R₂₁₁ to R₂₁₉, R_{217A}, R₃₁₁ to R₃₁₉, R_{317A}, R₄₁₁ to R₄₁₉, R_{417A}, R₅₁₁ to R₅₁₉, R_{517A}, R₆₁₁ to R₆₁₉, R_{617A} and R₃₁ to R₄₁ are preferably each independently a hydrogen atom or a substituted or unsubstituted aryl group having 6 to 30 ring carbon atoms, more preferably a hydrogen atom or a substituted or unsubstituted phenyl group, further preferably a hydrogen atom.

In the compound M3, it is preferable that L₃ is a single bond or a linking group, L₃ as a linking group is a substituted or unsubstituted arylene group having 6 to 40 ring carbon atoms, a group represented by -L₃₁-L₃₂-, or a group represented by -L₃₁-L₃₂-L₃₃-, and L₃₁, L₃₂ and L₃₃ are each independently a substituted or unsubstituted arylene group having 6 to 40 ring carbon atoms.

In the compound M3, L₃, L₃₁, L₃₂ and L₃₃ are each more preferably not a substituted or unsubstituted anthracenylene group.

In the compound M3, it is further preferable that L₃ is a single bond or a linking group, and L₃ as a linking group is a substituted or unsubstituted phenylene group, a substituted or unsubstituted biphenylene group, a substituted or unsubstituted terphenylene group, or a substituted or unsubstituted naphthylene group.

In the compound M3, L₃ as a linking group is also preferably a divalent group derived from a substituted or unsubstituted phenanthryl group, a divalent group derived from a substituted or unsubstituted triphenylenyl group, a divalent group derived from a substituted or unsubstituted fluorenyl group (preferably a 9,9′-dimethylfluorenyl group or a 9,9′-diphenylfluorenyl group), or a divalent group derived from a 9,9′-spirobifluorenyl group.

In the compound M3, when L₃ as a linking group is a group represented by -L₃₁-L₃₂- or a group represented by -L₃₁-L₃₂-L₃₃-, L₃₁, L₃₂ and L₃₃ are preferably each independently a substituted or unsubstituted phenylene group or a substituted or unsubstituted naphthylene group.

In the compound M3, L₃ is also preferably a single bond.

In the compound M3, a substituent, if present, for L₃ is preferably a substituted or unsubstituted aryl group having 6 to 30 ring carbon atoms, more preferably a substituted or unsubstituted phenyl group.

In the compound M3, L₃ as a linking group is also preferably at least one group selected from the group consisting of groups represented by formulae (L₃-1) to (L₃-18) below.

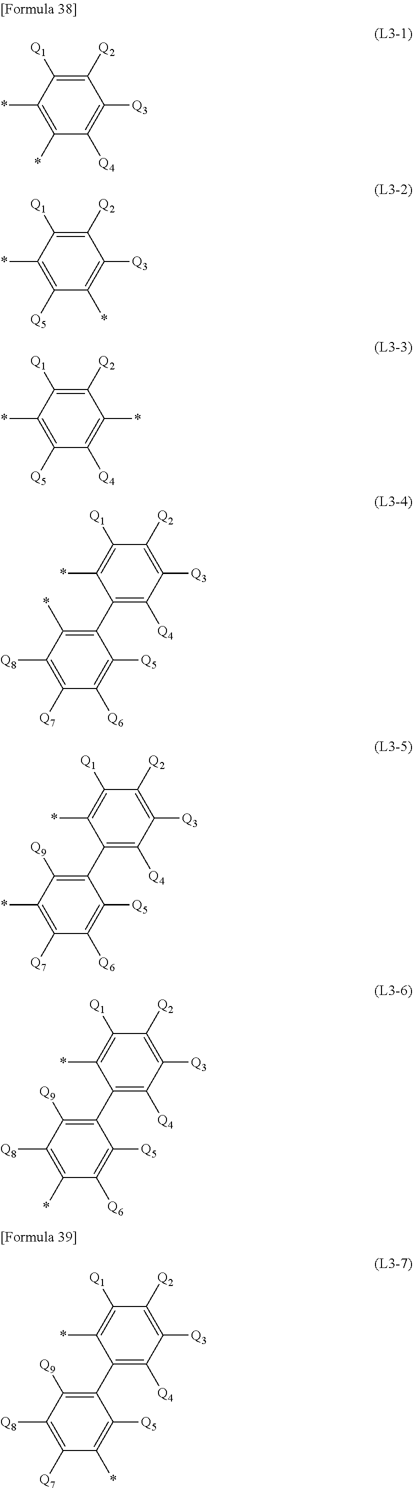

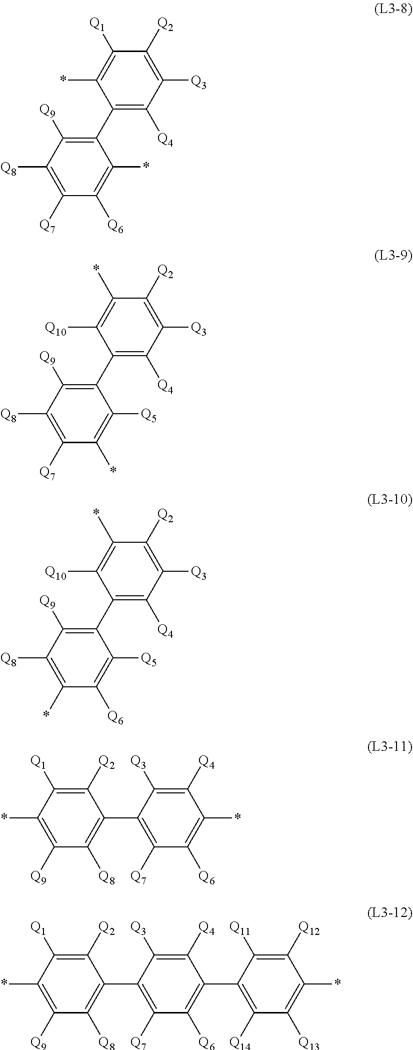

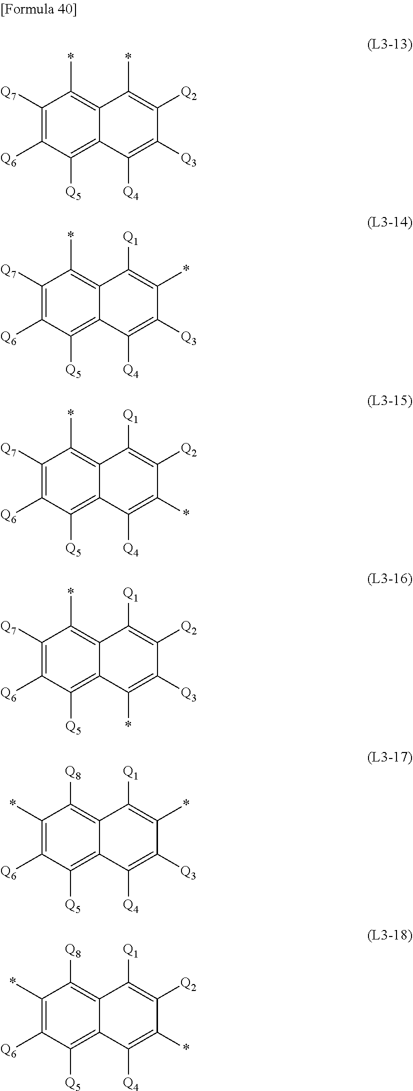

In the formulae (L₃-1) to (L₃-18), Q₁ to Q₁₄ are each independently a hydrogen atom or a substituent. Q₁ to Q₁₄ as a substituent are preferably each independently a hydrogen atom or a substituted or unsubstituted aryl group having 6 to 30 ring carbon atoms, more preferably a substituted or unsubstituted phenyl group, a substituted or unsubstituted naphthyl group, a substituted or unsubstituted phenanthryl group, a substituted or unsubstituted triphenylenyl group, a substituted or unsubstituted fluorenyl group (preferably a 9,9′-dimethylfluorenyl group or a 9,9′-diphenylfluorenyl group), or a 9,9′-spirobifluorenyl group, further preferably a substituted or unsubstituted phenyl group.

### Manufacturing Method of Compound M3 According to Exemplary Embodiment

The compound M3 of the exemplary embodiment can be manufactured, for instance, by a method described later in Examples. The compound M3 of the exemplary embodiment can be manufactured by reactions described in later-described Examples and using known alternative reactions or raw materials suitable for the desired substances.

### Specific Examples of Compound M3

Specific examples of the compound M3 of the exemplary embodiment include compounds below. It should however be noted that the invention is not limited to the specific examples of the compound.

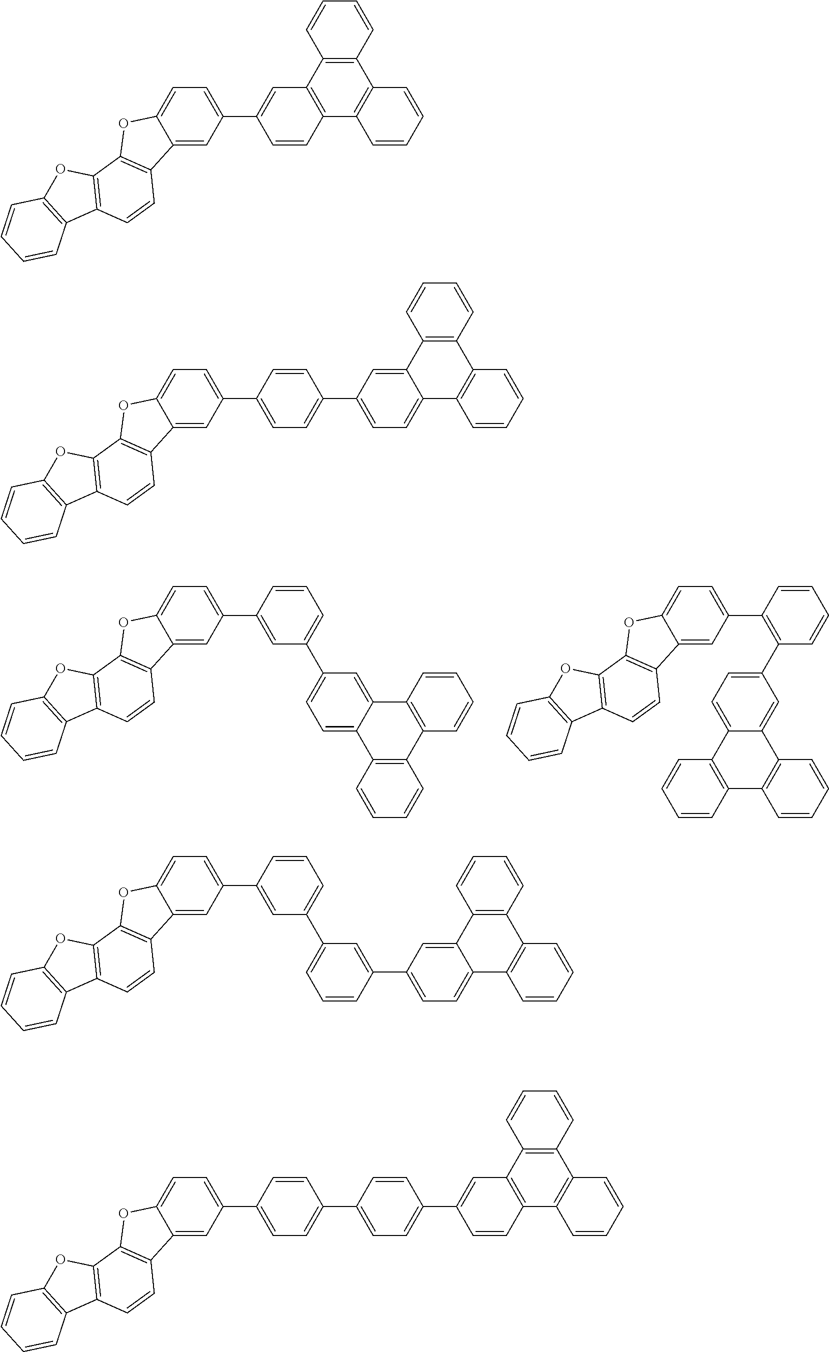

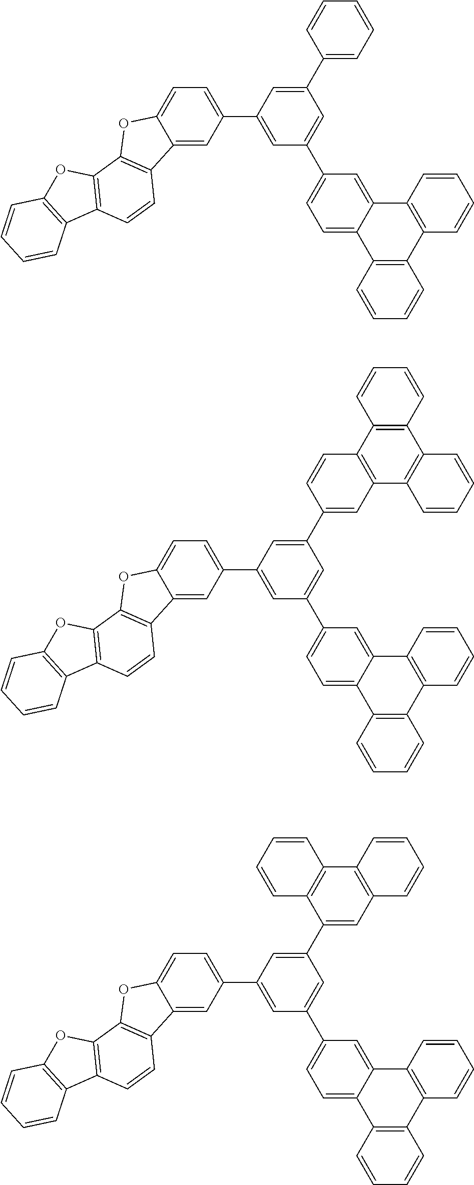 [SMILES: c1ccc(-c2cc(-c3ccc4oc5c(ccc6c7ccccc7oc65)c4c3)cc(-c3ccc4c5ccccc5c5ccccc5c4c3)c2)cc1 ; c1ccc2c(c1)oc1c2ccc2c3cc(-c4cc(-c5ccc6c7ccccc7c7ccccc7c6c5)cc(-c5ccc6c7ccccc7c7ccccc7c6c5)c4)ccc3oc21 ; c1ccc2c(c1)cc(-c1cc(-c3ccc4oc5c(ccc6c7ccccc7oc65)c4c3)cc(-c3ccc4c5ccccc5c5ccccc5c4c3)c1)c1ccccc12]

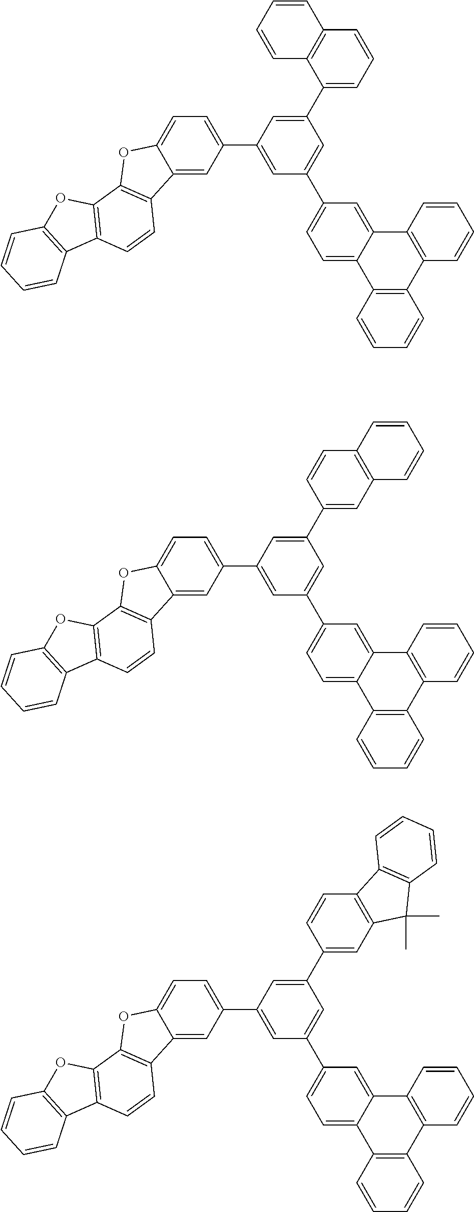 [SMILES: c1ccc2c(-c3cc(-c4ccc5oc6c(ccc7c8ccccc8oc76)c5c4)cc(-c4ccc5c6ccccc6c6ccccc6c5c4)c3)cccc2c1 ; c1ccc2cc(-c3cc(-c4ccc5oc6c(ccc7c8ccccc8oc76)c5c4)cc(-c4ccc5c6ccccc6c6ccccc6c5c4)c3)ccc2c1 ; CC1(C)c2ccccc2-c2ccc(-c3cc(-c4ccc5oc6c(ccc7c8ccccc8oc76)c5c4)cc(-c4ccc5c6ccccc6c6ccccc6c5c4)c3)cc21]

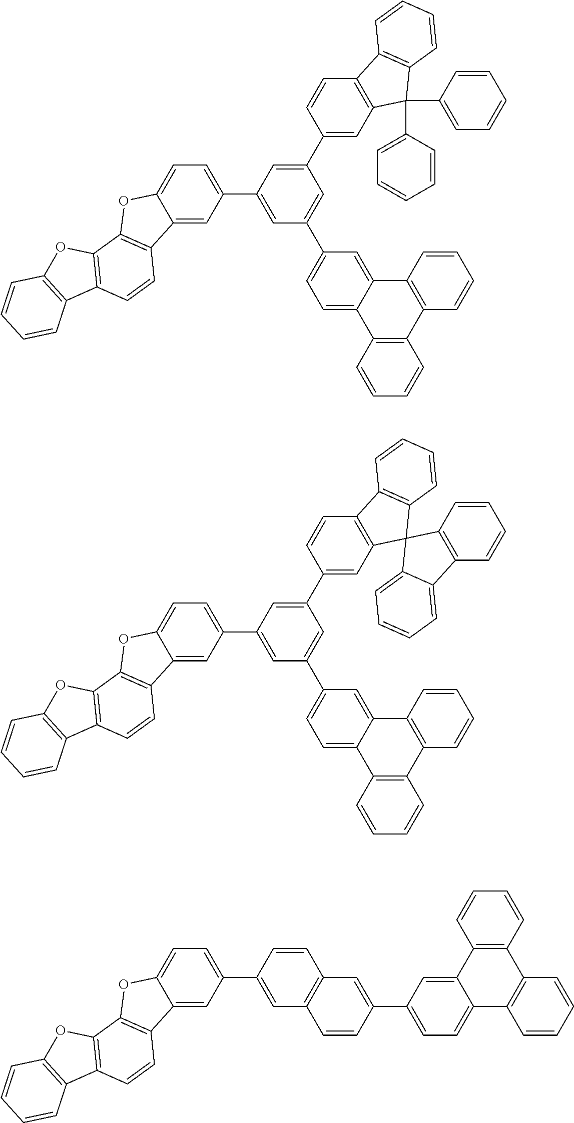

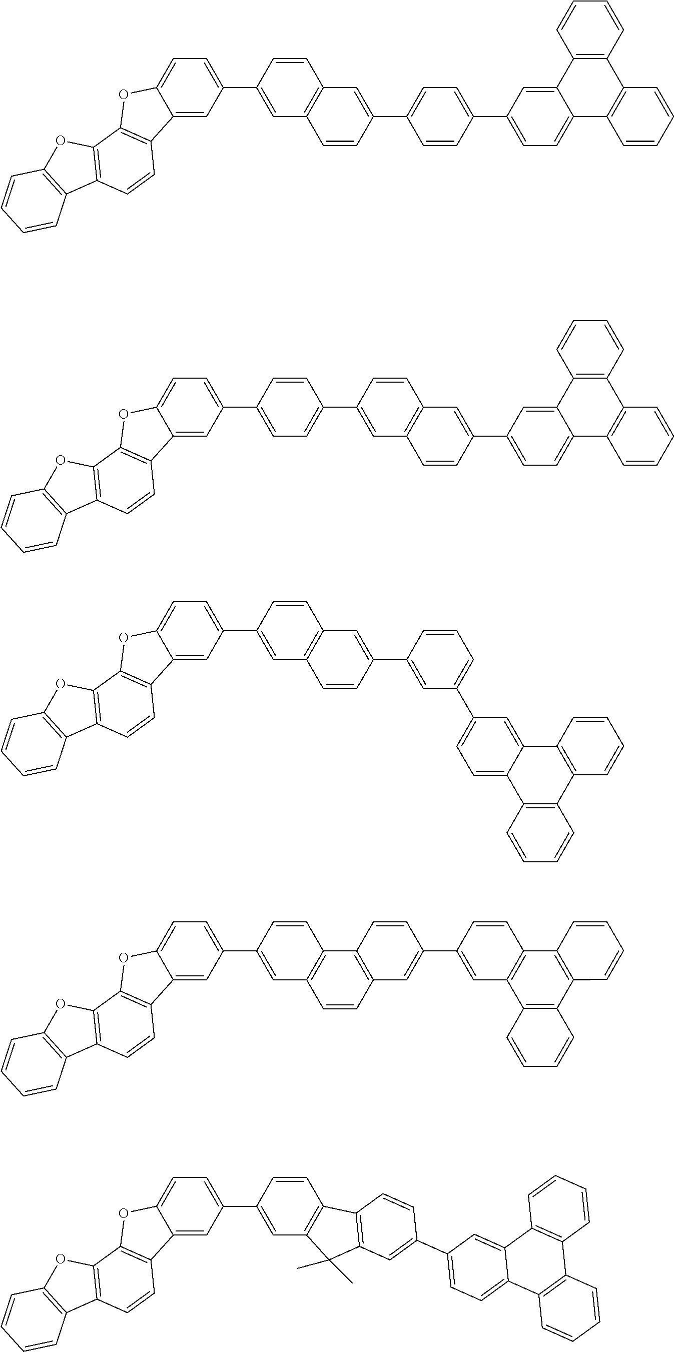 [SMILES: c1ccc2c(c1)oc1c2ccc2c3cc(-c4ccc5cc(-c6ccc(-c7ccc8c9ccccc9c9ccccc9c8c7)cc6)ccc5c4)ccc3oc21 ; c1ccc2c(c1)oc1c2ccc2c3cc(-c4ccc(-c5ccc6cc(-c7ccc8c9ccccc9c9ccccc9c8c7)ccc6c5)cc4)ccc3oc21 ; c1cc(-c2ccc3cc(-c4ccc5oc6c(ccc7c8ccccc8oc76)c5c4)ccc3c2)cc(-c2ccc3c4ccccc4c4ccccc4c3c2)c1 ; c1ccc2c(c1)oc1c2ccc2c3cc(-c4ccc5c(ccc6cc(-c7ccc8c9ccccc9c9ccccc9c8c7)ccc65)c4)ccc3oc21 ; CC1(C)c2cc(-c3ccc4oc5c(ccc6c7ccccc7oc65)c4c3)ccc2-c2ccc(-c3ccc4c5ccccc5c5ccccc5c4c3)cc21]

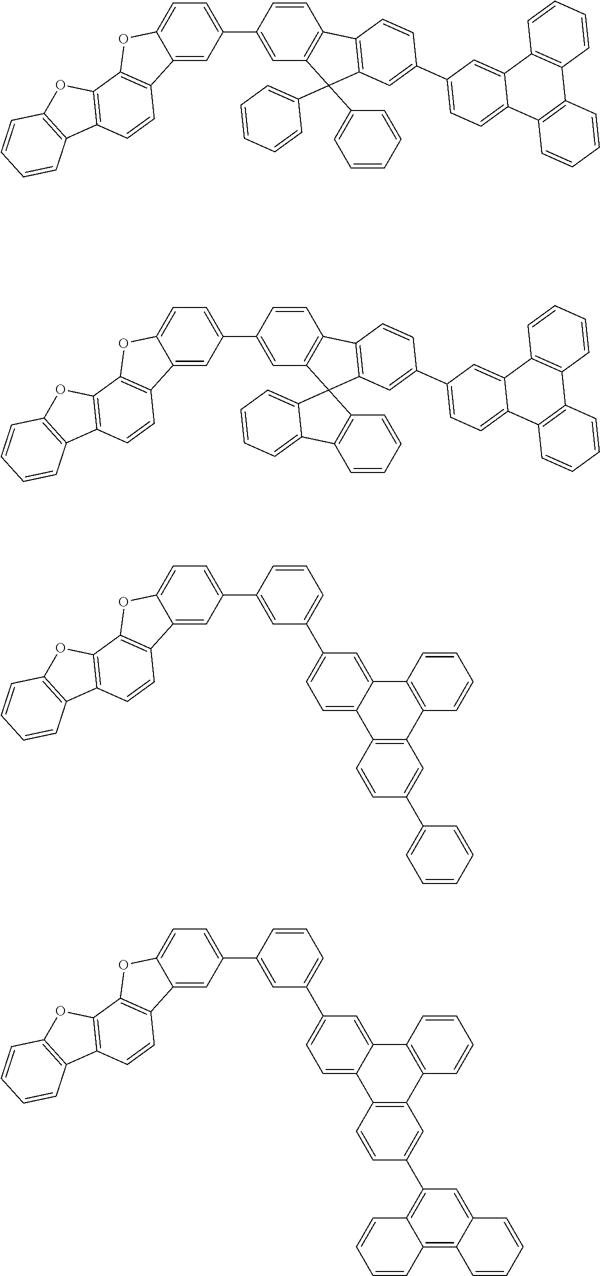 [SMILES: c1ccc(C2(c3ccccc3)c3cc(-c4ccc5oc6c(ccc7c8ccccc8oc76)c5c4)ccc3-c3ccc(-c4ccc5c6ccccc6c6ccccc6c5c4)cc32)cc1 ; c1ccc2c(c1)-c1ccccc1C21c2cc(-c3ccc4oc5c(ccc6c7ccccc7oc65)c4c3)ccc2-c2ccc(-c3ccc4c5ccccc5c5ccccc5c4c3)cc21 ; c1ccc(-c2ccc3c4ccc(-c5cccc(-c6ccc7oc8c(ccc9c%10ccccc%10oc98)c7c6)c5)cc4c4ccccc4c3c2)cc1 ; c1cc(-c2ccc3oc4c(ccc5c6ccccc6oc54)c3c2)cc(-c2ccc3c4ccc(-c5cc6ccccc6c6ccccc56)cc4c4ccccc4c3c2)c1]

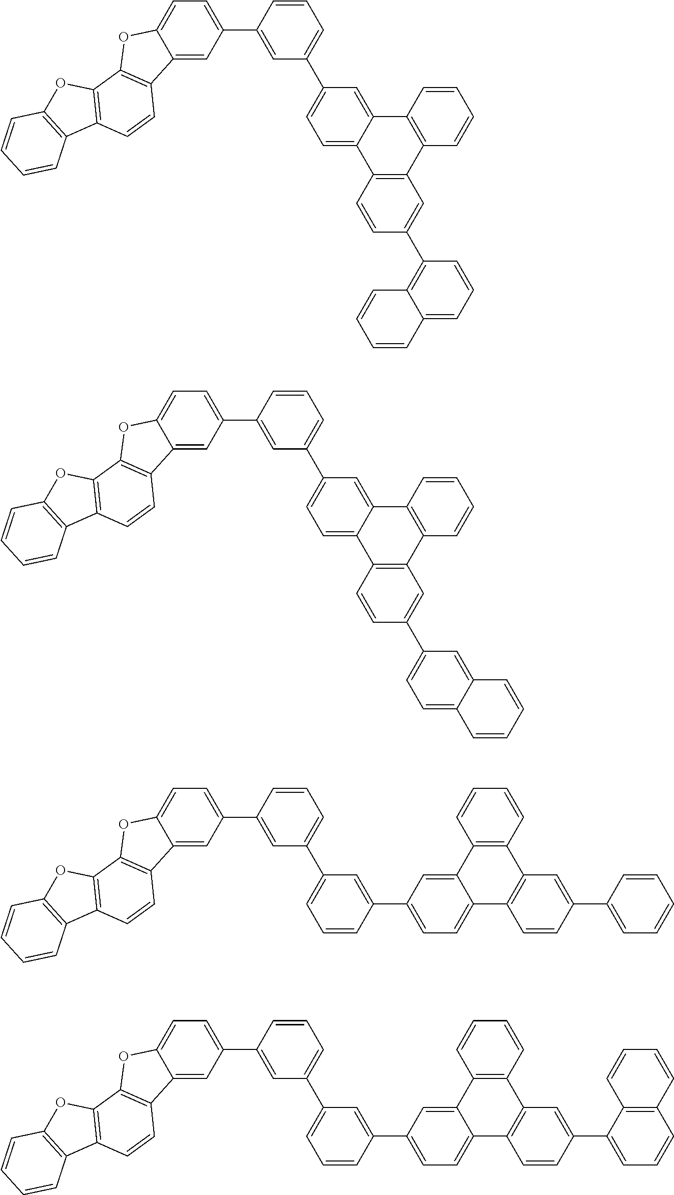 [SMILES: c1cc(-c2ccc3oc4c(ccc5c6ccccc6oc54)c3c2)cc(-c2ccc3c4ccc(-c5cccc6ccccc56)cc4c4ccccc4c3c2)c1 ; c1cc(-c2ccc3oc4c(ccc5c6ccccc6oc54)c3c2)cc(-c2ccc3c4ccc(-c5ccc6ccccc6c5)cc4c4ccccc4c3c2)c1 ; c1ccc(-c2ccc3c4ccc(-c5cccc(-c6cccc(-c7ccc8oc9c(ccc%10c%11ccccc%11oc%109)c8c7)c6)c5)cc4c4ccccc4c3c2)cc1 ; c1cc(-c2cccc(-c3ccc4c5ccc(-c6cccc7ccccc67)cc5c5ccccc5c4c3)c2)cc(-c2ccc3oc4c(ccc5c6ccccc6oc54)c3c2)c1]

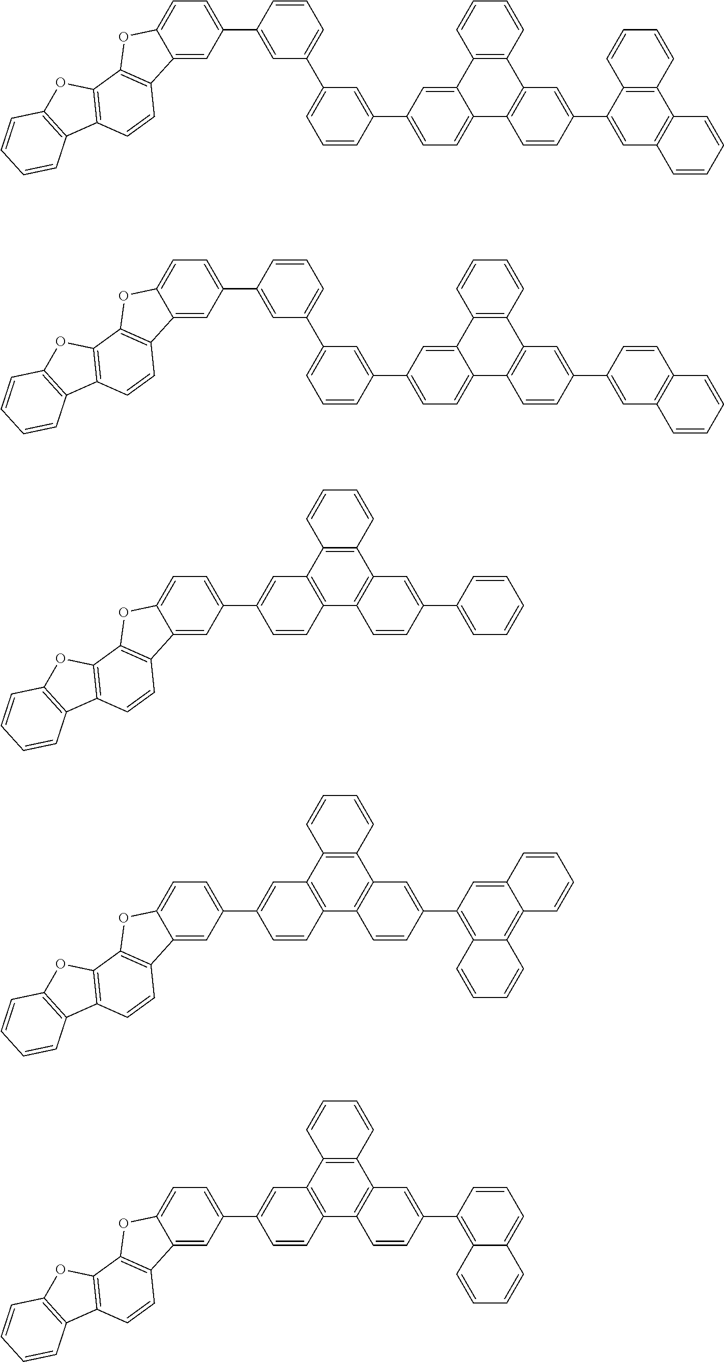 [SMILES: c1cc(-c2cccc(-c3ccc4c5ccc(-c6cc7ccccc7c7ccccc67)cc5c5ccccc5c4c3)c2)cc(-c2ccc3oc4c(ccc5c6ccccc6oc54)c3c2)c1 ; c1cc(-c2cccc(-c3ccc4c5ccc(-c6ccc7ccccc7c6)cc5c5ccccc5c4c3)c2)cc(-c2ccc3oc4c(ccc5c6ccccc6oc54)c3c2)c1 ; c1ccc(-c2ccc3c4ccc(-c5ccc6oc7c(ccc8c9ccccc9oc87)c6c5)cc4c4ccccc4c3c2)cc1 ; c1ccc2c(c1)cc(-c1ccc3c4ccc(-c5ccc6oc7c(ccc8c9ccccc9oc87)c6c5)cc4c4ccccc4c3c1)c1ccccc12 ; c1ccc2c(-c3ccc4c5ccc(-c6ccc7oc8c(ccc9c%10ccccc%10oc98)c7c6)cc5c5ccccc5c4c3)cccc2c1]

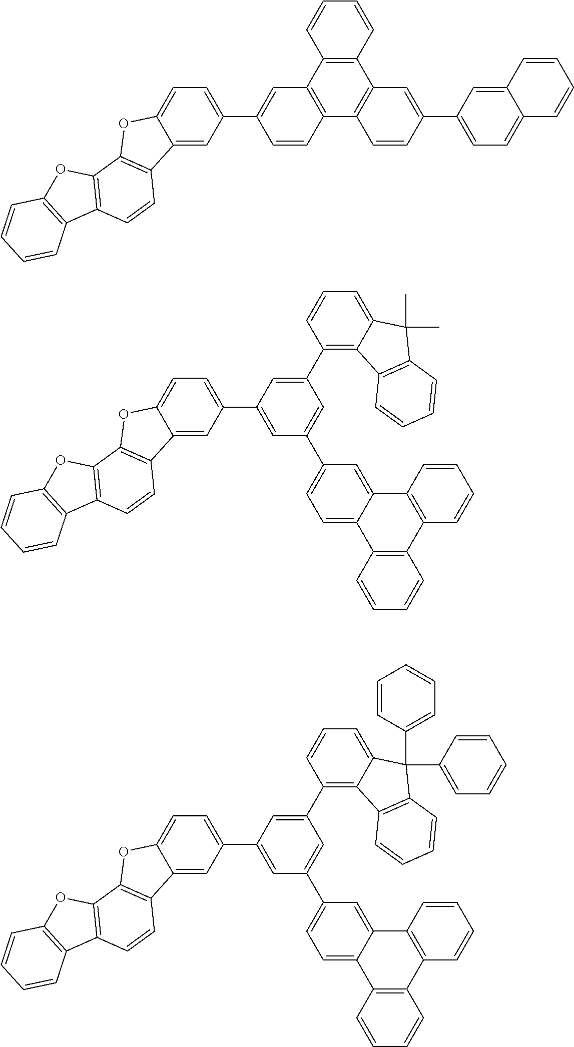 [SMILES: c1ccc2cc(-c3ccc4c5ccc(-c6ccc7oc8c(ccc9c%10ccccc%10oc98)c7c6)cc5c5ccccc5c4c3)ccc2c1 ; CC1(C)c2ccccc2-c2c(-c3cc(-c4ccc5oc6c(ccc7c8ccccc8oc76)c5c4)cc(-c4ccc5c6ccccc6c6ccccc6c5c4)c3)cccc21 ; c1ccc(C2(c3ccccc3)c3ccccc3-c3c(-c4cc(-c5ccc6oc7c(ccc8c9ccccc9oc87)c6c5)cc(-c5ccc6c7ccccc7c7ccccc7c6c5)c4)cccc32)cc1]

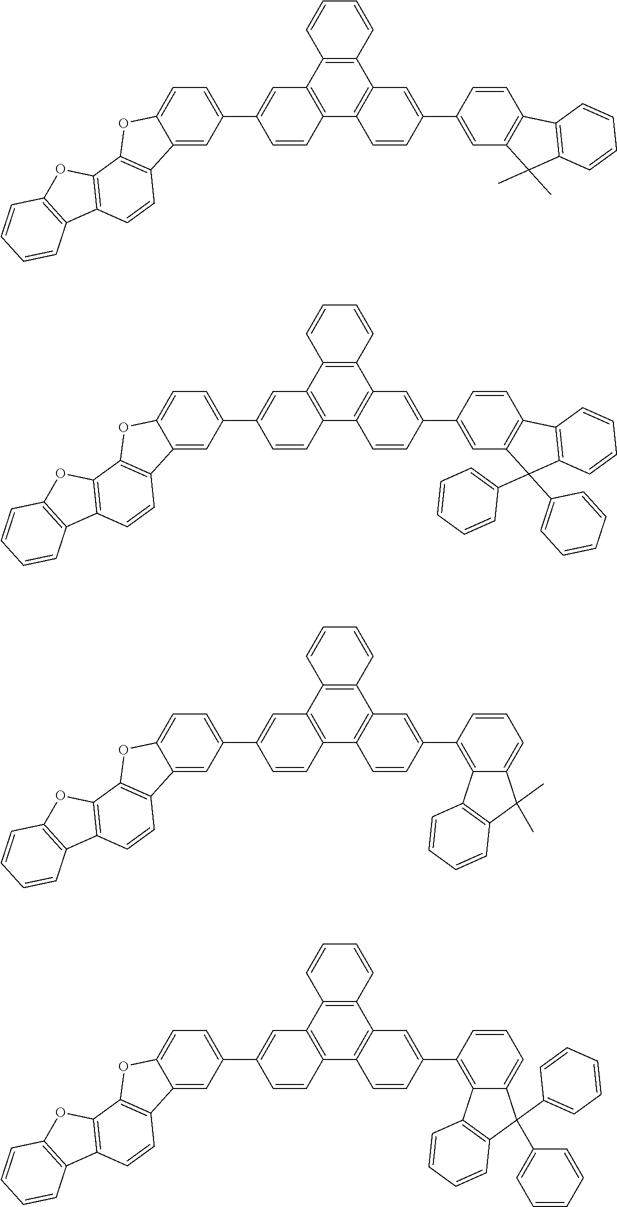 [SMILES: CC1(C)c2ccccc2-c2ccc(-c3ccc4c5ccc(-c6ccc7oc8c(ccc9c%10ccccc%10oc98)c7c6)cc5c5ccccc5c4c3)cc21 ; c1ccc(C2(c3ccccc3)c3ccccc3-c3ccc(-c4ccc5c6ccc(-c7ccc8oc9c(ccc%10c%11ccccc%11oc%109)c8c7)cc6c6ccccc6c5c4)cc32)cc1 ; CC1(C)c2ccccc2-c2c(-c3ccc4c5ccc(-c6ccc7oc8c(ccc9c%10ccccc%10oc98)c7c6)cc5c5ccccc5c4c3)cccc21 ; c1ccc(C2(c3ccccc3)c3ccccc3-c3c(-c4ccc5c6ccc(-c7ccc8oc9c(ccc%10c%11ccccc%11oc%109)c8c7)cc6c6ccccc6c5c4)cccc32)cc1]

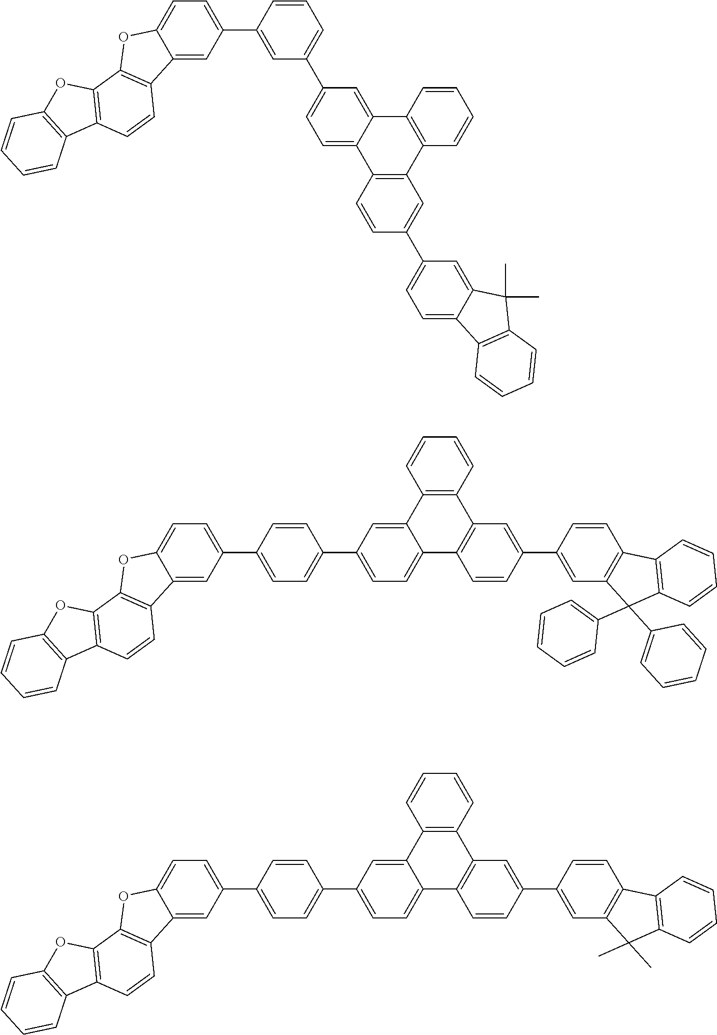 [SMILES: CC1(C)c2ccccc2-c2ccc(-c3ccc4c5ccc(-c6cccc(-c7ccc8oc9c(ccc%10c%11ccccc%11oc%109)c8c7)c6)cc5c5ccccc5c4c3)cc21 ; c1ccc(C2(c3ccccc3)c3ccccc3-c3ccc(-c4ccc5c6ccc(-c7ccc(-c8ccc9oc%10c(ccc%11c%12ccccc%12oc%11%10)c9c8)cc7)cc6c6ccccc6c5c4)cc32)cc1 ; CC1(C)c2ccccc2-c2ccc(-c3ccc4c5ccc(-c6ccc(-c7ccc8oc9c(ccc%10c%11ccccc%11oc%109)c8c7)cc6)cc5c5ccccc5c4c3)cc21]

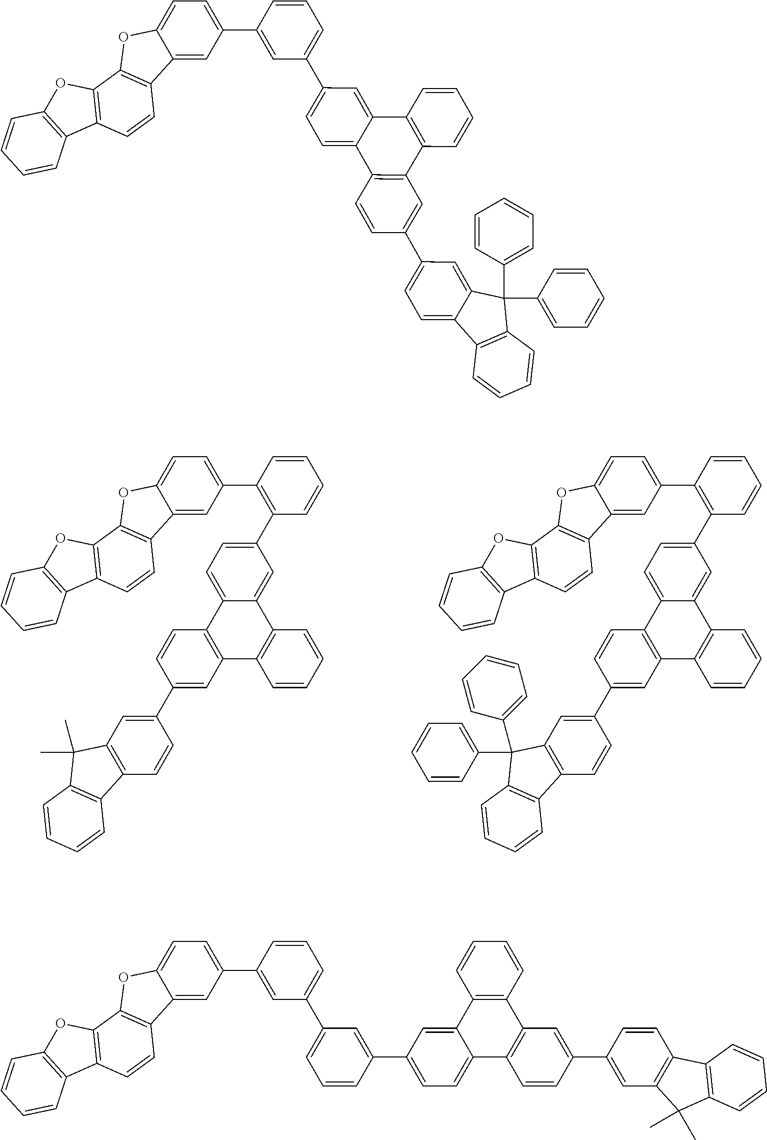 [SMILES: c1ccc(C2(c3ccccc3)c3ccccc3-c3ccc(-c4ccc5c6ccc(-c7cccc(-c8ccc9oc%10c(ccc%11c%12ccccc%12oc%11%10)c9c8)c7)cc6c6ccccc6c5c4)cc32)cc1 ; CC1(C)c2ccccc2-c2ccc(-c3ccc4c5ccc(-c6ccccc6-c6ccc7oc8c(ccc9c%10ccccc%10oc98)c7c6)cc5c5ccccc5c4c3)cc21 ; c1ccc(C2(c3ccccc3)c3ccccc3-c3ccc(-c4ccc5c6ccc(-c7ccccc7-c7ccc8oc9c(ccc%10c%11ccccc%11oc%109)c8c7)cc6c6ccccc6c5c4)cc32)cc1 ; CC1(C)c2ccccc2-c2ccc(-c3ccc4c5ccc(-c6cccc(-c7cccc(-c8ccc9oc%10c(ccc%11c%12ccccc%12oc%11%10)c9c8)c7)c6)cc5c5ccccc5c4c3)cc21]

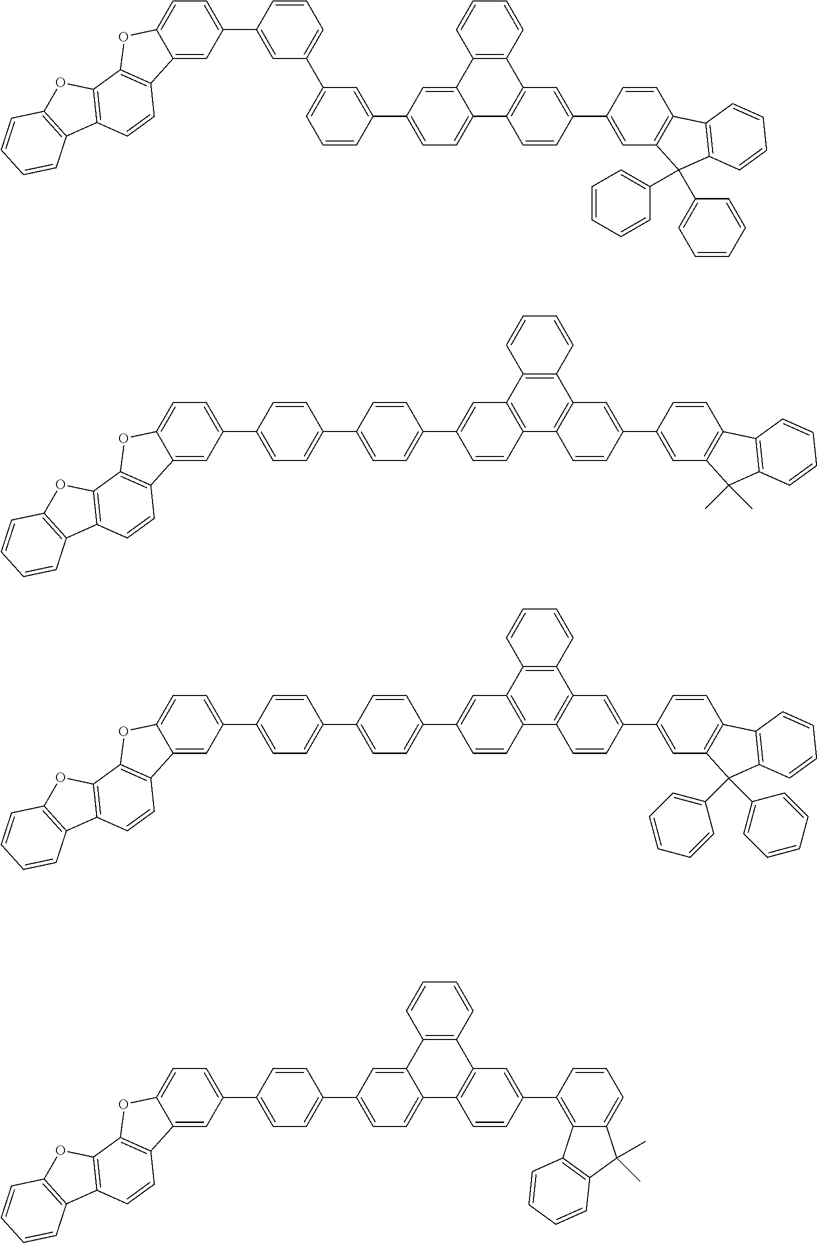

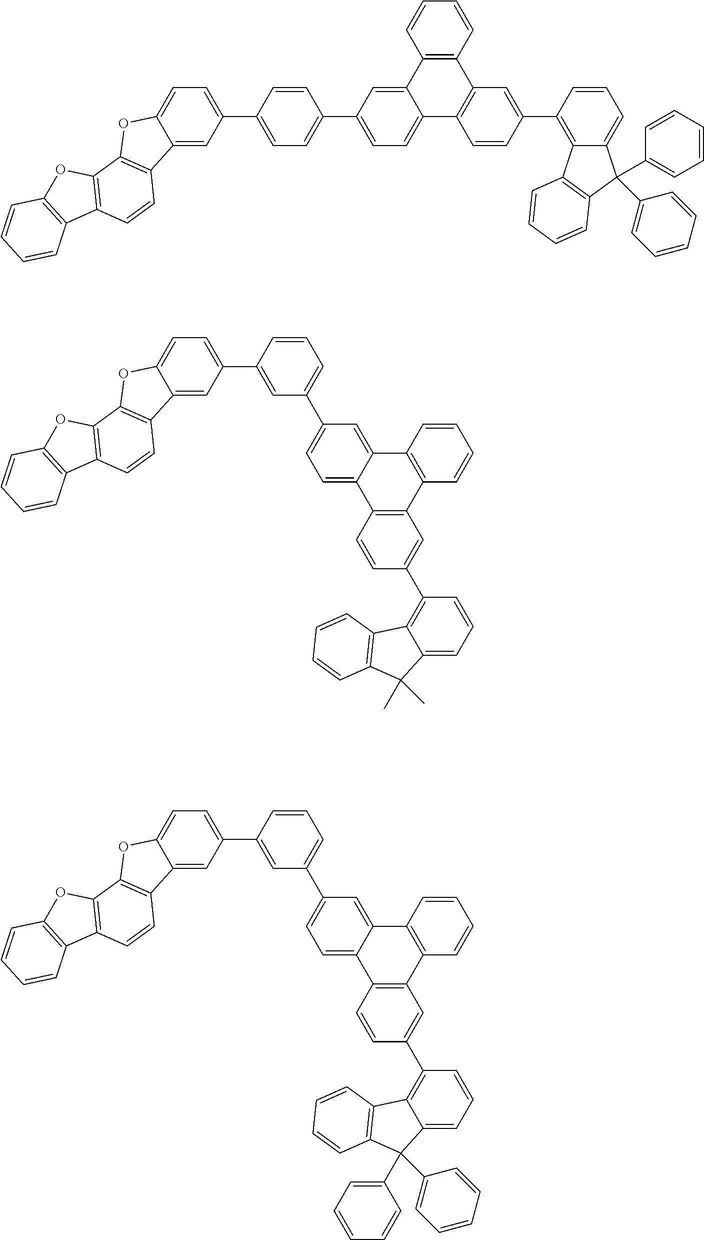

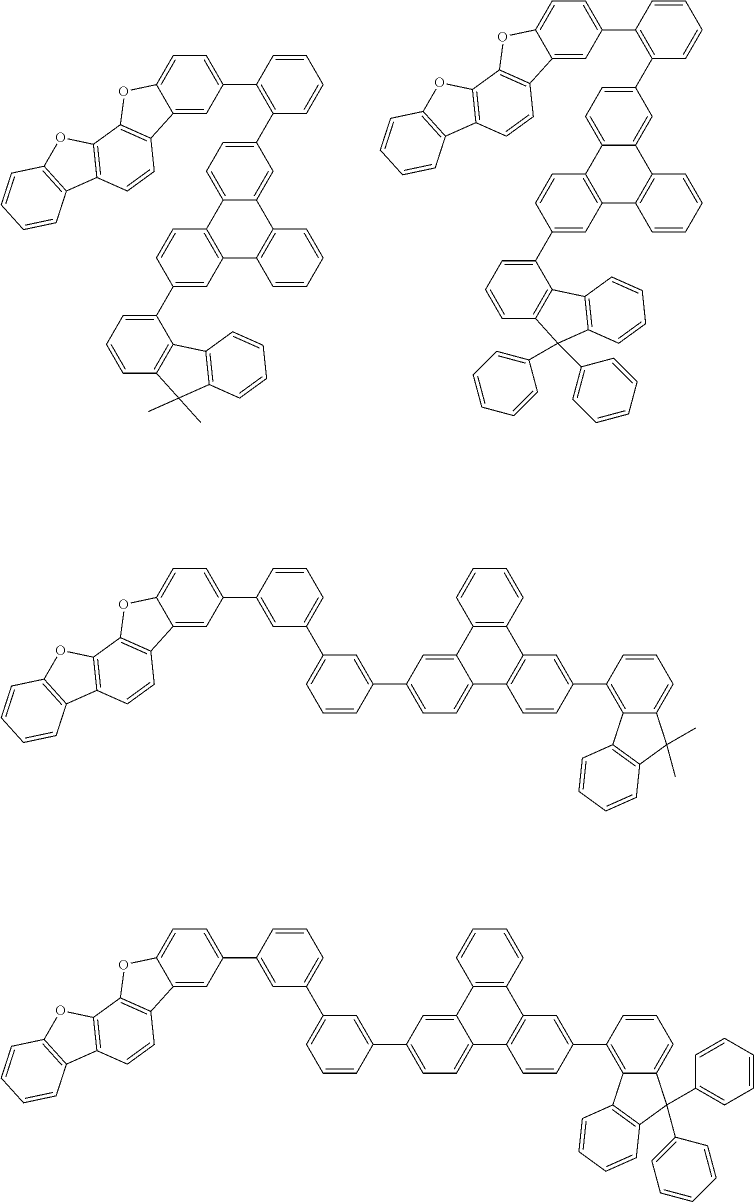 [SMILES: CC1(C)c2ccccc2-c2c(-c3ccc4c5ccc(-c6ccccc6-c6ccc7oc8c(ccc9c%10ccccc%10oc98)c7c6)cc5c5ccccc5c4c3)cccc21 ; c1ccc(C2(c3ccccc3)c3ccccc3-c3c(-c4ccc5c6ccc(-c7ccccc7-c7ccc8oc9c(ccc%10c%11ccccc%11oc%109)c8c7)cc6c6ccccc6c5c4)cccc32)cc1 ; CC1(C)c2ccccc2-c2c(-c3ccc4c5ccc(-c6cccc(-c7cccc(-c8ccc9oc%10c(ccc%11c%12ccccc%12oc%11%10)c9c8)c7)c6)cc5c5ccccc5c4c3)cccc21 ; c1ccc(C2(c3ccccc3)c3ccccc3-c3c(-c4ccc5c6ccc(-c7cccc(-c8cccc(-c9ccc%10oc%11c(ccc%12c%13ccccc%13oc%12%11)c%10c9)c8)c7)cc6c6ccccc6c5c4)cccc32)cc1]

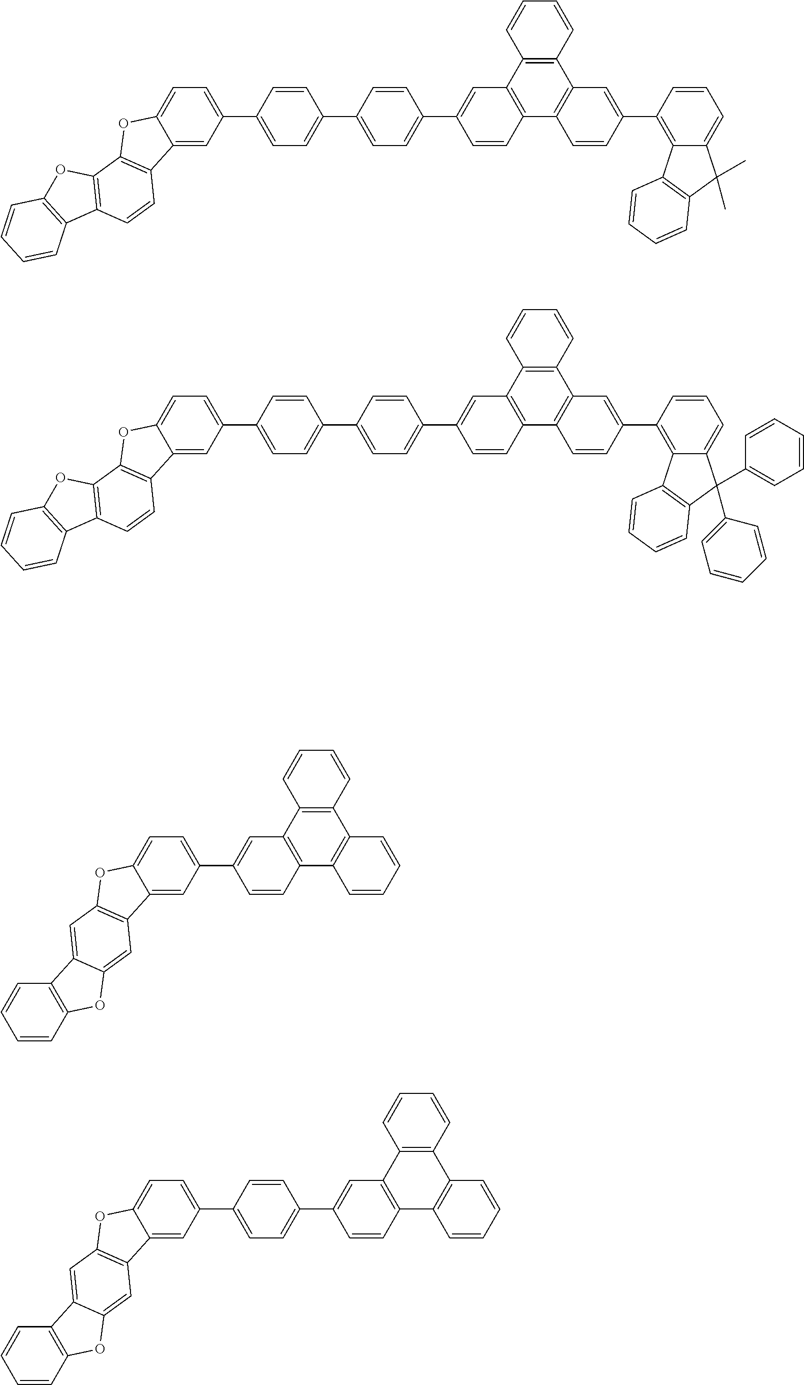 [SMILES: CC1(C)c2ccccc2-c2c(-c3ccc4c5ccc(-c6ccc(-c7ccc(-c8ccc9oc%10c(ccc%11c%12ccccc%12oc%11%10)c9c8)cc7)cc6)cc5c5ccccc5c4c3)cccc21 ; c1ccc(C2(c3ccccc3)c3ccccc3-c3c(-c4ccc5c6ccc(-c7ccc(-c8ccc(-c9ccc%10oc%11c(ccc%12c%13ccccc%13oc%12%11)c%10c9)cc8)cc7)cc6c6ccccc6c5c4)cccc32)cc1 ; c1ccc2c(c1)oc1cc3c(cc12)oc1ccc(-c2ccc4c5ccccc5c5ccccc5c4c2)cc13 ; c1ccc2c(c1)oc1cc3c(cc12)oc1ccc(-c2ccc(-c4ccc5c6ccccc6c6ccccc6c5c4)cc2)cc13]

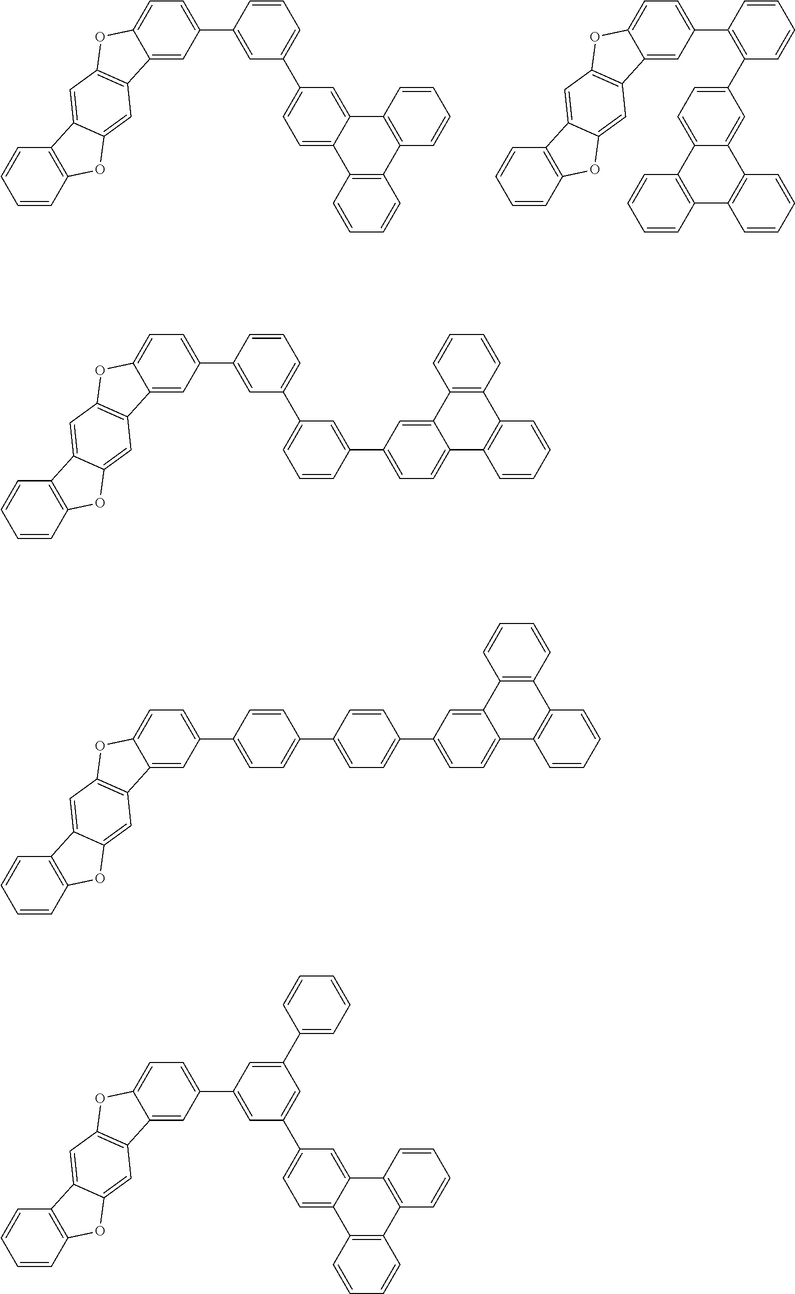 [SMILES: c1cc(-c2ccc3oc4cc5c(cc4c3c2)oc2ccccc25)cc(-c2ccc3c4ccccc4c4ccccc4c3c2)c1 ; c1ccc(-c2ccc3c4ccccc4c4ccccc4c3c2)c(-c2ccc3oc4cc5c(cc4c3c2)oc2ccccc25)c1 ; c1cc(-c2cccc(-c3ccc4c5ccccc5c5ccccc5c4c3)c2)cc(-c2ccc3oc4cc5c(cc4c3c2)oc2ccccc25)c1 ; c1ccc2c(c1)oc1cc3c(cc12)oc1ccc(-c2ccc(-c4ccc(-c5ccc6c7ccccc7c7ccccc7c6c5)cc4)cc2)cc13 ; c1ccc(-c2cc(-c3ccc4oc5cc6c(cc5c4c3)oc3ccccc36)cc(-c3ccc4c5ccccc5c5ccccc5c4c3)c2)cc1]

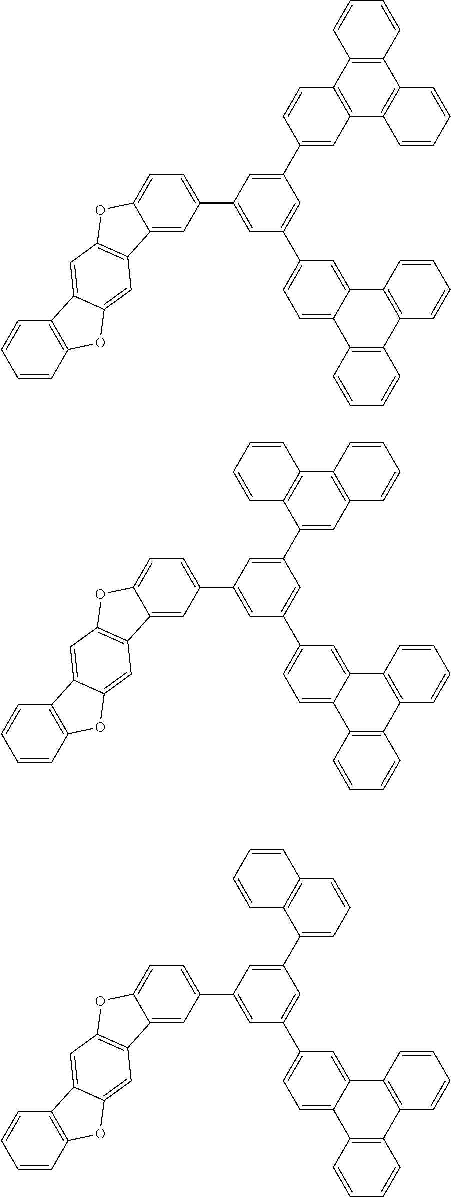

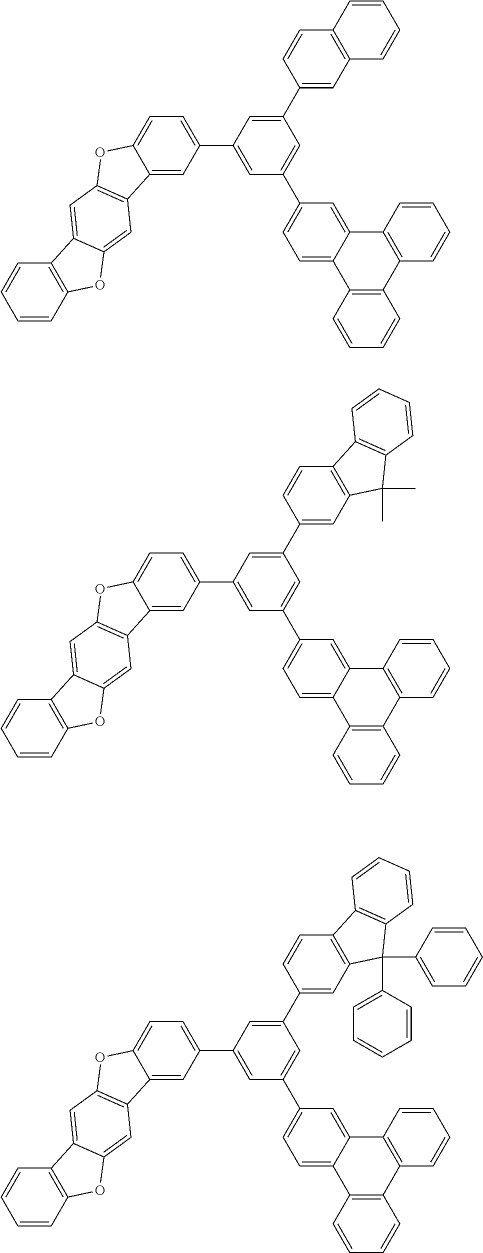

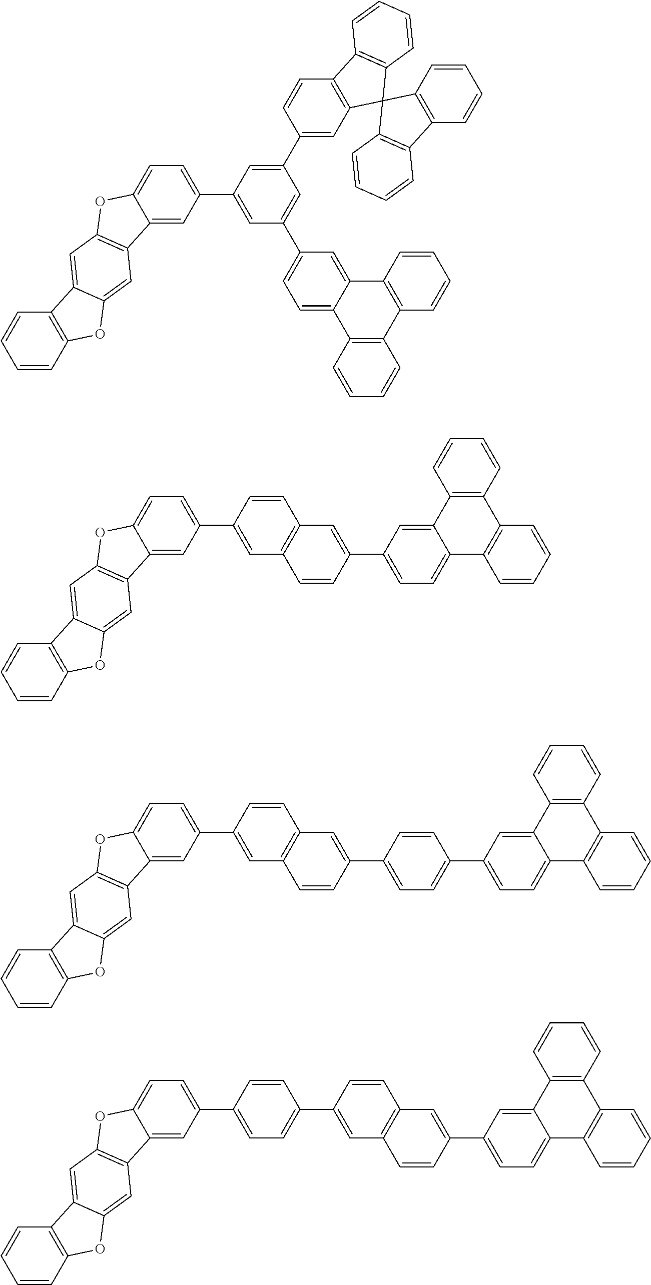 [SMILES: c1ccc2c(c1)-c1ccccc1C21c2ccccc2-c2ccc(-c3cc(-c4ccc5oc6cc7c(cc6c5c4)oc4ccccc47)cc(-c4ccc5c6ccccc6c6ccccc6c5c4)c3)cc21 ; c1ccc2c(c1)oc1cc3c(cc12)oc1ccc(-c2ccc4cc(-c5ccc6c7ccccc7c7ccccc7c6c5)ccc4c2)cc13 ; c1ccc2c(c1)oc1cc3c(cc12)oc1ccc(-c2ccc4cc(-c5ccc(-c6ccc7c8ccccc8c8ccccc8c7c6)cc5)ccc4c2)cc13 ; c1ccc2c(c1)oc1cc3c(cc12)oc1ccc(-c2ccc(-c4ccc5cc(-c6ccc7c8ccccc8c8ccccc8c7c6)ccc5c4)cc2)cc13]

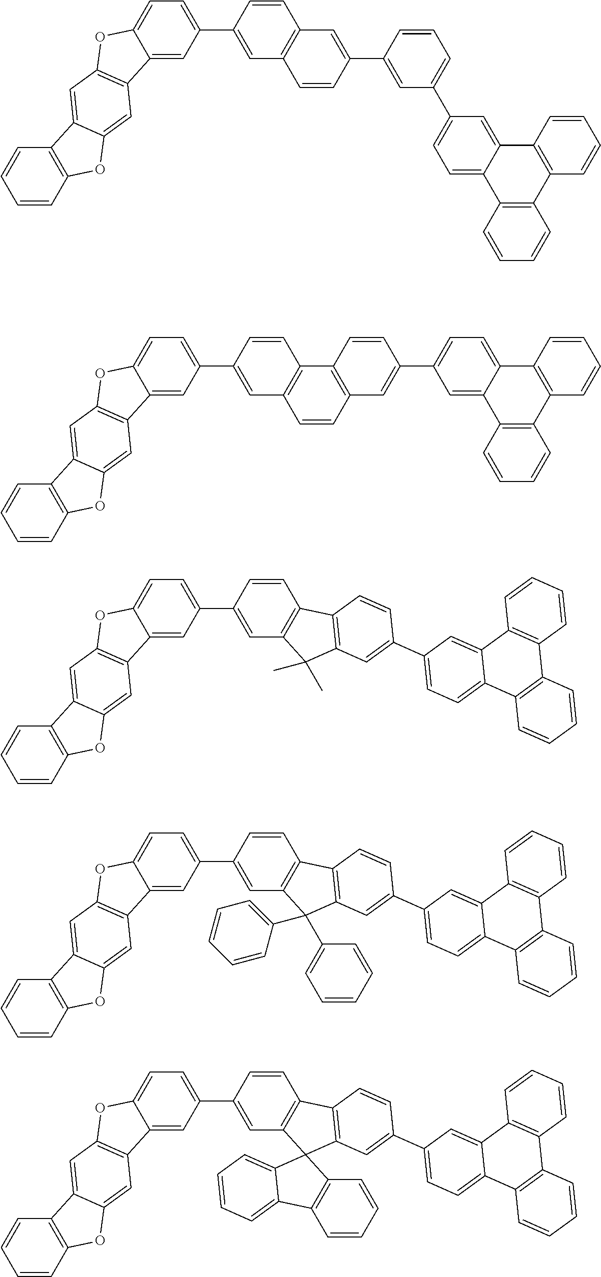 [SMILES: c1cc(-c2ccc3cc(-c4ccc5oc6cc7c(cc6c5c4)oc4ccccc47)ccc3c2)cc(-c2ccc3c4ccccc4c4ccccc4c3c2)c1 ; c1ccc2c(c1)oc1cc3c(cc12)oc1ccc(-c2ccc4c(ccc5cc(-c6ccc7c8ccccc8c8ccccc8c7c6)ccc54)c2)cc13 ; CC1(C)c2cc(-c3ccc4oc5cc6c(cc5c4c3)oc3ccccc36)ccc2-c2ccc(-c3ccc4c5ccccc5c5ccccc5c4c3)cc21 ; c1ccc(C2(c3ccccc3)c3cc(-c4ccc5oc6cc7c(cc6c5c4)oc4ccccc47)ccc3-c3ccc(-c4ccc5c6ccccc6c6ccccc6c5c4)cc32)cc1 ; c1ccc2c(c1)-c1ccccc1C21c2cc(-c3ccc4oc5cc6c(cc5c4c3)oc3ccccc36)ccc2-c2ccc(-c3ccc4c5ccccc5c5ccccc5c4c3)cc21]

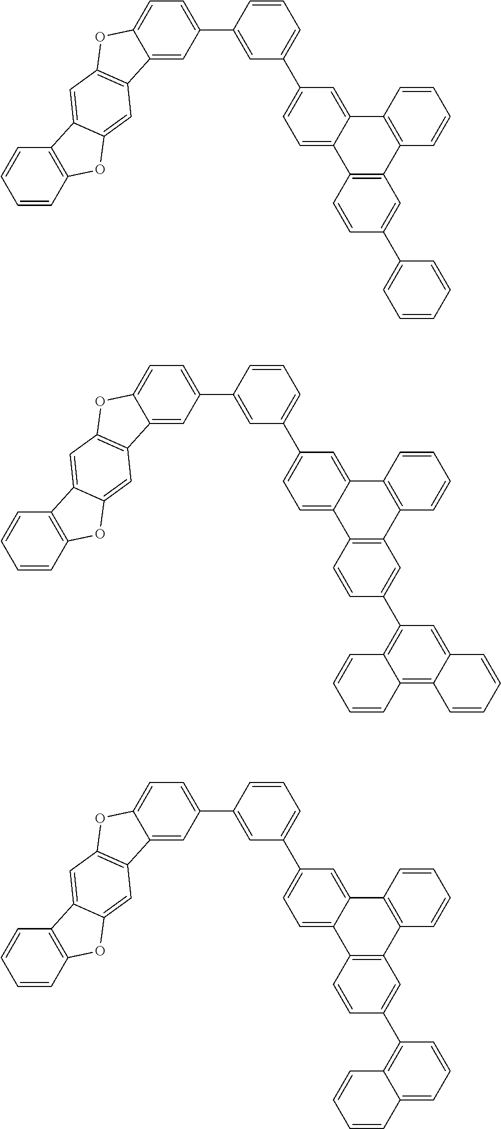 [SMILES: c1ccc(-c2ccc3c4ccc(-c5cccc(-c6ccc7oc8cc9c(cc8c7c6)oc6ccccc69)c5)cc4c4ccccc4c3c2)cc1 ; c1cc(-c2ccc3oc4cc5c(cc4c3c2)oc2ccccc25)cc(-c2ccc3c4ccc(-c5cc6ccccc6c6ccccc56)cc4c4ccccc4c3c2)c1 ; c1cc(-c2ccc3oc4cc5c(cc4c3c2)oc2ccccc25)cc(-c2ccc3c4ccc(-c5cccc6ccccc56)cc4c4ccccc4c3c2)c1]

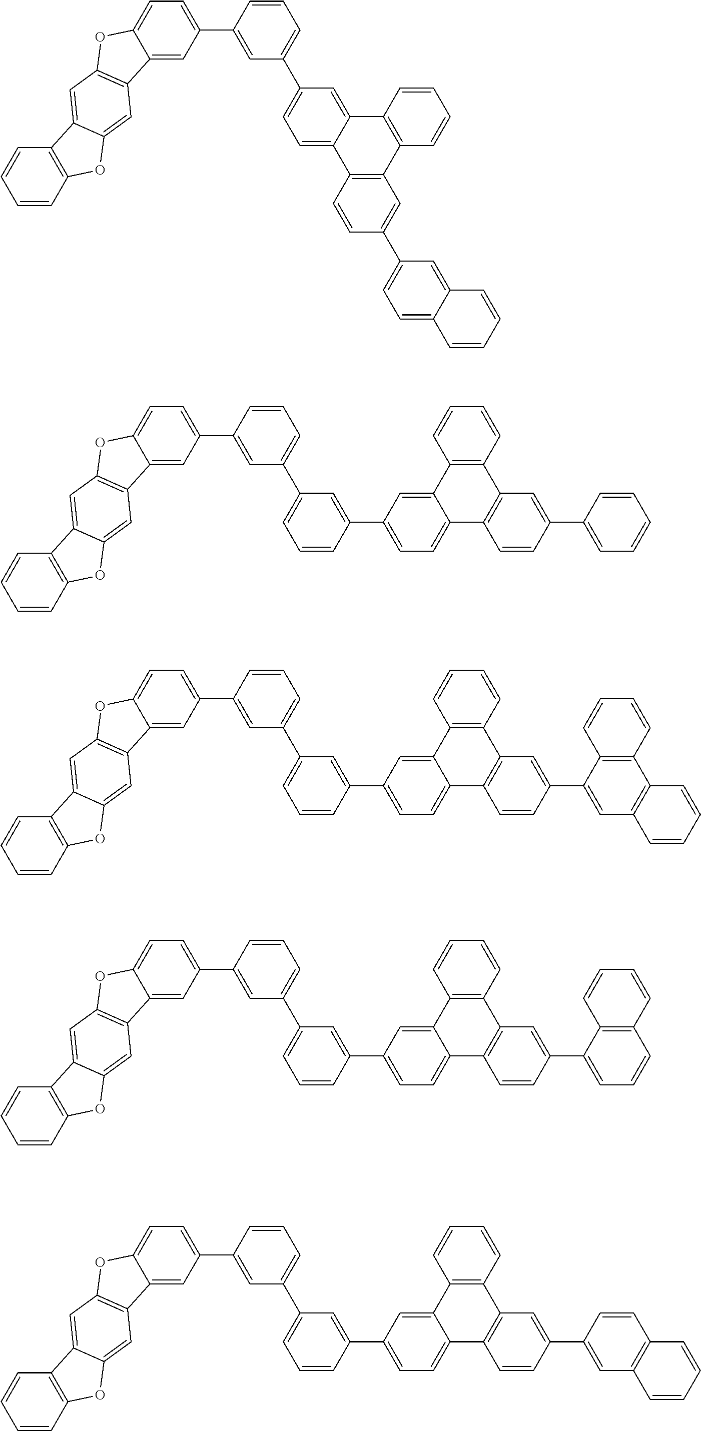

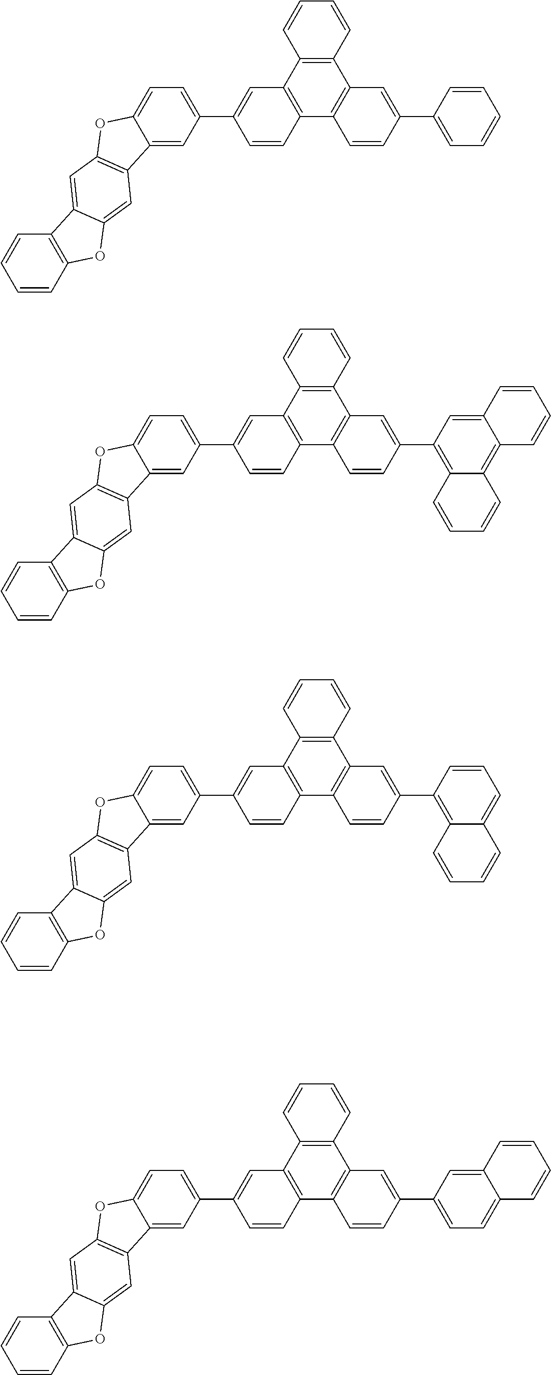 [SMILES: c1ccc(-c2ccc3c4ccc(-c5ccc6oc7cc8c(cc7c6c5)oc5ccccc58)cc4c4ccccc4c3c2)cc1 ; c1ccc2c(c1)cc(-c1ccc3c4ccc(-c5ccc6oc7cc8c(cc7c6c5)oc5ccccc58)cc4c4ccccc4c3c1)c1ccccc12 ; c1ccc2c(-c3ccc4c5ccc(-c6ccc7oc8cc9c(cc8c7c6)oc6ccccc69)cc5c5ccccc5c4c3)cccc2c1 ; c1ccc2cc(-c3ccc4c5ccc(-c6ccc7oc8cc9c(cc8c7c6)oc6ccccc69)cc5c5ccccc5c4c3)ccc2c1]

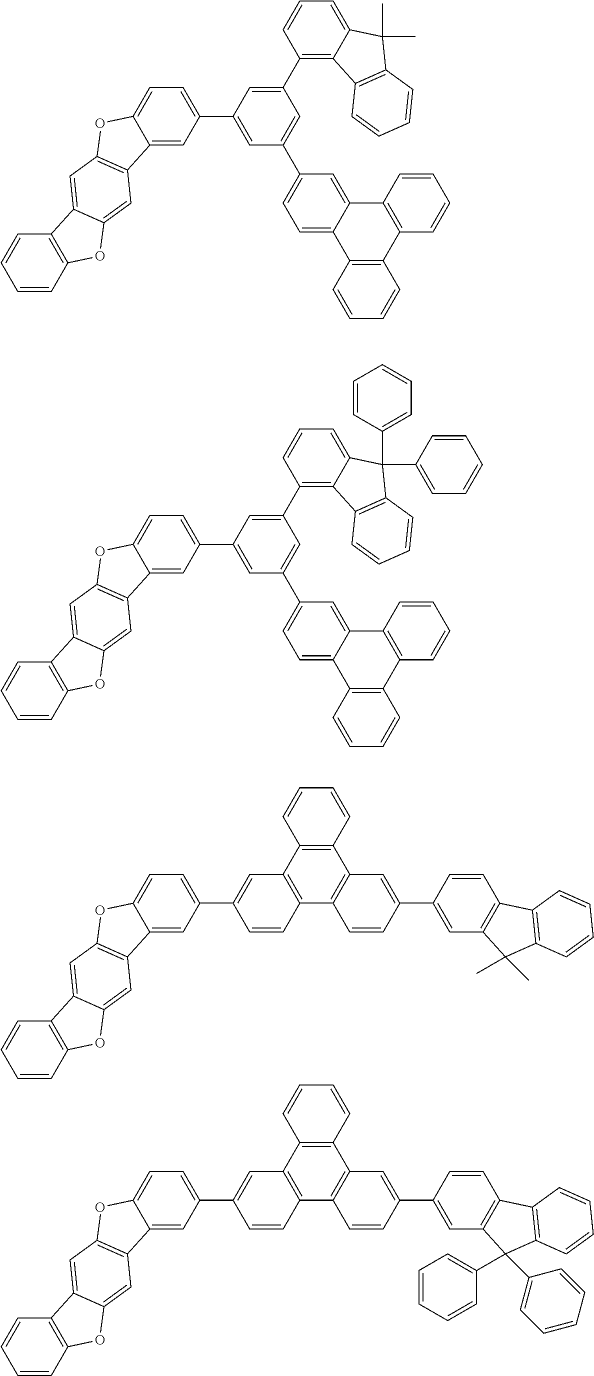

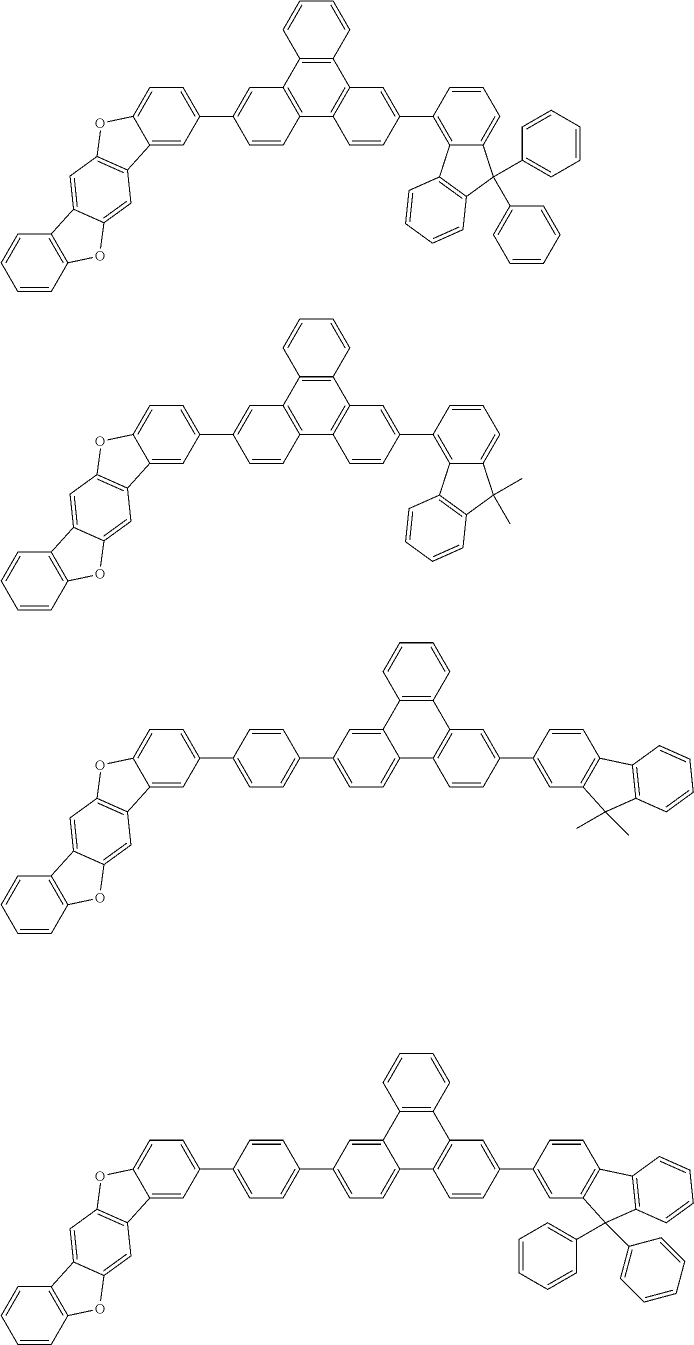 [SMILES: c1ccc(C2(c3ccccc3)c3ccccc3-c3c(-c4ccc5c6ccc(-c7ccc8oc9cc%10c(cc9c8c7)oc7ccccc7%10)cc6c6ccccc6c5c4)cccc32)cc1 ; CC1(C)c2ccccc2-c2c(-c3ccc4c5ccc(-c6ccc7oc8cc9c(cc8c7c6)oc6ccccc69)cc5c5ccccc5c4c3)cccc21 ; CC1(C)c2ccccc2-c2ccc(-c3ccc4c5ccc(-c6ccc(-c7ccc8oc9cc%10c(cc9c8c7)oc7ccccc7%10)cc6)cc5c5ccccc5c4c3)cc21 ; c1ccc(C2(c3ccccc3)c3ccccc3-c3ccc(-c4ccc5c6ccc(-c7ccc(-c8ccc9oc%10cc%11c(cc%10c9c8)oc8ccccc8%11)cc7)cc6c6ccccc6c5c4)cc32)cc1]

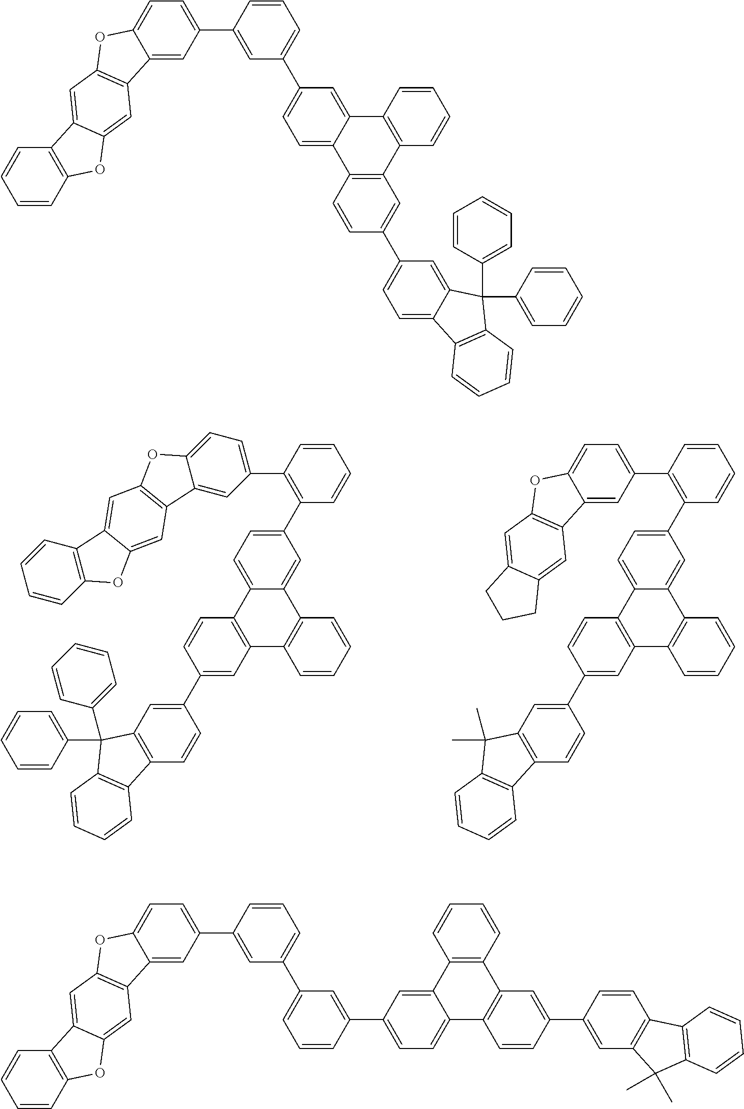 [SMILES: c1ccc(C2(c3ccccc3)c3ccccc3-c3ccc(-c4ccc5c6ccc(-c7cccc(-c8ccc9oc%10cc%11c(cc%10c9c8)oc8ccccc8%11)c7)cc6c6ccccc6c5c4)cc32)cc1 ; c1ccc(C2(c3ccccc3)c3ccccc3-c3ccc(-c4ccc5c6ccc(-c7ccccc7-c7ccc8oc9cc%10c(cc9c8c7)oc7ccccc7%10)cc6c6ccccc6c5c4)cc32)cc1 ; CC1(C)c2ccccc2-c2ccc(-c3ccc4c5ccc(-c6ccccc6-c6ccc7oc8cc9c(cc8c7c6)CCC9)cc5c5ccccc5c4c3)cc21 ; CC1(C)c2ccccc2-c2ccc(-c3ccc4c5ccc(-c6cccc(-c7cccc(-c8ccc9oc%10cc%11c(cc%10c9c8)oc8ccccc8%11)c7)c6)cc5c5ccccc5c4c3)cc21]

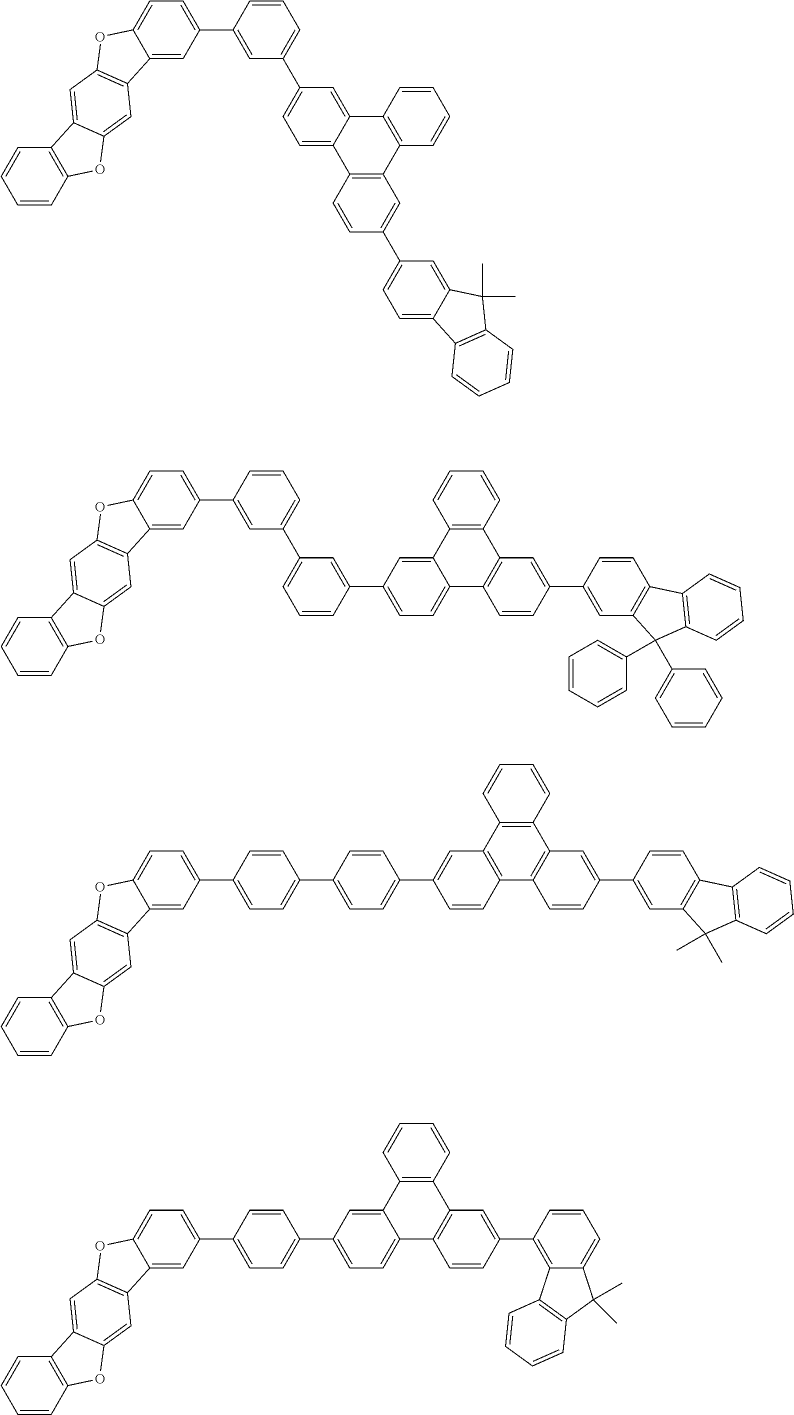 [SMILES: CC1(C)c2ccccc2-c2ccc(-c3ccc4c5ccc(-c6cccc(-c7ccc8oc9cc%10c(cc9c8c7)oc7ccccc7%10)c6)cc5c5ccccc5c4c3)cc21 ; c1ccc(C2(c3ccccc3)c3ccccc3-c3ccc(-c4ccc5c6ccc(-c7cccc(-c8cccc(-c9ccc%10oc%11cc%12c(cc%11c%10c9)oc9ccccc9%12)c8)c7)cc6c6ccccc6c5c4)cc32)cc1 ; CC1(C)c2ccccc2-c2ccc(-c3ccc4c5ccc(-c6ccc(-c7ccc(-c8ccc9oc%10cc%11c(cc%10c9c8)oc8ccccc8%11)cc7)cc6)cc5c5ccccc5c4c3)cc21 ; CC1(C)c2ccccc2-c2c(-c3ccc4c5ccc(-c6ccc(-c7ccc8oc9cc%10c(cc9c8c7)oc7ccccc7%10)cc6)cc5c5ccccc5c4c3)cccc21]

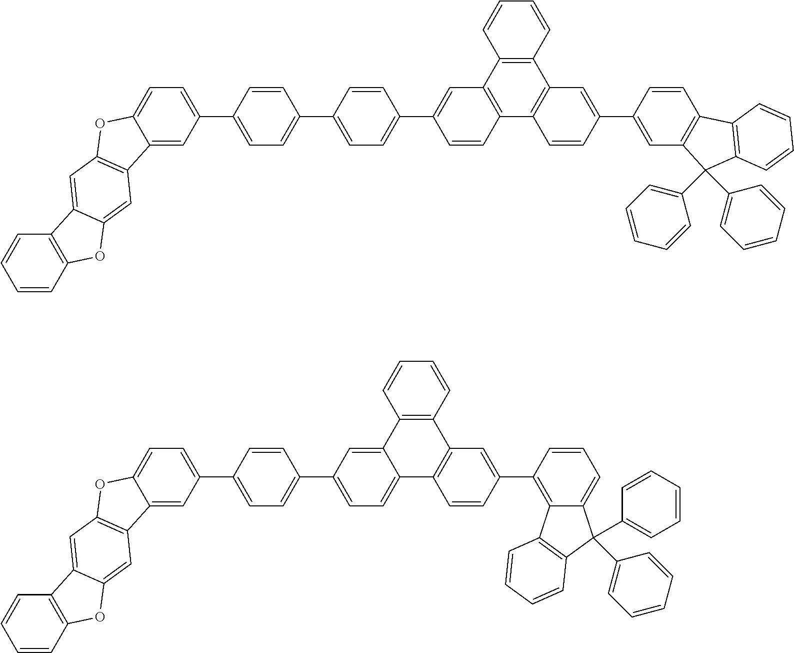 [SMILES: c1ccc(C2(c3ccccc3)c3ccccc3-c3ccc(-c4ccc5c6ccc(-c7ccc(-c8ccc(-c9ccc%10oc%11cc%12c(cc%11c%10c9)oc9ccccc9%12)cc8)cc7)cc6c6ccccc6c5c4)cc32)cc1 ; c1ccc(C2(c3ccccc3)c3ccccc3-c3c(-c4ccc5c6ccc(-c7ccc(-c8ccc9oc%10cc%11c(cc%10c9c8)oc8ccccc8%11)cc7)cc6c6ccccc6c5c4)cccc32)cc1]

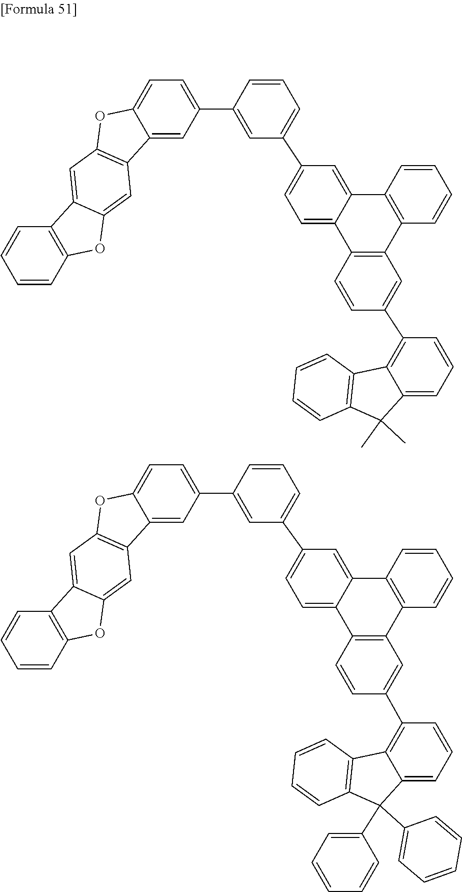 [SMILES: CC1(C)c2ccccc2-c2c(-c3ccc4c5ccc(-c6cccc(-c7ccc8oc9cc%10c(cc9c8c7)oc7ccccc7%10)c6)cc5c5ccccc5c4c3)cccc21 ; c1ccc(C2(c3ccccc3)c3ccccc3-c3c(-c4ccc5c6ccc(-c7cccc(-c8ccc9oc%10cc%11c(cc%10c9c8)oc8ccccc8%11)c7)cc6c6ccccc6c5c4)cccc32)cc1]

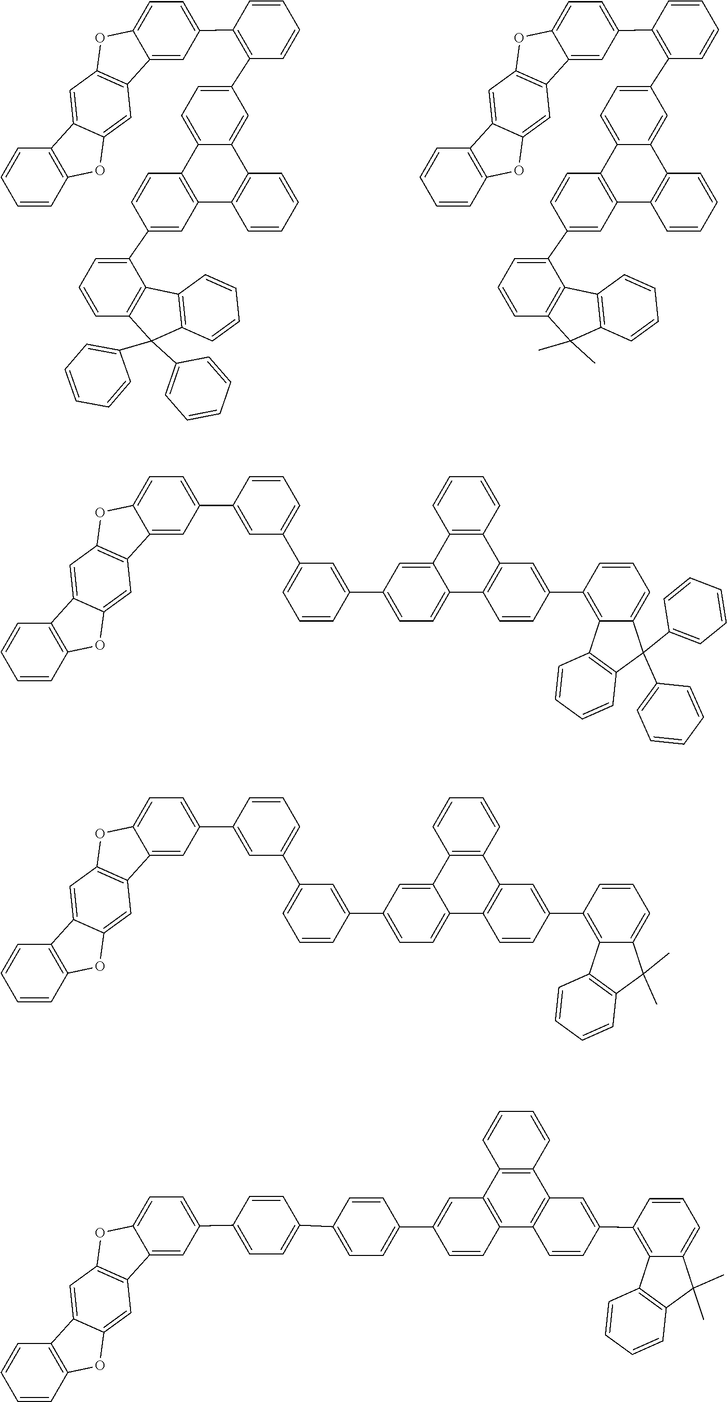

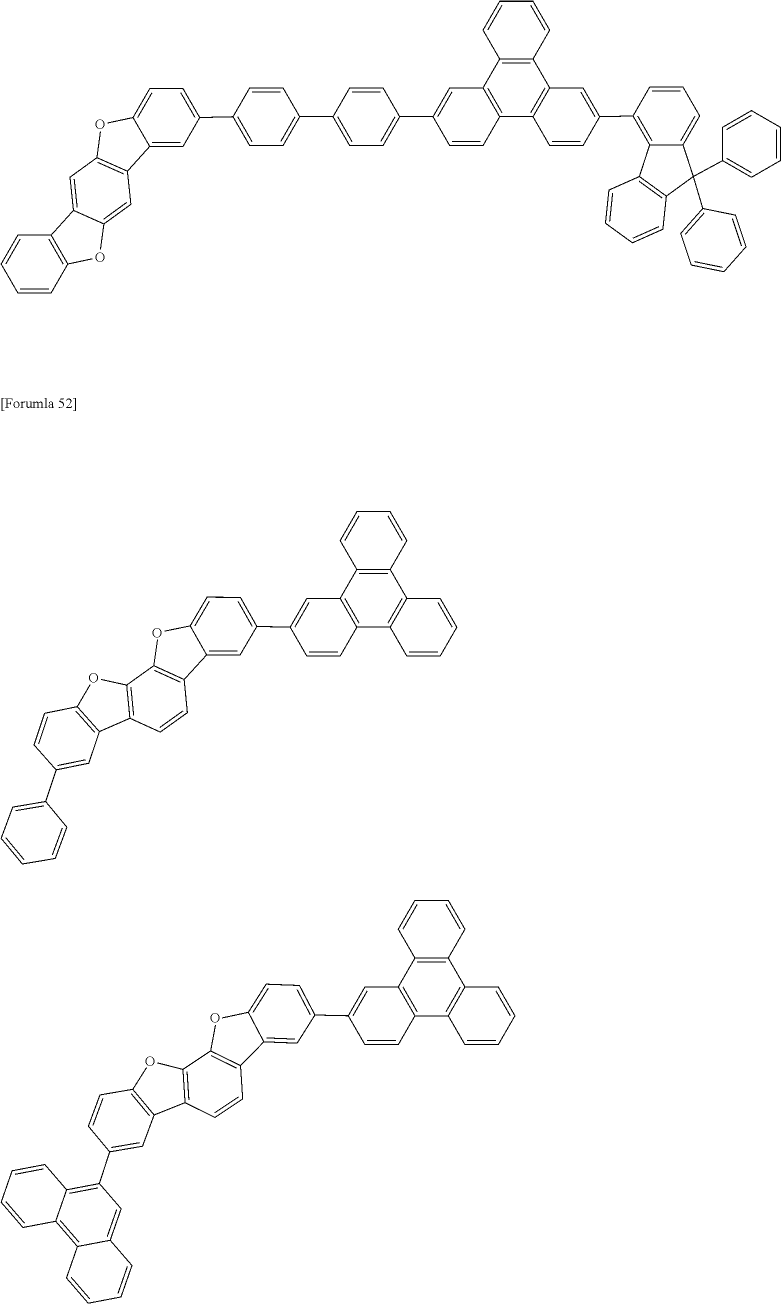 [SMILES: c1ccc(C2(c3ccccc3)c3ccccc3-c3c(-c4ccc5c6ccc(-c7ccc(-c8ccc(-c9ccc%10oc%11cc%12c(cc%11c%10c9)oc9ccccc9%12)cc8)cc7)cc6c6ccccc6c5c4)cccc32)cc1 ; c1ccc(-c2ccc3oc4c(ccc5c6cc(-c7ccc8c9ccccc9c9ccccc9c8c7)ccc6oc54)c3c2)cc1 ; c1ccc2c(c1)cc(-c1ccc3oc4c(ccc5c6cc(-c7ccc8c9ccccc9c9ccccc9c8c7)ccc6oc54)c3c1)c1ccccc12]

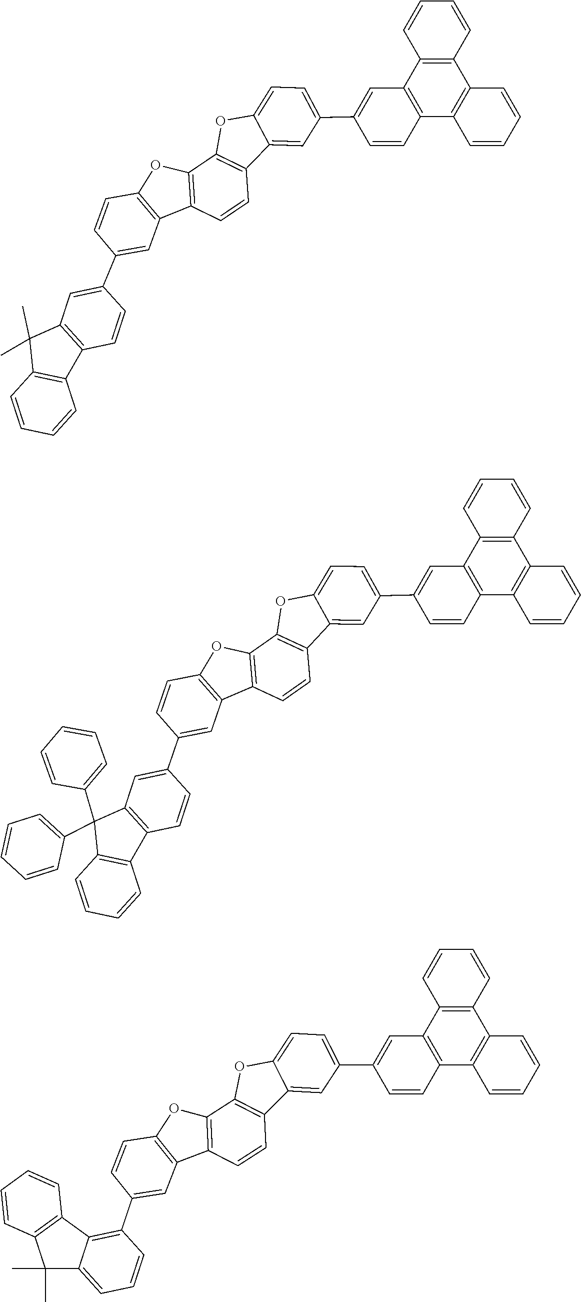 [SMILES: CC1(C)c2ccccc2-c2ccc(-c3ccc4oc5c(ccc6c7cc(-c8ccc9c%10ccccc%10c%10ccccc%10c9c8)ccc7oc65)c4c3)cc21 ; c1ccc(C2(c3ccccc3)c3ccccc3-c3ccc(-c4ccc5oc6c(ccc7c8cc(-c9ccc%10c%11ccccc%11c%11ccccc%11c%10c9)ccc8oc76)c5c4)cc32)cc1 ; CC1(C)c2ccccc2-c2c(-c3ccc4oc5c(ccc6c7cc(-c8ccc9c%10ccccc%10c%10ccccc%10c9c8)ccc7oc65)c4c3)cccc21]

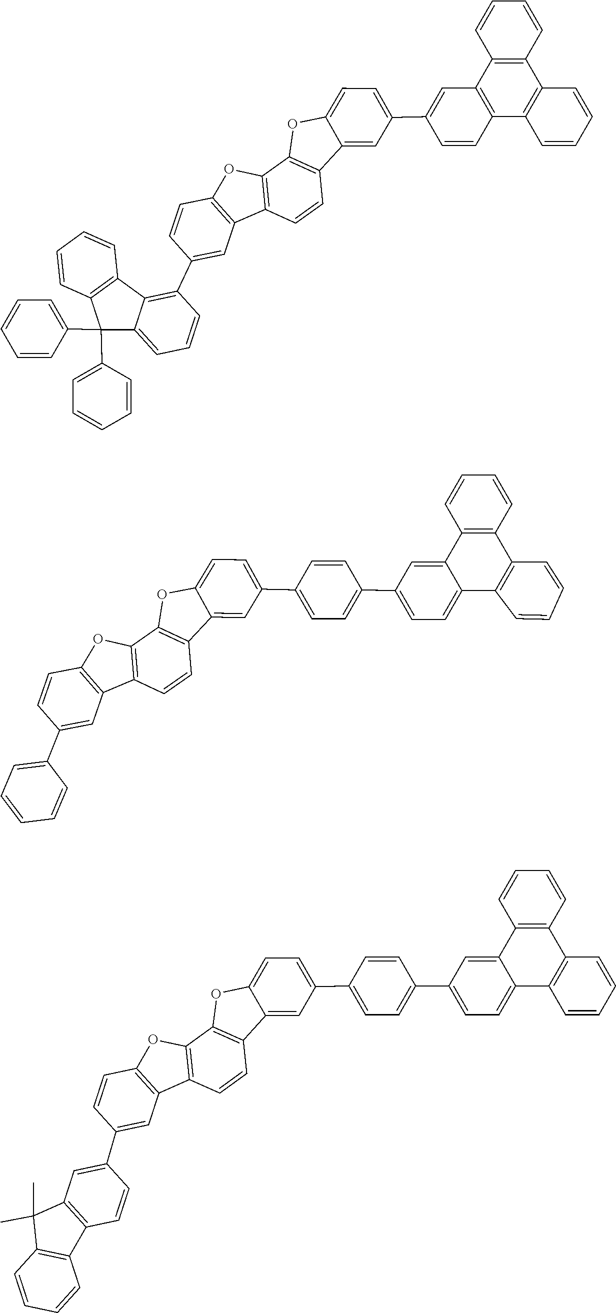 [SMILES: c1ccc(C2(c3ccccc3)c3ccccc3-c3c(-c4ccc5oc6c(ccc7c8cc(-c9ccc%10c%11ccccc%11c%11ccccc%11c%10c9)ccc8oc76)c5c4)cccc32)cc1 ; c1ccc(-c2ccc3oc4c(ccc5c6cc(-c7ccc(-c8ccc9c%10ccccc%10c%10ccccc%10c9c8)cc7)ccc6oc54)c3c2)cc1 ; CC1(C)c2ccccc2-c2ccc(-c3ccc4oc5c(ccc6c7cc(-c8ccc(-c9ccc%10c%11ccccc%11c%11ccccc%11c%10c9)cc8)ccc7oc65)c4c3)cc21]

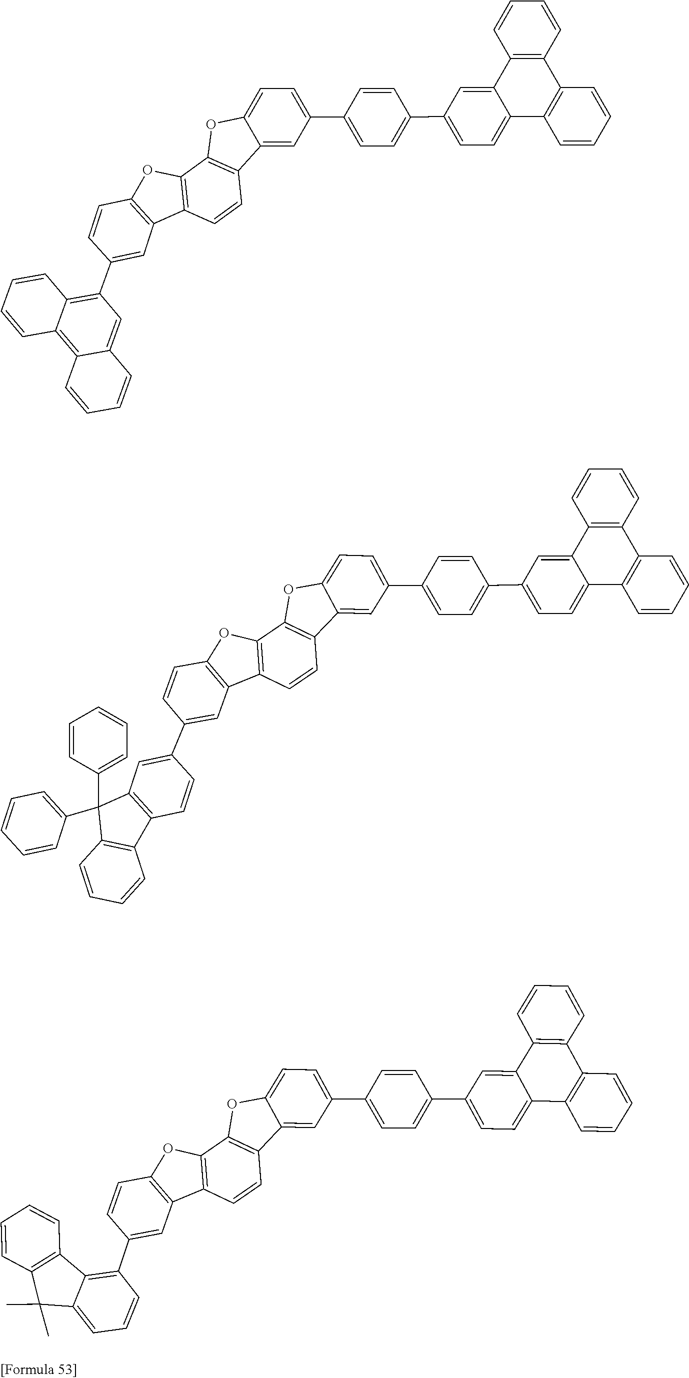 [SMILES: c1ccc2c(c1)cc(-c1ccc3oc4c(ccc5c6cc(-c7ccc(-c8ccc9c%10ccccc%10c%10ccccc%10c9c8)cc7)ccc6oc54)c3c1)c1ccccc12 ; c1ccc(C2(c3ccccc3)c3ccccc3-c3ccc(-c4ccc5oc6c(ccc7c8cc(-c9ccc(-c%10ccc%11c%12ccccc%12c%12ccccc%12c%11c%10)cc9)ccc8oc76)c5c4)cc32)cc1 ; CC1(C)c2ccccc2-c2c(-c3ccc4oc5c(ccc6c7cc(-c8ccc(-c9ccc%10c%11ccccc%11c%11ccccc%11c%10c9)cc8)ccc7oc65)c4c3)cccc21]

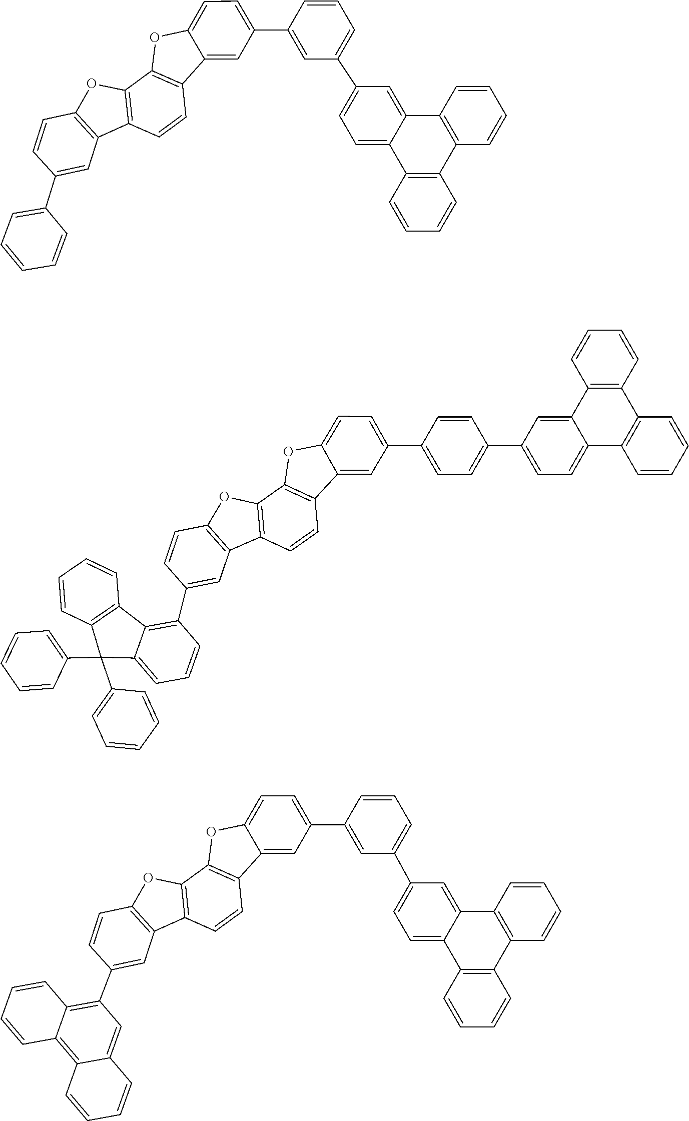 [SMILES: c1ccc(-c2ccc3oc4c(ccc5c6cc(-c7cccc(-c8ccc9c%10ccccc%10c%10ccccc%10c9c8)c7)ccc6oc54)c3c2)cc1 ; c1ccc(C2(c3ccccc3)c3ccccc3-c3c(-c4ccc5oc6c(ccc7c8cc(-c9ccc(-c%10ccc%11c%12ccccc%12c%12ccccc%12c%11c%10)cc9)ccc8oc76)c5c4)cccc32)cc1 ; c1cc(-c2ccc3oc4c(ccc5c6cc(-c7cc8ccccc8c8ccccc78)ccc6oc54)c3c2)cc(-c2ccc3c4ccccc4c4ccccc4c3c2)c1]

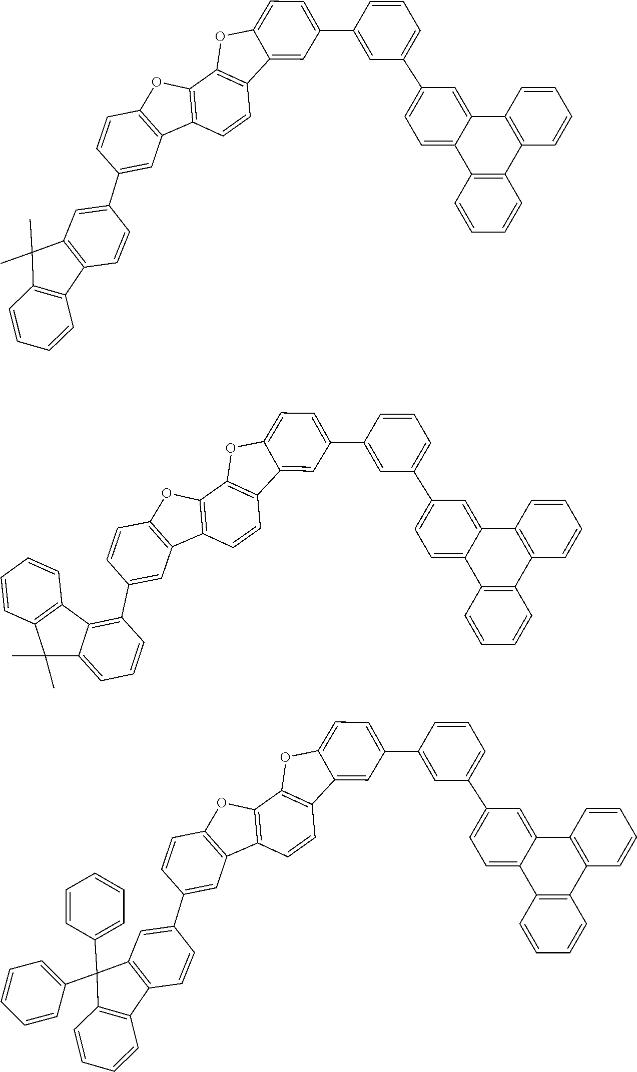 [SMILES: CC1(C)c2ccccc2-c2ccc(-c3ccc4oc5c(ccc6c7cc(-c8cccc(-c9ccc%10c%11ccccc%11c%11ccccc%11c%10c9)c8)ccc7oc65)c4c3)cc21 ; CC1(C)c2ccccc2-c2c(-c3ccc4oc5c(ccc6c7cc(-c8cccc(-c9ccc%10c%11ccccc%11c%11ccccc%11c%10c9)c8)ccc7oc65)c4c3)cccc21 ; c1ccc(C2(c3ccccc3)c3ccccc3-c3ccc(-c4ccc5oc6c(ccc7c8cc(-c9cccc(-c%10ccc%11c%12ccccc%12c%12ccccc%12c%11c%10)c9)ccc8oc76)c5c4)cc32)cc1]

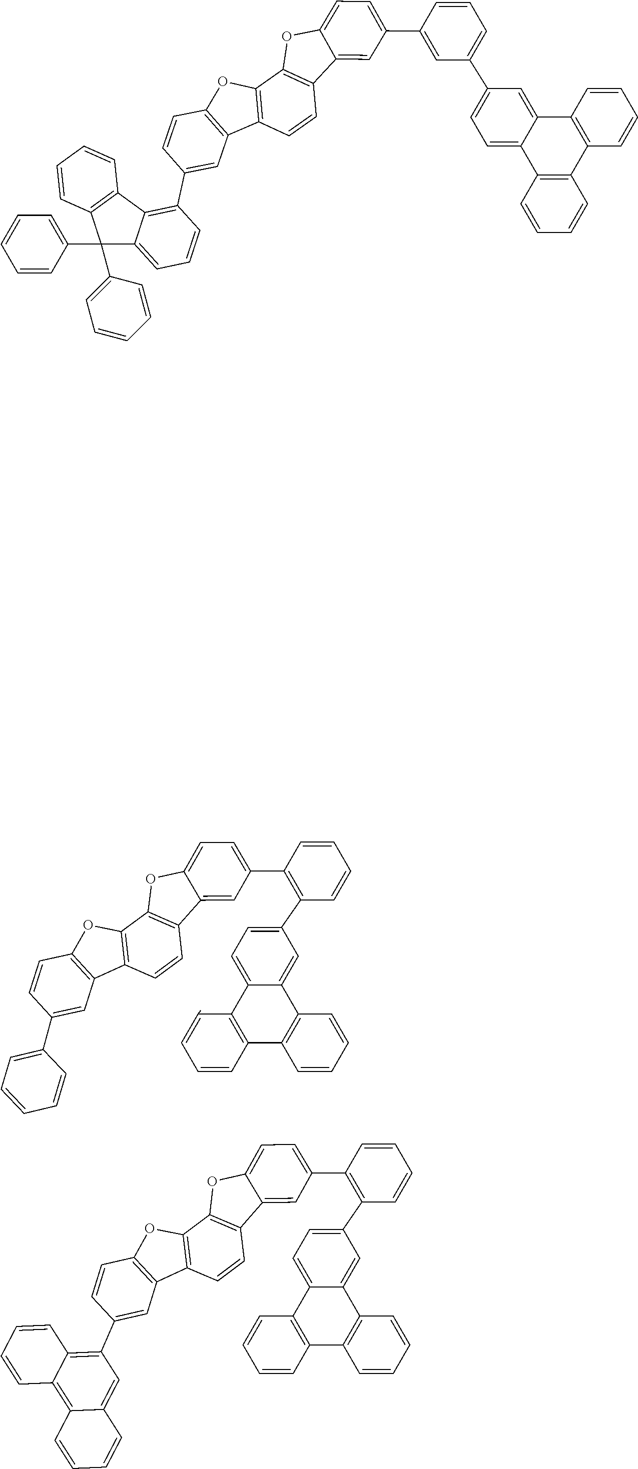 [SMILES: c1ccc(C2(c3ccccc3)c3ccccc3-c3c(-c4ccc5oc6c(ccc7c8cc(-c9cccc(-c%10ccc%11c%12ccccc%12c%12ccccc%12c%11c%10)c9)ccc8oc76)c5c4)cccc32)cc1 ; Cc1cccc2c3ccccc3c3cc(-c4ccccc4-c4ccc5oc6c(ccc7c8cc(-c9ccccc9)ccc8oc76)c5c4)ccc3c12 ; c1ccc(-c2ccc3c4ccccc4c4ccccc4c3c2)c(-c2ccc3oc4c(ccc5c6cc(-c7cc8ccccc8c8ccccc78)ccc6oc54)c3c2)c1]

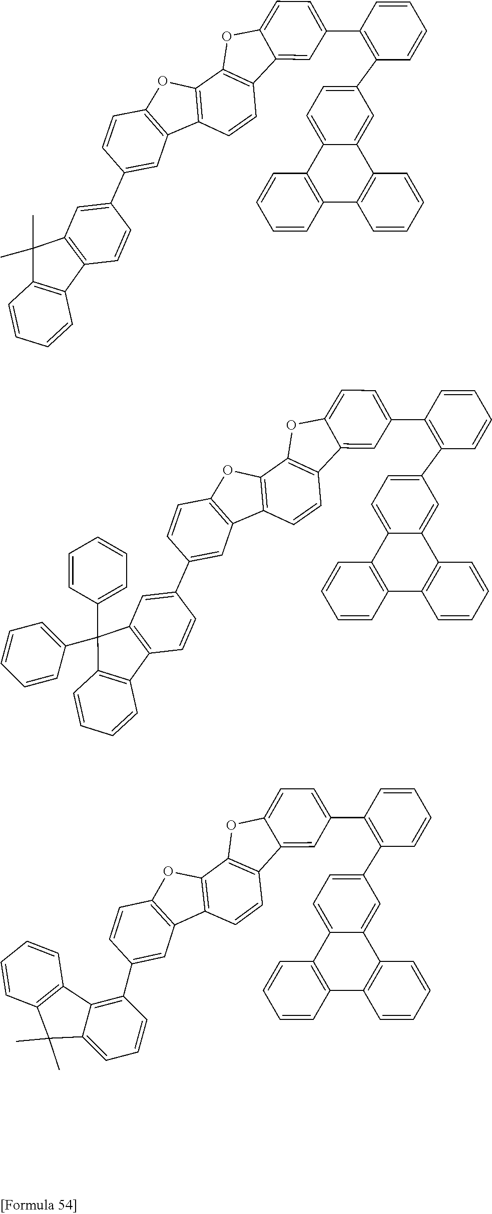 [SMILES: CC1(C)c2ccccc2-c2ccc(-c3ccc4oc5c(ccc6c7cc(-c8ccccc8-c8ccc9c%10ccccc%10c%10ccccc%10c9c8)ccc7oc65)c4c3)cc21 ; c1ccc(C2(c3ccccc3)c3ccccc3-c3ccc(-c4ccc5oc6c(ccc7c8cc(-c9ccccc9-c9ccc%10c%11ccccc%11c%11ccccc%11c%10c9)ccc8oc76)c5c4)cc32)cc1 ; CC1(C)c2ccccc2-c2c(-c3ccc4oc5c(ccc6c7cc(-c8ccccc8-c8ccc9c%10ccccc%10c%10ccccc%10c9c8)ccc7oc65)c4c3)cccc21]

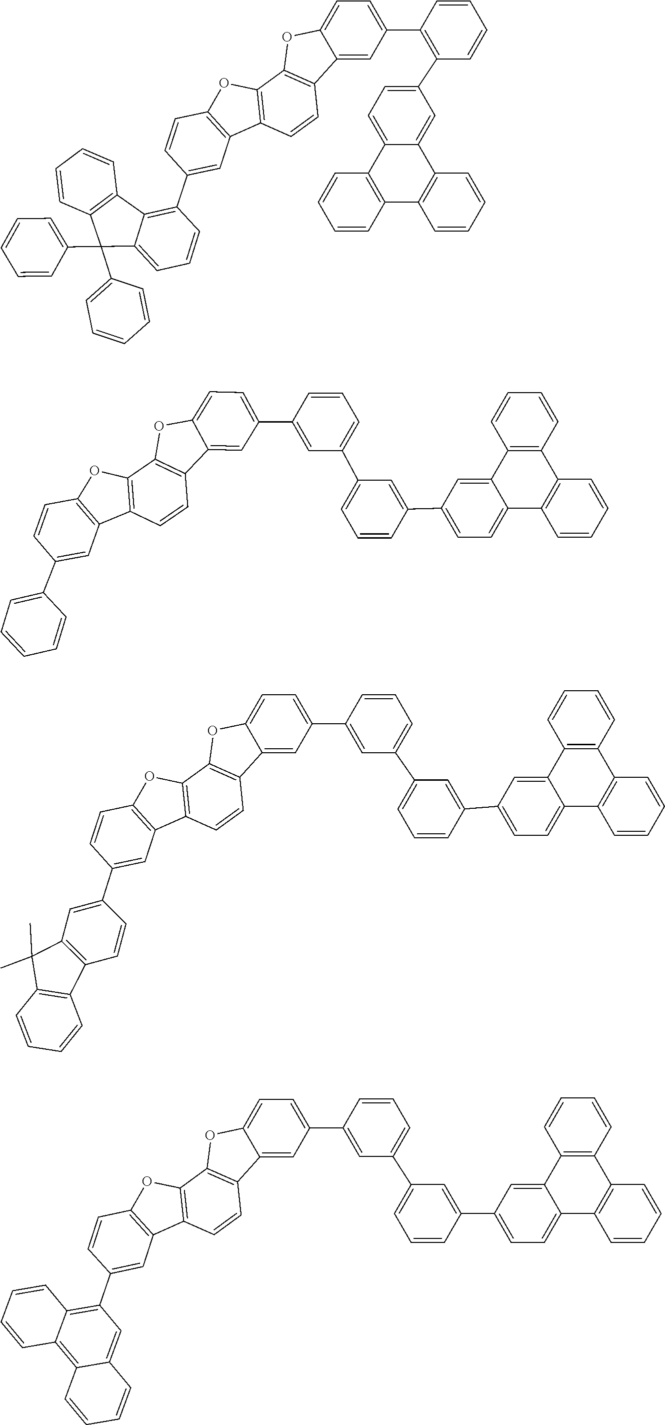 [SMILES: c1ccc(C2(c3ccccc3)c3ccccc3-c3c(-c4ccc5oc6c(ccc7c8cc(-c9ccccc9-c9ccc%10c%11ccccc%11c%11ccccc%11c%10c9)ccc8oc76)c5c4)cccc32)cc1 ; c1ccc(-c2ccc3oc4c(ccc5c6cc(-c7cccc(-c8cccc(-c9ccc%10c%11ccccc%11c%11ccccc%11c%10c9)c8)c7)ccc6oc54)c3c2)cc1 ; CC1(C)c2ccccc2-c2ccc(-c3ccc4oc5c(ccc6c7cc(-c8cccc(-c9cccc(-c%10ccc%11c%12ccccc%12c%12ccccc%12c%11c%10)c9)c8)ccc7oc65)c4c3)cc21 ; c1cc(-c2cccc(-c3ccc4c5ccccc5c5ccccc5c4c3)c2)cc(-c2ccc3oc4c(ccc5c6cc(-c7cc8ccccc8c8ccccc78)ccc6oc54)c3c2)c1]

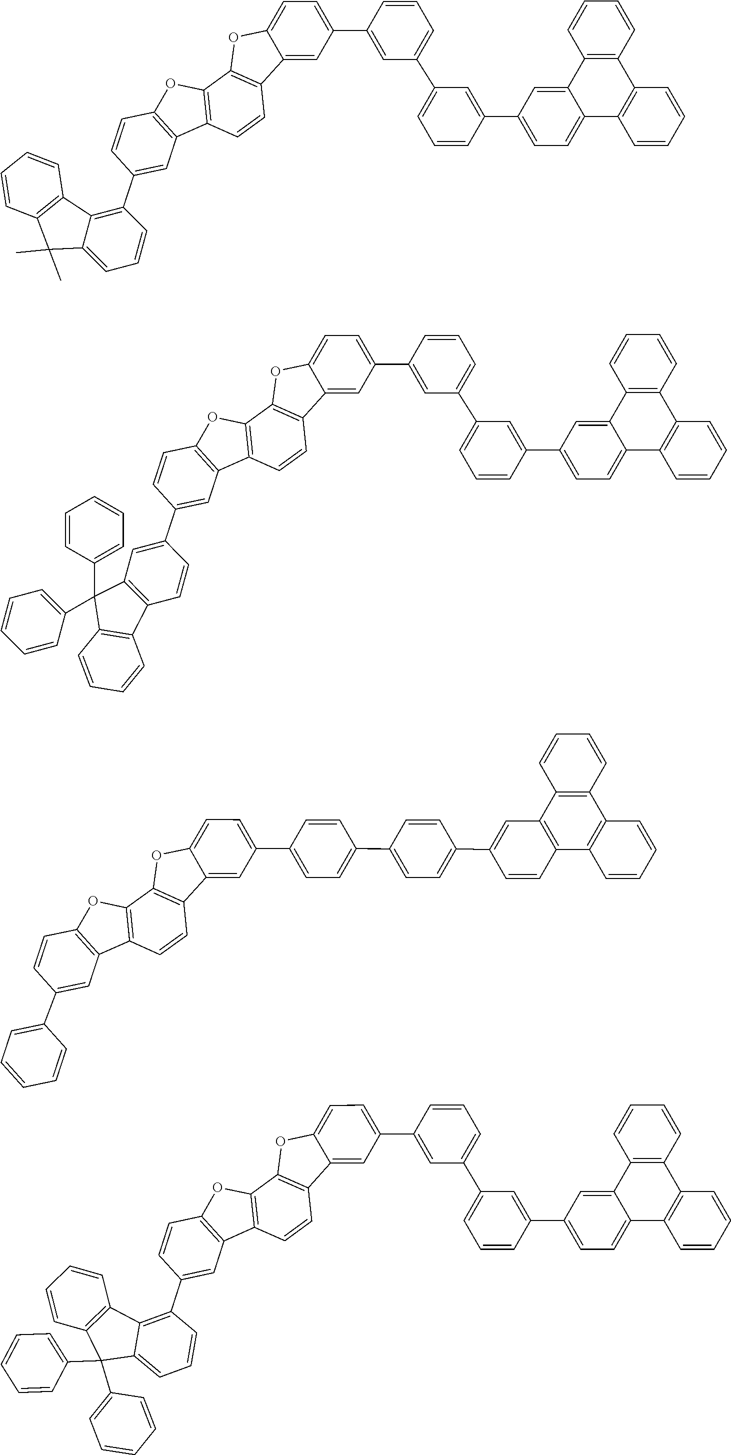

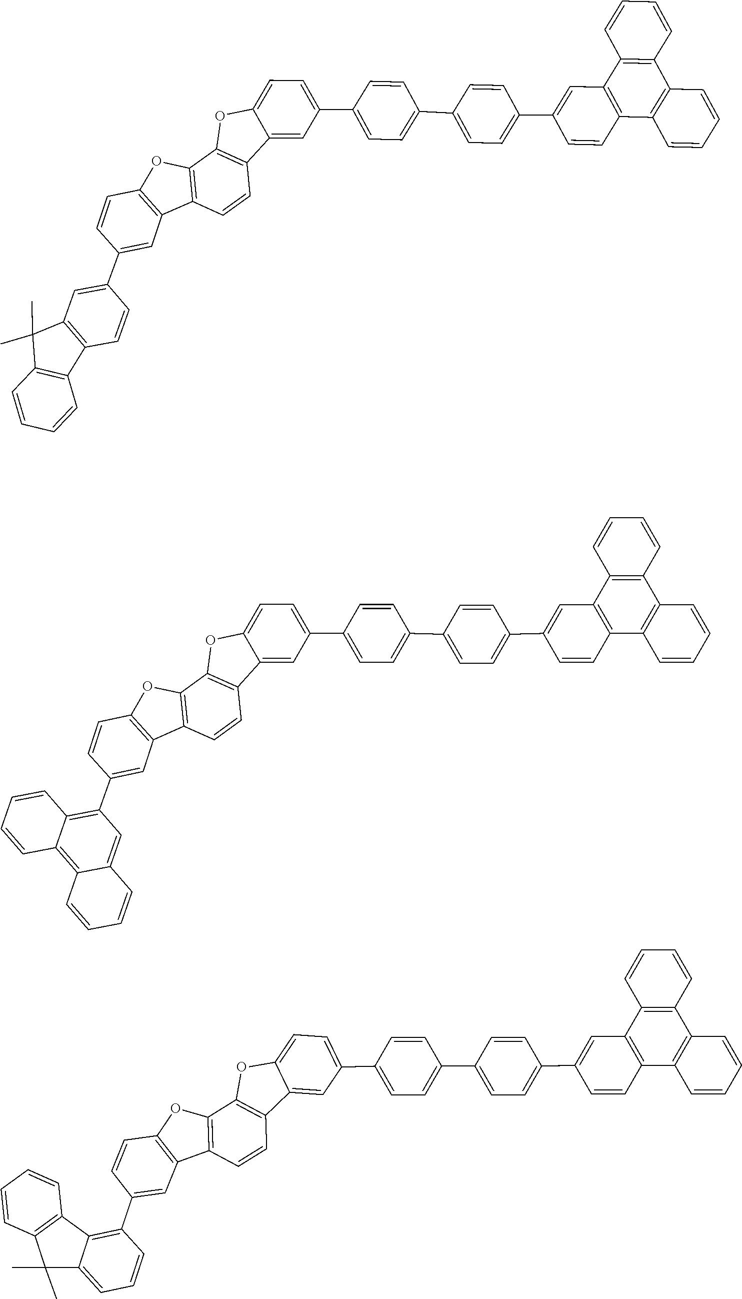 [SMILES: CC1(C)c2ccccc2-c2ccc(-c3ccc4oc5c(ccc6c7cc(-c8ccc(-c9ccc(-c%10ccc%11c%12ccccc%12c%12ccccc%12c%11c%10)cc9)cc8)ccc7oc65)c4c3)cc21 ; c1ccc2c(c1)cc(-c1ccc3oc4c(ccc5c6cc(-c7ccc(-c8ccc(-c9ccc%10c%11ccccc%11c%11ccccc%11c%10c9)cc8)cc7)ccc6oc54)c3c1)c1ccccc12 ; CC1(C)c2ccccc2-c2c(-c3ccc4oc5c(ccc6c7cc(-c8ccc(-c9ccc(-c%10ccc%11c%12ccccc%12c%12ccccc%12c%11c%10)cc9)cc8)ccc7oc65)c4c3)cccc21]

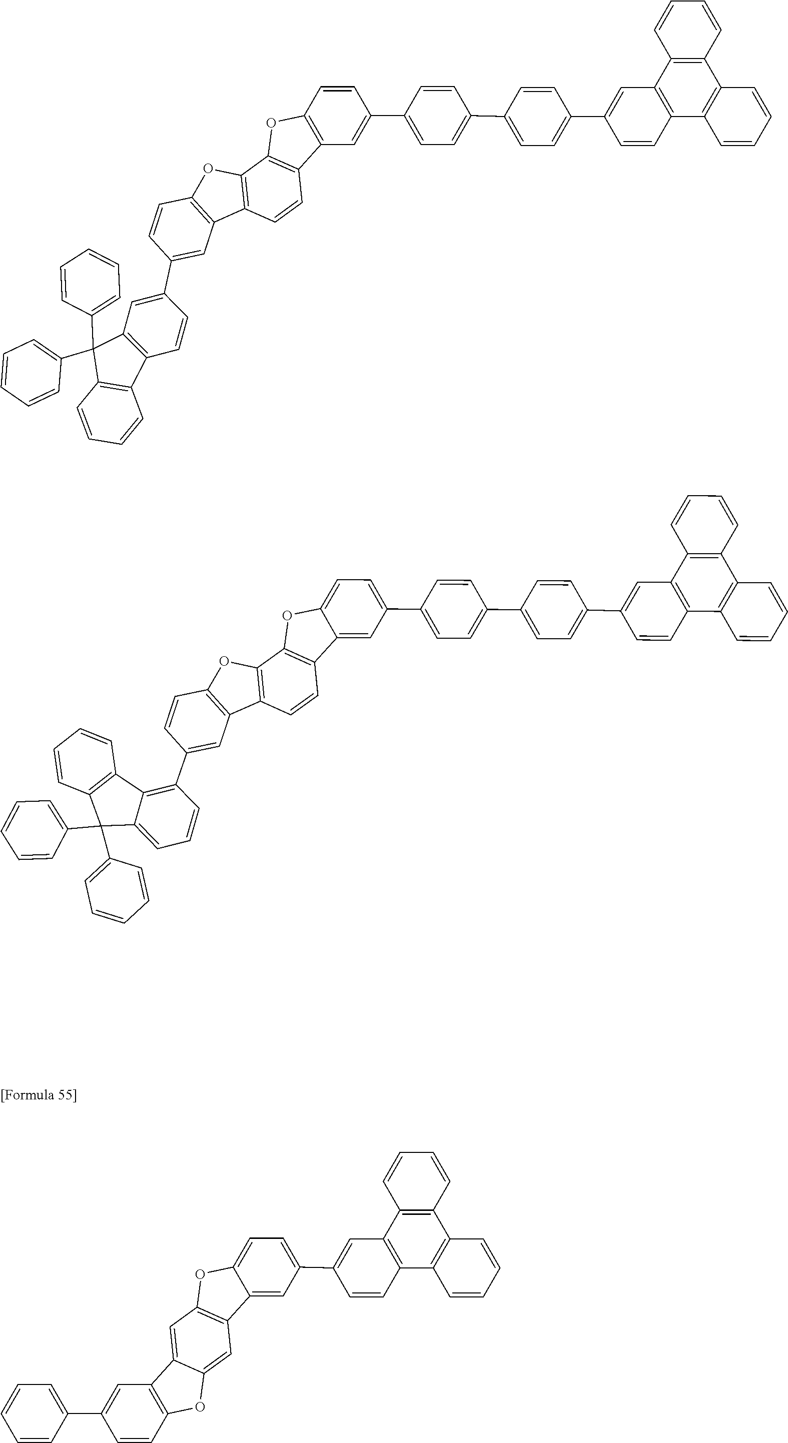 [SMILES: c1ccc(C2(c3ccccc3)c3ccccc3-c3ccc(-c4ccc5oc6c(ccc7c8cc(-c9ccc(-c%10ccc(-c%11ccc%12c%13ccccc%13c%13ccccc%13c%12c%11)cc%10)cc9)ccc8oc76)c5c4)cc32)cc1 ; c1ccc(C2(c3ccccc3)c3ccccc3-c3c(-c4ccc5oc6c(ccc7c8cc(-c9ccc(-c%10ccc(-c%11ccc%12c%13ccccc%13c%13ccccc%13c%12c%11)cc%10)cc9)ccc8oc76)c5c4)cccc32)cc1 ; c1ccc(-c2ccc3oc4cc5c(cc4c3c2)oc2ccc(-c3ccc4c6ccccc6c6ccccc6c4c3)cc25)cc1]

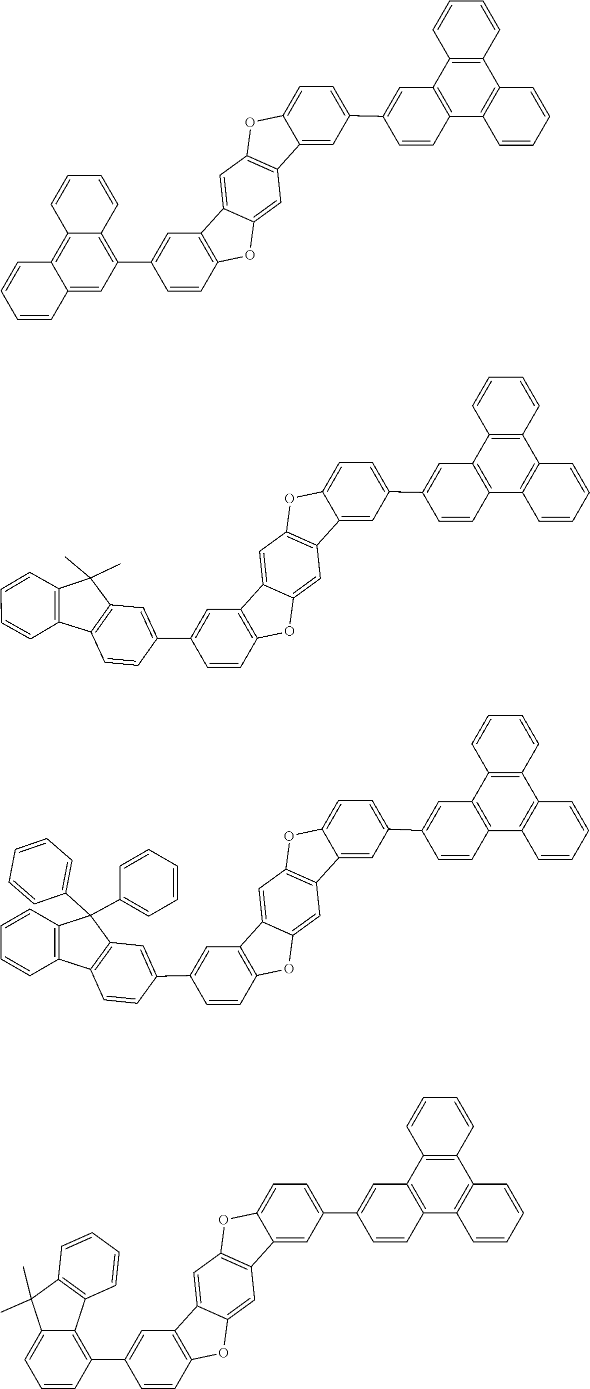 [SMILES: c1ccc2c(c1)cc(-c1ccc3oc4cc5c(cc4c3c1)oc1ccc(-c3ccc4c6ccccc6c6ccccc6c4c3)cc15)c1ccccc12 ; CC1(C)c2ccccc2-c2ccc(-c3ccc4oc5cc6c(cc5c4c3)oc3ccc(-c4ccc5c7ccccc7c7ccccc7c5c4)cc36)cc21 ; c1ccc(C2(c3ccccc3)c3ccccc3-c3ccc(-c4ccc5oc6cc7c(cc6c5c4)oc4ccc(-c5ccc6c8ccccc8c8ccccc8c6c5)cc47)cc32)cc1 ; CC1(C)c2ccccc2-c2c(-c3ccc4oc5cc6c(cc5c4c3)oc3ccc(-c4ccc5c7ccccc7c7ccccc7c5c4)cc36)cccc21]

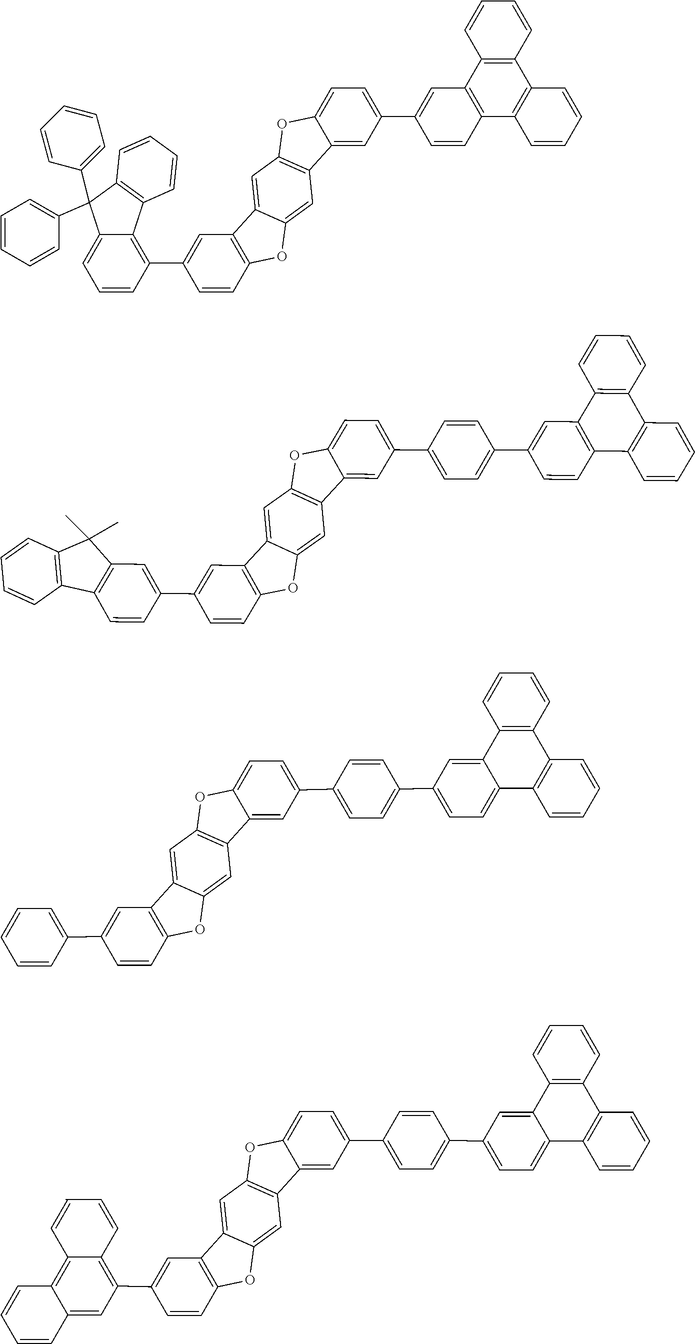 [SMILES: c1ccc(C2(c3ccccc3)c3ccccc3-c3c(-c4ccc5oc6cc7c(cc6c5c4)oc4ccc(-c5ccc6c8ccccc8c8ccccc8c6c5)cc47)cccc32)cc1 ; CC1(C)c2ccccc2-c2ccc(-c3ccc4oc5cc6c(cc5c4c3)oc3ccc(-c4ccc(-c5ccc7c8ccccc8c8ccccc8c7c5)cc4)cc36)cc21 ; c1ccc(-c2ccc3oc4cc5c(cc4c3c2)oc2ccc(-c3ccc(-c4ccc6c7ccccc7c7ccccc7c6c4)cc3)cc25)cc1 ; Cc1ccc2c(-c3ccc4oc5cc6c(cc5c4c3)oc3ccc(-c4ccc(-c5ccc7c8ccccc8c8ccccc8c7c5)cc4)cc36)cc3ccccc3c2c1]

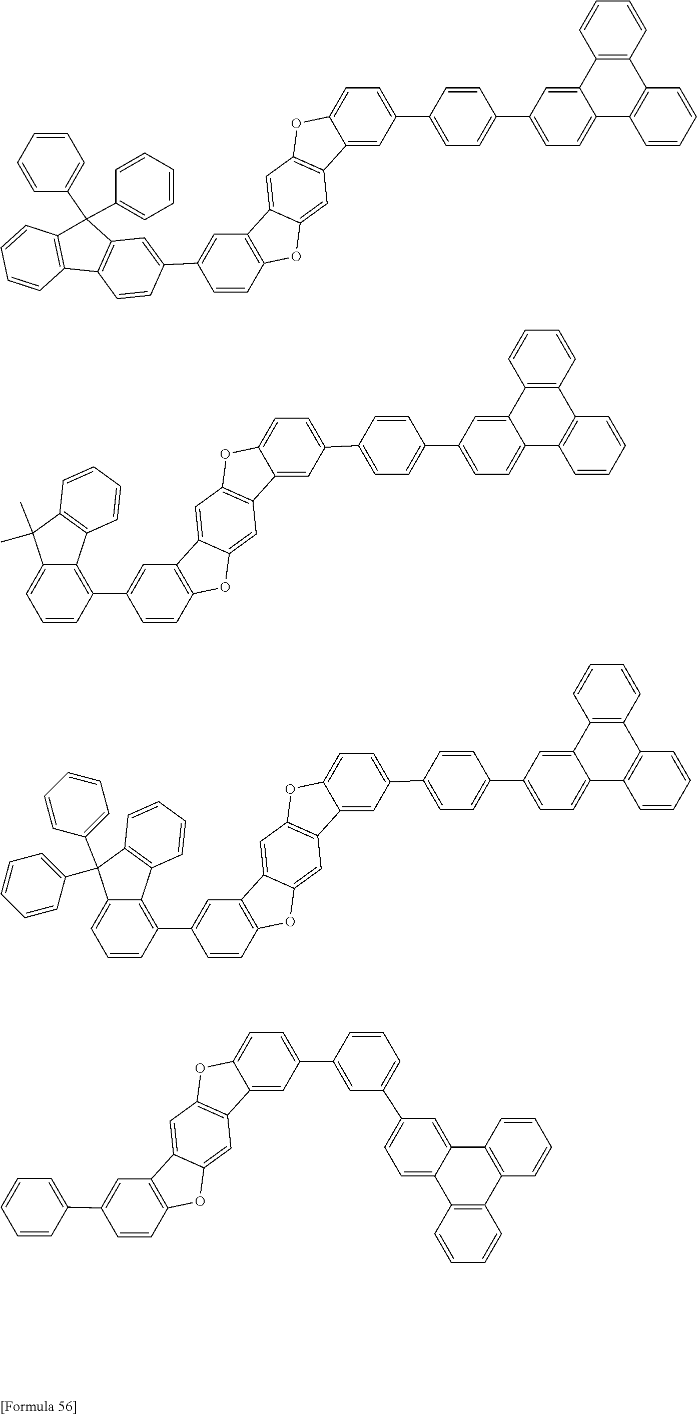 [SMILES: c1ccc(C2(c3ccccc3)c3ccccc3-c3ccc(-c4ccc5oc6cc7c(cc6c5c4)oc4ccc(-c5ccc(-c6ccc8c9ccccc9c9ccccc9c8c6)cc5)cc47)cc32)cc1 ; CC1(C)c2ccccc2-c2c(-c3ccc4oc5cc6c(cc5c4c3)oc3ccc(-c4ccc(-c5ccc7c8ccccc8c8ccccc8c7c5)cc4)cc36)cccc21 ; c1ccc(C2(c3ccccc3)c3ccccc3-c3c(-c4ccc5oc6cc7c(cc6c5c4)oc4ccc(-c5ccc(-c6ccc8c9ccccc9c9ccccc9c8c6)cc5)cc47)cccc32)cc1 ; c1ccc(-c2ccc3oc4cc5c(cc4c3c2)oc2ccc(-c3cccc(-c4ccc6c7ccccc7c7ccccc7c6c4)c3)cc25)cc1]

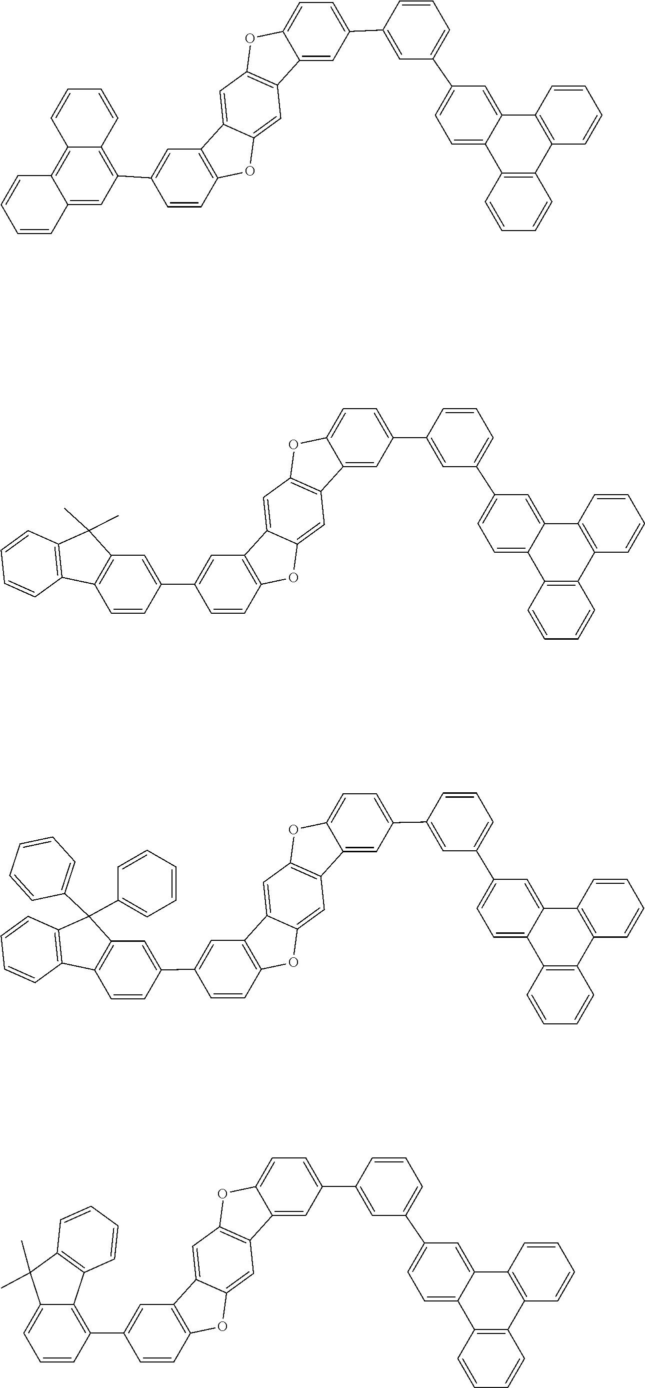 [SMILES: c1cc(-c2ccc3oc4cc5c(cc4c3c2)oc2ccc(-c3cc4ccccc4c4ccccc34)cc25)cc(-c2ccc3c4ccccc4c4ccccc4c3c2)c1 ; CC1(C)c2ccccc2-c2ccc(-c3ccc4oc5cc6c(cc5c4c3)oc3ccc(-c4cccc(-c5ccc7c8ccccc8c8ccccc8c7c5)c4)cc36)cc21 ; c1ccc(C2(c3ccccc3)c3ccccc3-c3ccc(-c4ccc5oc6cc7c(cc6c5c4)oc4ccc(-c5cccc(-c6ccc8c9ccccc9c9ccccc9c8c6)c5)cc47)cc32)cc1 ; CC1(C)c2ccccc2-c2c(-c3ccc4oc5cc6c(cc5c4c3)oc3ccc(-c4cccc(-c5ccc7c8ccccc8c8ccccc8c7c5)c4)cc36)cccc21]

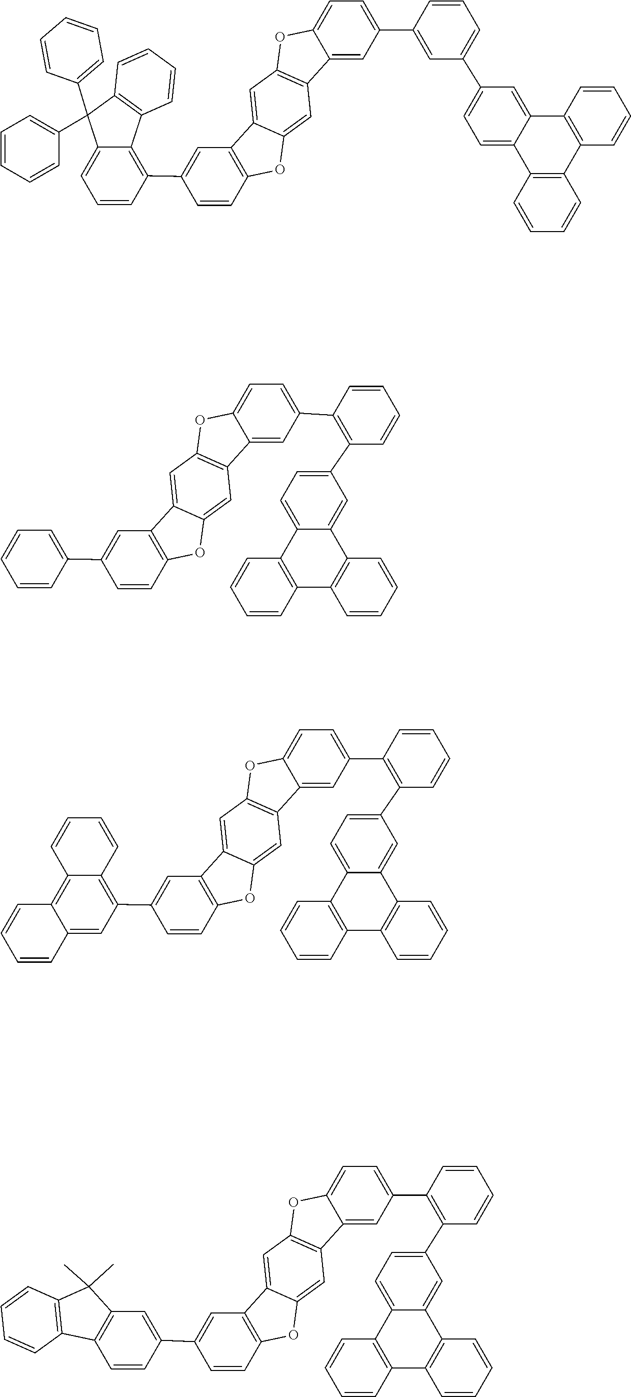 [SMILES: c1ccc(C2(c3ccccc3)c3ccccc3-c3c(-c4ccc5oc6cc7c(cc6c5c4)oc4ccc(-c5cccc(-c6ccc8c9ccccc9c9ccccc9c8c6)c5)cc47)cccc32)cc1 ; c1ccc(-c2ccc3oc4cc5c(cc4c3c2)oc2ccc(-c3ccccc3-c3ccc4c6ccccc6c6ccccc6c4c3)cc25)cc1 ; c1ccc(-c2ccc3c4ccccc4c4ccccc4c3c2)c(-c2ccc3oc4cc5c(cc4c3c2)oc2ccc(-c3cc4ccccc4c4ccccc34)cc25)c1 ; CC1(C)c2ccccc2-c2ccc(-c3ccc4oc5cc6c(cc5c4c3)oc3ccc(-c4ccccc4-c4ccc5c7ccccc7c7ccccc7c5c4)cc36)cc21]

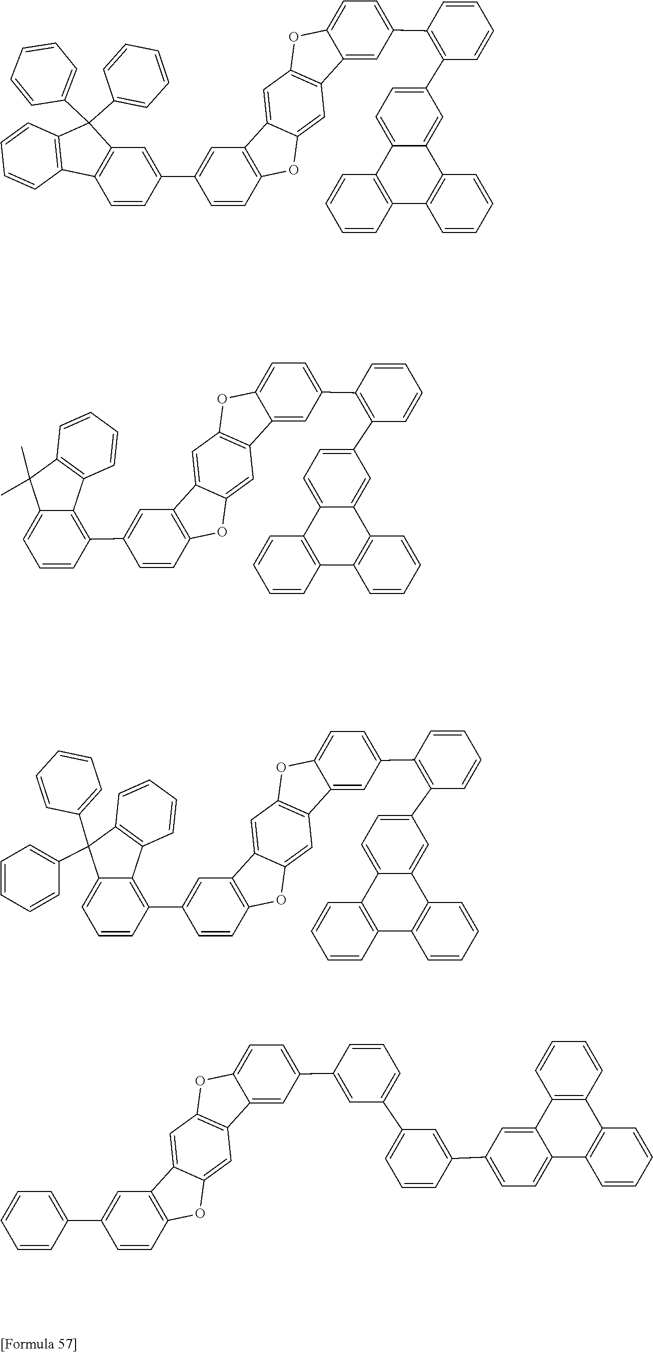 [SMILES: c1ccc(C2(c3ccccc3)c3ccccc3-c3ccc(-c4ccc5oc6cc7c(cc6c5c4)oc4ccc(-c5ccccc5-c5ccc6c8ccccc8c8ccccc8c6c5)cc47)cc32)cc1 ; CC1(C)c2ccccc2-c2c(-c3ccc4oc5cc6c(cc5c4c3)oc3ccc(-c4ccccc4-c4ccc5c7ccccc7c7ccccc7c5c4)cc36)cccc21 ; c1ccc(C2(c3ccccc3)c3ccccc3-c3c(-c4ccc5oc6cc7c(cc6c5c4)oc4ccc(-c5ccccc5-c5ccc6c8ccccc8c8ccccc8c6c5)cc47)cccc32)cc1 ; c1ccc(-c2ccc3oc4cc5c(cc4c3c2)oc2ccc(-c3cccc(-c4cccc(-c6ccc7c8ccccc8c8ccccc8c7c6)c4)c3)cc25)cc1]

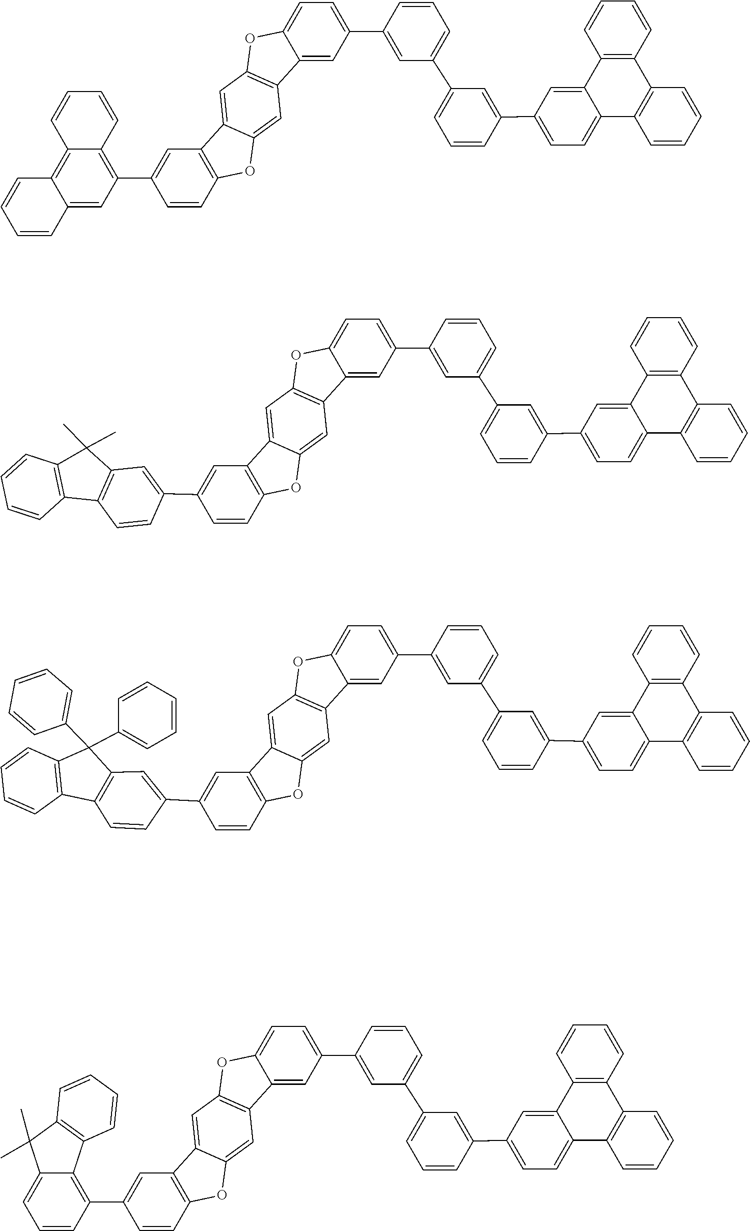 [SMILES: c1cc(-c2cccc(-c3ccc4c5ccccc5c5ccccc5c4c3)c2)cc(-c2ccc3oc4cc5c(cc4c3c2)oc2ccc(-c3cc4ccccc4c4ccccc34)cc25)c1 ; CC1(C)c2ccccc2-c2ccc(-c3ccc4oc5cc6c(cc5c4c3)oc3ccc(-c4cccc(-c5cccc(-c7ccc8c9ccccc9c9ccccc9c8c7)c5)c4)cc36)cc21 ; c1ccc(C2(c3ccccc3)c3ccccc3-c3ccc(-c4ccc5oc6cc7c(cc6c5c4)oc4ccc(-c5cccc(-c6cccc(-c8ccc9c%10ccccc%10c%10ccccc%10c9c8)c6)c5)cc47)cc32)cc1 ; CC1(C)c2ccccc2-c2c(-c3ccc4oc5cc6c(cc5c4c3)oc3ccc(-c4cccc(-c5cccc(-c7ccc8c9ccccc9c9ccccc9c8c7)c5)c4)cc36)cccc21]

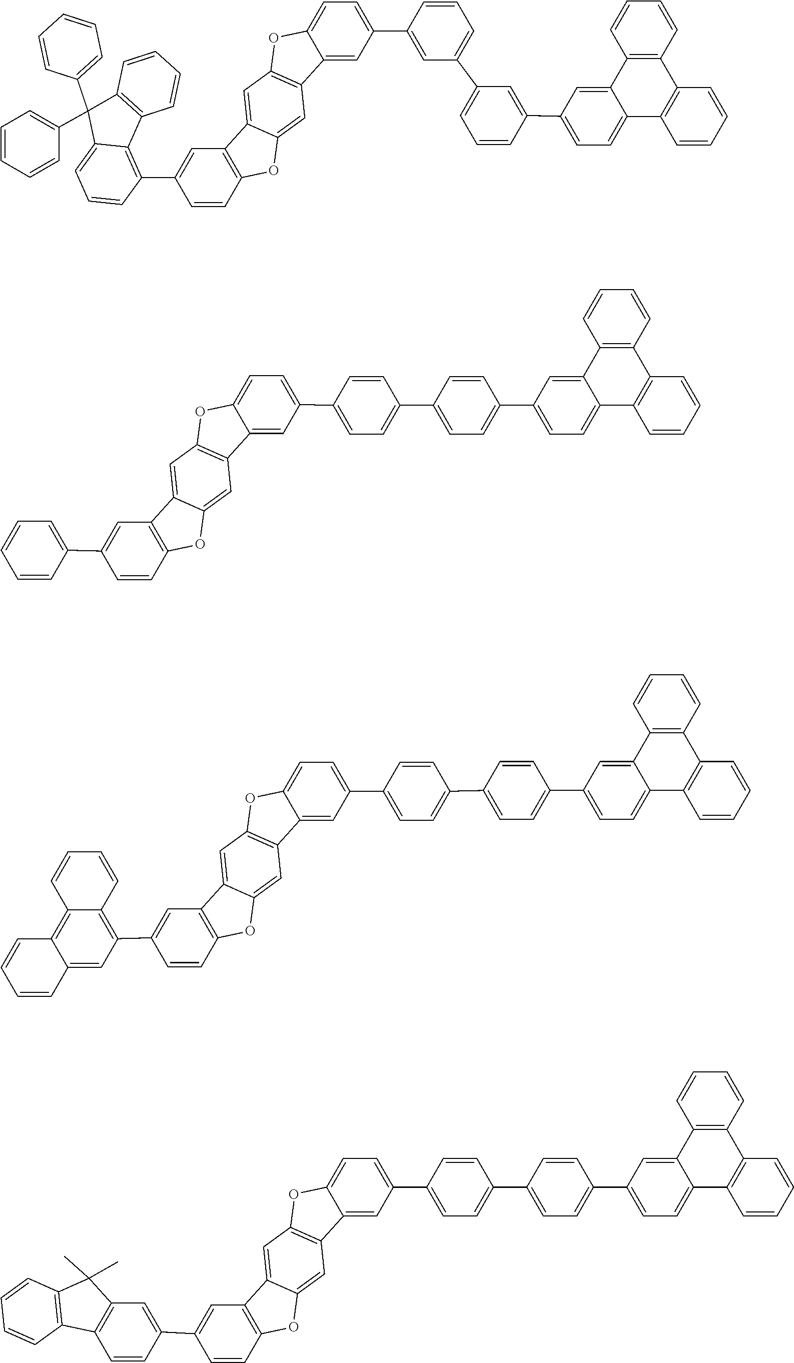 [SMILES: c1ccc(C2(c3ccccc3)c3ccccc3-c3c(-c4ccc5oc6cc7c(cc6c5c4)oc4ccc(-c5cccc(-c6cccc(-c8ccc9c%10ccccc%10c%10ccccc%10c9c8)c6)c5)cc47)cccc32)cc1 ; c1ccc(-c2ccc3oc4cc5c(cc4c3c2)oc2ccc(-c3ccc(-c4ccc(-c6ccc7c8ccccc8c8ccccc8c7c6)cc4)cc3)cc25)cc1 ; c1ccc2c(c1)cc(-c1ccc3oc4cc5c(cc4c3c1)oc1ccc(-c3ccc(-c4ccc(-c6ccc7c8ccccc8c8ccccc8c7c6)cc4)cc3)cc15)c1ccccc12 ; CC1(C)c2ccccc2-c2ccc(-c3ccc4oc5cc6c(cc5c4c3)oc3ccc(-c4ccc(-c5ccc(-c7ccc8c9ccccc9c9ccccc9c8c7)cc5)cc4)cc36)cc21]

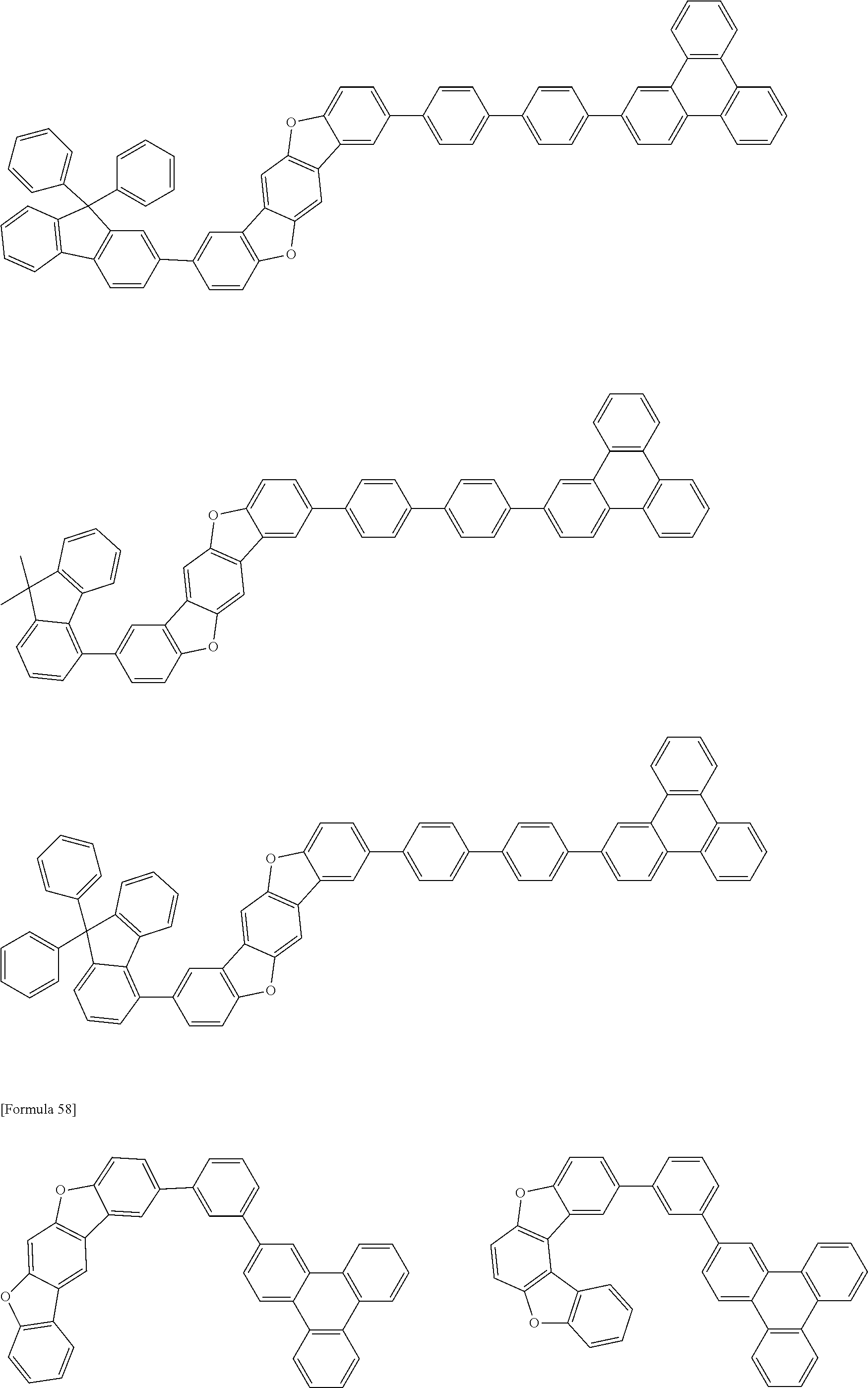

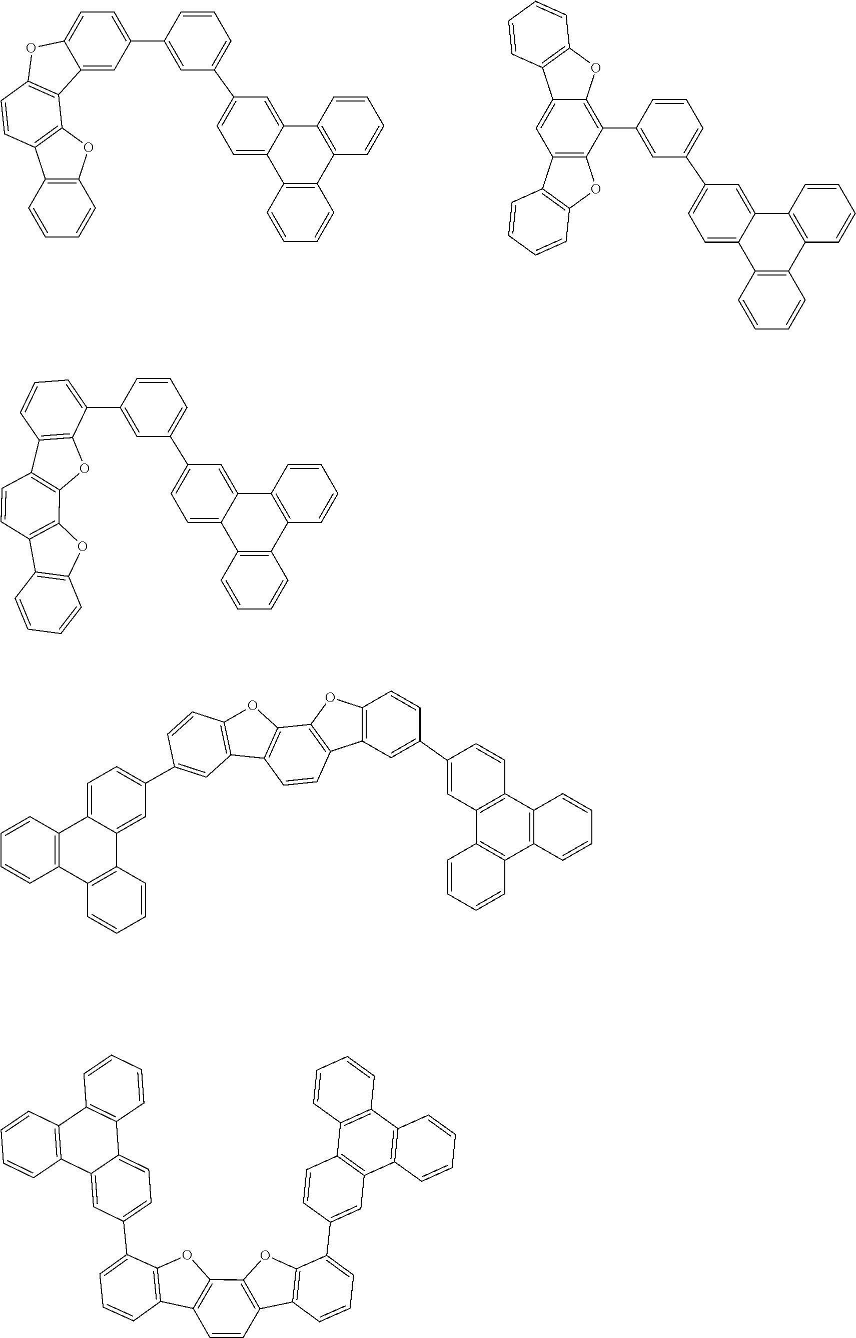 [SMILES: c1cc(-c2ccc3c4ccccc4c4ccccc4c3c2)cc(-c2ccc3oc4ccc5c6ccccc6oc5c4c3c2)c1 ; c1cc(-c2ccc3c4ccccc4c4ccccc4c3c2)cc(-c2c3oc4ccccc4c3cc3c2oc2ccccc23)c1 ; c1cc(-c2ccc3c4ccccc4c4ccccc4c3c2)cc(-c2cccc3c2oc2c3ccc3c4ccccc4oc32)c1 ; c1ccc2c(c1)c1ccccc1c1cc(-c3ccc4oc5c(ccc6c7cc(-c8ccc9c%10ccccc%10c%10ccccc%10c9c8)ccc7oc65)c4c3)ccc21 ; c1cc(-c2ccc3c4ccccc4c4ccccc4c3c2)c2oc3c(ccc4c5cccc(-c6ccc7c8ccccc8c8ccccc8c7c6)c5oc43)c2c1]

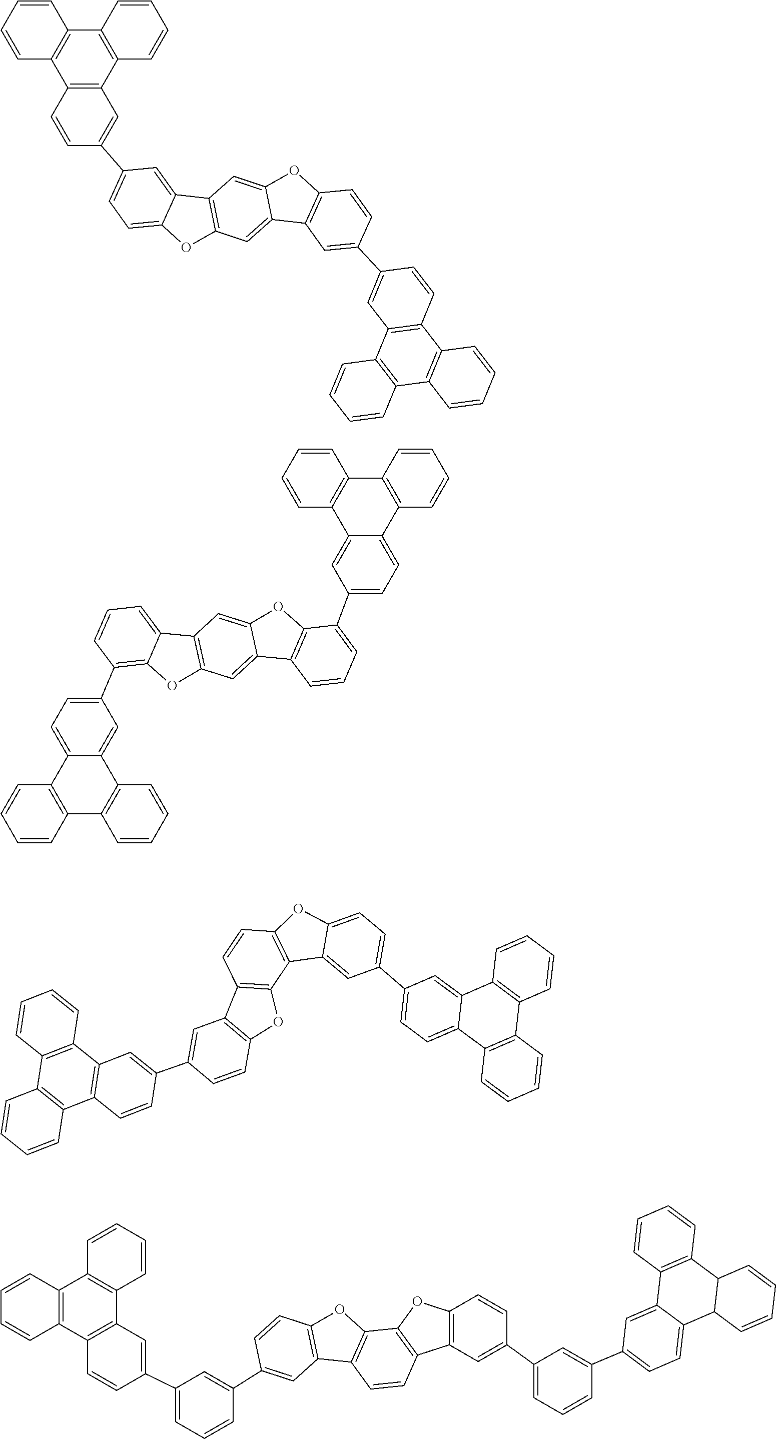

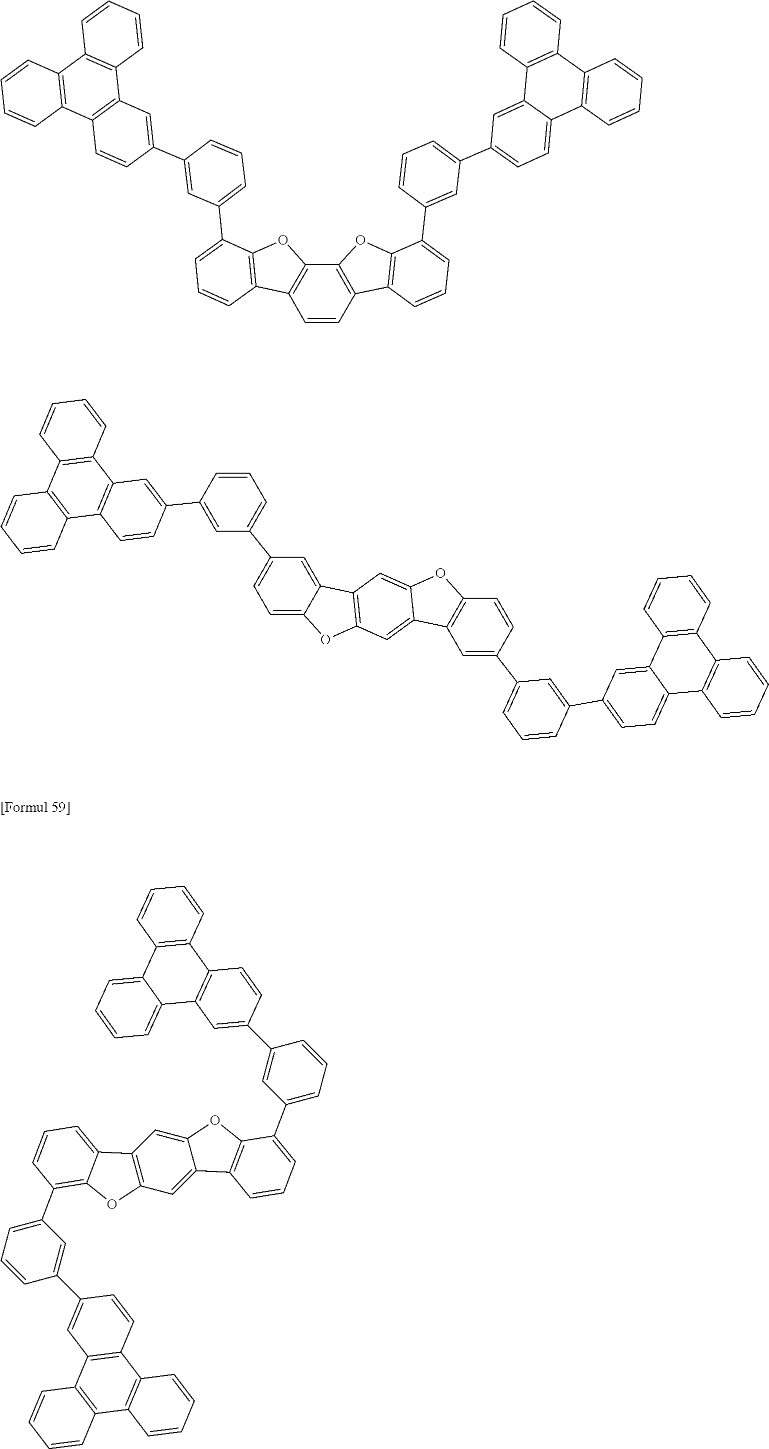 [SMILES: c1cc(-c2ccc3c4ccccc4c4ccccc4c3c2)cc(-c2cccc3c2oc2c3ccc3c4cccc(-c5cccc(-c6ccc7c8ccccc8c8ccccc8c7c6)c5)c4oc32)c1 ; c1cc(-c2ccc3oc4cc5c(cc4c3c2)oc2ccc(-c3cccc(-c4ccc6c7ccccc7c7ccccc7c6c4)c3)cc25)cc(-c2ccc3c4ccccc4c4ccccc4c3c2)c1 ; c1cc(-c2ccc3c4ccccc4c4ccccc4c3c2)cc(-c2cccc3c2oc2cc4c(cc23)oc2c(-c3cccc(-c5ccc6c7ccccc7c7ccccc7c6c5)c3)cccc24)c1]

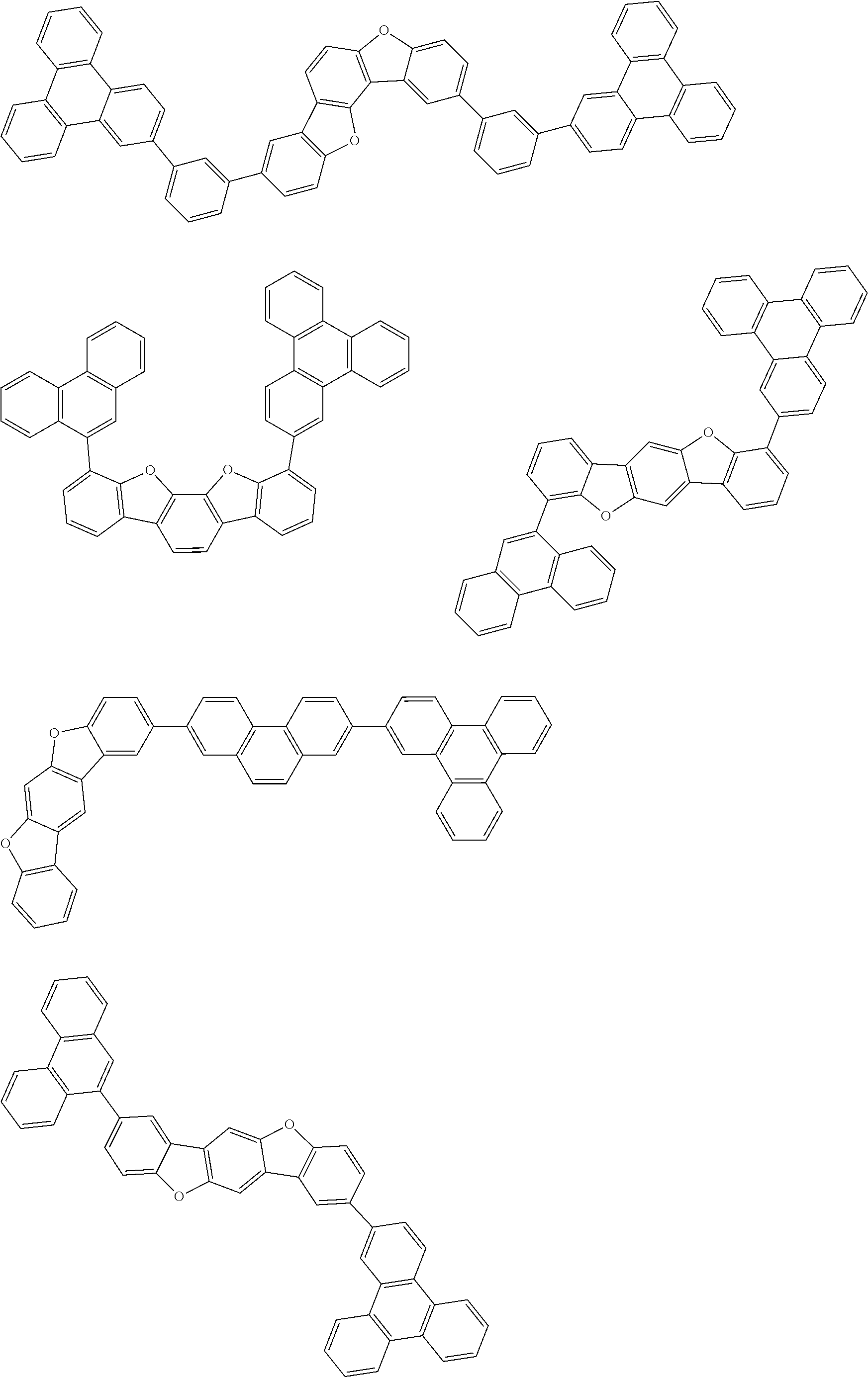 [SMILES: c1cc(-c2ccc3oc4c(ccc5oc6ccc(-c7cccc(-c8ccc9c%10ccccc%10c%10ccccc%10c9c8)c7)cc6c54)c3c2)cc(-c2ccc3c4ccccc4c4ccccc4c3c2)c1 ; c1ccc2c(c1)cc(-c1cccc3c1oc1c3ccc3c4cccc(-c5ccc6c7ccccc7c7ccccc7c6c5)c4oc31)c1ccccc12 ; c1ccc2c(c1)cc(-c1cccc3c1oc1cc4c(cc13)oc1c(-c3ccc5c6ccccc6c6ccccc6c5c3)cccc14)c1ccccc12 ; c1ccc2c(c1)oc1cc3oc4ccc(-c5ccc6c(ccc7cc(-c8ccc9c%10ccccc%10c%10ccccc%10c9c8)ccc76)c5)cc4c3cc12 ; c1ccc2c(c1)cc(-c1ccc3oc4cc5c(cc4c3c1)oc1ccc(-c3ccc4c6ccccc6c6ccccc6c4c3)cc15)c1ccccc12]

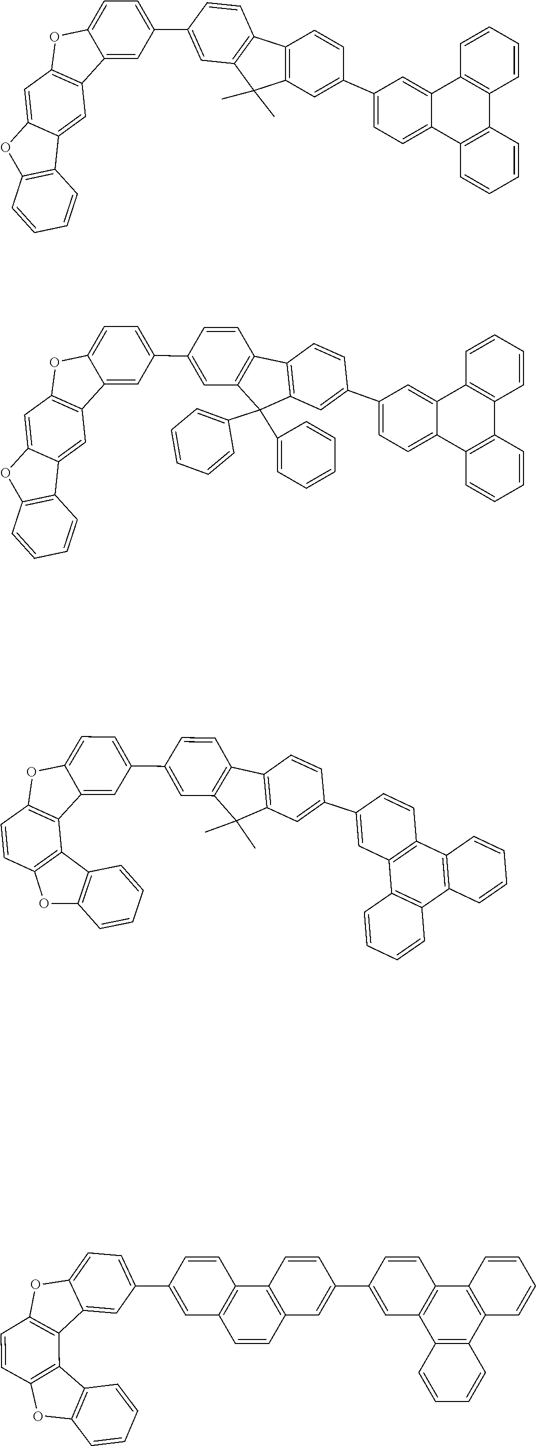 [SMILES: CC1(C)c2cc(-c3ccc4oc5cc6oc7ccccc7c6cc5c4c3)ccc2-c2ccc(-c3ccc4c5ccccc5c5ccccc5c4c3)cc21 ; c1ccc(C2(c3ccccc3)c3cc(-c4ccc5oc6cc7oc8ccccc8c7cc6c5c4)ccc3-c3ccc(-c4ccc5c6ccccc6c6ccccc6c5c4)cc32)cc1 ; CC1(C)c2cc(-c3ccc4c5ccccc5c5ccccc5c4c3)ccc2-c2ccc(-c3ccc4oc5ccc6oc7ccccc7c6c5c4c3)cc21 ; c1ccc2c(c1)oc1ccc3oc4ccc(-c5ccc6c(ccc7cc(-c8ccc9c%10ccccc%10c%10ccccc%10c9c8)ccc76)c5)cc4c3c12]

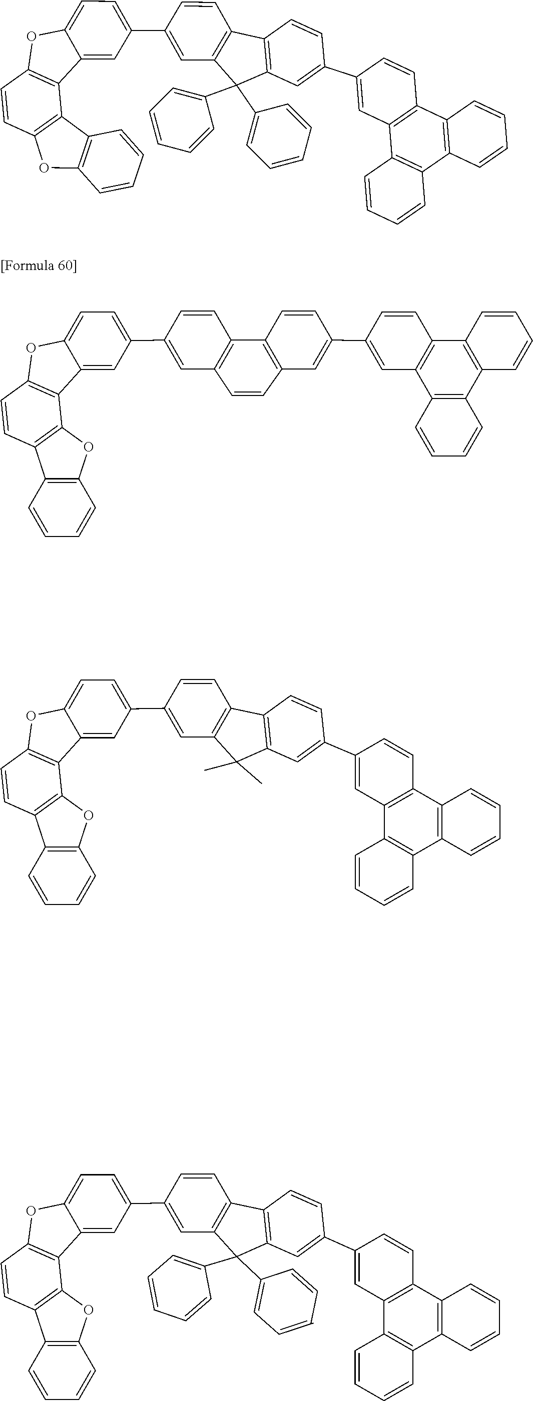 [SMILES: c1ccc(C2(c3ccccc3)c3cc(-c4ccc5c6ccccc6c6ccccc6c5c4)ccc3-c3ccc(-c4ccc5oc6ccc7oc8ccccc8c7c6c5c4)cc32)cc1 ; c1ccc2c(c1)oc1c2ccc2oc3ccc(-c4ccc5c(ccc6cc(-c7ccc8c9ccccc9c9ccccc9c8c7)ccc65)c4)cc3c21 ; CC1(C)c2cc(-c3ccc4c5ccccc5c5ccccc5c4c3)ccc2-c2ccc(-c3ccc4oc5ccc6c7ccccc7oc6c5c4c3)cc21 ; Cc1ccc(C2(c3ccccc3)c3cc(-c4ccc5c6ccccc6c6ccccc6c5c4)ccc3-c3ccc(-c4ccc5oc6ccc7c8ccccc8oc7c6c5c4)cc32)cc1]

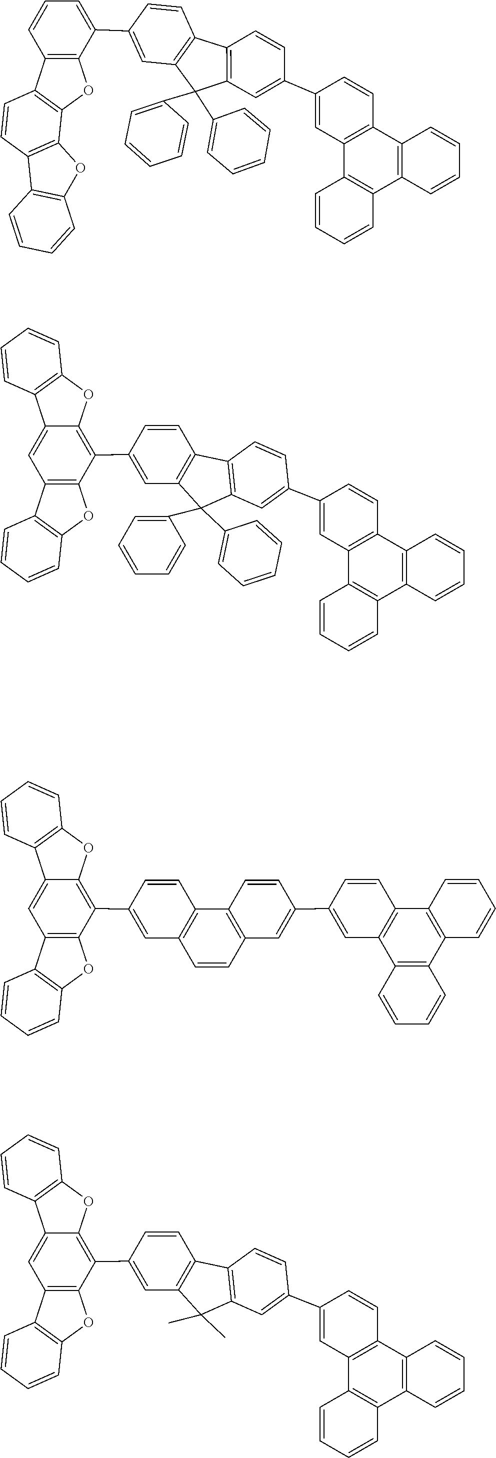 [SMILES: c1ccc(C2(c3ccccc3)c3cc(-c4ccc5c6ccccc6c6ccccc6c5c4)ccc3-c3ccc(-c4cccc5c4oc4c5ccc5c6ccccc6oc54)cc32)cc1 ; c1ccc(C2(c3ccccc3)c3cc(-c4ccc5c6ccccc6c6ccccc6c5c4)ccc3-c3ccc(-c4c5oc6ccccc6c5cc5c4oc4ccccc45)cc32)cc1 ; c1ccc2c(c1)oc1c(-c3ccc4c(ccc5cc(-c6ccc7c8ccccc8c8ccccc8c7c6)ccc54)c3)c3oc4ccccc4c3cc12 ; CC1(C)c2cc(-c3ccc4c5ccccc5c5ccccc5c4c3)ccc2-c2ccc(-c3c4oc5ccccc5c4cc4c3oc3ccccc34)cc21]

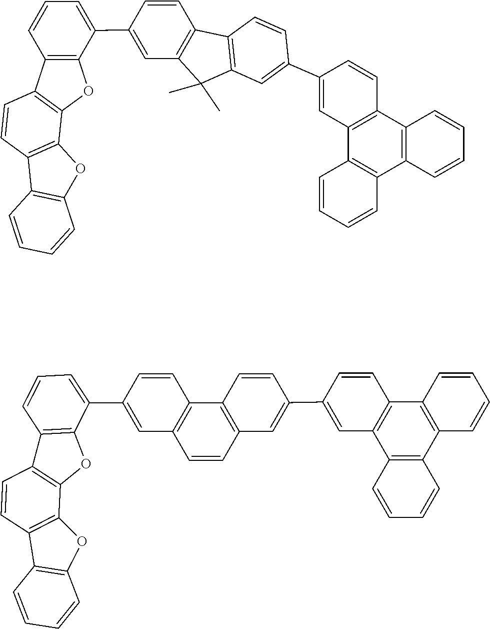 [SMILES: CC1(C)c2cc(-c3ccc4c5ccccc5c5ccccc5c4c3)ccc2-c2ccc(-c3cccc4c3oc3c4ccc4c5ccccc5oc43)cc21 ; c1ccc2c(c1)oc1c2ccc2c3cccc(-c4ccc5c(ccc6cc(-c7ccc8c9ccccc9c9ccccc9c8c7)ccc65)c4)c3oc21]

### Compound M2

The emitting layer of the exemplary embodiment includes the delayed fluorescent compound M2.

### Delayed Fluorescence

Delayed fluorescence is explained in “Yuki Hando-tai no Debaisu Bussei (Device Physics of Organic Semiconductors)” (edited by ADACHI, Chihaya, published by Kodansha, on pages 261-268). This document describes that, if an energy difference ΔE₁₃ of a fluorescent material between a singlet state and a triplet state is reducible, a reverse energy transfer from the triplet state to the singlet state, which usually occurs at a low transition probability, would occur at a high efficiency to express thermally activated delayed fluorescence (TADF). Further, a mechanism of generating delayed fluorescence is explained in FIG. 10.38 in the document. The compound M2 of the exemplary embodiment is preferably a compound exhibiting thermally activated delayed fluorescence generated by such a mechanism.

In general, emission of delayed fluorescence can be confirmed by measuring the transient PL (Photo Luminescence).

The behavior of delayed fluorescence can also be analyzed based on the decay curve obtained from the transient PL measurement. The transient PL measurement is a method of irradiating a sample with a pulse laser to excite the sample, and measuring the decay behavior (transient characteristics) of PL emission after the irradiation is stopped. PL emission in TADF materials is classified into a light emission component from a singlet exciton generated by the first PL excitation and a light emission component from a singlet exciton generated via a triplet exciton. The lifetime of the singlet exciton generated by the first PL excitation is on the order of nanoseconds and is very short. Therefore, light emission from the singlet exciton rapidly attenuates after irradiation with the pulse laser.

On the other hand, the delayed fluorescence is gradually attenuated due to light emission from a singlet exciton generated via a triplet exciton having a long lifetime. As described above, there is a large temporal difference between the light emission from the singlet exciton generated by the first PL excitation and the light emission from the singlet exciton generated via the triplet exciton. Therefore, the luminous intensity derived from delayed fluorescence can be determined.

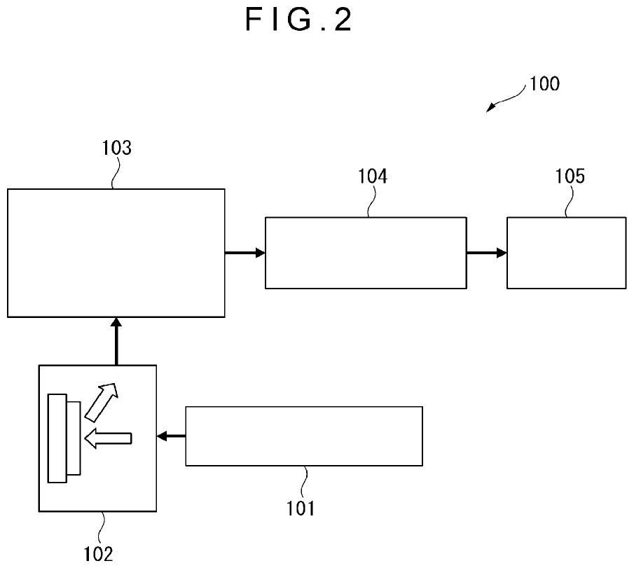

FIG. 2 shows a schematic diagram of an exemplary device for measuring the transient PL. An example of a method of measuring a transient PL using FIG. 2 and an example of behavior analysis of delayed fluorescence will be described.

A transient PL measuring device 100 in FIG. 2 includes: a pulse laser 101 capable of radiating a light having a predetermined wavelength; a sample chamber 102 configured to house a measurement sample; a spectrometer 103 configured to divide a light radiated from the measurement sample; a streak camera 104 configured to provide a two-dimensional image; and a personal computer 105 configured to import and analyze the two-dimensional image. A device for measuring the transient PL is not limited to the device shown in FIG. 2.

The sample housed in the sample chamber 102 is obtained by forming a thin film, in which a matrix material is doped with a doping material at a concentration of 12 mass %, on the quartz substrate.

The thin film sample housed in the sample chamber 102 is irradiated with the pulse laser from the pulse laser 101 to excite the doping material. Emission is extracted in a direction of 90 degrees with respect to a radiation direction of the excited light. The extracted emission is divided by the spectrometer 103 to form a two-dimensional image in the streak camera 104. As a result, the two-dimensional image is obtainable in which the ordinate axis represents a time, the abscissa axis represents a wavelength, and a bright spot represents a luminous intensity. When this two-dimensional image is taken out at a predetermined time axis, an emission spectrum in which the ordinate axis represents the luminous intensity and the abscissa axis represents the wavelength is obtainable. Moreover, when this two-dimensional image is taken out at the wavelength axis, a decay curve (transient PL) in which the ordinate axis represents a logarithm of the luminous intensity and the abscissa axis represents the time is obtainable.

For instance, a thin film sample A was prepared as described above from a reference compound H1 as the matrix material and a reference compound D1 as the doping material and was measured in terms of the transient PL.

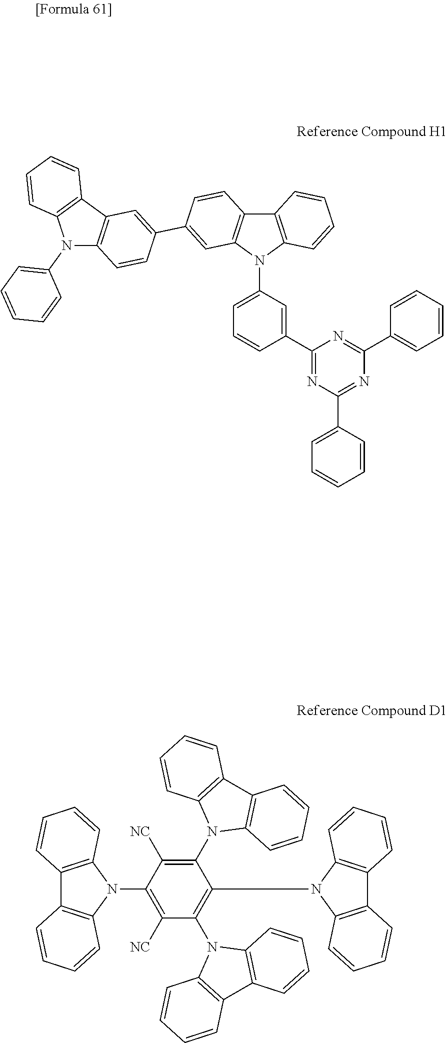 [SMILES: c1ccc(-c2nc(-c3ccccc3)nc(-c3cccc(-n4c5ccccc5c5ccc(-c6ccc7c(c6)c6ccccc6n7-c6ccccc6)cc54)c3)n2)cc1 ; N#Cc1c(-n2c3ccccc3c3ccccc32)c(C#N)c(-n2c3ccccc3c3ccccc32)c(-n2c3ccccc3c3ccccc32)c1-n1c2ccccc2c2ccccc21]

The decay curve was analyzed with respect to the above thin film sample A and a thin film sample B. The thin film sample B was manufactured in the same manner as described above from a reference compound H2 as the matrix material and the reference compound D1 as the doping material.

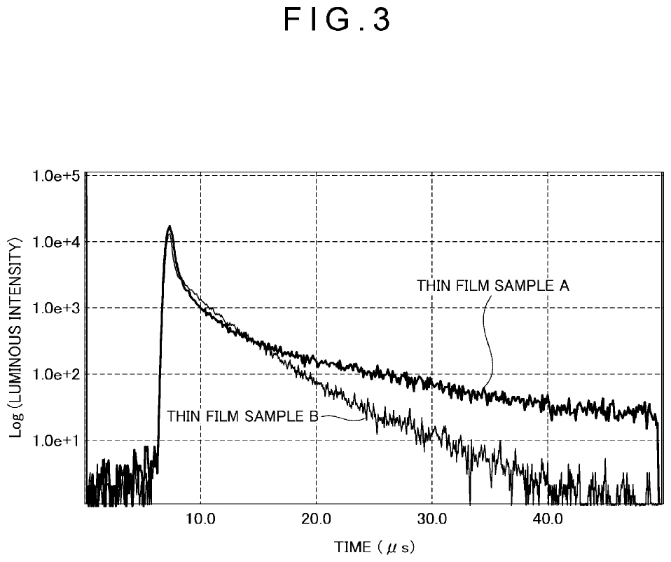

FIG. 3 shows decay curves obtained from transient PL obtained by measuring the thin film samples A and B.

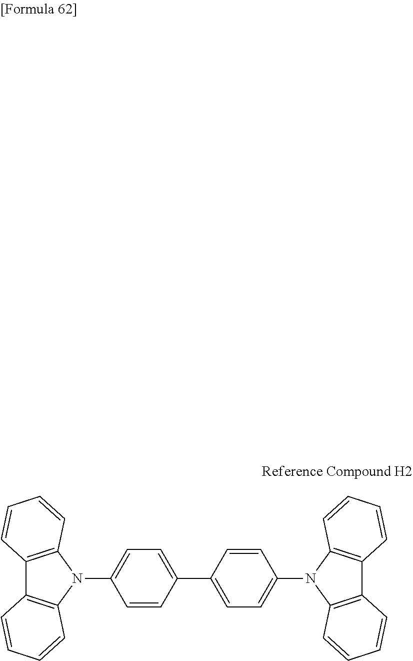 [SMILES: c1ccc2c(c1)c1ccccc1n2-c1ccc(-c2ccc(-n3c4ccccc4c4ccccc43)cc2)cc1]

As described above, an emission decay curve in which the ordinate axis represents the luminous intensity and the abscissa axis represents the time can be obtained by the transient PL measurement. Based on the emission decay curve, a fluorescence intensity ratio between fluorescence emitted from a singlet state generated by photo-excitation and delayed fluorescence emitted from a singlet state generated by inverse energy transfer via a triplet state can be estimated. In a delayed fluorescent material, a ratio of the intensity of the slowly decaying delayed fluorescence to the intensity of the promptly decaying fluorescence is relatively large.

Specifically, Prompt emission and Delay emission are present as emission from the delayed fluorescent material. Prompt emission is observed promptly when the excited state is achieved by exciting the compound of the exemplary embodiment with a pulse beam (i.e., a beam emitted from a pulse laser) having a wavelength absorbable by the delayed fluorescent material. Delay emission is observed not promptly when the excited state is achieved but after the excited state is achieved.

An amount of Prompt emission, an amount of Delay emission and a ratio between the amounts thereof can be obtained according to the method as described in “Nature 492, 234-238, 2012” (Reference Document 1). The amount of Prompt emission and the amount of Delay emission may be calculated using a device different from one described in Reference Document 1 or one shown in FIG. 2.

Herein, a sample manufactured by the following method is used for measuring delayed fluorescence of the compound M2. For instance, the compound M2 is dissolved in toluene to prepare a dilute solution with an absorbance of 0.05 or less at the excitation wavelength to eliminate the contribution of self-absorption. In order to prevent quenching due to oxygen, the sample solution is frozen and degassed and then sealed in a cell with a lid under an argon atmosphere to obtain an oxygen-free sample solution saturated with argon.

The fluorescence spectrum of the sample solution is measured with a spectrofluorometer FP-8600 (manufactured by JASCO Corporation), and the fluorescence spectrum of a 9,10-diphenylanthracene ethanol solution is measured under the same conditions. Using the fluorescence area intensities of both spectra, the total fluorescence quantum yield is calculated by an equation (1) in Morris et al. J. Phys. Chem. 80 (1976) 969.

In the exemplary embodiment, provided that an amount of Prompt emission of a measurement target compound (compound M2) is denoted by X_{P} and the amount of Delay emission is denoted by X_{D}, a value of X_{D}/X_{P} is preferably 0.05 or more.

The amounts of Prompt emission and Delay emission and a ratio of the amounts thereof in compounds other than the compound M2 herein are measured in the same manner as those of the compound M2.

The compound M2 is exemplified by a compound represented by a formula (2) below.

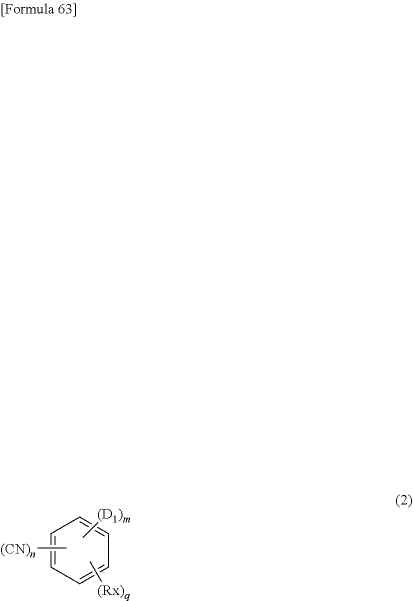

In the formula (2):

n is 1, 2, 3 or 4;

m is 1, 2, 3 or 4;

q is 0, 1, 2, 3 or 4;

m+n+q=6 is satisfied;

CN is a cyano group;

D₁ is a group represented by a formula (2a), (2b) or (2c) below, and when a plurality of D₁ are present, the plurality of D₁ are mutually the same or different;

Rx is a hydrogen atom or a substituent, or a pair of adjacent ones of Rx are mutually bonded to form a ring, and when a plurality of Rx are present, the plurality of Rx are mutually the same or different;

Rx as a substituent is each independently a halogen atom, a substituted or unsubstituted aryl group having 6 to 30 ring carbon atoms, a substituted or unsubstituted heterocyclic group having 5 to 30 ring atoms, a substituted or unsubstituted amino group, a substituted or unsubstituted carbonyl group, a substituted or unsubstituted alkyl group having 1 to 30 carbon atoms, a substituted or unsubstituted alkyl halide group having 1 to 30 carbon atoms, a substituted or unsubstituted alkylsilyl group having 3 to 30 carbon atoms, or a substituted or unsubstituted arylsilyl group having 6 to 60 ring carbon atoms; and CN, D₁ and Rx are bonded to respective carbon atoms of a six-membered ring.

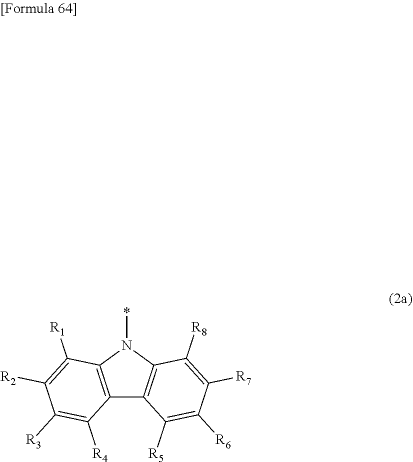

In the formula (2a):

R₁ to R₈ are each independently a hydrogen atom or a substituent, or at least one pair of a pair of R₁ and R₂, a pair of R₂ and R₃, a pair of R₃ and R₄, a pair of R₅ and R₆, a pair of R₆ and R₇, or a pair of R₇ and R₈ are mutually bonded to form a ring;

R₁ to R₈ as a substituent are each independently a halogen atom, a substituted or unsubstituted aryl group having 6 to 30 ring carbon atoms, a substituted or unsubstituted heterocyclic group having 5 to 30 ring atoms, a substituted or unsubstituted alkyl group having 1 to 30 carbon atoms, a substituted or unsubstituted alkyl halide group having 1 to 30 carbon atoms, a substituted or unsubstituted alkylsilyl group having 3 to 30 carbon atoms, a substituted or unsubstituted arylsilyl group having 6 to 60 ring carbon atoms, a hydroxy group, a substituted or unsubstituted alkoxy group having 1 to 30 carbon atoms, a substituted or unsubstituted alkoxy halide group having 1 to 30 carbon atoms, a substituted or unsubstituted aryloxy group having 6 to 30 ring carbon atoms, a substituted or unsubstituted alkylamino group having 2 to 30 carbon atoms, a substituted or unsubstituted arylamino group having 6 to 60 ring carbon atoms, a thiol group, a substituted or unsubstituted alkylthio group having 1 to 30 carbon atoms, or a substituted or unsubstituted arylthio group having 6 to 30 ring carbon atoms; and

* represents a bonding position to a carbon atom of a benzene ring in the formula (2).

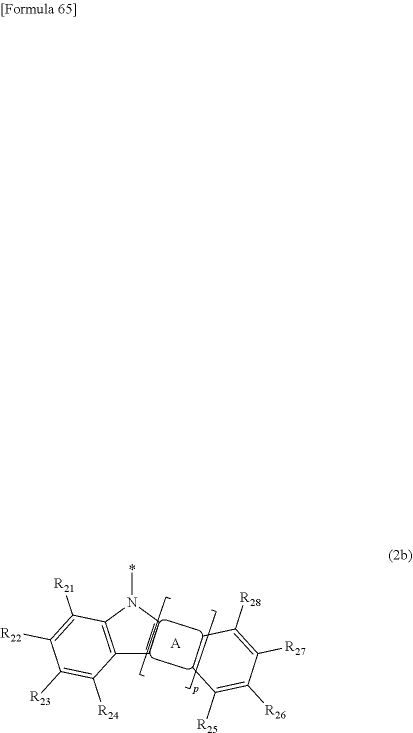

In the formula (2b):

R₂₁ to R₂₈ are each independently a hydrogen atom or a substituent, or at least one pair of a pair of R₂₁ and R₂₂, a pair of R₂₂ and R₂₃, a pair of R₂₃ and R₂₄, a pair of R₂₅ and R₂₆, a pair of R₂₆ and R₂₇, or a pair of R₂₇ and R₂₈ are mutually bonded to form a ring;

R₂₁ to R₂₈ as a substituent each independently represent the same as R₁ to R₈ in the formula (2a);

A represents a cyclic structure represented by a formula (211) or (212) below, and the cyclic structure A is fused with adjacent cyclic structure(s) at any position(s);

p is 1, 2, 3 or 4;

when p is 2, 3 or 4, a plurality of cyclic structures A are mutually the same or different; and

* represents a bonding position to a carbon atom of a benzene ring in the formula (2).

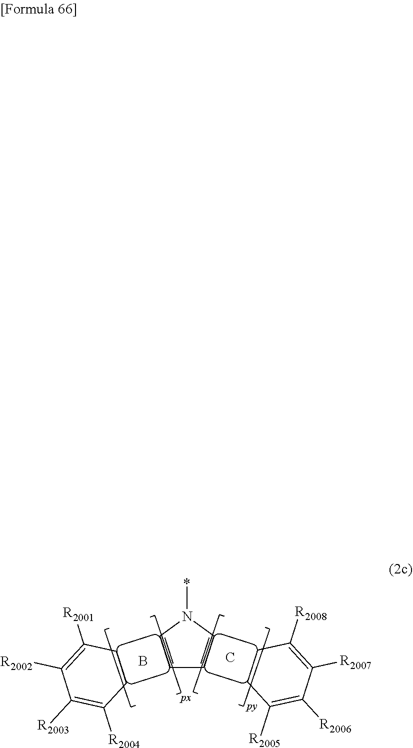

In the formula (2c):

R₂₀₀₁ to R₂₀₀₈ are each independently a hydrogen atom or a substituent, or at least one pair of a pair of R₂₀₀₁ and R₂₀₀₂, a pair of R₂₀₀₂ and R₂₀₀₃, a pair of R₂₀₀₃ and R₂₀₀₄, a pair of R₂₀₀₅ and R₂₀₀₆, a pair of R₂₀₀₆ and R₂₀₀₇, or a pair of R₂₀₀₇ and R₂₀₀₈ are mutually bonded to form a ring;

R₂₀₀₁ to R₂₀₀₈ as a substituent each independently represent the same as R₁ to R₈ as a substituent in the formula (2a);

B represents a cyclic structure represented by the formula (211) or (212), and the cyclic structure B is fused with adjacent cyclic structure(s) at any position(s);

px is 1, 2, 3 or 4;

when px is 2, 3 or 4, a plurality of cyclic structures B are mutually the same or different;

C represents a cyclic structure represented by the formula (211) or (212), and the cyclic structure C is fused with adjacent cyclic structure(s) at any position(s);

py is 1, 2, 3 or 4;

when py is 2, 3 or 4, a plurality of cyclic structures C are mutually the same or different; and

* represents a bonding position to a carbon atom of a benzene ring in the formula (2).

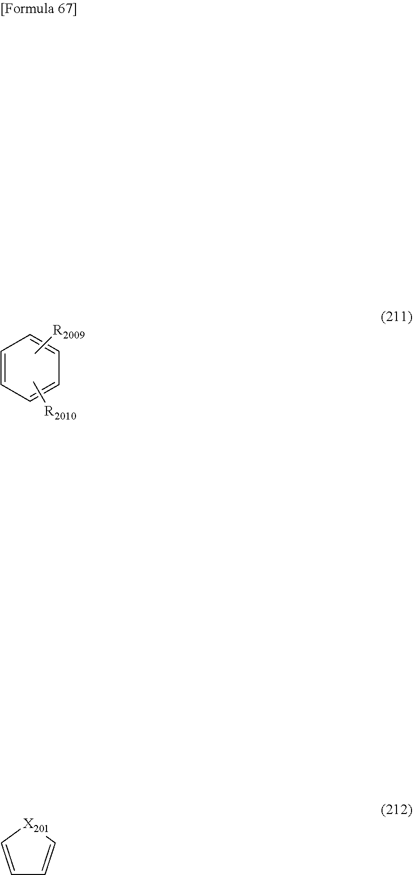

In the formula (211), R₂₀₀₉ and R₂₀₁₀ are each independently a hydrogen atom or a substituent, or bonded to a part of an adjacent cyclic structure to form a ring, or a pair of R₂₀₀₉ and R₂₀₁₀ are mutually bonded to form a ring;

in the formula (212), X₂₀₁ is CR₂₀₁₁R₂₀₁₂, NR₂₀₁₃, a sulfur atom, or an oxygen atom, and R₂₀₁₁, R₂₀₁₂ and R₂₀₁₃ are each independently a hydrogen atom or a substituent, or R₂₀₁₁ and R₂₀₁₂ are mutually bonded to form a ring; and

R₂₀₀₉, R₂₀₁₀, R₂₀₁₁, R₂₀₁₂ and R₂₀₁₃ as a substituent each independently represent the same as R₁ to R₈ as a substituent in the formula (2a).

In the formula (211), R₂₀₀₉ and R₂₀₁₀ are each independently bonded to a part of an adjacent cyclic structure to form a ring, which specifically means any of (I) to (IV) below.

In the formula (211), a pair of R₂₀₀₉ and R₂₀₁₀ are mutually bonded to form a ring, which specifically means (V) below.

(I) When the cyclic structures represented by the formula (211) are adjacent to each other, between the two adjacent rings, at least one pair of the following are mutually bonded to form a ring: R₂₀₀₉ of one of the rings and R₂₀₀₉ of the other of the rings; R₂₀₀₉ of one of the rings and R₂₀₁₀ of the other of the rings; or R₂₀₁₀ of one of the rings and R₂₀₁₀ of the other of the rings.

(II) When the cyclic structure represented by the formula (211) and the benzene ring having R₂₅ to R₂₈ in the formula (2b) are adjacent to each other, between the two adjacent rings, at least one pair of the following are mutually bonded to form a ring: R₂₀₉ of one of the rings and R₂₅ of the other of the rings; R₂₀₉ of one of the rings and R₂₈ of the other of the rings; R₂₁₀ of one of the rings and R₂₅ of the other of the rings; or R₂₁₀ of one of the rings and R₂₈ of the other of the rings.

(III) When the cyclic structure represented by the formula (211) and the benzene ring having R₂₀₀₁ to R₂₀₀₄ in the formula (2c) are adjacent to each other, between the two adjacent rings, at least one pair of the following are mutually bonded to form a ring: R₂₀₀₉ of one of the rings and R₂₀₀₁ of the other of the rings; R₂₀₀₉ of one of the rings and R₂₀₀₄ of the other of the rings; R₂₀₁₀ of one of the rings and R₂₀₀₁ of the other of the rings; or R₂₀₁₀ of one of the rings and R₂₀₀₄ of the other of the rings.

(IV) When the cyclic structure represented by the formula (211) and the benzene ring having R₂₀₀₅ to R₂₀₀₈ in the formula (2c) are adjacent to each other, between the two adjacent rings, at least one pair of the following are mutually bonded to form a ring: R₂₀₀₉ of one of the rings and R₂₀₀₅ of the other of the rings; R₂₀₀₉ of one of the rings and R₂₀₀₀ of the other of the rings; R₂₀₁₀ of one of the rings and R₂₀₀₅ of the other of the rings; or R₂₀₁₀ of one of the rings and R₂₀₀₈ of the other of the rings.

(V) The pair of R₂₀₀₉ and R₂₀₁₀ of the cyclic structure represented by the formula (211) are mutually bonded to form a ring. In other words, (V) means that the pair of R₂₀₀₉ and R₂₀₁₀, which are bonded to the same ring, are mutually bonded to form a ring.

In the compound M2 of the exemplary embodiment, it is preferable that Rx is each independently a hydrogen atom, an unsubstituted aryl group having 6 to 30 ring carbon atoms, an unsubstituted heterocyclic group having 5 to 30 ring atoms, or an unsubstituted alkyl group having 1 to 30 carbon atoms; and when Rx is an unsubstituted heterocyclic group having 5 to 30 ring atoms, Rx as the unsubstituted heterocyclic group having 5 to 30 ring atoms is a pyridyl group, pyrimidinyl group, triazinyl group, dibenzofuranyl group, or dibenzothienyl group.

Herein, the triazinyl group refers to a group obtained by excluding one hydrogen atom from 1,3,5-triazine, 1,2,4-triazine, or 1,2,3-triazine.

The triazinyl group is preferably a group obtained by excluding one hydrogen atom from 1,3,5-triazine.

In the compound M2 of the exemplary embodiment, Rx is more preferably each independently a hydrogen atom, an unsubstituted aryl group having 6 to 30 ring carbon atoms, an unsubstituted dibenzofuranyl group, or an unsubstituted dibenzothienyl group.

In the compound M2 of the exemplary embodiment, Rx is further preferably a hydrogen atom.

In the compound M2 of the exemplary embodiment, it is preferable that R₁ to R₈, R₂₁ to R₂₈, R₂₀₀₁ to R₂₀₀₈, R₂₀₀₉ to R₂₀₁₀ and R₂₀₁₁ to R₂₀₁₃ as a substituent are each independently an unsubstituted aryl group having 6 to 30 ring carbon atoms, an unsubstituted heterocyclic group having 5 to 30 ring atoms, or an unsubstituted alkyl group having 1 to 30 carbon atoms.

In the compound M2 of the exemplary embodiment, it is preferable that R₁₀₁ to R₁₅₀ and R₆₁ to R₇₀ as a substituent are each independently an unsubstituted aryl group having 6 to 14 ring carbon atoms, an unsubstituted heterocyclic group having 5 to 14 ring atoms, or an unsubstituted alkyl group having 1 to 6 carbon atoms, and R_{X21} to R_{X26} as a substituent are each independently an unsubstituted aryl group having 6 to 14 ring carbon atoms, or an unsubstituted heterocyclic group having 5 to 14 ring atoms.

In the compound M2 of the exemplary embodiment, it is also preferable that R₁₀₁ to R₁₅₀ and R₆₁ to R₇₀ are each a hydrogen atom, and R_{X21} to R_{X26} as a substituent are each independently an unsubstituted aryl group having 6 to 14 ring carbon atoms, or an unsubstituted heterocyclic group having 5 to 14 ring atoms.

In the compound M2 of the exemplary embodiment, it is preferable that R₂₀₁ to R₂₆₀ as a substituent are each independently a halogen atom, an unsubstituted aryl group having 6 to 14 ring carbon atoms, an unsubstituted heterocyclic group having 5 to 14 ring atoms, or an unsubstituted alkyl group having 1 to 6 carbon atoms, and R_{X27} and R_{X28} as a substituent are each independently an unsubstituted aryl group having 6 to 14 ring carbon atoms, or an unsubstituted alkyl group having 1 to 6 carbon atoms.

In the compound M2 of the exemplary embodiment, it is more preferable that R₂₀₁ to R₂₆₀ as a substituent are each independently an unsubstituted aryl group having 6 to 14 ring carbon atoms, an unsubstituted heterocyclic group having 5 to 14 ring atoms, or an unsubstituted alkyl group having 1 to 6 carbon atoms, R_{X27} and R_{X28} as a substituent are each independently an unsubstituted aryl group having 6 to 14 ring carbon atoms, or an unsubstituted alkyl group having 1 to 6 carbon atoms.

In the compound M2 of the exemplary embodiment, it is also preferable that R₂₀₁ to R₂₆₀ are each a hydrogen atom, and R_{X27} and R_{X28} as a substituent are each independently an unsubstituted aryl group having 6 to 14 ring carbon atoms, or an unsubstituted alkyl group having 1 to 6 carbon atoms.

In the compound M2 of the exemplary embodiment, D₁ is preferably a group represented by one of formulae (D-21) to (D-25) below.

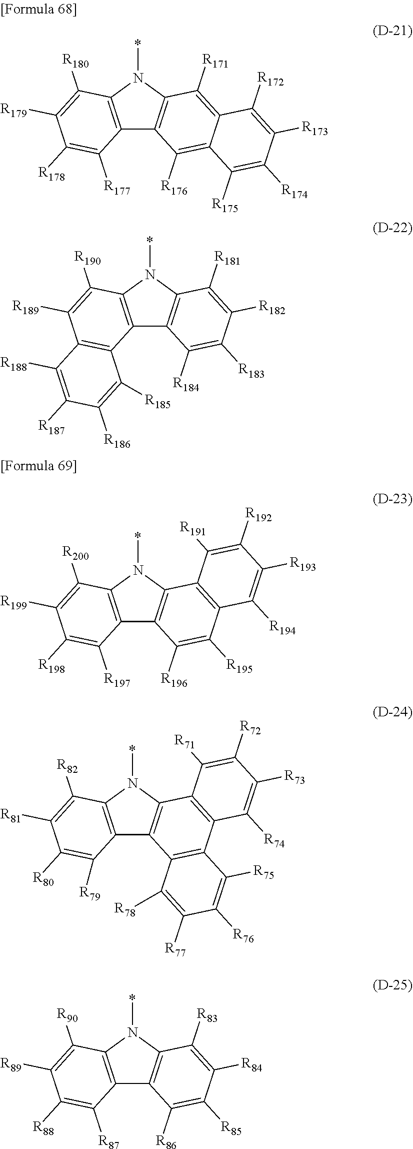

In the formulae (D-21) to (D-25):

R₁₇₁ to R₂₀₀ and R₇₁ to R₉₀ are each independently a hydrogen atom or a substituent, or at least one pair of a pair of R₁₇₁ and R₁₇₂, a pair of R₁₇₂ and R₁₇₃, a pair of R₁₇₃ and R₁₇₄, a pair of R₁₇₄ and R₁₇₅, a pair of R₁₇₅ and R₁₇₆, a pair of R₁₇₇ and R₁₇₈, a pair of R₁₇₈ and R₁₇₉, a pair of R₁₇₉ and R₁₈₀, a pair of R₁₈₁ and R₁₈₂, a pair of R₁₈₂ and R₁₈₃, a pair of R₁₈₃ and R₁₈₄, a pair of R₁₈₅ and R₁₈₆, a pair of R₁₈₆ and R₁₈₇, a pair of R₁₈₇ and R₁₈₈, a pair of R₁₈₈ and R₁₈₉, a pair of R₁₈₉ and R₁₉₀, a pair of R₁₉₁ and R₁₉₂, a pair of R₁₉₂ and R₁₉₃, a pair of R₁₉₃ and R₁₉₄, a pair of R₁₉₄ and R₁₉₅, a pair of R₁₉₅ and R₁₉₆, a pair of R₁₉₇ and R₁₉₈, a pair of R₁₉₈ and R₁₉₉, a pair of R₁₉₉ and R₂₀₀, a pair of R₇₁ and R₇₂, a pair of R₇₂ and R₇₃, a pair of R₇₃ and R₇₄, a pair of R₇₅ and R₇₆, a pair of R₇₆ and R₇₇, a pair of R₇₇ and R₇₈, a pair of R₇₉ and R₈₀, a pair of R₈₀ and R₈₁, or a pair of R₈₁ and R₈₂ are mutually bonded to form a ring;

R₁₇₁ to R₂₀₀ and R₇₁ to R₉₀ as a substituent are each independently a halogen atom, a substituted or unsubstituted aryl group having 6 to 14 ring carbon atoms, a substituted or unsubstituted heterocyclic group having 5 to 14 ring atoms, a substituted or unsubstituted alkyl group having 1 to 6 carbon atoms, a substituted or unsubstituted alkyl halide group having 1 to 6 carbon atoms, a substituted or unsubstituted alkylsilyl group having 3 to 6 carbon atoms, a hydroxy group, a substituted or unsubstituted alkoxy group having 1 to 6 carbon atoms, a substituted or unsubstituted alkoxy halide group having 1 to 6 carbon atoms, a substituted or unsubstituted aryloxy group having 6 to 14 ring carbon atoms, a substituted or unsubstituted alkylamino group having 2 to 12 carbon atoms, a thiol group, a substituted or unsubstituted alkylthio group having 1 to 6 carbon atoms, or a substituted or unsubstituted arylthio group having 6 to 14 ring carbon atoms; and

* represents a bonding position to a carbon atom of a benzene ring in the formula (2).

In the compound M2 of the exemplary embodiment, it is preferable that R₁₇₁ to R₂₀₀ and R₇₁ to R₉₀ as a substituent are each independently an unsubstituted aryl group having 6 to 14 ring carbon atoms, an unsubstituted heterocyclic group having 5 to 14 ring atoms, or an unsubstituted alkyl group having 1 to 6 carbon atoms.

In the compound M2 of the exemplary embodiment, it is also preferable that R₁₇₁ to R₂₀₀ and R₇₁ to R₉₀ are each a hydrogen atom.

In the compound M2 of the exemplary embodiment, D₁ is also preferably a group represented by one of formulae (D-26) to (D-31) below.

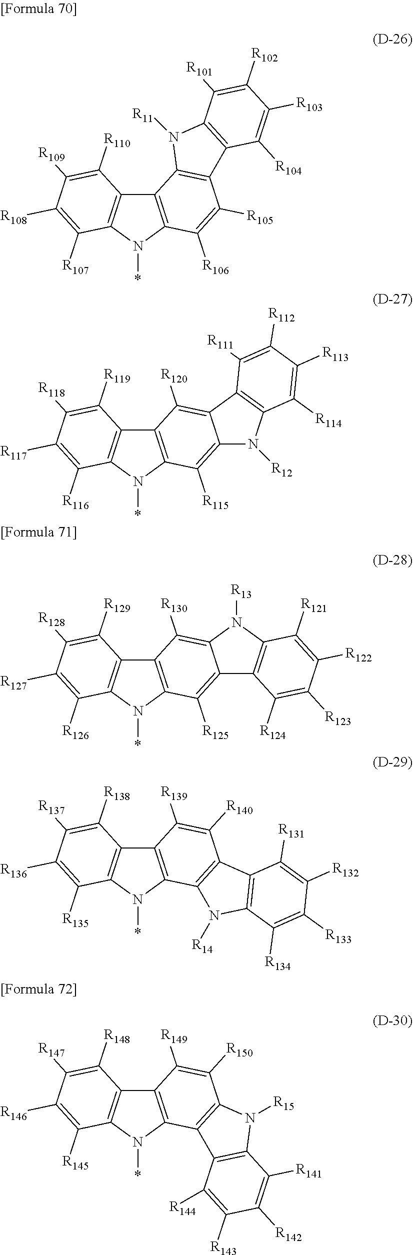

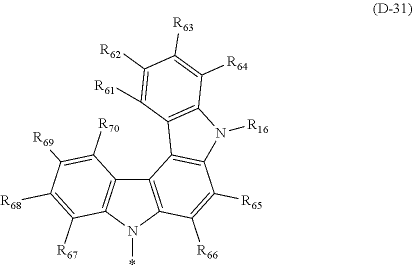

In the formulae (D-26) to (D-31):

R₁₁ to R₁₆ are each a substituent, and R₁₀₁ to R₁₅₀ and R₆₁ to R₇₀ are each independently a hydrogen atom or a substituent;

R₁₀₁ to R₁₅₀ and R₆₁ to R₇₀ as a substituent are each independently a substituted or unsubstituted aryl group having 6 to 14 ring carbon atoms, a substituted or unsubstituted heterocyclic group having 5 to 14 ring atoms, a substituted or unsubstituted alkyl group having 1 to 6 carbon atoms, a substituted or unsubstituted alkylsilyl group having 3 to 6 carbon atoms, a hydroxy group, a substituted or unsubstituted alkoxy group having 1 to 6 carbon atoms, a substituted or unsubstituted aryloxy group having 6 to 14 ring carbon atoms, a substituted or unsubstituted arylamino group having 6 to 28 ring carbon atoms, a substituted or unsubstituted alkylamino group having 2 to 12 carbon atoms, a thiol group, a substituted or unsubstituted alkylthio group having 1 to 6 carbon atoms, or a substituted or unsubstituted arylthio group having 6 to 14 ring carbon atoms;

R₁₁ to R₁₆ as a substituent are each independently a substituted or unsubstituted alkyl group having 1 to 6 carbon atoms, a substituted or unsubstituted aryl group having 6 to 14 ring carbon atoms, a substituted or unsubstituted heterocyclic group having 5 to 14 ring atoms, a substituted or unsubstituted alkylsilyl group having 3 to 6 carbon atoms, a substituted or unsubstituted aryloxy group having 6 to 14 ring carbon atoms, a substituted or unsubstituted alkylamino group having 2 to 12 carbon atoms, a substituted or unsubstituted alkylthio group having 1 to 6 carbon atoms, or a substituted or unsubstituted arylthio group having 6 to 14 ring carbon atoms; and

* represents a bonding position to a carbon atom of a benzene ring in the formula (2).

In the compound M2 of the exemplary embodiment, it is preferable that R₁₀₁ to R₁₅₀ and R₆₁ to R₇₀ as a substituent are each independently an unsubstituted aryl group having 6 to 14 ring carbon atoms, an unsubstituted heterocyclic group having 5 to 14 ring atoms, or an unsubstituted alkyl group having 1 to 6 carbon atoms, and R₁₁ to R₁₆ as a substituent are each independently an unsubstituted aryl group having 6 to 14 ring carbon atoms or an unsubstituted heterocyclic group having 5 to 14 ring atoms.

In the compound M2 of the exemplary embodiment, it is also preferable that R₁₀₁ to R₁₅₀ and R₆₁ to R₇₀ are each a hydrogen atom and R₁₁ to R₁₆ as a substituent are each independently an unsubstituted aryl group having 6 to 14 ring carbon atoms or an unsubstituted heterocyclic group having 5 to 14 ring atoms.

In the compound M2 of the exemplary embodiment, D₁ is also preferably a group represented by one of formulae (D-32) to (D-37) below.

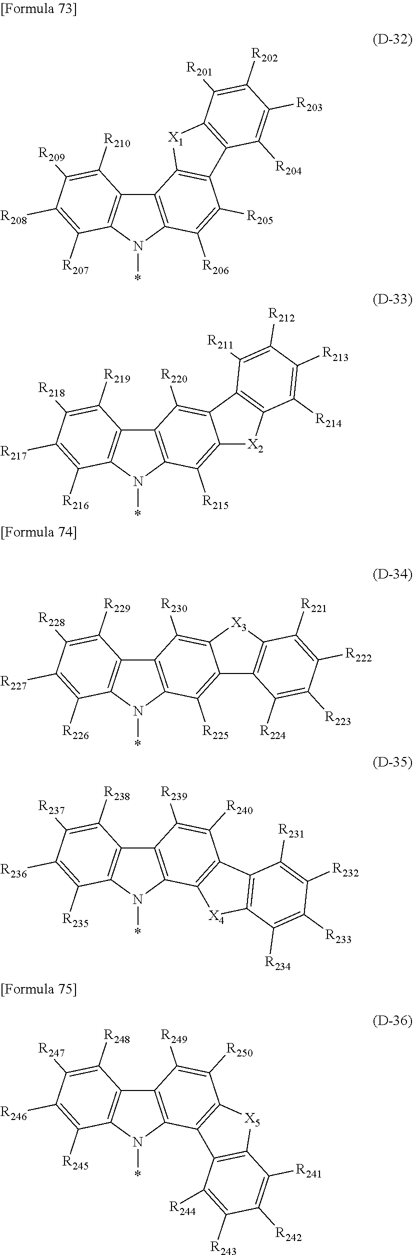

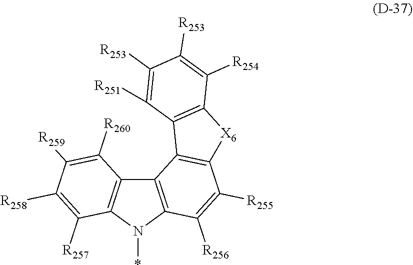

In the formulae (D-32) to (D-37):

X₁ to X₆ are each independently an oxygen atom, a sulfur atom, or CR₁₅₁R₁₅₂; R₂₀₁ to R₂₆₀ are each independently a hydrogen atom or a substituent; and R₁₅₁ and R₁₅₂ are each independently a hydrogen atom or a substituent or R₁₅₁ and R₁₅₂ are mutually bonded to form a ring;

R₂₀₁ to R₂₆₀, R₁₅₁ and R₁₅₂ as a substituent are each independently a halogen atom, a substituted or unsubstituted aryl group having 6 to 14 ring carbon atoms, a substituted or unsubstituted heterocyclic group having 5 to 14 ring atoms, a substituted or unsubstituted alkyl group having 1 to 6 carbon atoms, a substituted or unsubstituted alkyl halide group having 1 to 6 carbon atoms, a substituted or unsubstituted alkylsilyl group having 3 to 6 carbon atoms, a hydroxy group, a substituted or unsubstituted alkoxy group having 1 to 6 carbon atoms, a substituted or unsubstituted alkoxy halide group having 1 to 6 carbon atoms, a substituted or unsubstituted aryloxy group having 6 to 14 ring carbon atoms, a substituted or unsubstituted arylamino group having 6 to 28 ring carbon atoms, a substituted or unsubstituted alkylamino group having 2 to 12 carbon atoms, a thiol group, a substituted or unsubstituted alkylthio group having 1 to 6 carbon atoms, or a substituted or unsubstituted arylthio group having 6 to 14 ring carbon atoms; and

* represents a bonding position to a carbon atom of a benzene ring in the formula (2).

In the compound M2 of the exemplary embodiment, it is preferable that R₂₀₁ to R₂₆₀ as a substituent are each independently a halogen atom, an unsubstituted aryl group having 6 to 14 ring carbon atoms, an unsubstituted heterocyclic group having 5 to 14 ring atoms, or an unsubstituted alkyl group having 1 to 6 carbon atoms, and R₁₅₁ and R₁₅₂ as a substituent are each independently an unsubstituted aryl group having 6 to 14 ring carbon atoms or an unsubstituted alkyl group having 1 to 6 carbon atoms.

In the compound M2 of the exemplary embodiment, it is more preferable that R₂₀₁ to R₂₆₀ as a substituent are each independently an unsubstituted aryl group having 6 to 14 ring carbon atoms, an unsubstituted heterocyclic group having 5 to 14 ring atoms, or an unsubstituted alkyl group having 1 to 6 carbon atoms, and R₁₅₁ and R₁₅₂ as a substituent are each independently an unsubstituted aryl group having 6 to 14 ring carbon atoms or an unsubstituted alkyl group having 1 to 6 carbon atoms.

In the compound M2 of the exemplary embodiment, it is also preferable that R₂₀₁ to R₂₆₀ are each a hydrogen atom, and R₁₅₁ and R₁₅₂ as a substituent are each independently an unsubstituted aryl group having 6 to 14 ring carbon atoms or an unsubstituted alkyl group having 1 to 6 carbon atoms.

The compound M2 is exemplified by a compound represented by a formula (22) below.

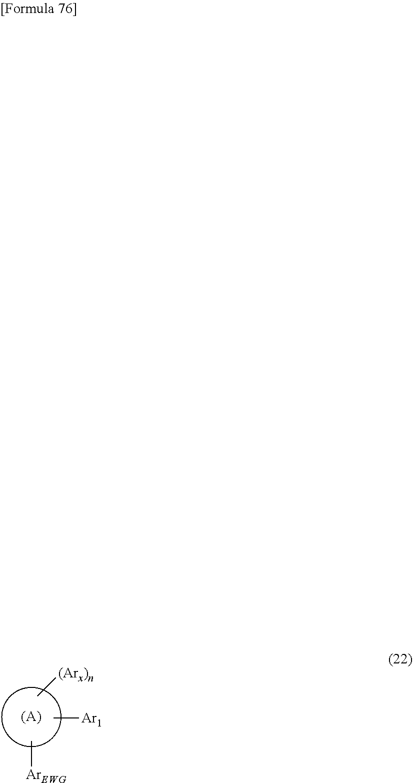

In the formula (22), Ar₁ is a group selected from the group consisting of a substituted or unsubstituted aryl group having 6 to 30 ring carbon atoms, a substituted or unsubstituted heteroaryl group having 5 to 30 ring atoms, a substituted or unsubstituted alkyl group having 1 to 30 carbon atoms, a substituted or unsubstituted fluoroalkyl group having 1 to 30 carbon atoms, a substituted or unsubstituted cycloalkyl group having 3 to 30 ring carbon atoms, a substituted or unsubstituted aralkyl group having 7 to 30 carbon atoms, a substituted phosphoryl group, a substituted silyl group, a cyano group, a nitro group, a carboxy group, and groups represented by formulae (1a) to (1j) below.

In the formula (22), Ar_{EWG} is a substituted or unsubstituted heteroaryl group having 5 to 30 ring atoms that includes at least one nitrogen atom in a ring, or an aryl group having 6 to 30 ring carbon atoms and substituted by at least one cyano group.

In the formula (22), Ar_{X} is each independently a hydrogen atom or a substituent, and Ar_{X} as a substituent is a group selected from the group consisting of a substituted or unsubstituted aryl group having 6 to 30 ring carbon atoms, a substituted or unsubstituted heteroaryl group having 5 to 30 ring atoms, a substituted or unsubstituted alkyl group having 1 to 30 carbon atoms, a substituted or unsubstituted fluoroalkyl group having 1 to 30 carbon atoms, a substituted or unsubstituted cycloalkyl group having 3 to 30 ring carbon atoms, a substituted or unsubstituted aralkyl group having 7 to 30 carbon atoms, a substituted phosphoryl group, a substituted silyl group, a cyano group, a nitro group, a carboxy group, and groups represented by the formulae (1a) to (1j).

In the formula (22), n is 0, 1, 2, 3, 4 or 5, and when n is 2, 3, 4 or 5, a plurality of Ar_{X} are mutually the same or different.

In the formula (22), a ring (A) is a five-membered ring, a six-membered ring, or a seven-membered ring. The ring (A) may be an aromatic hydrocarbon ring or a heterocycle. Ar₁ and Ar_{X} are bonded to respective ones of elements forming the ring (A).

In the formula (22), at least one of Ar₁ or Ar_{X} is a group selected from the group consisting of groups represented by the formulae (1a) to (1j).

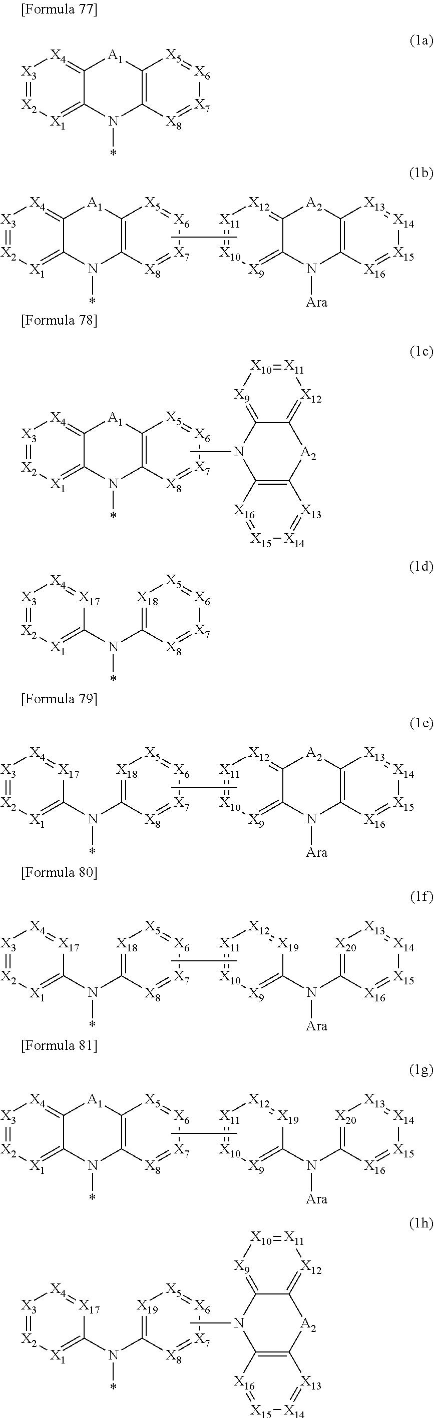

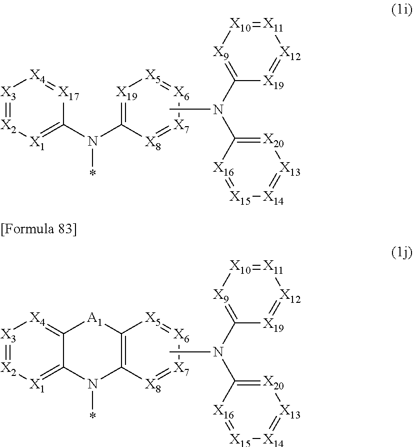

In the formulae (1a) to (1j), X₁ to X₂₀ are each independently a nitrogen atom (N) or a carbon atom bonded with R_{A1} (C—R_{A1}).

In the formula (1b), one of X₅ to X₈ is a carbon atom bonded to one of X₉ to X₁₂, and one of X₉ to X₁₂ is a carbon atom bonded to one of X₅ to X₈.

In the formula (1c), one of X₅ to X₈ is a carbon atom bonded to a nitrogen atom in a ring including A₂.

In the formula (1e), one of X₅ to X₈ and X₁₈ is a carbon atom bonded to one of X₉ to X₁₂, and one of X₉ to X₁₂ is a carbon atom bonded to one of X₅ to X₈ and X₁₈.

In the formula (1f), one of X₅ to X₈ and X₁₈ is a carbon atom bonded to one of X₉ to X₁₂ and X₁₉, and one of X₉ to X₁₂ and X₁₉ is a carbon atom bonded to one of X₅ to X₈ and X₁₈.

In the formula (1g), one of X₅ to X₈ is a carbon atom bonded to one of X₉ to X₁₂ and X₁₉, and one of X₉ to X₁₂ and X₁₉ is a carbon atom bonded to one of X₅ to X₈.

In the formula (1h), one of X₅ to X₈ and X₁₈ is a carbon atom bonded to a nitrogen atom in a ring including A₂.

In the formula (1i), one of X₅ to X₈ and X₁₈ is a carbon atom bonded to a nitrogen atom linking a ring including X₉ to X₁₂ and X₁₉ with a ring including X₁₃ to X₁₆ and X₂₀.

In the formula (1j), one of X₅ to X₈ is a carbon atom bonded to a nitrogen atom linking a ring including X₉ to X₁₂ and X₁₉ with a ring including X₁₃ to X₁₆ and X₂₀.

R_{A1} is each independently a hydrogen atom or a substituent, or at least one pair of pairs among a plurality of R_{A1} are mutually directly bonded to form a ring or bonded via a hetero atom to form a ring; and

R_{A1} as a substituent is a group selected from the group consisting of a substituted or unsubstituted aryl group having 6 to 30 ring carbon atoms, a substituted or unsubstituted heteroaryl group having 5 to 30 ring atoms, a substituted or unsubstituted alkyl group having 1 to 30 carbon atoms, a substituted or unsubstituted fluoroalkyl group having 1 to 30 carbon atoms, a substituted or unsubstituted cycloalkyl group having 3 to 30 ring carbon atoms, a substituted or unsubstituted aralkyl group having 7 to 30 carbon atoms, a substituted phosphoryl group, a substituted silyl group, a cyano group, a nitro group, and a carboxy group.

When a plurality of R_{A1} as a substituent are present, the plurality of R_{A1} are mutually the same or different.

In the formula (1a), when X₁ to X₈ are each a carbon atom bonded with R_{A1} (C—R_{A1}), a plurality of R_{A1} preferably form no ring.

In the formulae (1a) to (1j), * represents a bonding position to the ring (A).

In the formulae (1a) to (1j), A₁ and A₂ are each independently a single bond, an oxygen atom (O), a sulfur atom (S), C(R₂₀₂₁)(R₂₀₂₂), Si(R₂₀₂₃)(R₂₀₂₄), C(═O), S(═O), SO₂ or N(R₂₀₂₅). R₂₀₂₁ to R₂₀₂₅ are each independently a hydrogen atom or a substituent, and R₂₀₂₁ to R₂₀₂₅ as a substituent are each independently a group selected from the group consisting of a substituted or unsubstituted aryl group having 6 to 30 ring carbon atoms, a substituted or unsubstituted heteroaryl group having 5 to 30 ring atoms, a substituted or unsubstituted alkyl group having 1 to 30 carbon atoms, a substituted or unsubstituted fluoroalkyl group having 1 to 30 carbon atoms, a substituted or unsubstituted cycloalkyl group having 3 to 30 ring carbon atoms, a substituted or unsubstituted aralkyl group having 7 to 30 carbon atoms, a substituted phosphoryl group, a substituted silyl group, a cyano group, a nitro group, and a carboxy group.

In the formulae (1a) to (1j), Ara is a group selected from the group consisting of a substituted or unsubstituted aryl group having 6 to 30 ring carbon atoms, a substituted or unsubstituted heteroaryl group having 5 to 30 ring atoms, a substituted or unsubstituted alkyl group having 1 to 30 carbon atoms, a substituted or unsubstituted fluoroalkyl group having 1 to 30 carbon atoms, a substituted or unsubstituted cycloalkyl group having 3 to 30 ring carbon atoms, a substituted or unsubstituted aralkyl group having 7 to 30 carbon atoms, a substituted phosphoryl group, and a substituted silyl group. Ara is preferably a substituted or unsubstituted aryl group having 6 to 30 ring carbon atoms or a substituted or unsubstituted heteroaryl group having 5 to 30 ring atoms.

The formula (1a) is represented by a formula (1 aa) below when A₁ is a single bond, represented by a formula (1ab) below when A₁ is 0, represented by a formula (1ac) below when A₁ is S, represented by a formula (1ad) below when A₁ is C(R₂₀₂₁)(R₂₀₂₂), represented by a formula (1ae) below when A₁ is Si(R₂₀₂₃)(R₂₀₂₄), represented by a formula (1af) below when A₁ is C(═O), represented by a formula (1ag) below when A₁ is S(═O), represented by a formula (1 ah) below when A₁ is SO₂, and represented by a formula (1 ai) below when A₁ is N(R₂₀₂₅). In the formulae (1 aa) to (1 ai), X₁ to X₈ and R₂₀₂₁ to R₂₀₂₅ represent the same as described above. Linkages between rings via A₁ and A₂ in the formulae (1b), (1c), (1e) and (1g) to (1j) are the same as those in the formulae (1 aa) to (1 ai). In the formula (1 aa), when X₁ to X₈ are each a carbon atom bonded with R_{A1} (C—R_{A1}), a plurality of R_{A1} as a substituent preferably form no ring.

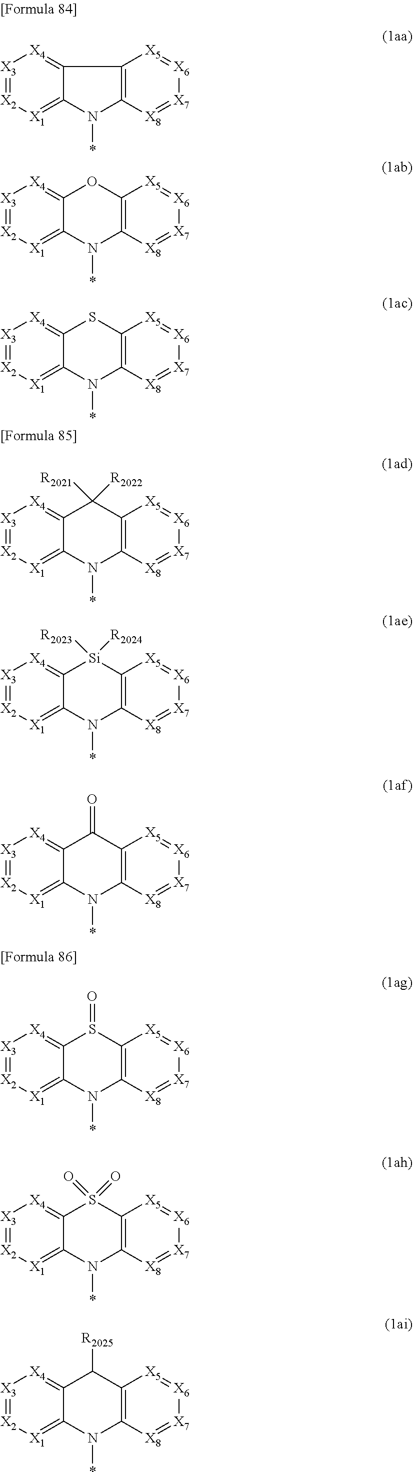

The compound M2 is preferably represented by a formula (221) below.

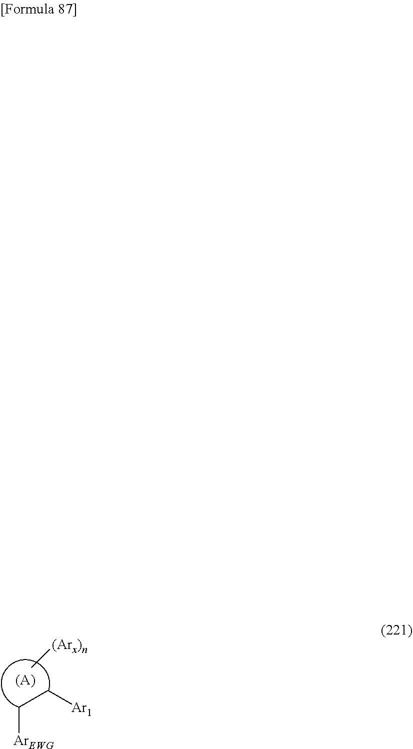

Ar₁, Ar_{EWG}, Arₓ, n and a ring (A) in the formula (221) respectively represent the same as Ar₁, Ar_{EWG}, Arₓ, n and the ring (A) in the formula (22).

The compound M2 is also preferably represented by a formula (222) below.

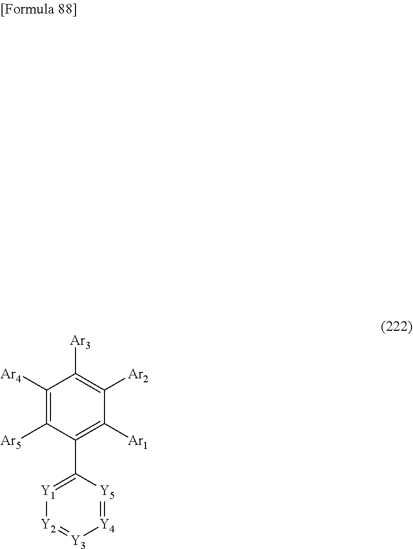

In the formula (222), Y₁ to Y₅ are each independently a nitrogen atom (N), a carbon atom bonded with a cyano group (C—CN), or a carbon atom bonded with R_{A2} (C—R_{A2}), and at least one of Y₁ to Y₅ is N or C—CN. A plurality of R_{A2} are mutually the same or different. R_{A2} is each independently a hydrogen atom or a substituent, R_{A2} as a substituent is a group selected from the group consisting of a substituted or unsubstituted aryl group having 6 to 30 ring carbon atoms, a substituted or unsubstituted heteroaryl group having 5 to 30 ring atoms, a substituted or unsubstituted alkyl group having 1 to 30 carbon atoms, a substituted or unsubstituted fluoroalkyl group having 1 to 30 carbon atoms, a substituted or unsubstituted cycloalkyl group having 3 to 30 ring carbon atoms, a substituted or unsubstituted aralkyl group having 7 to 30 carbon atoms, a substituted phosphoryl group, a substituted silyl group, a cyano group, a nitro group, and a carboxy group, and a plurality of R_{A2} are mutually the same or different.

In the formula (222), Ar₁ represents the same as Ar₁ in the formula (22).

In the formula (222), Ar₂ to Ar₅ are each independently a hydrogen atom or a substituent, and Ar₂ to Ar₅ as a substituent are each independently a group selected from the group consisting of a substituted or unsubstituted aryl group having 6 to 30 ring carbon atoms, a substituted or unsubstituted heteroaryl group having 5 to 30 ring atoms, a substituted or unsubstituted alkyl group having 1 to 30 carbon atoms, a substituted or unsubstituted fluoroalkyl group having 1 to 30 carbon atoms, a substituted or unsubstituted cycloalkyl group having 3 to 30 ring carbon atoms, a substituted or unsubstituted aralkyl group having 7 to 30 carbon atoms, a substituted phosphoryl group, a substituted silyl group, a cyano group, a nitro group, a carboxy group, and groups represented by the formulae (1a) to (1c).

In the formula (222), at least one of Ar₁ to Ar₅ is a group selected from the group consisting of groups represented by the formulae (1a) to (1c).

The compound M2 is also preferably a compound represented by a formula (11aa), (11bb) or (11cc) below.

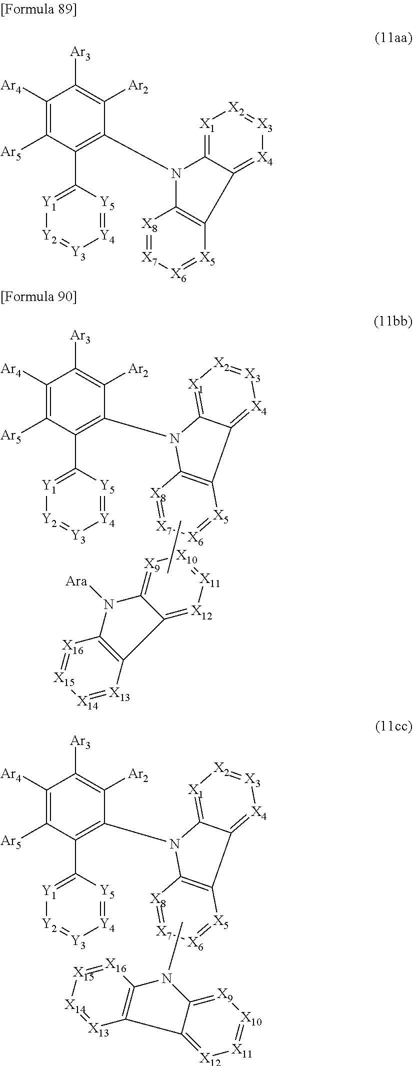

In the formulae (11aa), (11bb) and (11cc), Y₁ to Y₅, R_{A2}, Ar₂ to Ar₅, X₁ to X₁₆, R_{A1} and Ara respectively represent the same as the above-described Y₁ to Y₅, R_{A2}, Ar₂ to Ar₅, X₁ to X₁₆, R_{A1} and Ara.

The compound M2 is exemplified by a compound represented by a formula (23) below.

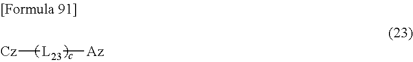

In the formula (23):

Az is a cyclic structure selected from the group consisting of a substituted or unsubstituted pyridine ring, a substituted or unsubstituted pyrimidine ring, a substituted or unsubstituted triazine ring, and a substituted or unsubstituted pyrazine ring;

c is 0, 1, 2, 3, 4 or 5;

when c is 0, Cz and Az are bonded by a single bond;

when c is 1, 2, 3, 4 or 5, L₂₃ is a linking group selected from the group consisting of a substituted or unsubstituted arylene group having 6 to 30 ring carbon atoms, and a substituted or unsubstituted heteroarylene group having 5 to 30 ring atoms;

when c is 2, 3, 4 or 5, a plurality of L₂₃ are mutually the same or different;

the plurality of L₂₃ are mutually bonded to form a ring or not bonded to form no ring; and

Cz is represented by a formula (23a) below.

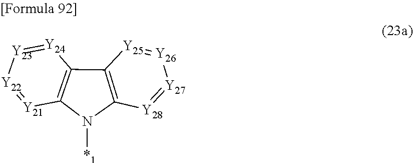

In the formula (23a):

Y₂₁ to Y₂₈ are each independently a nitrogen atom or CR_{A3};

R_{A3} is each independently a hydrogen atom or a substituent, or at least one pair of pairs among a plurality of R_{A3} are mutually bonded to form a ring;

R_{A3} as a substituent is each independently a group selected from the group consisting of a substituted or unsubstituted aryl group having 6 to 30 ring carbon atoms, a substituted or unsubstituted heteroaryl group having 5 to 30 ring atoms, a substituted or unsubstituted alkyl group having 1 to 30 carbon atoms, a substituted or unsubstituted fluoroalkyl group having 1 to 30 carbon atoms, a substituted or unsubstituted cycloalkyl group having 3 to 30 ring carbon atoms, a substituted or unsubstituted aralkyl group having 7 to 30 carbon atoms, a substituted phosphoryl group, a substituted silyl group, a cyano group, a nitro group, and a carboxy group;

a plurality of R_{A3} are mutually the same or different; and

*1 represents a bonding position to a carbon atom in a structure of a linking group represented by L₂₃, or a bonding position to a carbon atom in a cyclic structure represented by Az.

Y₂₁ to Y₂₈ are also preferably CR_{A3}.

c in the formula (23) is preferably 0 or 1.

Cz is also preferably represented by a formula (23b), (23c) or (23d) below.

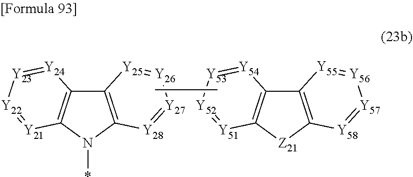

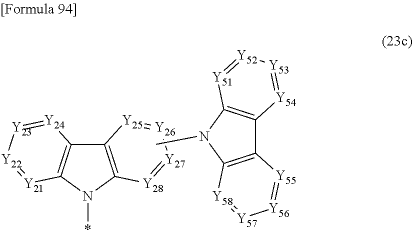

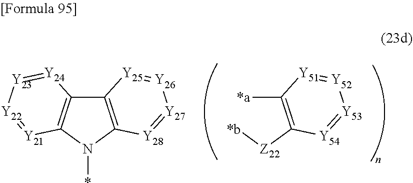

In the formulae (23b), (23c) and (23d), Y₂₁ to Y₂₈ and Y₅₁ to Y₅₈ are each independently a nitrogen atom or CR_{A4};

in the formula (23b), at least one of Y₂₅ to Y₂₈ is a carbon atom bonded to one of Y₅₁ to Y₅₄, and at least one of Y₅₁ to Y₅₄ is a carbon atom bonded to one of Y₂₅ to Y₂₈;

in the formula (23c), at least one of Y₂₅ to Y₂₈ is a carbon atom bonded to a nitrogen atom in a five-membered ring of a nitrogen-containing fused ring including Y₅₁ to Y₅₈;

in the formula (23d), *a and *b each represent a bonding position to one of Y₂₁ to Y₂₈, at least one of Y₂₅ to Y₂₈ is the bonding position represented by *a, and at least one of Y₂₅ to Y₂₈ is the bonding position represented by *b;

n is 1, 2, 3 or 4;

R_{A4} is each independently a hydrogen atom or a substituent, or at least one pair of pairs among a plurality of R_{A4} are mutually bonded to form a ring;

R_{A4} as a substituent is each independently a substituent selected from the group consisting of a substituted or unsubstituted aryl group having 6 to 30 ring carbon atoms, a substituted or unsubstituted heteroaryl group having 5 to 30 ring atoms, a substituted or unsubstituted alkyl group having 1 to 30 carbon atoms, a substituted or unsubstituted fluoroalkyl group having 1 to 30 carbon atoms, a substituted or unsubstituted cycloalkyl group having 3 to 30 ring carbon atoms, a substituted or unsubstituted aralkyl group having 7 to 30 carbon atoms, a substituted phosphoryl group, a substituted silyl group, a cyano group, a nitro group, and a carboxy group;

a plurality of R_{A4} are mutually the same or different;

Z₂₁ and Z₂₂ are each independently any one selected from the group consisting of an oxygen atom, a sulfur atom, NR₄₅ and CR₄₆R₄₇;

R₄₅ is a hydrogen atom or a substituent;

R₄₆ and R₄₇ are each independently a hydrogen atom or a substituent, or a pair of R₄₆ and R₄₇ are mutually bonded to form a ring;

R₄₅, R₄₆ and R₄₇ as a substituent are each independently a substituent selected from the group consisting of a substituted or unsubstituted aryl group having 6 to 30 ring carbon atoms, a substituted or unsubstituted heteroaryl group having 5 to 30 ring atoms, a substituted or unsubstituted alkyl group having 1 to 30 carbon atoms, a substituted or unsubstituted fluoroalkyl group having 1 to 30 carbon atoms, a substituted or unsubstituted cycloalkyl group having 3 to 30 ring carbon atoms, a substituted or unsubstituted aralkyl group having 7 to 30 carbon atoms, a substituted phosphoryl group, a substituted silyl group, a cyano group, a nitro group, and a carboxy group;

a plurality of R₄₅ are mutually the same or different;

a plurality of R₄₆ are mutually the same or different;

a plurality of R₄₇ are mutually the same or different; and

* represents a bonding position to a carbon atom in a structure of a linking group represented by L₂₃, or a bonding position to a carbon atom in a cyclic structure represented by Az.

Z₂₁ is preferably NR₄₅.

When Z₂₁ is NR₄₅, R₄₅ is preferably a substituted or unsubstituted aryl group having 6 to 30 ring carbon atoms.

Z₂₂ is preferably NR₄₅.

When Z₂₂ is NR₄₅, R₄₅ is preferably a substituted or unsubstituted aryl group having 6 to 30 ring carbon atoms.

Y₅₁ to Y₅₈ are preferably CR_{A4}, provided that at least one of Y₅₁ to Y₅₈ is a carbon atom bonded to a cyclic structure represented by the formula (23a).

It is also preferable that Cz is represented by the formula (23d) and n is 1.

Az is preferably a cyclic structure selected from the group consisting of a substituted or unsubstituted pyrimidine group and a substituted or unsubstituted triazine group.

Az is a cyclic structure selected from the group consisting of a substituted pyrimidine ring and a substituted triazine ring, in which a substituent of each of the substituted pyrimidine ring and the substituted triazine ring is more preferably a group selected from the group consisting of a substituted or unsubstituted aryl group having 6 to 30 ring carbon atoms and a substituted or unsubstituted heteroaryl group having 5 to 30 ring atoms, further preferably a substituted or unsubstituted aryl group having 6 to 30 ring carbon atoms.

When the pyrimidine ring and the triazine ring as Az have a substituted or unsubstituted aryl group as a substituent, the aryl group preferably has 6 to 20 ring carbon atoms, more preferably 6 to 14 ring carbon atoms, further preferably 6 to 12 ring carbon atoms.

When Az has a substituted or unsubstituted aryl group as a substituent, the substituent is preferably a group selected from the group consisting of a substituted or unsubstituted phenyl group, a substituted or unsubstituted biphenyl group, a substituted or unsubstituted naphthyl group, a substituted or unsubstituted phenanthryl group, a substituted or unsubstituted terphenyl group, and a substituted or unsubstituted fluorenyl group, more preferably a group selected from the group consisting of a substituted or unsubstituted phenyl group, a substituted or unsubstituted biphenyl group, and a substituted or unsubstituted naphthyl group.

When Az has a substituted or unsubstituted heteroaryl group as a substituent, the substituent is preferably a substituent selected from the group consisting of a substituted or unsubstituted carbazolyl group, a substituted or unsubstituted dibenzofuranyl group, and a substituted or unsubstituted dibenzothiophenyl group.

It is preferable that R_{A4} is each independently a hydrogen atom or a substituent, and R_{A4} as a substituent is a substituent selected from the group consisting of a substituted or unsubstituted aryl group having 6 to 30 ring carbon atoms and a substituted or unsubstituted heteroaryl group having 5 to 30 ring atoms.

When R_{A4} as a substituent is a substituted or unsubstituted aryl group having 6 to 30 ring carbon atoms, R_{A4} as a substituent is preferably a substituent selected from the group consisting of a substituted or unsubstituted phenyl group, a substituted or unsubstituted biphenyl group, a substituted or unsubstituted naphthyl group, a substituted or unsubstituted phenanthryl group, a substituted or unsubstituted terphenyl group, and a substituted or unsubstituted fluorenyl group, more preferably a substituent selected from the group consisting of a substituted or unsubstituted phenyl group, a substituted or unsubstituted biphenyl group, and a substituted or unsubstituted naphthyl group.

When R_{A4} as a substituent is a substituted or unsubstituted heteroaryl group having 5 to 30 ring atoms, R_{A4} as a substituent is preferably a substituent selected from the group consisting of a substituted or unsubstituted carbazolyl group, a substituted or unsubstituted dibenzofuranyl group, and a substituted or unsubstituted dibenzothiophenyl group.

R₄₅, R₄₆ and R₄₇ as a substituent are preferably each independently a substituent selected from the group consisting of a substituted or unsubstituted aryl group having 6 to 30 ring carbon atoms, a substituted or unsubstituted heteroaryl group having 5 to 30 ring atoms, and a substituted or unsubstituted alkyl group having 1 to 30 carbon atoms.

### Manufacturing Method of Compound M2 According to Exemplary Embodiment

The compound M2 of the exemplary embodiment can be manufactured by a known method.

Specific examples of the compound M2 of the exemplary embodiment include compounds below. It should however be noted that the invention is not limited to the specific examples of the compound.

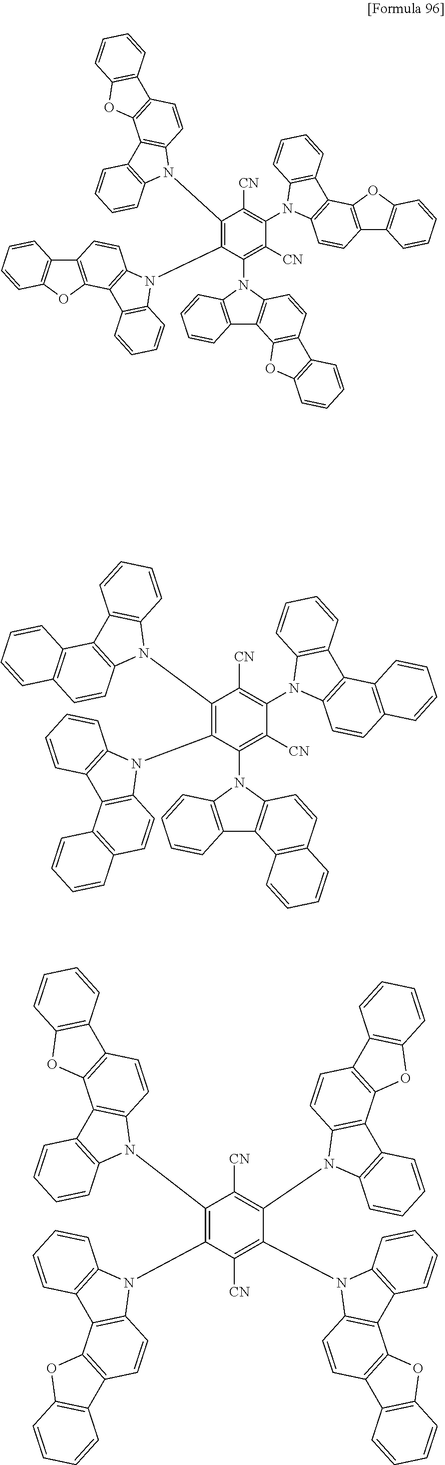 [SMILES: N#Cc1c(-n2c3ccccc3c3c4oc5ccccc5c4ccc32)c(C#N)c(-n2c3ccccc3c3c4oc5ccccc5c4ccc32)c(-n2c3ccccc3c3c4oc5ccccc5c4ccc32)c1-n1c2ccccc2c2c3oc4ccccc4c3ccc21 ; N#Cc1c(-n2c3ccccc3c3c4ccccc4ccc32)c(C#N)c(-n2c3ccccc3c3c4ccccc4ccc32)c(-n2c3ccccc3c3c4ccccc4ccc32)c1-n1c2ccccc2c2c3ccccc3ccc21 ; N#Cc1c(-n2c3ccccc3c3c4oc5ccccc5c4ccc32)c(-n2c3ccccc3c3c4oc5ccccc5c4ccc32)c(C#N)c(-n2c3ccccc3c3c4oc5ccccc5c4ccc32)c1-n1c2ccccc2c2c3oc4ccccc4c3ccc21]

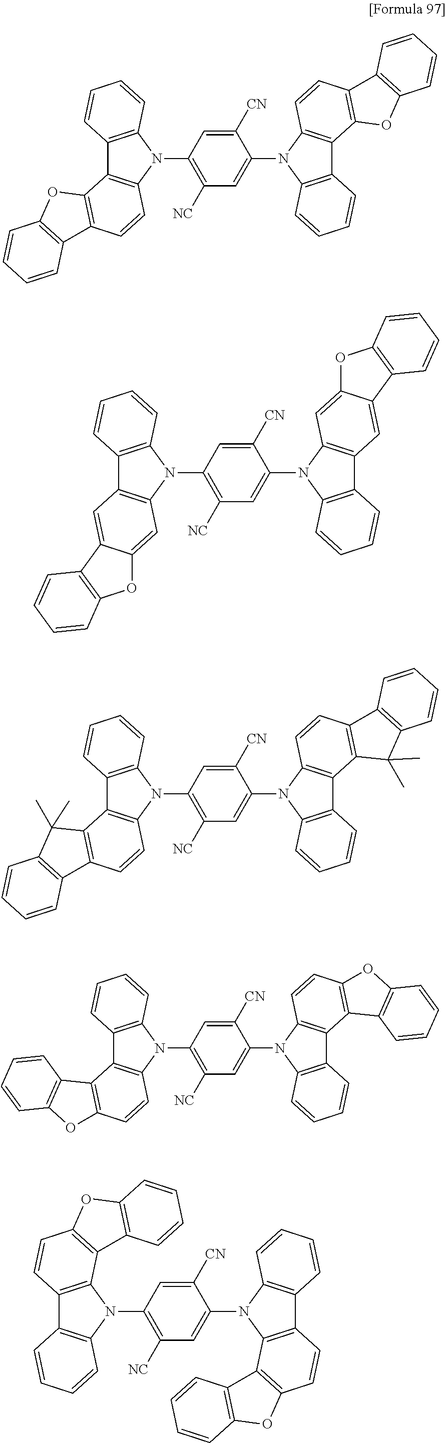 [SMILES: N#Cc1cc(-n2c3ccccc3c3c4oc5ccccc5c4ccc32)c(C#N)cc1-n1c2ccccc2c2c3oc4ccccc4c3ccc21 ; N#Cc1cc(-n2c3ccccc3c3cc4c(cc32)oc2ccccc24)c(C#N)cc1-n1c2ccccc2c2cc3c(cc21)oc1ccccc13 ; CC1(C)c2ccccc2-c2ccc3c(c21)c1ccccc1n3-c1cc(C#N)c(-n2c3ccccc3c3c4c(ccc32)-c2ccccc2C4(C)C)cc1C#N ; N#Cc1cc(-n2c3ccccc3c3c4c(ccc32)oc2ccccc24)c(C#N)cc1-n1c2ccccc2c2c3c(ccc21)oc1ccccc13 ; N#Cc1cc(-n2c3ccccc3c3ccc4oc5ccccc5c4c32)c(C#N)cc1-n1c2ccccc2c2ccc3oc4ccccc4c3c21]

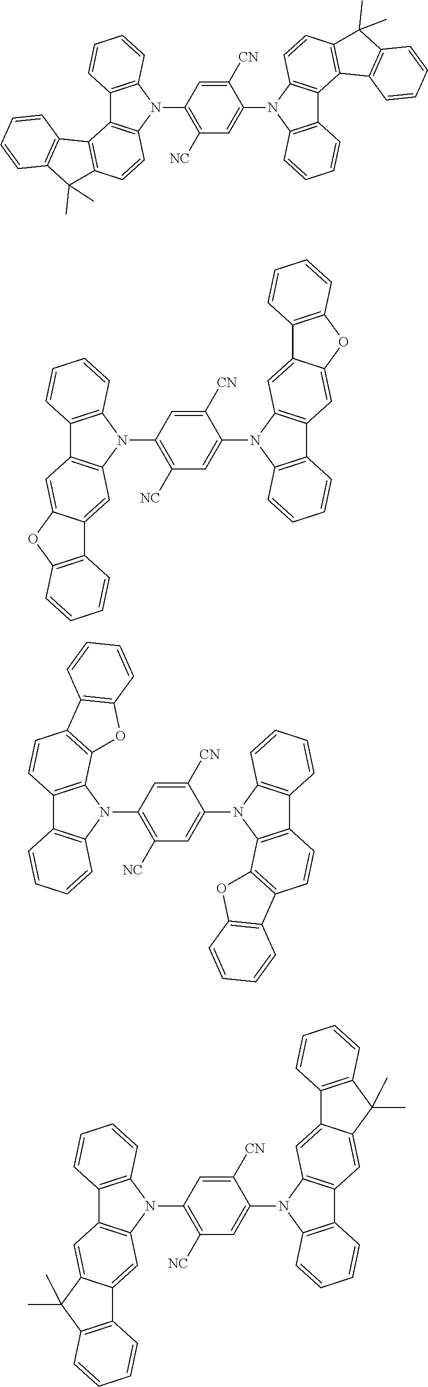

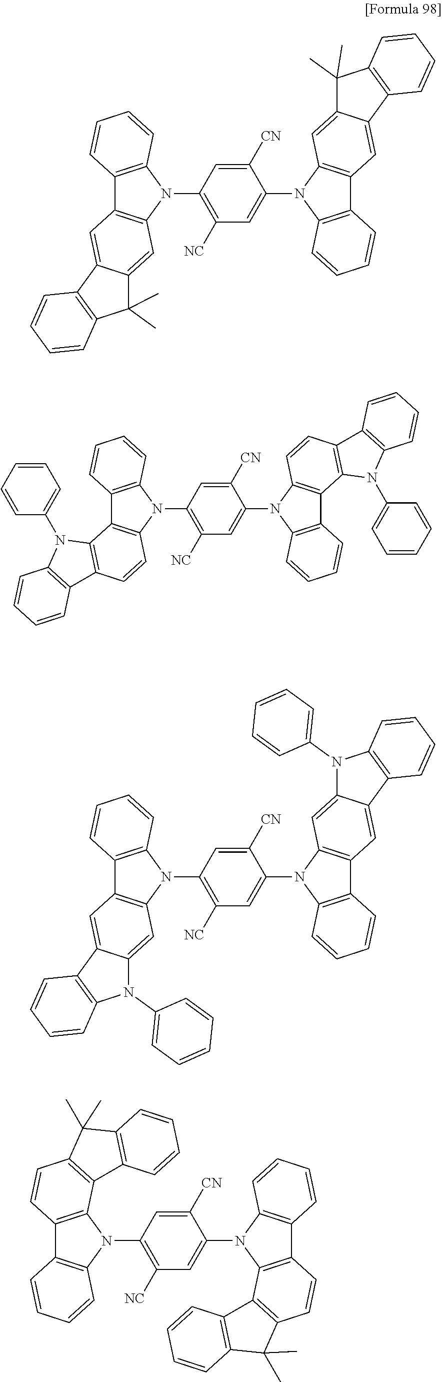 [SMILES: CC1(C)c2ccccc2-c2cc3c4ccccc4n(-c4cc(C#N)c(-n5c6ccccc6c6cc7c(cc65)C(C)(C)c5ccccc5-7)cc4C#N)c3cc21 ; N#Cc1cc(-n2c3ccccc3c3c2ccc2c4ccccc4n(-c4ccccc4)c23)c(C#N)cc1-n1c2ccccc2c2c1ccc1c3ccccc3n(-c3ccccc3)c12 ; N#Cc1cc(-n2c3ccccc3c3cc4c5ccccc5n(-c5ccccc5)c4cc32)c(C#N)cc1-n1c2ccccc2c2cc3c4ccccc4n(-c4ccccc4)c3cc21 ; CC1(C)c2ccccc2-c2c1ccc1c3ccccc3n(-c3cc(C#N)c(-n4c5ccccc5c5ccc6c(c54)-c4ccccc4C6(C)C)cc3C#N)c21]

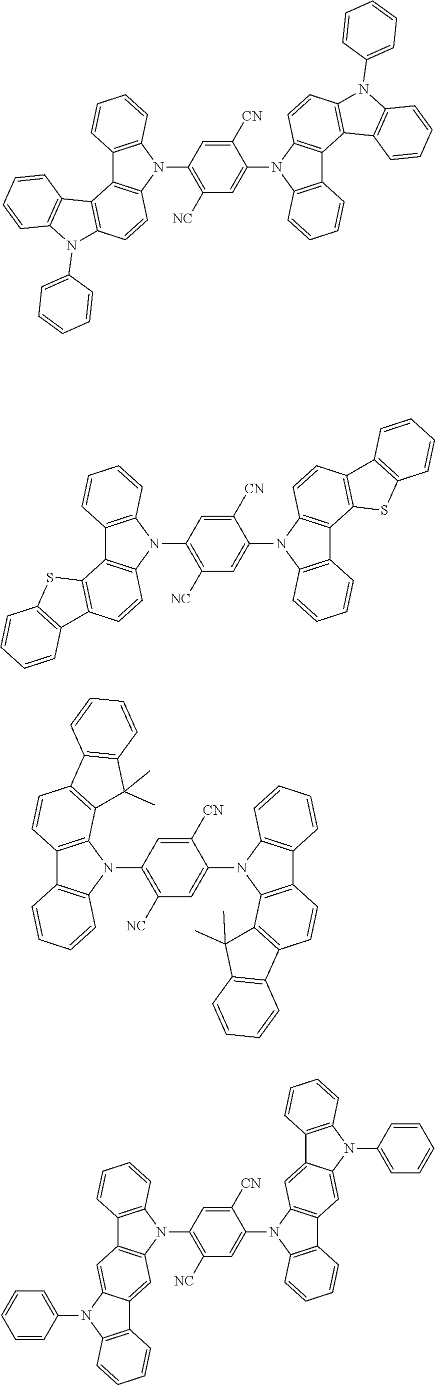 [SMILES: N#Cc1cc(-n2c3ccccc3c3c4c5ccccc5n(-c5ccccc5)c4ccc32)c(C#N)cc1-n1c2ccccc2c2c3c4ccccc4n(-c4ccccc4)c3ccc21 ; N#Cc1cc(-n2c3ccccc3c3c4sc5ccccc5c4ccc32)c(C#N)cc1-n1c2ccccc2c2c3sc4ccccc4c3ccc21 ; CC1(C)c2ccccc2-c2ccc3c4ccccc4n(-c4cc(C#N)c(-n5c6ccccc6c6ccc7c(c65)C(C)(C)c5ccccc5-7)cc4C#N)c3c21 ; N#Cc1cc(-n2c3ccccc3c3cc4c(cc32)c2ccccc2n4-c2ccccc2)c(C#N)cc1-n1c2ccccc2c2cc3c(cc21)c1ccccc1n3-c1ccccc1]

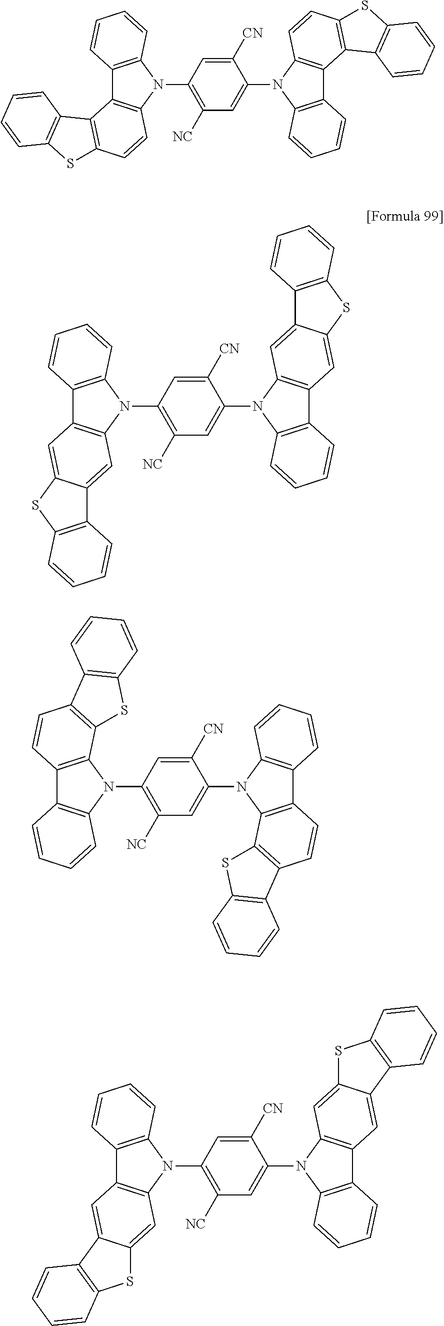

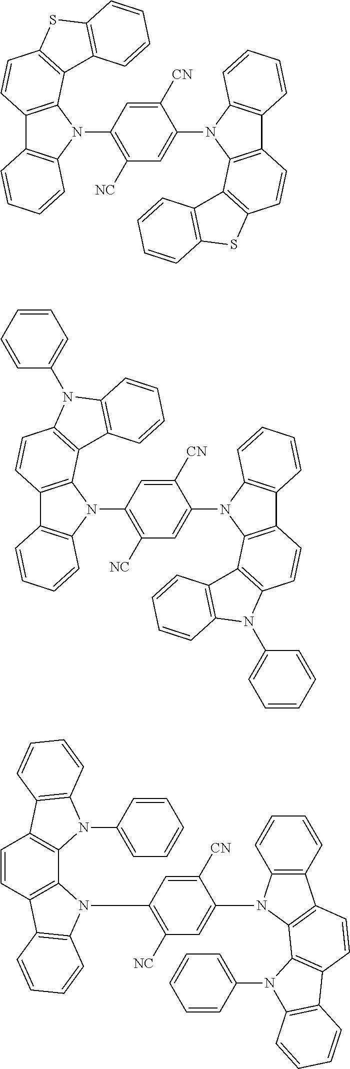 [SMILES: N#Cc1cc(-n2c3ccccc3c3ccc4sc5ccccc5c4c32)c(C#N)cc1-n1c2ccccc2c2ccc3sc4ccccc4c3c21 ; N#Cc1cc(-n2c3ccccc3c3ccc4c(c5ccccc5n4-c4ccccc4)c32)c(C#N)cc1-n1c2ccccc2c2ccc3c(c4ccccc4n3-c3ccccc3)c21 ; N#Cc1cc(-n2c3ccccc3c3ccc4c5ccccc5n(-c5ccccc5)c4c32)c(C#N)cc1-n1c2ccccc2c2ccc3c4ccccc4n(-c4ccccc4)c3c21]

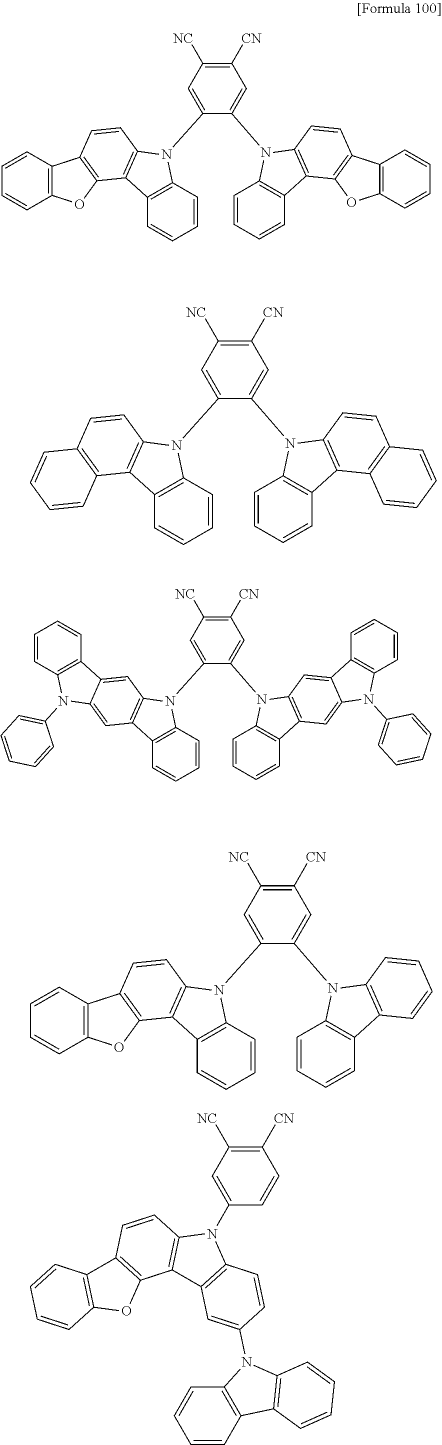

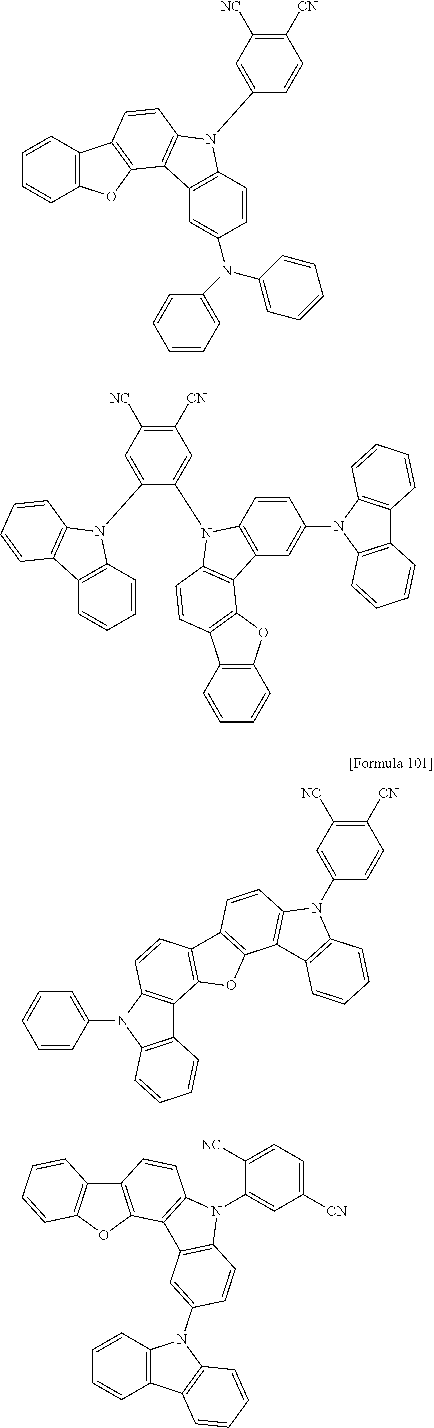 [SMILES: N#Cc1ccc(-n2c3ccc(N(c4ccccc4)c4ccccc4)cc3c3c4oc5ccccc5c4ccc32)cc1C#N ; N#Cc1cc(-n2c3ccccc3c3ccccc32)c(-n2c3ccc(-n4c5ccccc5c5ccccc54)cc3c3c4oc5ccccc5c4ccc32)cc1C#N ; N#Cc1ccc(-n2c3ccccc3c3c4oc5c(ccc6c5c5ccccc5n6-c5ccccc5)c4ccc32)cc1C#N ; N#Cc1ccc(C#N)c(-n2c3ccc(-n4c5ccccc5c5ccccc54)cc3c3c4oc5ccccc5c4ccc32)c1]

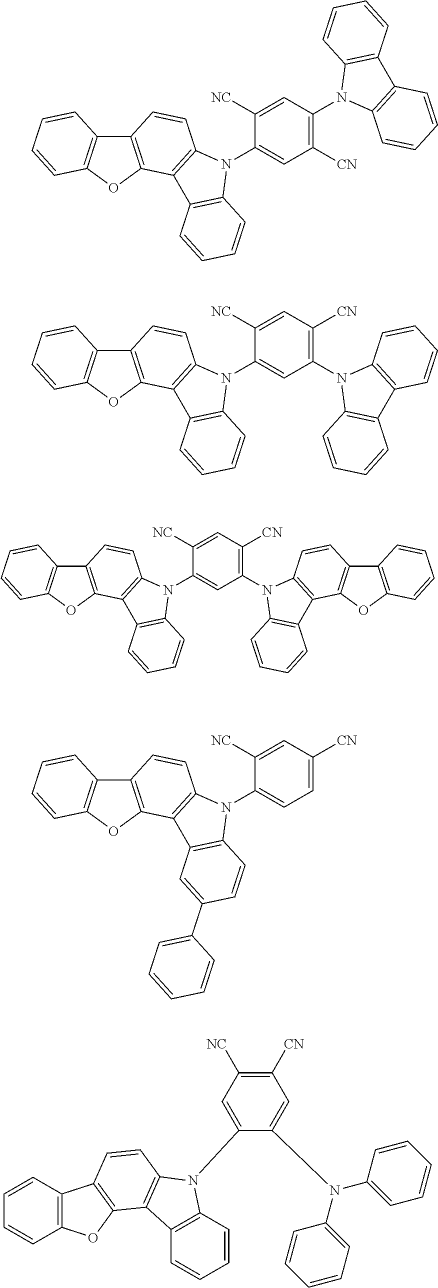 [SMILES: N#Cc1cc(-n2c3ccccc3c3c4oc5ccccc5c4ccc32)c(C#N)cc1-n1c2ccccc2c2ccccc21 ; N#Cc1cc(C#N)c(-n2c3ccccc3c3c4oc5ccccc5c4ccc32)cc1-n1c2ccccc2c2ccccc21 ; N#Cc1cc(C#N)c(-n2c3ccccc3c3c4oc5ccccc5c4ccc32)cc1-n1c2ccccc2c2c3oc4ccccc4c3ccc21 ; N#Cc1ccc(-n2c3ccc(-c4ccccc4)cc3c3c4oc5ccccc5c4ccc32)c(C#N)c1 ; N#Cc1cc(N(c2ccccc2)c2ccccc2)c(-n2c3ccccc3c3c4oc5ccccc5c4ccc32)cc1C#N]

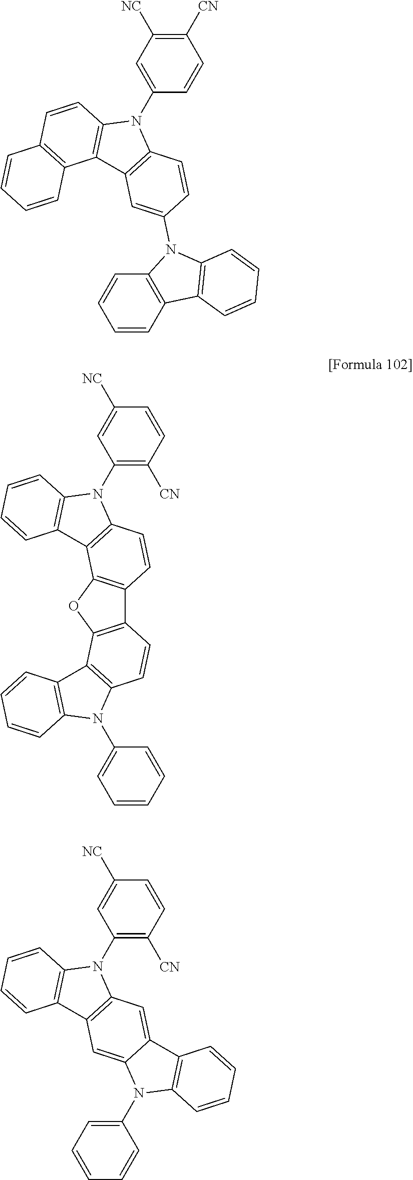 [SMILES: N#Cc1ccc(-n2c3ccc(-n4c5ccccc5c5ccccc54)cc3c3c4ccccc4ccc32)cc1C#N ; N#Cc1ccc(C#N)c(-n2c3ccccc3c3c4oc5c(ccc6c5c5ccccc5n6-c5ccccc5)c4ccc32)c1 ; N#Cc1ccc(C#N)c(-n2c3ccccc3c3cc4c(cc32)c2ccccc2n4-c2ccccc2)c1]

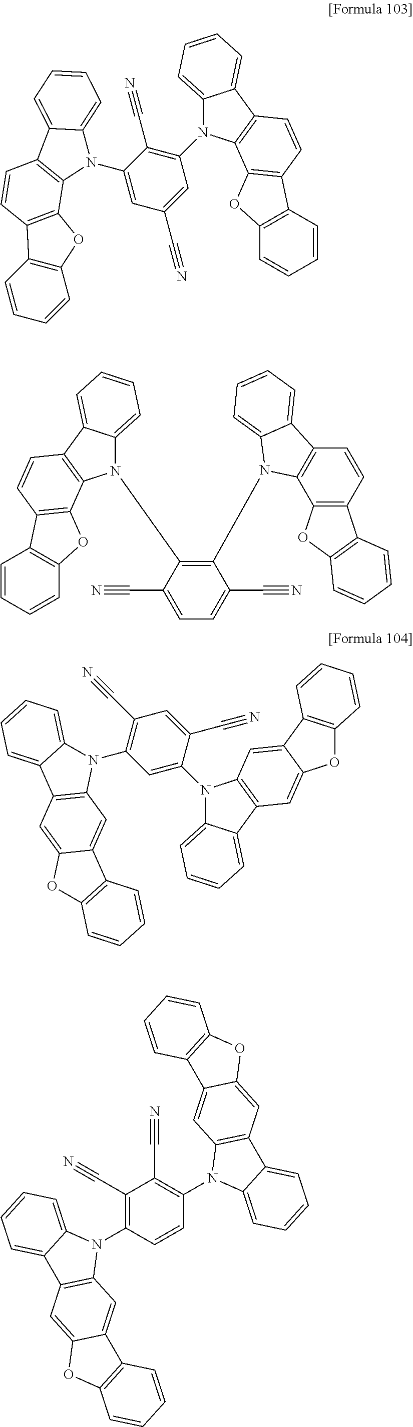 [SMILES: N#Cc1cc(-n2c3ccccc3c3ccc4c5ccccc5oc4c32)c(C#N)c(-n2c3ccccc3c3ccc4c5ccccc5oc4c32)c1 ; N#Cc1ccc(C#N)c(-n2c3ccccc3c3ccc4c5ccccc5oc4c32)c1-n1c2ccccc2c2ccc3c4ccccc4oc3c21 ; N#Cc1cc(C#N)c(-n2c3ccccc3c3cc4oc5ccccc5c4cc32)cc1-n1c2ccccc2c2cc3oc4ccccc4c3cc21 ; N#Cc1c(-n2c3ccccc3c3cc4oc5ccccc5c4cc32)ccc(-n2c3ccccc3c3cc4oc5ccccc5c4cc32)c1C#N]

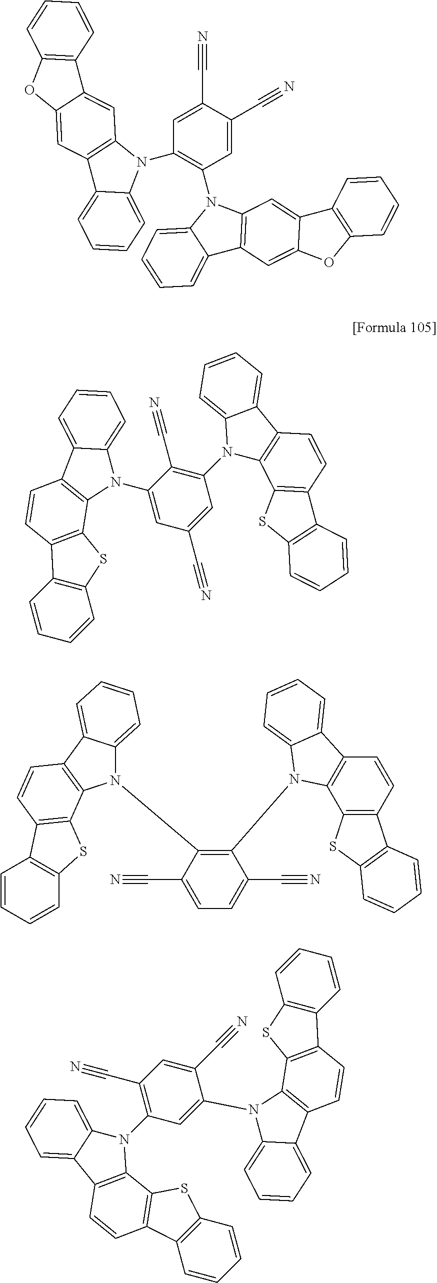 [SMILES: N#Cc1cc(-n2c3ccccc3c3cc4oc5ccccc5c4cc32)c(-n2c3ccccc3c3cc4oc5ccccc5c4cc32)cc1C#N ; N#Cc1cc(-n2c3ccccc3c3ccc4c5ccccc5sc4c32)c(C#N)c(-n2c3ccccc3c3ccc4c5ccccc5sc4c32)c1 ; N#Cc1ccc(C#N)c(-n2c3ccccc3c3ccc4c5ccccc5sc4c32)c1-n1c2ccccc2c2ccc3c4ccccc4sc3c21 ; N#Cc1cc(C#N)c(-n2c3ccccc3c3ccc4c5ccccc5sc4c32)cc1-n1c2ccccc2c2ccc3c4ccccc4sc3c21]

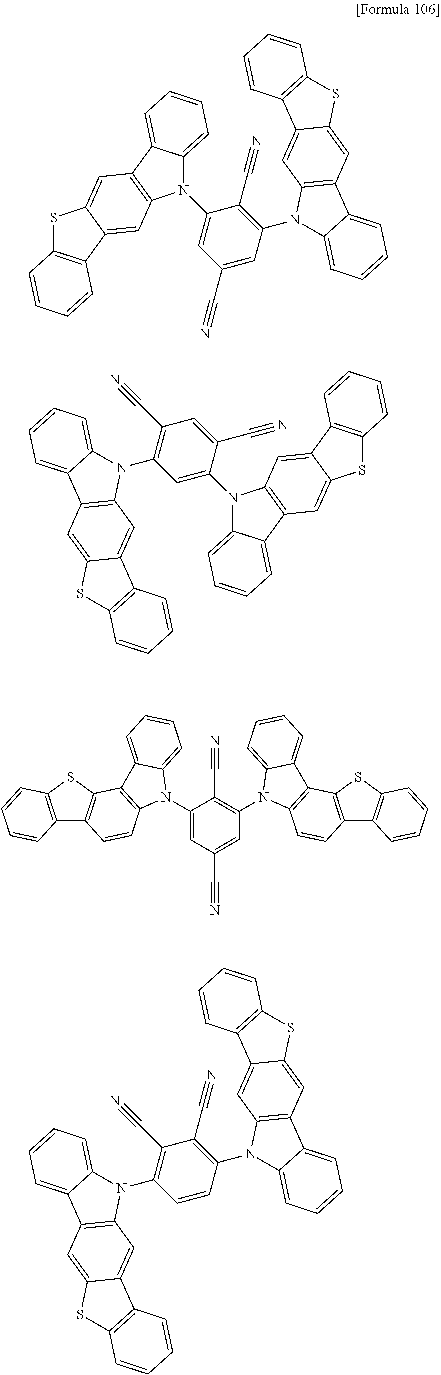 [SMILES: N#Cc1cc(-n2c3ccccc3c3cc4sc5ccccc5c4cc32)c(C#N)c(-n2c3ccccc3c3cc4sc5ccccc5c4cc32)c1 ; N#Cc1cc(C#N)c(-n2c3ccccc3c3cc4sc5ccccc5c4cc32)cc1-n1c2ccccc2c2cc3sc4ccccc4c3cc21 ; N#Cc1cc(-n2c3ccccc3c3c4sc5ccccc5c4ccc32)c(C#N)c(-n2c3ccccc3c3c4sc5ccccc5c4ccc32)c1 ; N#Cc1c(-n2c3ccccc3c3cc4sc5ccccc5c4cc32)ccc(-n2c3ccccc3c3cc4sc5ccccc5c4cc32)c1C#N]

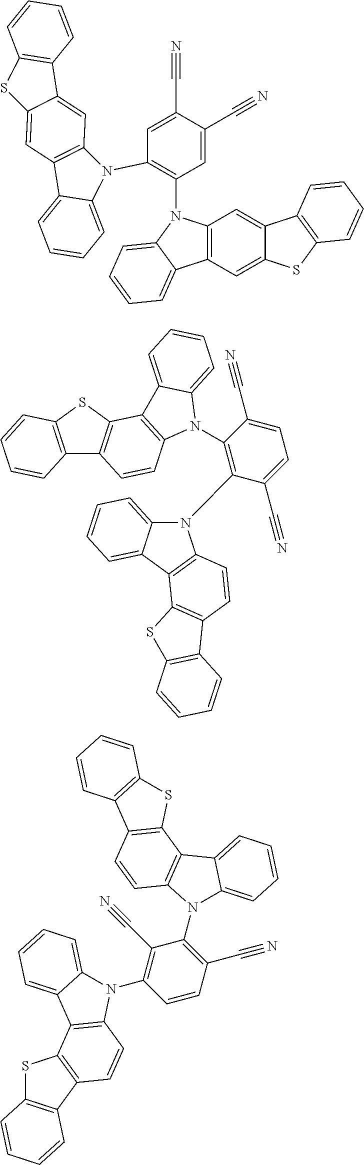

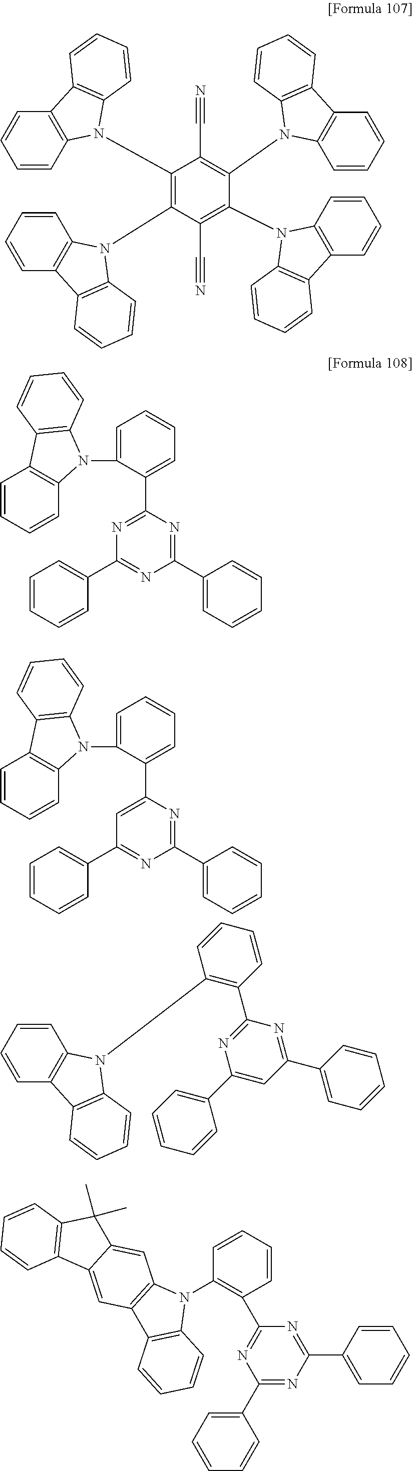

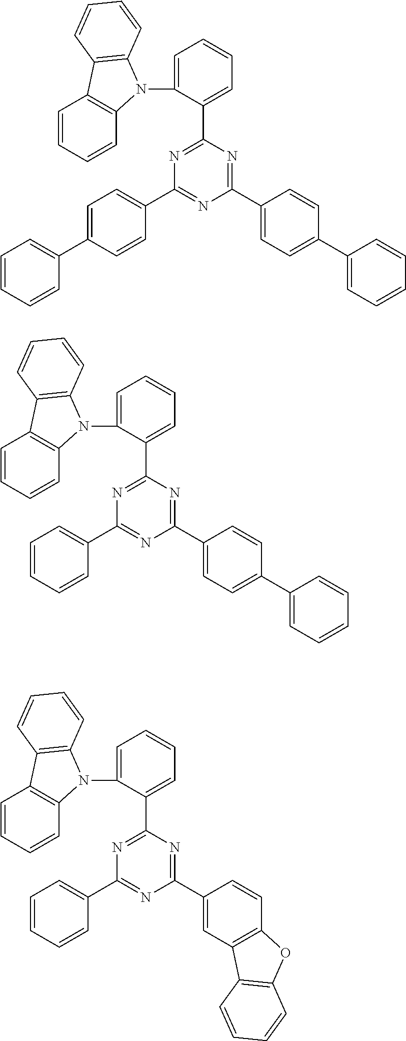

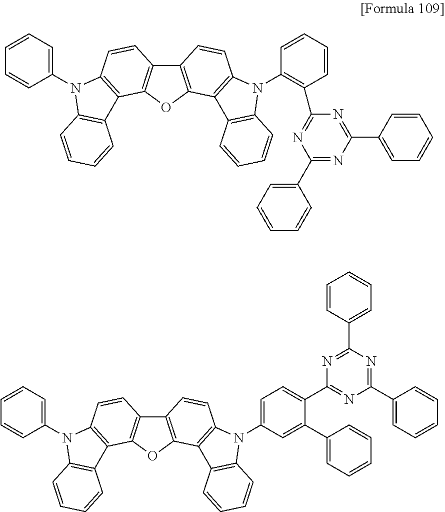 [SMILES: c1ccc(-c2nc(-c3ccccc3)nc(-c3ccccc3-n3c4ccccc4c4c5oc6c(ccc7c6c6ccccc6n7-c6ccccc6)c5ccc43)n2)cc1 ; c1ccc(-c2nc(-c3ccccc3)nc(-c3ccc(-n4c5ccccc5c5c6oc7c(ccc8c7c7ccccc7n8-c7ccccc7)c6ccc54)cc3-c3ccccc3)n2)cc1]

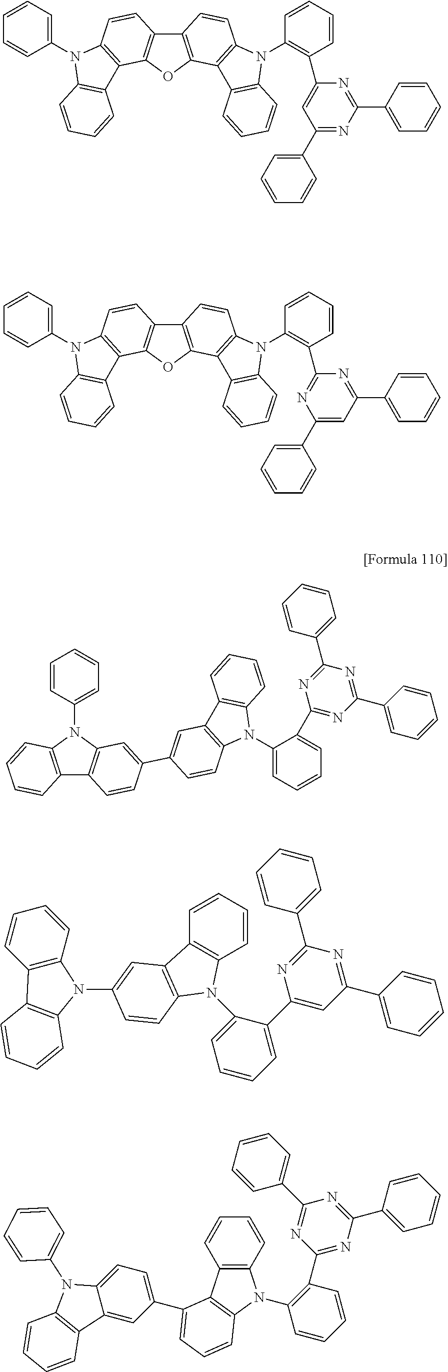 [SMILES: c1ccc(-c2cc(-c3ccccc3-n3c4ccccc4c4c5oc6c(ccc7c6c6ccccc6n7-c6ccccc6)c5ccc43)nc(-c3ccccc3)n2)cc1 ; c1ccc(-c2cc(-c3ccccc3)nc(-c3ccccc3-n3c4ccccc4c4c5oc6c(ccc7c6c6ccccc6n7-c6ccccc6)c5ccc43)n2)cc1 ; c1ccc(-c2nc(-c3ccccc3)nc(-c3ccccc3-n3c4ccccc4c4cc(-c5ccc6c7ccccc7n(-c7ccccc7)c6c5)ccc43)n2)cc1 ; c1ccc(-c2cc(-c3ccccc3-n3c4ccccc4c4cc(-n5c6ccccc6c6ccccc65)ccc43)nc(-c3ccccc3)n2)cc1 ; c1ccc(-c2nc(-c3ccccc3)nc(-c3ccccc3-n3c4ccccc4c4c(-c5ccc6c(c5)c5ccccc5n6-c5ccccc5)cccc43)n2)cc1]

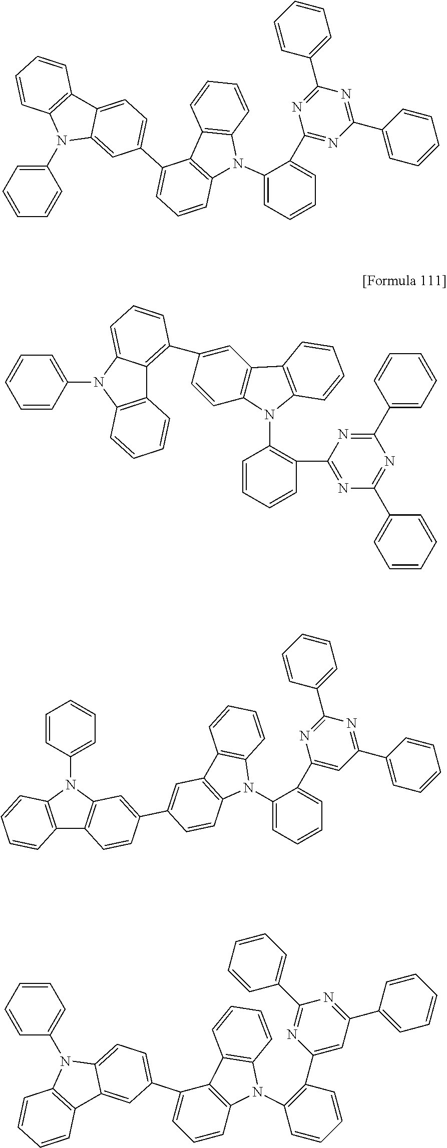 [SMILES: c1ccc(-c2nc(-c3ccccc3)nc(-c3ccccc3-n3c4ccccc4c4c(-c5ccc6c7ccccc7n(-c7ccccc7)c6c5)cccc43)n2)cc1 ; c1ccc(-c2nc(-c3ccccc3)nc(-c3ccccc3-n3c4ccccc4c4cc(-c5cccc6c5c5ccccc5n6-c5ccccc5)ccc43)n2)cc1 ; c1ccc(-c2cc(-c3ccccc3-n3c4ccccc4c4cc(-c5ccc6c7ccccc7n(-c7ccccc7)c6c5)ccc43)nc(-c3ccccc3)n2)cc1 ; c1ccc(-c2cc(-c3ccccc3-n3c4ccccc4c4c(-c5ccc6c(c5)c5ccccc5n6-c5ccccc5)cccc43)nc(-c3ccccc3)n2)cc1]

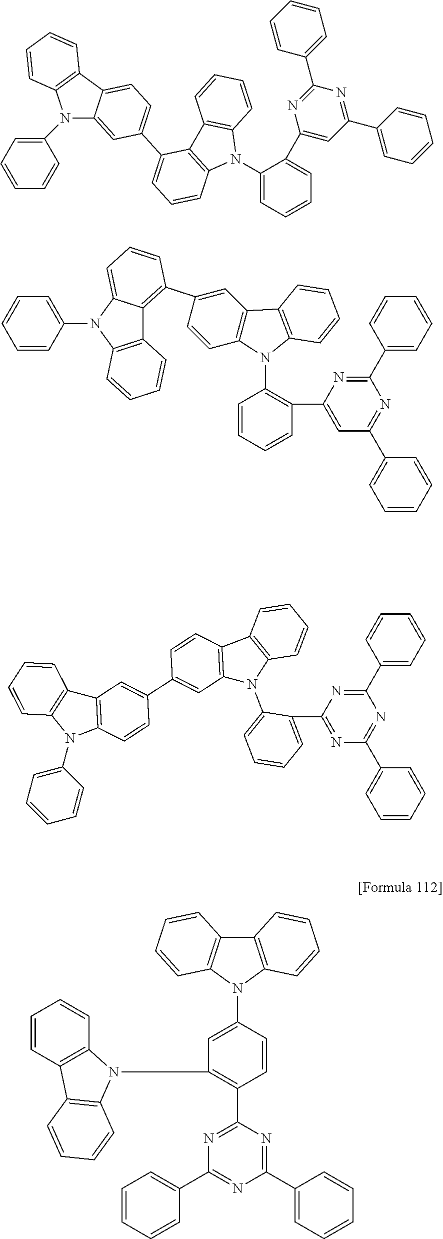 [SMILES: c1ccc(-c2cc(-c3ccccc3-n3c4ccccc4c4c(-c5ccc6c7ccccc7n(-c7ccccc7)c6c5)cccc43)nc(-c3ccccc3)n2)cc1 ; c1ccc(-c2cc(-c3ccccc3-n3c4ccccc4c4cc(-c5cccc6c5c5ccccc5n6-c5ccccc5)ccc43)nc(-c3ccccc3)n2)cc1 ; c1ccc(-c2nc(-c3ccccc3)nc(-c3ccccc3-n3c4ccccc4c4ccc(-c5ccc6c(c5)c5ccccc5n6-c5ccccc5)cc43)n2)cc1 ; c1ccc(-c2nc(-c3ccccc3)nc(-c3ccc(-n4c5ccccc5c5ccccc54)cc3-n3c4ccccc4c4ccccc43)n2)cc1]

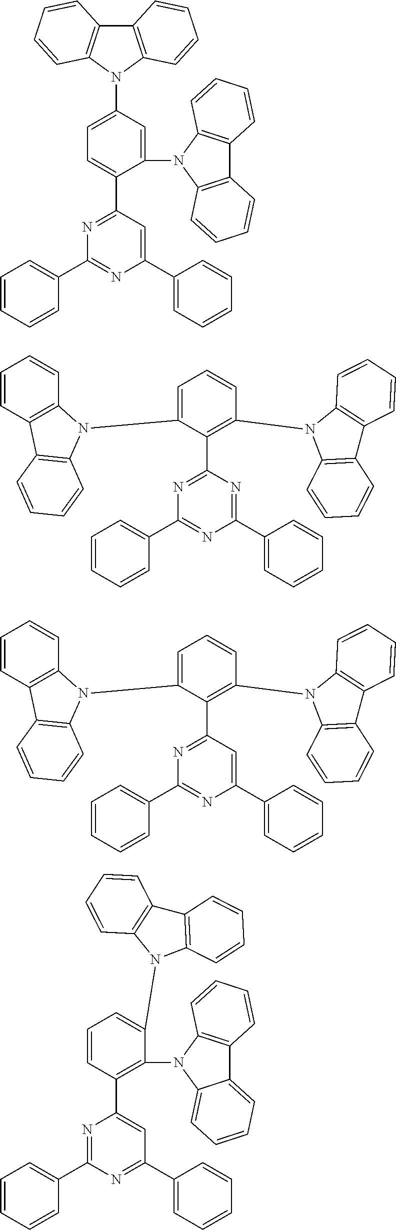

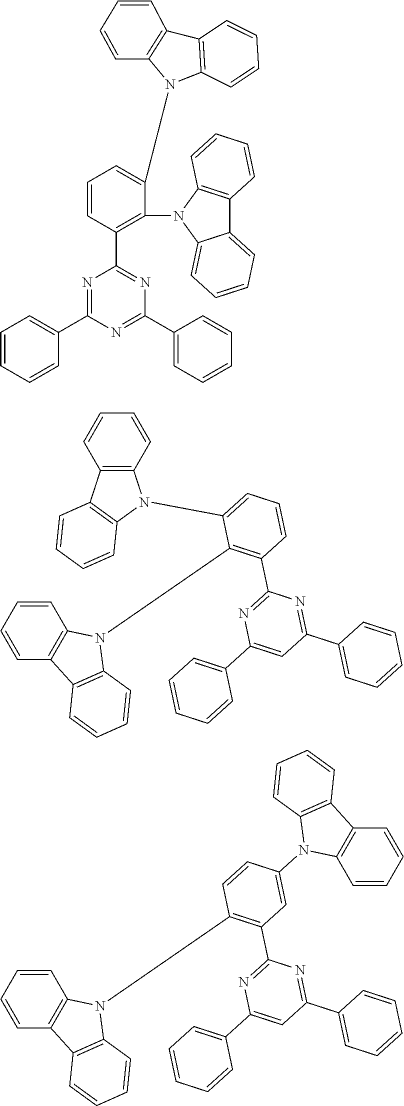 [SMILES: c1ccc(-c2nc(-c3ccccc3)nc(-c3cccc(-n4c5ccccc5c5ccccc54)c3-n3c4ccccc4c4ccccc43)n2)cc1 ; c1ccc(-c2cc(-c3ccccc3)nc(-c3cccc(-n4c5ccccc5c5ccccc54)c3-n3c4ccccc4c4ccccc43)n2)cc1 ; c1ccc(-c2cc(-c3ccccc3)nc(-c3cc(-n4c5ccccc5c5ccccc54)ccc3-n3c4ccccc4c4ccccc43)n2)cc1]

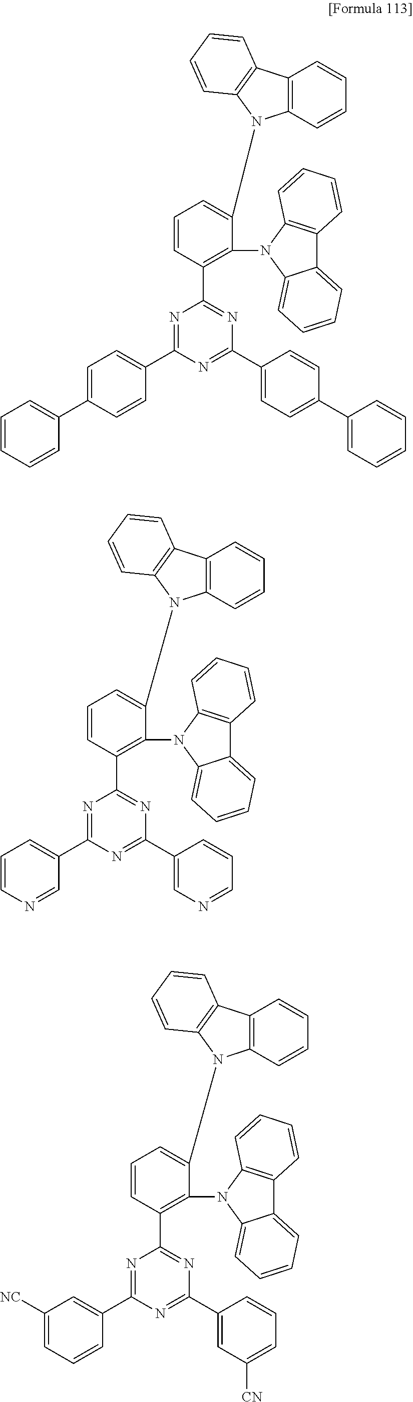 [SMILES: c1ccc(-c2ccc(-c3nc(-c4ccc(-c5ccccc5)cc4)nc(-c4cccc(-n5c6ccccc6c6ccccc65)c4-n4c5ccccc5c5ccccc54)n3)cc2)cc1 ; c1cncc(-c2nc(-c3cccnc3)nc(-c3cccc(-n4c5ccccc5c5ccccc54)c3-n3c4ccccc4c4ccccc43)n2)c1 ; N#Cc1cccc(-c2nc(-c3cccc(C#N)c3)nc(-c3cccc(-n4c5ccccc5c5ccccc54)c3-n3c4ccccc4c4ccccc43)n2)c1]

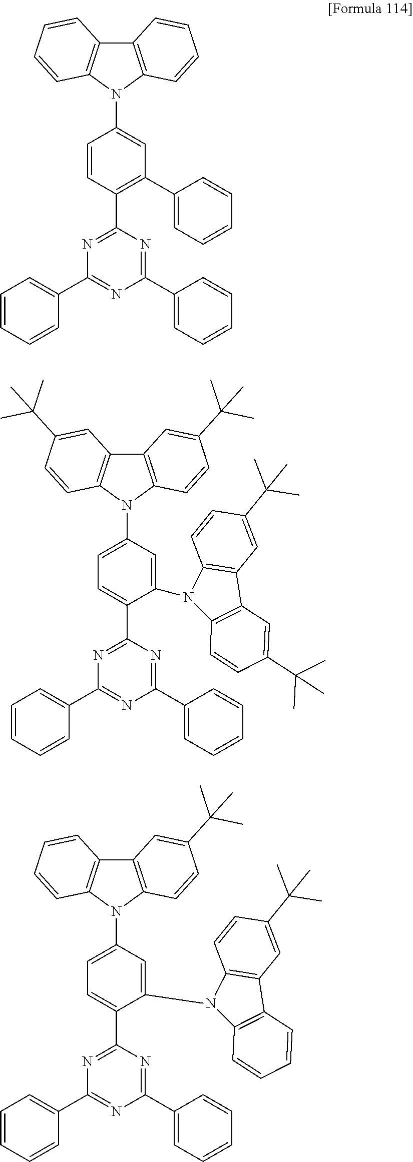

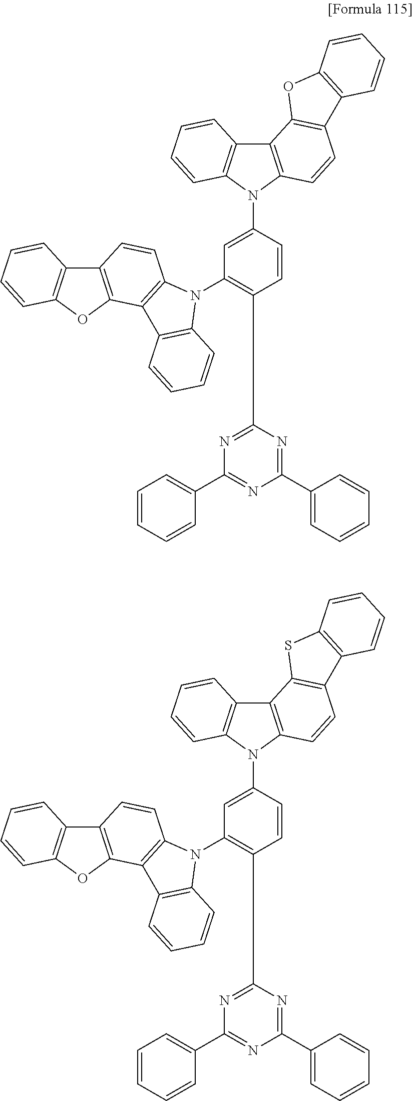 [SMILES: c1ccc(-c2nc(-c3ccccc3)nc(-c3ccc(-n4c5ccccc5c5c6oc7ccccc7c6ccc54)cc3-n3c4ccccc4c4c5oc6ccccc6c5ccc43)n2)cc1 ; c1ccc(-c2nc(-c3ccccc3)nc(-c3ccc(-n4c5ccccc5c5c6sc7ccccc7c6ccc54)cc3-n3c4ccccc4c4c5oc6ccccc6c5ccc43)n2)cc1]

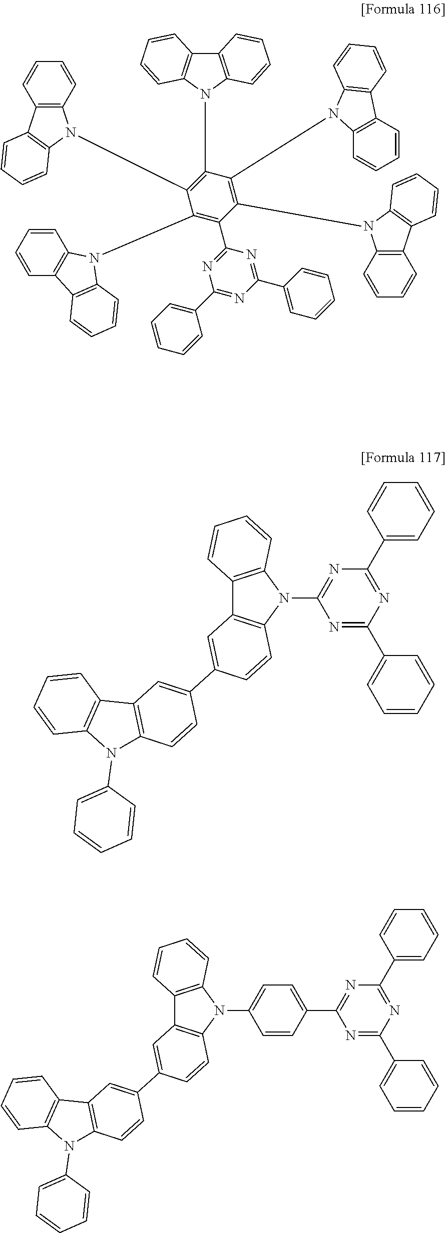 [SMILES: c1ccc(-c2nc(-c3ccccc3)nc(-c3c(-n4c5ccccc5c5ccccc54)c(-n4c5ccccc5c5ccccc54)c(-n4c5ccccc5c5ccccc54)c(-n4c5ccccc5c5ccccc54)c3-n3c4ccccc4c4ccccc43)n2)cc1 ; c1ccc(-c2nc(-c3ccccc3)nc(-n3c4ccccc4c4cc(-c5ccc6c(c5)c5ccccc5n6-c5ccccc5)ccc43)n2)cc1 ; c1ccc(-c2nc(-c3ccccc3)nc(-c3ccc(-n4c5ccccc5c5cc(-c6ccc7c(c6)c6ccccc6n7-c6ccccc6)ccc54)cc3)n2)cc1]

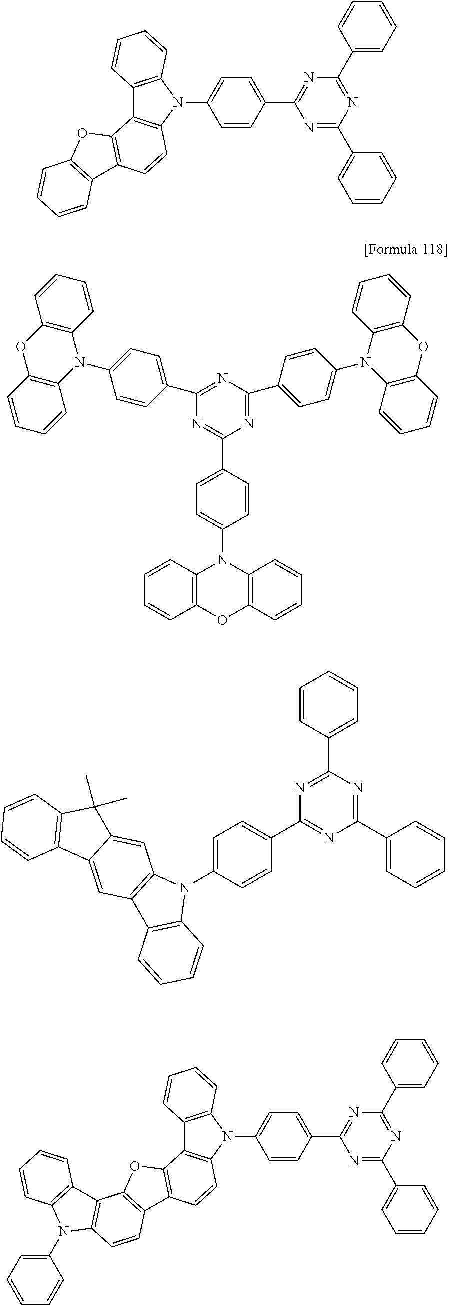 [SMILES: c1ccc(-c2nc(-c3ccccc3)nc(-c3ccc(-n4c5ccccc5c5c6oc7ccccc7c6ccc54)cc3)n2)cc1 ; c1ccc2c(c1)Oc1ccccc1N2c1ccc(-c2nc(-c3ccc(N4c5ccccc5Oc5ccccc54)cc3)nc(-c3ccc(N4c5ccccc5Oc5ccccc54)cc3)n2)cc1 ; CC1(C)c2ccccc2-c2cc3c4ccccc4n(-c4ccc(-c5nc(-c6ccccc6)nc(-c6ccccc6)n5)cc4)c3cc21 ; c1ccc(-c2nc(-c3ccccc3)nc(-c3ccc(-n4c5ccccc5c5c6oc7c(ccc8c7c7ccccc7n8-c7ccccc7)c6ccc54)cc3)n2)cc1]

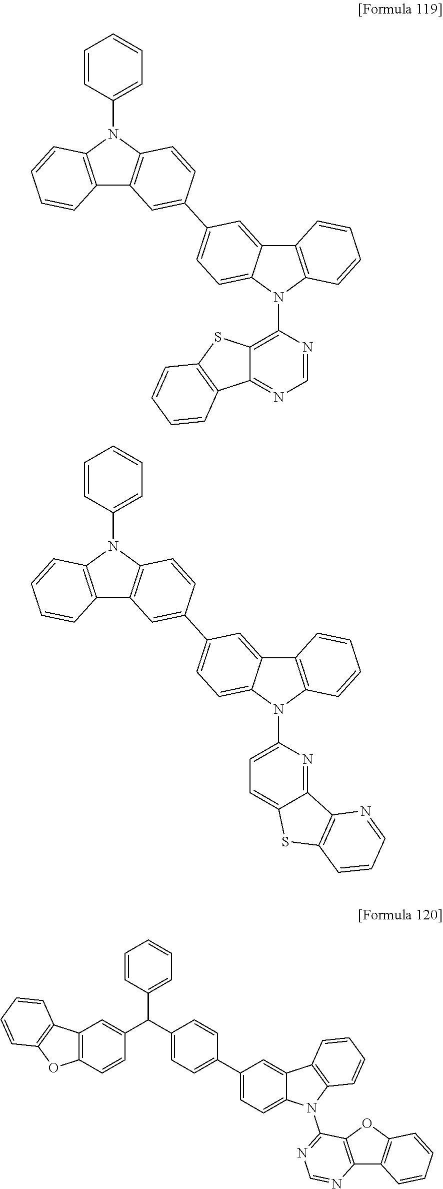 [SMILES: c1ccc(-n2c3ccccc3c3cc(-c4ccc5c(c4)c4ccccc4n5-c4ncnc5c4sc4ccccc45)ccc32)cc1 ; c1ccc(-n2c3ccccc3c3cc(-c4ccc5c(c4)c4ccccc4n5-c4ccc5sc6cccnc6c5n4)ccc32)cc1 ; c1ccc(C(c2ccc(-c3ccc4c(c3)c3ccccc3n4-c3ncnc4c3oc3ccccc34)cc2)c2ccc3oc4ccccc4c3c2)cc1]

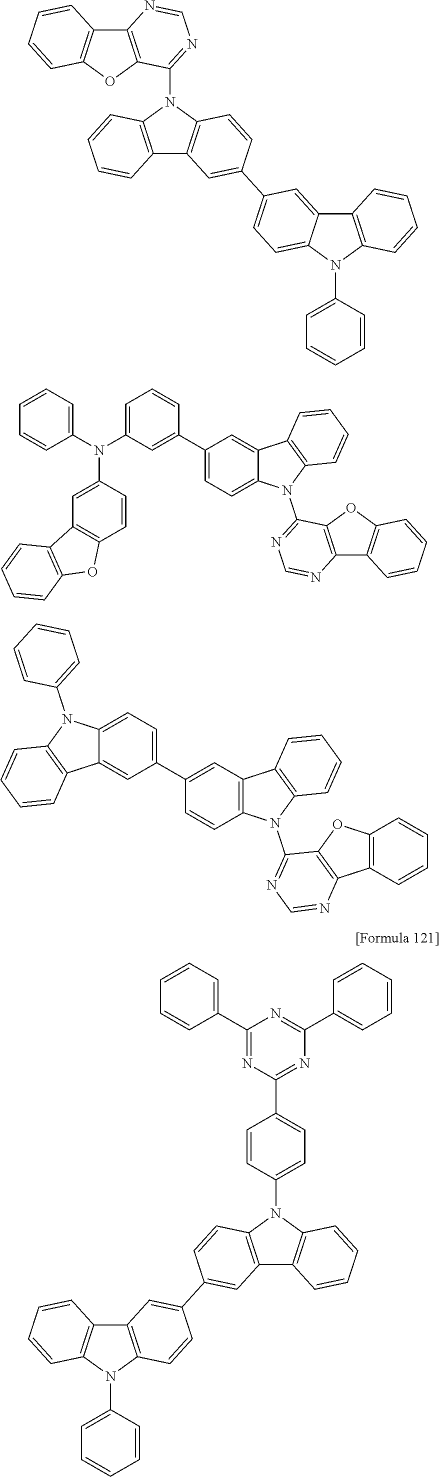

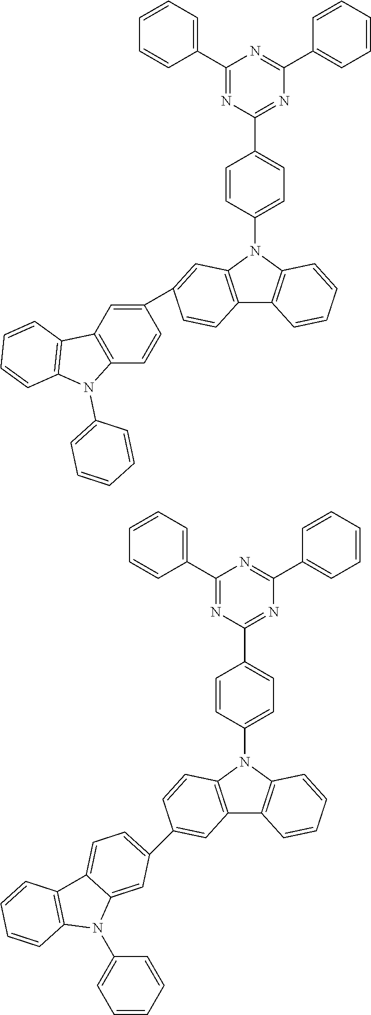 [SMILES: c1ccc(-c2nc(-c3ccccc3)nc(-c3ccc(-n4c5ccccc5c5ccc(-c6ccc7c(c6)c6ccccc6n7-c6ccccc6)cc54)cc3)n2)cc1 ; c1ccc(-c2nc(-c3ccccc3)nc(-c3ccc(-n4c5ccccc5c5cc(-c6ccc7c8ccccc8n(-c8ccccc8)c7c6)ccc54)cc3)n2)cc1]

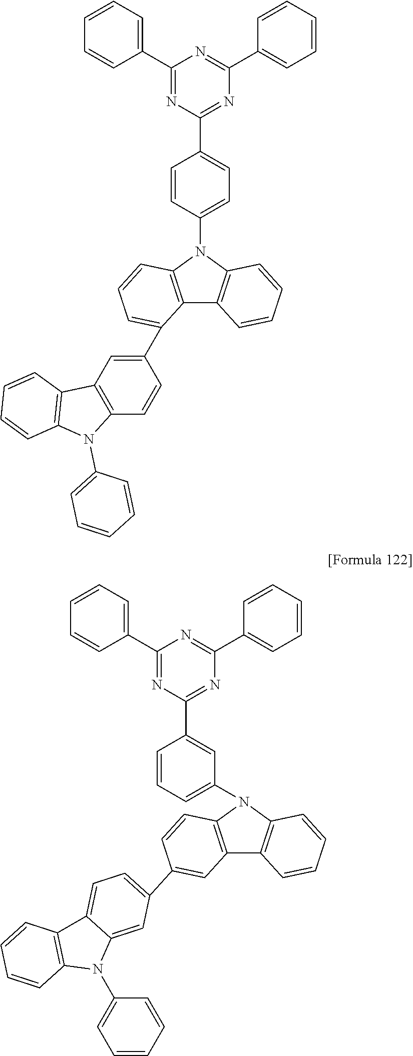 [SMILES: c1ccc(-c2nc(-c3ccccc3)nc(-c3ccc(-n4c5ccccc5c5c(-c6ccc7c(c6)c6ccccc6n7-c6ccccc6)cccc54)cc3)n2)cc1 ; c1ccc(-c2nc(-c3ccccc3)nc(-c3cccc(-n4c5ccccc5c5cc(-c6ccc7c8ccccc8n(-c8ccccc8)c7c6)ccc54)c3)n2)cc1]

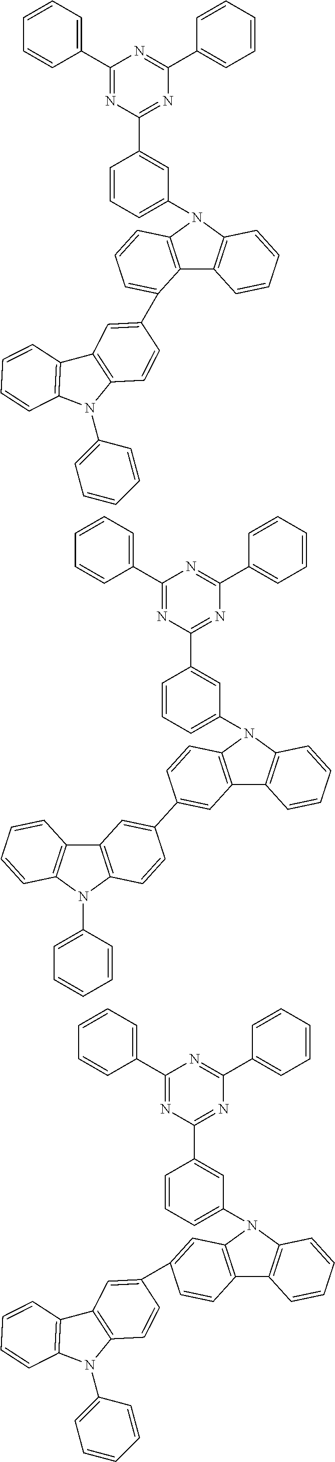

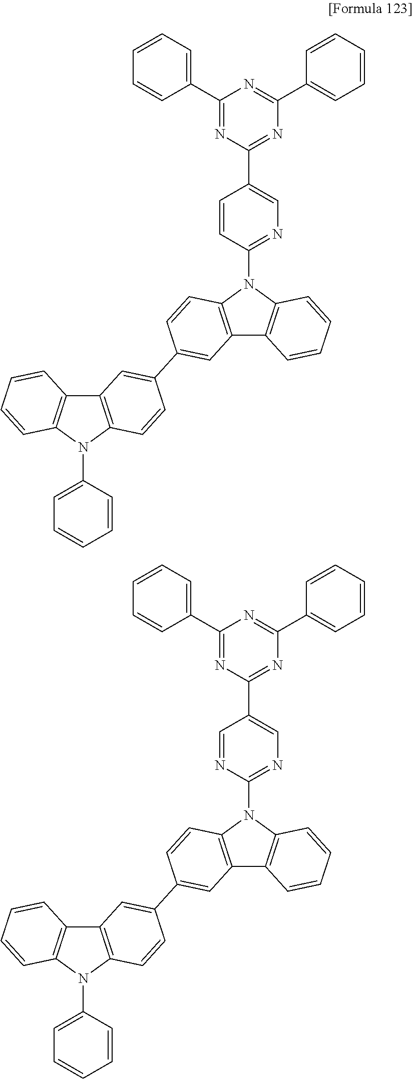 [SMILES: c1ccc(-c2nc(-c3ccccc3)nc(-c3ccc(-n4c5ccccc5c5cc(-c6ccc7c(c6)c6ccccc6n7-c6ccccc6)ccc54)nc3)n2)cc1 ; c1ccc(-c2nc(-c3ccccc3)nc(-c3cnc(-n4c5ccccc5c5cc(-c6ccc7c(c6)c6ccccc6n7-c6ccccc6)ccc54)nc3)n2)cc1]

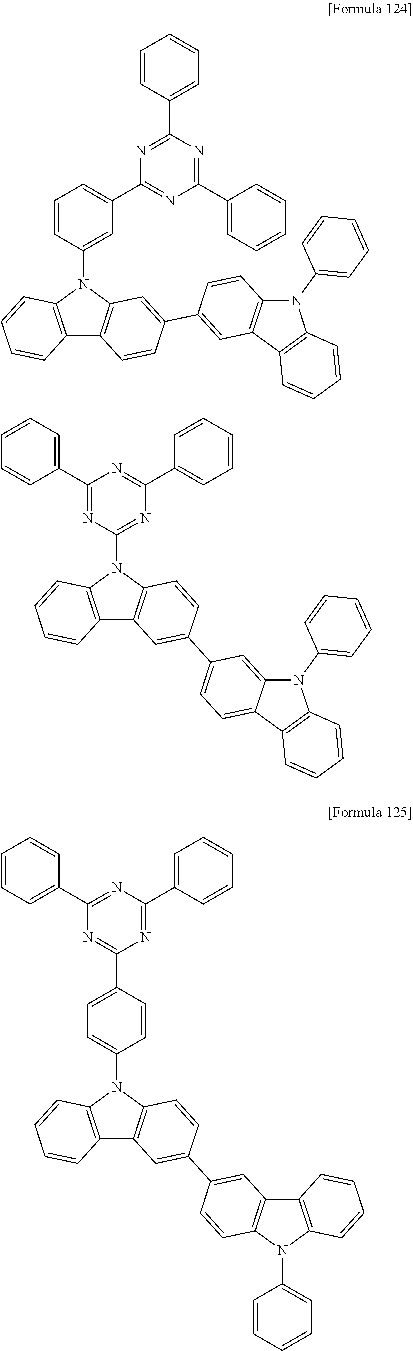 [SMILES: c1ccc(-c2nc(-c3ccccc3)nc(-c3cccc(-n4c5ccccc5c5ccc(-c6ccc7c(c6)c6ccccc6n7-c6ccccc6)cc54)c3)n2)cc1 ; c1ccc(-c2nc(-c3ccccc3)nc(-n3c4ccccc4c4cc(-c5ccc6c7ccccc7n(-c7ccccc7)c6c5)ccc43)n2)cc1 ; c1ccc(-c2nc(-c3ccccc3)nc(-c3ccc(-n4c5ccccc5c5cc(-c6ccc7c(c6)c6ccccc6n7-c6ccccc6)ccc54)cc3)n2)cc1]

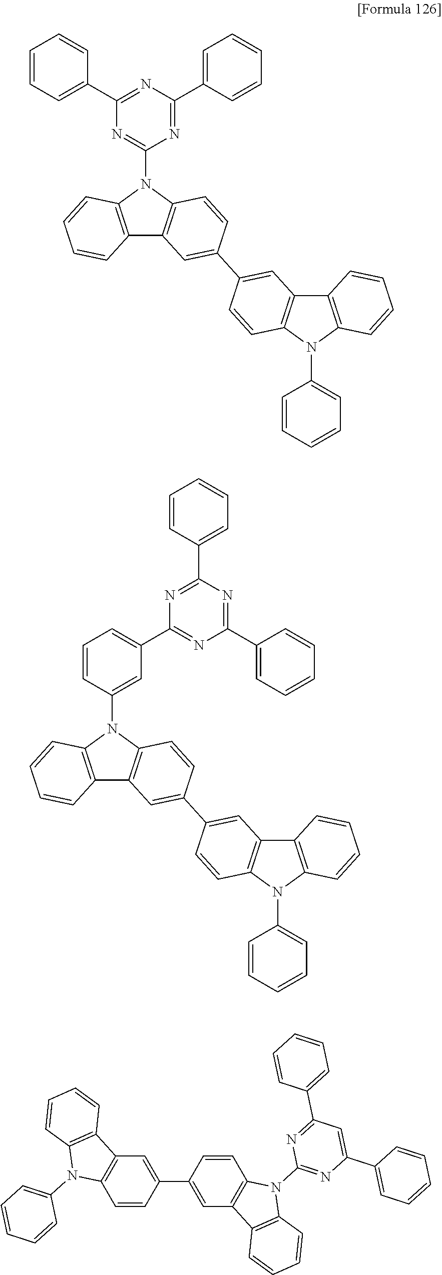

### Relationship Between Compound M3 and Compound M2 in Emitting Layer

In the organic EL device according to the exemplary embodiment, a singlet energy S₁(M2) of the compound M2 and a singlet energy S₁(M3) of the compound M3 satisfy a relationship of a numerical formula (Numerical Formula 1) below.

S₁(M3)>S₁(M2) (Numerical Formula 1)

An energy gap T_{77K}(M3) at 77K of the compound M3 is preferably larger than an energy gap T_{77K}(M2) at 77K of the compound M2. In other words, a relationship of the following numerical formula (Numerical Formula 11) is preferably satisfied.

T_{77K}(M3)>T_{77K}(M2) (Numerical Formula 11)

When the organic EL device according to the exemplary embodiment emits light, it is preferable that the compound M3 does not mainly emit light in the emitting layer.

Relationship between Triplet Energy and Energy Gap at 77K

Here, a relationship between a triplet energy and an energy gap at 77K will be described. In the exemplary embodiment, the energy gap at 77K is different from a typical triplet energy in some aspects.

The triplet energy is measured as follows. First, a solution in which a compound (measurement target) is dissolved in an appropriate solvent is encapsulated in a quartz glass tube to prepare a sample. A phosphorescence spectrum (ordinate axis: phosphorescent luminous intensity, abscissa axis: wavelength) of the sample is measured at a low temperature (77K). A tangent is drawn to the rise of the phosphorescence spectrum close to the short-wavelength region. The triplet energy is calculated by a predetermined conversion equation based on a wavelength value at an intersection of the tangent and the abscissa axis.

Herein, the thermally activated delayed fluorescent compound M2 used in the exemplary embodiment is preferably a compound having a small ΔST. When ΔST is small, intersystem crossing and inverse intersystem crossing are likely to occur even at a low temperature (77K), so that the singlet state and the triplet state coexist. As a result, the spectrum to be measured in the same manner as the above includes emission from both the singlet state and the triplet state. Although it is difficult to distinguish the emission from the singlet state from the emission from the triplet state, the value of the triplet energy is basically considered dominant.

Accordingly, in the exemplary embodiment, the triplet energy is measured by the same method as a typical triplet energy T, but a value measured in the following manner is referred to as an energy gap T_{77K} in order to differentiate the measured energy from the typical triplet energy in a strict meaning. The measurement target compound is dissolved in EPA (diethylether:isopentane:ethanol=5:5:2 in volume ratio) at a concentration of 10 μmol/L, and the obtained solution is put in a quartz cell to provide a measurement sample. A phosphorescence spectrum (ordinate axis: phosphorescent luminous intensity, abscissa axis: wavelength) of the sample is measured at a low temperature (77K). A tangent is drawn to the rise of the phosphorescence spectrum close to the short-wavelength region. An energy amount is calculated by a conversion equation (F1) below based on a wavelength value λedge [nm] at an intersection of the tangent and the abscissa axis and is defined as an energy gap T_{77K} at 77K.

T_{77K} [eV]=1239.85/λedge Conversion Equation (F1):

The tangent to the rise of the phosphorescence spectrum close to the short-wavelength region is drawn as follows. While moving on a curve of the phosphorescence spectrum from the short-wavelength region to the local maximum value closest to the short-wavelength region among the local maximum values of the phosphorescence spectrum, a tangent is checked at each point on the curve toward the long-wavelength of the phosphorescence spectrum. An inclination of the tangent is increased along the rise of the curve (i.e., a value of the ordinate axis is increased). A tangent drawn at a point of the local maximum inclination (i.e., a tangent at an inflection point) is defined as the tangent to the rise of the phosphorescence spectrum close to the short-wavelength region.

A local maximum point where a peak intensity is 15% or less of the maximum peak intensity of the spectrum is not counted as the above-mentioned local maximum peak intensity closest to the short-wavelength region. The tangent drawn at a point that is closest to the local maximum peak intensity closest to the short-wavelength region and where the inclination of the curve is the local maximum is defined as a tangent to the rise of the phosphorescence spectrum close to the short-wavelength region.

For phosphorescence measurement, a spectrophotofluorometer body F-4500 (manufactured by Hitachi High-Technologies Corporation) is usable. Any device for phosphorescence measurement is usable. A combination of a cooling unit, a low temperature container, an excitation light source and a light-receiving unit may be used for phosphorescence measurement.

### Singlet Energy S₁

A method of measuring a singlet energy S₁ with use of a solution (occasionally referred to as a solution method) is exemplified by a method below.

A toluene solution of a measurement target compound at a concentration of 10 μmol/L is prepared and put in a quartz cell. An absorption spectrum (ordinate axis: absorption intensity, abscissa axis: wavelength) of the thus-obtained sample is measured at a normal temperature (300K). A tangent is drawn to the fall of the absorption spectrum close to the long-wavelength region, and a wavelength value λedge (nm) at an intersection of the tangent and the abscissa axis is assigned to a conversion equation (F2) below to calculate singlet energy.

S₁ [eV]=1239.85/λedge Conversion Equation (F2):

Any device for measuring absorption spectrum is usable. For instance, a spectrophotometer (U3310 manufactured by Hitachi, Ltd.) is usable.

The tangent to the fall of the absorption spectrum close to the long-wavelength region is drawn as follows. While moving on a curve of the absorption spectrum from the local maximum value closest to the long-wavelength region, among the local maximum values of the absorption spectrum, in a long-wavelength direction, a tangent at each point on the curve is checked. An inclination of the tangent is decreased and increased in a repeated manner as the curve falls (i.e., a value of the ordinate axis is decreased). A tangent drawn at a point where the inclination of the curve is the local minimum closest to the long-wavelength region (except when absorbance is 0.1 or less) is defined as the tangent to the fall of the absorption spectrum close to the long-wavelength region.

The local maximum absorbance of 0.2 or less is not counted as the above-mentioned local maximum absorbance closest to the long-wavelength region.

In the exemplary embodiment, a difference (S₁-T_{77K}) between the singlet energy S₁ and the energy gap T_{77K} at 77K is defined as ΔST.

In the exemplary embodiment, a difference ΔST(M2) between the singlet energy S₁(M2) of the compound M2 and the energy gap T_{77K}(M2) at 77K of the compound M2 is preferably less than 0.3 eV, more preferably less than 0.2 eV, further preferably less than 0.1 eV, more further preferably less than 0.01 eV. That is, ΔST(M2) preferably satisfies a relationship of one of numerical formulae (Numerical Formula 1A) to (Numerical Formula 1D) below.

ΔST(M2)=S₁(M2)−T_{77K}(M2)<0.3 eV (Numerical Formula 1A)

ΔST(M2)=S₁(M2)−T_{77K}(M2)<0.2 eV (Numerical Formula 1B)

ΔST(M2)=S₁(M2)−T_{77K}(M2)<0.1 eV (Numerical Formula 1C)

ΔST(M2)=S₁(M2)−T_{77K}(M2)<0.01 eV (Numerical Formula 1 D)

### Film Thickness of Emitting Layer

A film thickness of the emitting layer of the organic EL device in the exemplary embodiment is preferably in a range from 5 nm to 50 nm, more preferably in a range from 7 nm to 50 nm, most preferably in a range from 10 nm to 50 nm. When the film thickness of the emitting layer is 5 nm or more, the formation of the emitting layer and the adjustment of the chromaticity are easy. When the film thickness of the emitting layer is 50 nm or less, an increase in the drive voltage is likely to be reducible.

### Content Ratios of Compounds in Emitting Layer

Content ratios of the compounds M2 and M3 in the emitting layer preferably fall, for instance, within a range below.

The content ratio of the compound M2 is preferably in a range from 10 mass % to 80 mass %, more preferably in a range from 10 mass % to 60 mass %, further preferably in a range from 20 mass % to 60 mass %.

The content ratio of the compound M3 is preferably in a range from 20 mass % to 90 mass %, more preferably in a range from 40 mass % to 90 mass %, further preferably in a range from 40 mass % to 80 mass %.

It should be noted that the emitting layer of the exemplary embodiment may further contain material(s) other than the compounds M2 and M3.

The emitting layer may include a single type of the compound M2 or may include two or more types of the compound M2. The emitting layer may include a single type of the compound M3 or may include two or more types of the compound M3.

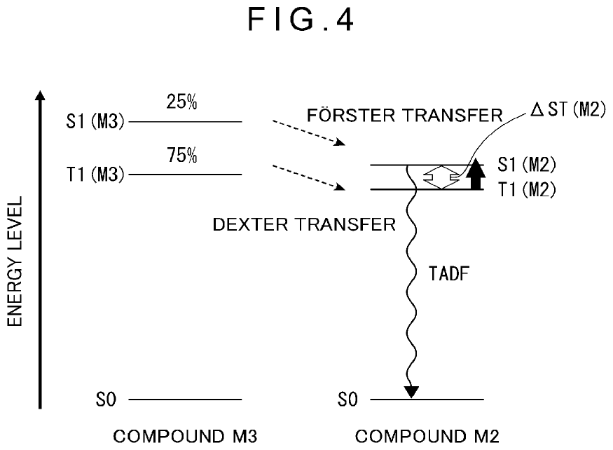

FIG. 4 shows a relationship in energy level and energy transfer between the compound M3 and the compound M2 in the emitting layer.

In FIG. 4, S0 represents a ground state. S1 (M2) represents the lowest singlet state of the compound M2. T1 (M2) represents the lowest triplet state of the compound M2. S1(M3) represents the lowest singlet state of the compound M3. T1 (M3) represents the lowest triplet state of the compound M3.

Dashed arrows in FIG. 4 show energy transfer between the excited states. An energy transfer occurs by Forster transfer from the lowest singlet state S₁ of the compound M3 to the lowest singlet state S₁ of the compound M2 or an energy transfer occurs by Dexter transfer from the lowest triplet state T1 of the compound M3 to the lowest triplet state T1 of the compound M2.

Further, when a material having a small ΔST(M2) is used as the compound M2, inverse intersystem crossing can be caused by a heat energy from the lowest triplet state T1 to the lowest singlet state S₁ in the compound M2. Consequently, fluorescence from the lowest singlet state S₁ of the compound M2 can be observed. It is inferred that the internal quantum efficiency can be theoretically raised up to 100% also by using delayed fluorescence by the TADF mechanism.

The organic EL device according to the exemplary embodiment contains the delayed fluorescent compound M2 and the compound M3 having the singlet energy larger than that of the compound M2 in the emitting layer.

According to the exemplary embodiment, an organic EL device with high performance, for instance, an organic EL device emitting light with a long lifetime can be achieved.

The organic EL device according to the exemplary embodiment is applicable to an electronic device such as a display device and a light-emitting device.

An arrangement of an organic EL device will be further described below.

### Substrate

The substrate is used as a support for the organic EL device. For instance, glass, quartz, plastics and the like are usable for the substrate. A flexible substrate is also usable. The flexible substrate is a bendable substrate, which is exemplified by a plastic substrate. Examples of the material for the plastic substrate include polycarbonate, polyarylate, polyethersulfone, polypropylene, polyester, polyvinyl fluoride, polyvinyl chloride, polyimide, and polyethylene naphthalate. Moreover, an inorganic vapor deposition film is also usable.

### Anode

Metal, an alloy, an electrically conductive compound, a mixture thereof, or the like having a large work function (specifically, 4.0 eV or more) is preferably used as the anode formed on the substrate. Specific examples of the material include ITO (Indium Tin Oxide), indium oxide-tin oxide containing silicon or silicon oxide, indium oxide-zinc oxide, indium oxide containing tungsten oxide and zinc oxide, and graphene. In addition, gold (Au), platinum (Pt), nickel (Ni), tungsten (W), chrome (Cr), molybdenum (Mo), iron (Fe), cobalt (Co), copper (Cu), palladium (Pd), titanium (Ti), and nitrides of a metal material (e.g., titanium nitride) are usable.

The material is typically formed into a film by a sputtering method. For instance, the indium oxide-zinc oxide can be formed into a film by the sputtering method using a target in which zinc oxide in a range from 1 mass % to 10 mass % is added to indium oxide. Moreover, for instance, the indium oxide containing tungsten oxide and zinc oxide can be formed by the sputtering method using a target in which tungsten oxide in a range from 0.5 mass % to 5 mass % and zinc oxide in a range from 0.1 mass % to 1 mass % are added to indium oxide. In addition, the anode may be formed by a vacuum deposition method, a coating method, an inkjet method, a spin coating method or the like.

Among the organic layers formed on the anode, since the hole injecting layer adjacent to the anode is formed of a composite material into which holes are easily injectable irrespective of the work function of the anode, a material usable as an electrode material (e.g., metal, an alloy, an electrically conductive compound, a mixture thereof, and the elements belonging to the group 1 or 2 of the periodic table) is also usable for the anode.

The elements belonging to the group 1 or 2 of the periodic table, which are a material having a small work function, specifically, an alkali metal such as lithium (Li) and cesium (Cs), an alkaline earth metal such as magnesium (Mg), calcium (Ca) and strontium (Sr), an alloy containing the alkali metal and the alkaline earth metal (e.g., MgAg, AlLi), a rare earth metal such as europium (Eu) and ytterbium (Yb), and an alloy containing the rare earth metal are usable for the anode. It should be noted that the vacuum deposition method and the sputtering method are usable for forming the anode using the alkali metal, alkaline earth metal and the alloy thereof. Further, when a silver paste is used for the anode, the coating method and the inkjet method are usable.

### Cathode

It is preferable to use metal, an alloy, an electroconductive compound, a mixture thereof, or the like having a small work function (specifically, 3.8 eV or less) for the cathode. Examples of materials for the cathode include elements belonging to the group 1 or 2 of the periodic table, specifically, an alkali metal such as lithium (Li) and cesium (Cs), an alkaline earth metal such as magnesium (Mg), calcium (Ca) and strontium (Sr), an alloy containing the alkali metal and the alkaline earth metal (e.g., MgAg, AlLi), a rare earth metal such as europium (Eu) and ytterbium (Yb), and an alloy containing the rare earth metal.

It should be noted that the vacuum deposition method and the sputtering method are usable for forming the cathode using the alkali metal, alkaline earth metal and the alloy thereof. Further, when a silver paste is used for the cathode, the coating method and the inkjet method are usable.

By providing the electron injecting layer, various conductive materials such as Al, Ag, ITO, graphene, and indium oxide-tin oxide containing silicon or silicon oxide may be used for forming the cathode regardless of the work function. The conductive materials can be formed into a film using the sputtering method, inkjet method, spin coating method and the like.

### Hole Injecting Layer

The hole injecting layer is a layer containing a substance exhibiting a high hole injectability. Examples of the substance exhibiting a high hole injectability include molybdenum oxide, titanium oxide, vanadium oxide, rhenium oxide, ruthenium oxide, chrome oxide, zirconium oxide, hafnium oxide, tantalum oxide, silver oxide, tungsten oxide, and manganese oxide.

In addition, the examples of the highly hole-injectable substance further include: an aromatic amine compound, which is a low-molecule organic compound, such as 4,4′,4″-tris(N,N-diphenylamino)triphenylamine (abbreviation: TDATA), 4,4′,4″-tris[N-(3-methylphenyl)-N-phenylamino]triphenylamine (abbreviation: MTDATA), 4,4′-bis[N-(4-diphenylaminophenyl)-N-phenylamino]biphenyl (abbreviation: DPAB), 4,4′-bis(N-{4-[N′-(3-methylphenyl)-N′-phenylamino]phenyl}-N-phenylamino)biphenyl (abbreviation: DNTPD), 1,3,5-tris[N-(4-diphenylaminophenyl)-N-phenylamino]benzene (abbreviation: DPA3B), 3-[N-(9-phenylcarbazole-3-yl)-N-phenylamino]-9-phenylcarbazole (abbreviation: PCzPCA1), 3,6-bis[N-(9-phenylcarbazole-3-yl)-N-phenylamino]-9-phenylcarbazole (abbreviation: PCzPCA2), and 3-[N-(1-naphthyl)-N-(9-phenylcarbazole-3-yl)amino]-9-phenylcarbazole (abbreviation: PCzPCN1); and dipyrazino[2,3-f:20,30-h]quinoxaline-2,3,6,7,10,11-hexacarbonitrile (HAT-CN).

In addition, a high polymer compound (e.g., oligomer, dendrimer and polymer) is usable as the substance exhibiting a high hole injectability. Examples of the high-molecule compound include poly(N-vinylcarbazole) (abbreviation: PVK), poly(4-vinyltriphenylamine) (abbreviation: PVTPA), poly[N-(4-{N′-[4-(4-diphenylamino)phenyl]phenyl-N′-phenylamino}phenyl)methacrylamide](abbreviation: PTPDMA), and poly[N,N′-bis(4-butylphenyl)-N,N′-bis(phenyl)benzidine] (abbreviation: Poly-TPD). Moreover, an acid-added high polymer compound such as poly(3,4-ethylenedioxythiophene)/poly(styrene sulfonic acid) (PEDOT/PSS) and polyaniline/poly(styrene sulfonic acid) (PAni/PSS) are also usable.

### Hole Transporting Layer

The hole transporting layer is a layer containing a highly hole-transporting substance. An aromatic amine compound, carbazole derivative, anthracene derivative and the like are usable for the hole transporting layer. Specific examples of a material for the hole transporting layer include 4,4′-bis[N-(1-naphthyl)-N-phenylamino]biphenyl (abbreviation: NPB), N,N′-bis(3-methylphenyl)-N,N′-diphenyl-[1,1′-biphenyl]-4,4′-diamine (abbreviation: TPD), 4-phenyl-4′-(9-phenylfluorene-9-yl)triphenylamine (abbreviation: BAFLP), 4,4′-bis[N-(9,9-dimethylfluorene-2-yl)-N-phenylamino]biphenyl (abbreviation: DFLDPBi), 4,4′,4″-tris(N,N-diphenylamino)triphenylamine (abbreviation: TDATA), 4,4′,4″-tris[N-(3-methylphenyl)-N-phenylamino]triphenylamine (abbreviation: MTDATA), and 4,4′-bis[N-(spiro-9,9′-bifluorene-2-yl)-N-phenylamino]biphenyl (abbreviation: BSPB). The above-described substances mostly have a hole mobility of 10⁻⁶ cm²/(V·s) or more.

For the hole transporting layer, a carbazole derivative such as CBP, 9-[4-(N-carbazolyl)]phenyl-10-phenylanthracene (CzPA), and 9-phenyl-3-[4-(10-phenyl-9-anthryl)phenyl]-9H-carbazole (PCzPA) and an anthracene derivative such as t-BuDNA, DNA, and DPAnth may be used. A high polymer compound such as poly(N-vinylcarbazole) (abbreviation: PVK) and poly(4-vinyltriphenylamine) (abbreviation: PVTPA) is also usable.

However, in addition to the above substances, any substance exhibiting a higher hole transportability than an electron transportability may be used. It should be noted that the layer containing the substance exhibiting a high hole transportability may be not only a single layer but also a laminate of two or more layers formed of the above substance(s).

When the hole transporting layer includes two or more layers, one of the layers with a larger energy gap is preferably provided closer to the emitting layer. An example of the material with a larger energy gap is HT-2 used in later-described Examples.

### Electron Transporting Layer

The electron transporting layer is a layer containing a highly electron-transporting substance. For the electron transporting layer, 1) a metal complex such as an aluminum complex, beryllium complex, and zinc complex, 2) a hetero aromatic compound such as imidazole derivative, benzimidazole derivative, azine derivative, carbazole derivative, and phenanthroline derivative, and 3) a high polymer compound are usable. Specifically, as a low-molecule organic compound, a metal complex such as Alq, tris(4-methyl-8-quinolinato)aluminum (abbreviation: Almq₃), bis(10-hydroxybenzo[h]quinolinato)beryllium (abbreviation: BeBq₂), BAIq, Znq, ZnPBO and ZnBTZ is usable. In addition to the metal complex, a heteroaromatic compound such as 2-(4-biphenylyl)-5-(4-tert-butylphenyl)-1,3,4-oxadiazole (abbreviation: PBD), 1,3-bis[5-(ptert-butylphenyl)-1,3,4-oxadiazole-2-yl]benzene (abbreviation: OXD-7), 3-(4-tert-butylphenyl)-4-phenyl-5-(4-biphenylyl)-1,2,4-triazole (abbreviation: TAZ), 3-(4-tert-butylphenyl)-4-(4-ethylphenyl)-5-(4-biphenylyl)-1,2,4-triazole (abbreviation: p-EtTAZ), bathophenanthroline (abbreviation: BPhen), bathocuproine (abbreviation: BCP), and 4,4′-bis(5-methylbenzoxazole-2-yl)stilbene (abbreviation: BzOs) is usable. In the exemplary embodiment, a benzimidazole compound is preferably usable. The above-described substances mostly have an electron mobility of 10⁻⁶ cm²/Vs or more. It should be noted that any substance other than the above substance may be used for the electron transporting layer as long as the substance exhibits a higher electron transportability than the hole transportability. The electron transporting layer may be provided in the form of a single layer or a laminate of two or more layers of the above substance(s).

Further, a high polymer compound is usable for the electron transporting layer. For instance, poly[(9,9-dihexylfluorene-2,7-diyl)-co-(pyridine-3,5-diyl)](abbreviation: PF-Py), poly[(9,9-dioctylfluorene-2,7-diyl)-co-(2,2′-bipyridine-6,6′-diyl)](abbreviation: PF-BPy) and the like are usable.

### Electron Injecting Layer

The electron injecting layer is a layer containing a highly electron-injectable substance. Examples of a material for the electron injecting layer include an alkali metal, alkaline earth metal and a compound thereof, examples of which include lithium (Li), cesium (Cs), calcium (Ca), lithium fluoride (LiF), cesium fluoride (CsF), calcium fluoride (CaF₂), and lithium oxide (LiOx). In addition, the alkali metal, alkaline earth metal or the compound thereof may be added to the substance exhibiting the electron transportability in use. Specifically, for instance, magnesium (Mg) added to Alq may be used. In this case, the electrons can be more efficiently injected from the cathode.

Alternatively, the electron injecting layer may be provided by a composite material in a form of a mixture of the organic compound and the electron donor. Such a composite material exhibits excellent electron injectability and electron transportability since electrons are generated in the organic compound by the electron donor. In this case, the organic compound is preferably a material excellent in transporting the generated electrons. Specifically, the above examples (e.g., the metal complex and the hetero aromatic compound) of the substance forming the electron transporting layer are usable. As the electron donor, any substance exhibiting electron donating property to the organic compound is usable. Specifically, the electron donor is preferably alkali metal, alkaline earth metal and rare earth metal such as lithium, cesium, magnesium, calcium, erbium and ytterbium. The electron donor is also preferably alkali metal oxide and alkaline earth metal oxide such as lithium oxide, calcium oxide, and barium oxide. Moreover, a Lewis base such as magnesium oxide is usable. Further, the organic compound such as tetrathiafulvalene (abbreviation: TTF) is usable.

### Layer Formation Method

A method for forming each layer of the organic EL device in the exemplary embodiment is subject to no limitation except for the above particular description. However, known methods of dry film-forming such as vacuum deposition, sputtering, plasma or ion plating and wet film-forming such as spin coating, dipping, flow coating or ink-jet are applicable.

### Film Thickness

A thickness of each of the organic layers in the organic EL device according to the exemplary embodiment is not limited except for the above particular description. In general, the thickness preferably ranges from several nanometers to 1 μm because excessively small film thickness is likely to cause defects (e.g. pin holes) and excessively large thickness leads to the necessity of applying high voltage and consequent reduction in efficiency.

### Second Exemplary Embodiment

An arrangement of an organic EL device according to a second exemplary embodiment will be described below. In the description of the second exemplary embodiment, the same components as those in the first exemplary embodiment are denoted by the same reference signs and names to simplify or omit an explanation of the components. In the second exemplary embodiment, any materials and compounds that are not specified may be the same as those in the first exemplary embodiment.

The organic EL device according to the second exemplary embodiment is different from the organic EL device according to the first exemplary embodiment in that the emitting layer further includes a fluorescent compound M1. The second exemplary embodiment is the same as the first exemplary embodiment in other respects.

In other words, in the second exemplary embodiment, the emitting layer contains the compound M3 represented by the formula (3), the delayed fluorescent compound M2, and the fluorescent compound M1.

In this arrangement, the compound M1 is preferably a dopant material, the compound M2 is preferably a host material, and the compound M3 is preferably a host material. One of the compound M2 and the compound M3 may be referred to as a first host material, and the other may be referred to as a second host material.

### Compound M1

The emitting layer of the exemplary embodiment includes the fluorescent compound M1.

The compound M1 of the exemplary embodiment is not a phosphorescent metal complex. The compound M1 of the exemplary embodiment is preferably not a heavy-metal complex. The compound M1 of the exemplary embodiment is preferably not a metal complex.

A fluorescent material is usable as the compound M1 of the exemplary embodiment. Specific examples of the fluorescent material include a bisarylaminonaphthalene derivative, aryl-substituted naphthalene derivative, bisarylaminoanthracene derivative, aryl-substituted anthracene derivative, bisarylaminopyrene derivative, aryl-substituted pyrene derivative, bisarylamino chrysene derivative, aryl-substituted chrysene derivative, bisarylaminofluoranthene derivative, aryl-substituted fluoranthene derivative, indenoperylene derivative, acenaphthofluoranthene derivative, compound including a boron atom, pyromethene boron complex compound, compound having a pyromethene skeleton, metal complex of the compound having a pyrromethene skeleton, diketopyrrolopyrrole derivative, perylene derivative, and naphthacene derivative.

The compound M1 of the exemplary embodiment is preferably a compound represented by a formula (10) below.

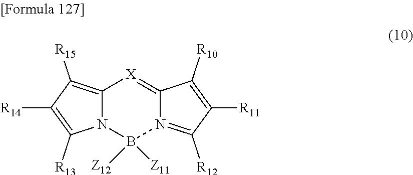

In the formula (10):

X is a nitrogen atom, or a carbon atom bonded to Y;

Y is a hydrogen atom or a substituent;

R₁₀ to R₁₅ are each independently a hydrogen atom or a substituent, or at least one pair of a pair of R₁₀ and R₁₁, a pair of R₁₁ and R₁₂, a pair of R₁₃ and R₁₄, or a pair of R₁₄ and R₁₅ are mutually bonded to form a ring; Y and R₁₀ to R₁₅ as a substituent are each independently selected from the group consisting of a substituted or unsubstituted alkyl group having 1 to 30 carbon atoms, a substituted or unsubstituted alkyl halide group having 1 to 30 carbon atoms, a substituted or unsubstituted cycloalkyl group having 3 to 30 ring carbon atoms, a substituted or unsubstituted aryl group having 6 to 30 ring carbon atoms, a substituted or unsubstituted alkoxy group having 1 to 30 carbon atoms, a substituted or unsubstituted alkoxy halide group having 1 to 30 carbon atoms, a substituted or unsubstituted alkylthio group having 1 to 30 carbon atoms, a substituted or unsubstituted aryloxy group having 6 to 30 ring carbon atoms, a substituted or unsubstituted arylthio group having 6 to 30 ring carbon atoms, a substituted or unsubstituted alkenyl group having 2 to 30 carbon atoms, a substituted or unsubstituted aralkyl group having 7 to 30 carbon atoms, a substituted or unsubstituted heteroaryl group having 5 to 30 ring atoms, a halogen atom, a carboxy group, a substituted or unsubstituted ester group, a substituted or unsubstituted carbamoyl group, a substituted or unsubstituted amino group, a nitro group, a cyano group, a substituted or unsubstituted silyl group, and a substituted or unsubstituted siloxanyl group;

Z₁₁ and Z₁₂ are each independently a substituent, or Z₁₁ and Z₁₂ are mutually bonded to form a ring; and

Z₁₁ and Z₁₂ as a substituent are each independently selected from the group consisting of a halogen atom, a substituted or unsubstituted alkyl group having 1 to 30 carbon atoms, a substituted or unsubstituted alkyl halide group having 1 to 30 carbon atoms, a substituted or unsubstituted aryl group having 6 to 30 ring carbon atoms, a substituted or unsubstituted alkoxy group having 1 to 30 carbon atoms, a substituted or unsubstituted alkoxy halide group having 1 to 30 carbon atoms, and a substituted or unsubstituted aryloxy group having 6 to 30 ring carbon atoms.

For instance, when a pair of R₁₄ and R₁₅ in the formula (10) are mutually bonded to form a ring, the compound M1 is represented by a formula (11) below.

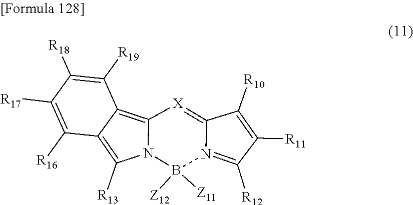

In the formula (11): X, Y, R₁₀ to R₁₃, Z₁₁ and Z₁₂ respectively represent the same as X, Y, R₁₀ to R₁₃, Z₁₁ and Z₁₂ in the formula (10); R₁₆ to R₁₉ are each independently a hydrogen atom or a substituent; and R₁₆ to R₁₉ as a substituent each independently represent the same as R₁₀ to R₁₃ as a substituent.

When Z₁₁ and Z₁₂ in the formula (10) are mutually bonded to form a ring, the compound M1 is represented by, for instance, a formula (10A) or (10B) below. However, a structure of the compound M1 is not limited to structures below.

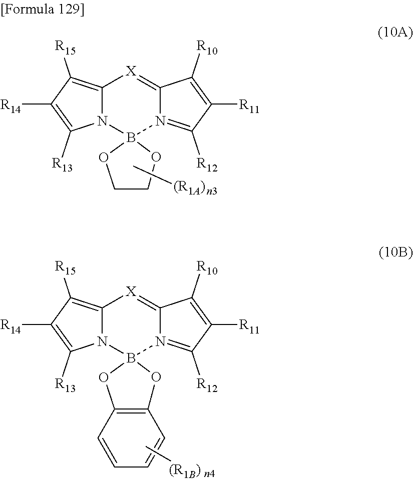

In the formula (10A): X, Y and R₁₀ to R₁₅ respectively represent the same as X, Y and R₁₀ to R₁₅ in the formula (10); R_{1A} is each independently a hydrogen atom or a substituent; R_{1A} as a substituent represents the same as R₁₀ to R₁₅ as a substituent; and n3 is 4.

In the formula (10B): X, Y and R₁₀ to R₁₅ respectively represent the same as X, Y and R₁₀ to R₁₅ in the formula (10); R_{1B} is each independently a hydrogen atom or a substituent; R_{1B} as a substituent represents the same as R₁₀ to R₁₅ as a substituent; and n4 is 4.

It is preferable that at least one of Z₁₁ or Z₁₂ (preferably both of Z₁₁ and Z₁₂) is a group selected from the group consisting of a substituted or unsubstituted alkyl group having 1 to 30 carbon atoms, a substituted or unsubstituted alkyl halide group having 1 to 30 carbon atoms, a substituted or unsubstituted aryl group having 6 to 30 ring carbon atoms, a substituted or unsubstituted alkoxy group having 1 to 30 carbon atoms, a substituted or unsubstituted alkoxy halide group having 1 to 30 carbon atoms, and a substituted or unsubstituted aryloxy group having 6 to 30 ring carbon atoms.

It is more preferable that at least one of Z₁₁ or Z₁₂ is a group selected from the group consisting of a fluorine-substituted alkoxy group having 1 to 30 carbon atoms, a fluorine-substituted aryloxy group having 6 to 30 ring carbon atoms, and an aryloxy group having 6 to 30 ring carbon atoms and substituted with a fluoroalkyl group having 1 to 30 carbon atoms.

Further preferably, at least one of Z₁₁ or Z₁₂ is a fluorine-substituted alkoxy group having 1 to 30 carbon atoms. Further more preferably, both of Z₁₁ and Z₁₂ are each a fluorine-substituted alkoxy group having 1 to 30 carbon atoms.

It is also preferable that both of Z₁₁ and Z₁₂ are the same.

Meanwhile, it is also preferable that at least one of Z₁₁ or Z₁₂ is a fluorine atom. It is also more preferable that both of Z₁₁ and Z₁₂ are fluorine atoms.

It is also preferable that at least one of Z₁₁ or Z₁₂ is a group represented by a formula (10a) below.

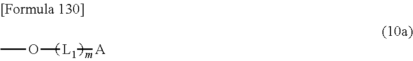

In the formula (10a): A is a substituted or unsubstituted alkyl group having 1 to 6 carbon atoms, a substituted or unsubstituted alkyl halide group having 1 to 6 carbon atoms, or a substituted or unsubstituted aryl group having 6 to 12 ring carbon atoms; L₁ is a substituted or unsubstituted alkylene group having 1 to 6 carbon atoms, or a substituted or unsubstituted arylene group having 6 to 12 ring carbon atoms; m is 0, 1, 2, 3, 4, 5, 6 or 7; and when m is 2, 3, 4, 5, 6 or 7, a plurality of L₁₂ are mutually the same or different. m is preferably 0, 1 or 2. When m is 0, A is directly bonded to O (oxygen atom).

When Z₁₁ and Z₁₂ in the formula (10) are each a group represented by the formula (10a), the compound M1 is a compound represented by a formula (12) below. The compound M1 is also preferably a compound represented by the formula (12).

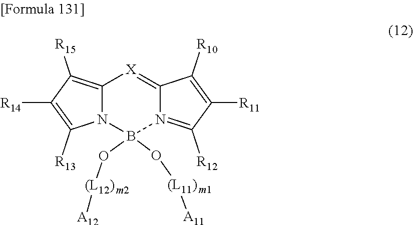

In the formula (12), X, Y bonded to a carbon atom as X, and R₁₀ to R₁₅ respectively represent the same as X, Y and R₁₀ to R₁₅ in the formula (10). A₁₁ and A₁₂ represent the same as A in the formula (10a) and may be mutually the same or different. L₁₁ and L₁₂ represent the same as L₁ in the formula (10a) and may be mutually the same or different. m1 and m2 are each independently 0, 1, 2, 3, 4, 5, 6 or 7, preferably 0, 1 or 2. When m1 is 2, 3, 4, 5, 6 or 7, a plurality of L₁₁ are mutually the same or different. When m2 is 2, 3, 4, 5, 6 or 7, a plurality of L₁₂ are mutually the same or different. When m1 is 0, A₁₁ is directly bonded to O (oxygen atom). When m2 is 0, A₁₂ is directly bonded to O (oxygen atom).

At least one of A or L₁ in the formula (10a) is preferably substituted with a halogen atom, more preferably substituted with a fluorine atom.

A in the formula (10a) is more preferably a perfluoroalkyl group having 1 to 6 carbon atoms or a perfluoroaryl group having 6 to 12 ring carbon atoms, further preferably a perfluoroalkyl group having 1 to 6 carbon atoms.

L₁ in the formula (10a) is more preferably a perfluoroalkylene group having 1 to 6 carbon atoms or a perfluoroarylene group having 6 to 12 ring carbon atoms, further preferably a perfluoroalkylene group having 1 to 6 carbon atoms.

In other words, the compound M1 is also preferably a compound represented by a formula (12a) below.

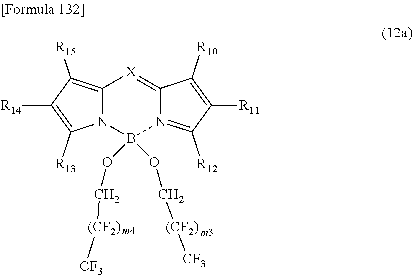

In the formula (12a):

X represents the same as X in the formula (10);

Y bonded to a carbon atom as X represents the same as Y in the formula (10);

R₁₀ to R₁₅ each independently represent the same as R₁₀ to R₁₅ in the formula (10);

m3 is 0, 1, 2, 3 or 4;

m4 is 0, 1, 2, 3 or 4; and

m3 and m4 are mutually the same or different.

In the formulae (10), (11), (12) and (12a), X is a carbon atom bonded to Y; Y is a hydrogen atom or a substituent; Y as a substituent is preferably a substituent selected from the group consisting of a substituted or unsubstituted alkyl group having 1 to 30 carbon atoms, a substituted or unsubstituted alkyl halide group having 1 to 30 carbon atoms and a substituted or unsubstituted aryl group having 6 to 30 ring carbon atoms, more preferably a substituted or unsubstituted aryl group having 6 to 30 ring carbon atoms.

In the formulae (10), (11), (12) and (12a), it is more preferable that: X is a carbon atom bonded to Y; Y is a hydrogen atom or a substituent; Y as a substituent is a substituted or unsubstituted aryl group having 6 to 30 ring carbon atoms; and when Y as a substituent is an aryl group having 6 to 30 ring carbon atoms and having a substituent, the substituent is a substituted or unsubstituted alkyl group having 1 to 30 carbon atoms, a substituted or unsubstituted alkyl halide group having 1 to 30 carbon atoms, a substituted or unsubstituted alkoxy group having 1 to 30 carbon atoms, a substituted or unsubstituted alkoxy halide group having 1 to 30 carbon atoms, or an aryl group having 6 to 30 ring carbon atoms and substituted by an alkyl group having 1 to 30 carbon atoms.

In the compound M1, Z₁₁ and Z₁₂ may be mutually bonded to form a ring. However, it is preferable that Z₁₁ and Z₁₂ are not mutually bonded to form no ring.

In the formulae (10), (12) and (12a), at least one of R₁₀, R₁₂, R₁₃ or R₁₅ is preferably a substituted or unsubstituted alkyl group having 1 to 30 carbon atoms or a substituted or unsubstituted alkyl halide group having 1 to 30 carbon atoms.

In the formulae (10), (12) and (12a), R₁₀, R₁₂, R₁₃ and R₁₅ are each more preferably a substituted or unsubstituted alkyl group having 1 to 30 carbon atoms or a substituted or unsubstituted alkyl halide group having 1 to 30 carbon atoms. In this case, R₁₁ and R₁₄ are preferably hydrogen atoms.

In the formulae (10), (12) and (12a), at least one of R₁₀, R₁₂, R₁₃ or R₁₅ is preferably a substituted or unsubstituted aryl group having 6 to 30 ring carbon atoms. In the formulae (10), (12) and (12a), R₁₀, R₁₂, R₁₃ and R₁₅ are each more preferably a substituted or unsubstituted aryl group having 6 to 30 ring carbon atoms. In this case, R₁₁ and R₁₄ are preferably hydrogen atoms.

In the formulae (10), (12) and (12a), it is more preferable that: R₁₀, R₁₂, R₁₃ and R₁₅ are each independently a substituted or unsubstituted alkyl group having 1 to 30 carbon atoms (preferably 1 to 6 carbon atoms), a substituted or unsubstituted alkyl halide group having 1 to 30 carbon atoms (preferably 1 to 6 carbon atoms), or an aryl group having 6 to 30 ring carbon atoms (preferably 6 to 12 ring carbon atoms) and substituted with an alkyl group having 1 to 30 carbon atoms; and R₁₁ and R₁₄ are hydrogen atoms.

In the formula (11), at least one of R₁₀, R₁₂ or R₁₃ is preferably a substituted or unsubstituted alkyl group having 1 to 30 carbon atoms or a substituted or unsubstituted alkyl halide group having 1 to 30 carbon atoms.

In the formula (11), R₁₀, R₁₂ and R₁₃ are each more preferably a substituted or unsubstituted alkyl group having 1 to 30 carbon atoms or a substituted or unsubstituted alkyl halide group having 1 to 30 carbon atoms. In this case, R₁₁ is preferably a hydrogen atom.

In the formula (11), at least one of R₁₀, R₁₂ or R₁₃ is preferably a substituted or unsubstituted aryl group having 6 to 30 ring carbon atoms.

In the formula (11), R₁₀, R₁₂ and R₁₃ are each more preferably a substituted or unsubstituted aryl group having 6 to 30 ring carbon atoms. In this case, R₁₁ is preferably a hydrogen atom.

In the formula (11), it is more preferable that: R₁₀, R₁₂ and R₁₃ are each independently a substituted or unsubstituted alkyl group having 1 to 30 carbon atoms (preferably 1 to 6 carbon atoms), a substituted or unsubstituted alkyl halide group having 1 to 30 carbon atoms (preferably 1 to 6 carbon atoms), or an aryl group having 6 to 30 ring carbon atoms (preferably 6 to 12 ring carbon atoms) and substituted with an alkyl group having 1 to 30 carbon atoms, and R₁₁ is a hydrogen atom.

In the compound M1, examples of the fluorine-substituted alkoxy group include 2,2,2-trifluoroethoxy group, 2,2-difluoroethoxy group, 2,2,3,3,3-pentafluoro-1-propoxy group, 2,2,3,3-tetrafluoro-1-propoxy group, 1,1,1,3,3,3-hexafluoro-2-propoxy group, 2,2,3,3,4,4,4-heptafluoro-1-butyloxy group, 2,2,3,3,4,4-hexafluoro-1-butyloxy group, nonafluoro-tertiary-butyloxy group, 2,2,3,3,4,4,5,5,5-nonafluoropentanoxy group, 2,2,3,3,4,4,5,5,6,6,6-undecafluorohexanoxy group, 2,3-bis(trifluoromethyl)-2,3-butanedioxy group, 1,1,2,2-tetra(trifluoromethyl)ethylene glycoxy group, 4,4,5,5,6,6,6-heptafluorohexane-1,2-dioxy group, and 4,4,5,5,6,6,7,7,8,8,9,9,9-tridecafluorononane-1,2-dioxy group.

In the compound M1, examples of the fluorine-substituted aryloxy group or the aryloxy group substituted with a fluoroalkyl group include a pentafluorophenoxy group, 3,4,5-trifluorophenoxy group, 4-trifluoromethylphenoxy group, 3,5-bistrifluoromethylphenoxy group, 3-fluoro-4-trifluoromethylphenoxy group, 2,3,5,6-tetrafluoro-4-trifluoromethylphenoxy group, 4-fluorocatecholato group, 4-trifluoromethylcatecholato group, and 3,5-bistrifluoromethylcatecholato group.

When the compound M1 is a fluorescent compound, the compound M1 preferably emits light having a main peak wavelength in a range from 400 nm to 700 nm.

Herein, the main peak wavelength means a peak wavelength of a fluorescence spectrum exhibiting a maximum luminous intensity among fluorescence spectra measured in a toluene solution in which a measurement target compound is dissolved at a concentration ranging from 10⁻⁶ mol/l to 10⁻⁵ mol/l. A spectrophotofluorometer (F-7000 manufactured by Hitachi High-Tech Science Corporation) is used as a measurement device.

The compound M1 preferably exhibits red or green light emission.

Herein, the red light emission refers to light emission whose main peak wavelength of fluorescence spectrum is in a range from 600 nm to 660 nm.

When the compound M1 is a red fluorescent compound, the main peak wavelength of the compound M1 is preferably in a range from 600 nm to 660 nm, more preferably in a range from 600 nm to 640 nm, further preferably in a range from 610 nm to 630 nm.

Herein, the green light emission refers to light emission whose main peak wavelength of fluorescence spectrum is in a range from 500 nm to 560 nm.

When the compound M1 is a green fluorescent compound, the main peak wavelength of the compound M1 is preferably in a range from 500 nm to 560 nm, more preferably in a range from 500 nm to 540 nm, further preferably in a range from 510 nm to 540 nm.

Herein, the blue light emission refers to light emission whose main peak wavelength of fluorescence spectrum is in a range from 430 nm to 480 nm.

When the compound M1 is a blue fluorescent compound, the main peak wavelength of the compound M1 is preferably in a range from 430 nm to 480 nm, more preferably in a range from 440 nm to 480 nm.

A main peak wavelength of the light emitted from the organic EL device is measured as follows.

Voltage is applied on the organic EL devices such that a current density becomes 10 mA/cm², where spectral radiance spectrum is measured by a spectroradiometer CS-2000 (manufactured by Konica Minolta, Inc.).

A peak wavelength of an emission spectrum, at which the luminous intensity of the resultant spectral radiance spectrum is at the maximum, is measured and defined as the main peak wavelength (unit: nm).

### Manufacturing Method of Compound M1

The compound M1 can be manufactured by a known method.

Specific examples of the compound M1 according to the exemplary embodiment are shown below. It should however be noted that the invention is not limited to the specific examples of the compound.

A coordinate bond between a boron atom and a nitrogen atom in a pyrromethene skeleton is shown by various means such as a solid line, a broken line, an arrow, and omission. Herein, the coordinate bond is shown by a solid line or a broken line, or the description of the coordinate bond is omitted.

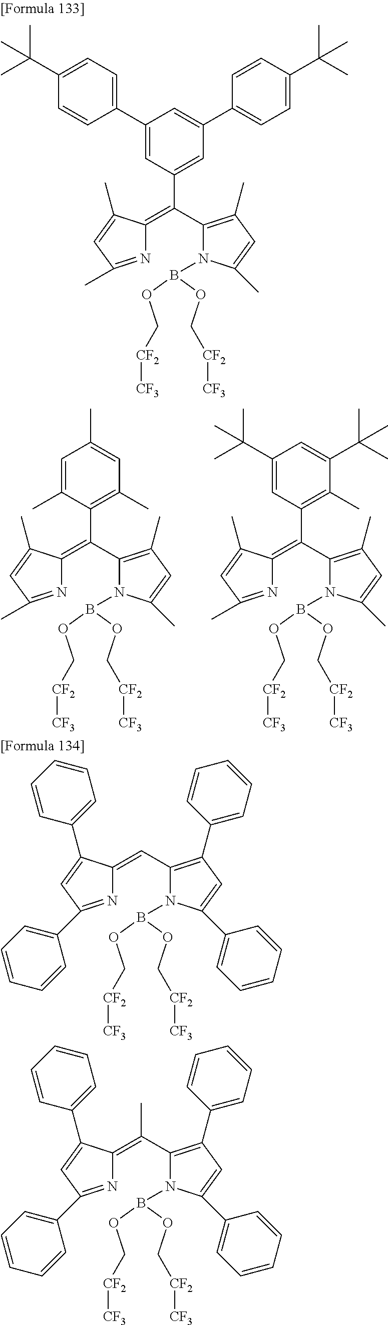

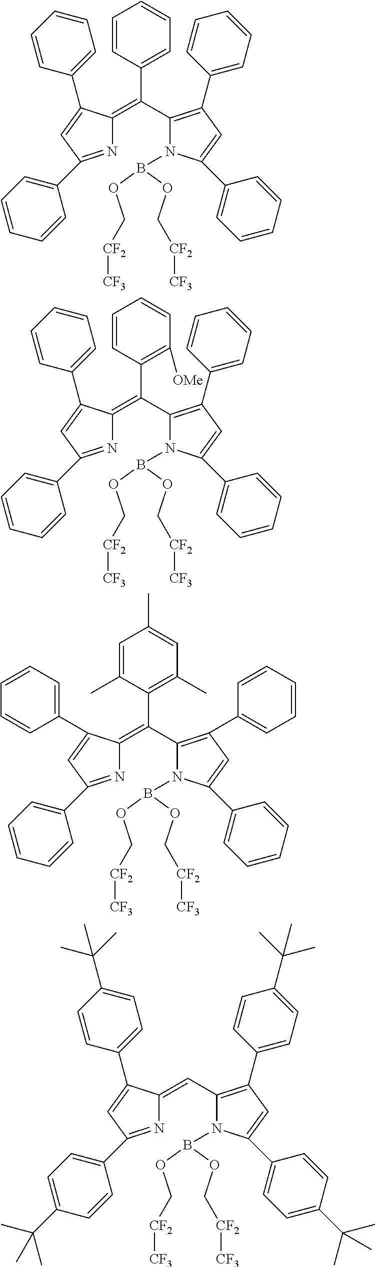

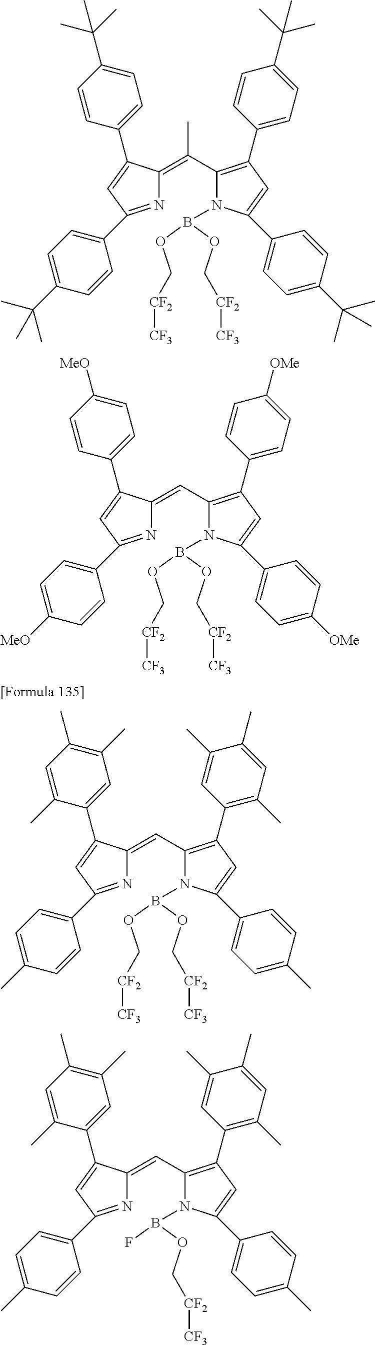

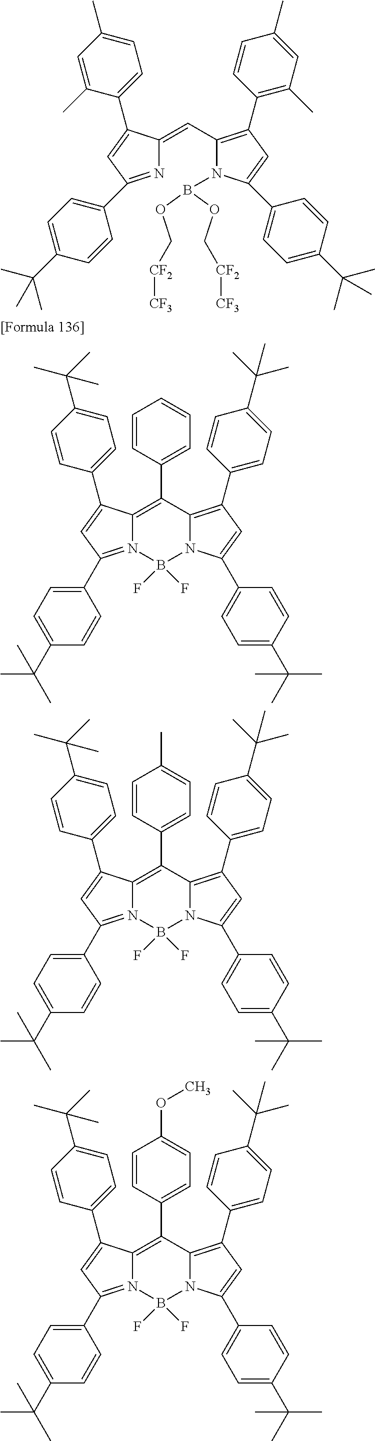

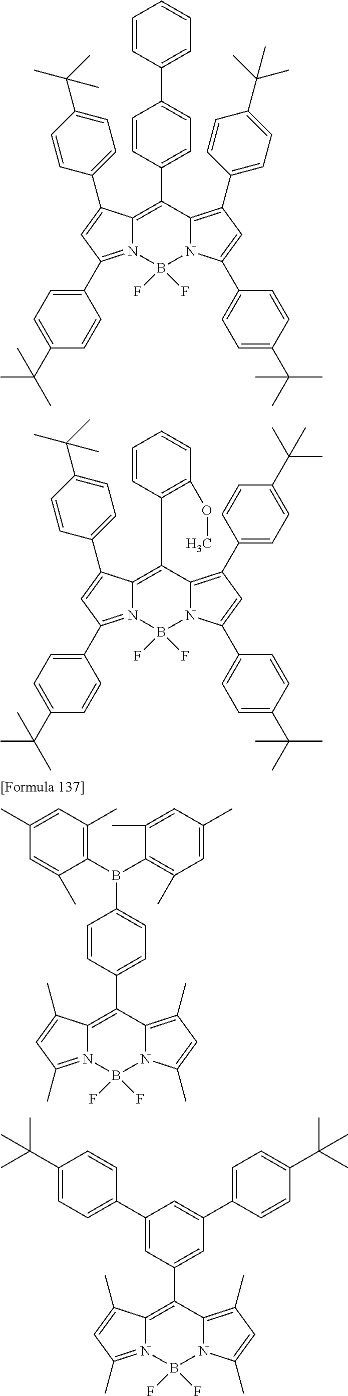

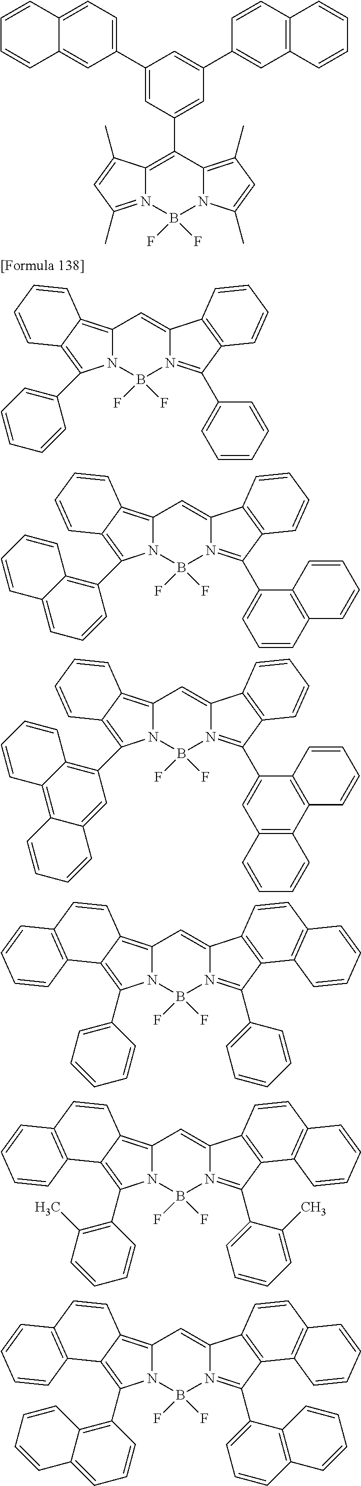

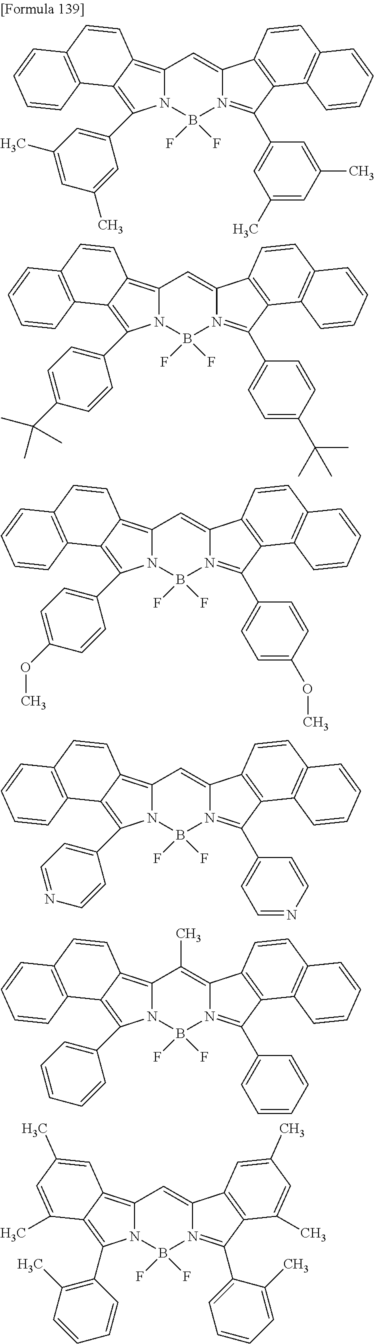

Relationship between Compound M3, Compound M2 and Compound M1 in Emitting Layer

In the organic EL device according to the exemplary embodiment, the singlet energy S₁(M2) of the compound M2 and a singlet energy S₁(M1) of the compound M1 preferably satisfy a relationship of a numerical formula (Numerical Formula 2) below.

S₁(M2)>S₁(M1) (Numerical Formula 2)

Moreover, the singlet energy S₁(M3) of the compound M3 is preferably larger than the singlet energy S₁(M1) of the compound M1.

S₁(M3)>S₁(M1) (Numerical Formula 2A)

The singlet energy S₁(M3) of the compound M3, the singlet energy S₁(M2) of the compound M2, and the singlet energy S₁(M1) of the compound M1 preferably satisfy a relationship of a numerical formula (Numerical Formula 2B) below.

S₁(M3)>S₁(M2)>S₁(M1) (Numerical Formula 2B)

It is preferable that mainly the fluorescent compound M1 emits light in the emitting layer when the organic EL device of the exemplary embodiment emits light.

The organic EL device according to the exemplary embodiment preferably emits red light or green light.

### Content Ratios of Compounds in Emitting Layer

Content ratios of the compound M3, the compound M2, and the compound M1 in the emitting layer preferably fall, for instance, within a range below.

The content ratio of the compound M3 is preferably in a range from 10 mass % to 80 mass %.

The content ratio of the compound M2 is preferably in a range from 10 mass % to 80 mass %, more preferably in a range from 10 mass % to 60 mass %, further preferably in a range from 20 mass % to 60 mass %.

The content ratio of the compound M1 is preferably in a range from 0.01 mass % to 10 mass %, more preferably in a range from 0.01 mass % to 5 mass %, further preferably in a range from 0.01 mass % to 1 mass %.

The upper limit of a total of the content ratios of the compound M3, the compound M2, and the compound M1 in the emitting layer is 100 mass %. It should be noted that the emitting layer of the exemplary embodiment may further contain material(s) other than the compounds M3, M2 and M1.

The emitting layer may include a single type of the compound M3 or may include two or more types of the compound M3. The emitting layer may include a single type of the compound M2 or may include two or more types of the compound M2. The emitting layer may include a single type of the compound M1 or may include two or more types of the compound M1.

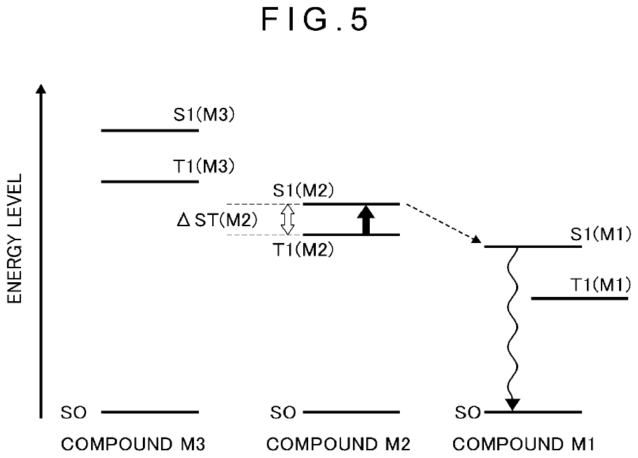

FIG. 5 shows an example of a relationship between energy levels of the compound M3, the compound M2, and the compound M1 in the emitting layer. In FIG. 5, S0 represents a ground state. S1 (M1) represents the lowest singlet state of the compound M1. T1(M1) represents the lowest triplet state of the compound M1. S1 (M2) represents the lowest singlet state of the compound M2. T1 (M2) represents the lowest triplet state of the compound M1. S1(M3) represents the lowest singlet state of the compound M3. T1 (M3) represents the lowest triplet state of the compound M3. A dashed arrow directed from S1 (M2) to S1 (M1) in FIG. 5 represents Forster energy transfer from the lowest singlet state of the compound M2 to the lowest singlet state of the compound M1.

As shown in FIG. 5, when a compound having a small ΔST(M2) is used as the compound M2, inverse intersystem crossing from the lowest triplet state T1 (M2) to the lowest singlet state S1(M2) can be caused by a heat energy. Subsequently, Forster energy transfer from the lowest singlet state S1 (M2) of the compound M2 to the compound M1 occurs to generate the lowest singlet state S1 (M1). Consequently, fluorescence from the lowest singlet state S1(M1) of the compound M1 can be observed. It is inferred that the internal quantum efficiency can be theoretically raised up to 100% also by using delayed fluorescence by the TADF mechanism.

The organic EL device according to the second exemplary embodiment contains the delayed fluorescent compound M2, the compound M3 having the singlet energy larger than that of the compound M2, and the compound M1 having the singlet energy smaller than that of the delayed fluorescent compound M2 in the emitting layer.

According to the second exemplary embodiment, an organic EL device with high performance, for instance, an organic EL device emitting light with a long lifetime can be achieved.

The organic EL device according to the second exemplary embodiment is applicable to an electronic device such as a display device and a light-emitting device.

### Third Exemplary Embodiment

### Compound

A compound of a third exemplary embodiment is a compound represented by a formula (300) below. A compound falling under the formula (300) among the specific examples of the compound M3 of the first embodiment is also used as a specific example of the compound of the third exemplary embodiment.

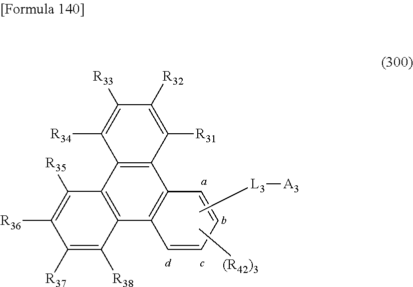

In the formula (300):

A₃ is a group represented by a formula (301a), (301b), (301c), (301d), (301e), (301f), (301g), (301h), (301i), (301j), (301k) or (301m) below;

L₃ is a single bond or a linking group, and L₃ as a linking group is a substituted or unsubstituted arylene group having 6 to 40 ring carbon atoms, a group represented by -L₃₁-L₃₂-, or a group represented by -L₃₁-L₃₂-L₃₃-;

L₃₁, L₃₂ and L₃₃ are each independently a substituted or unsubstituted arylene group having 6 to 40 ring carbon atoms;

L₃₁, L₃₂ and L₃₃ are mutually the same or different;

L₃, L₃₁, L₃₂ and L₃₃ are each not a substituted or unsubstituted anthracenylene group;

R₃₁ to R₃₈ and R₄₂ are each independently a hydrogen atom or a substituent;

a substituent, if present, for L₃, and R₃₁ to R₃₈ and R₄₂ as a substituent are each independently a halogen atom, a cyano group, a substituted or unsubstituted aryl group having 6 to 30 ring carbon atoms, a substituted or unsubstituted heterocyclic group having 5 to 30 ring atoms, a substituted or unsubstituted alkyl group having 1 to 30 carbon atoms, a substituted or unsubstituted cycloalkyl group having 3 to 30 ring carbon atoms, a substituted or unsubstituted alkyl halide group having 1 to 30 carbon atoms, a substituted or unsubstituted alkenyl group having 2 to 30 carbon atoms, a substituted or unsubstituted alkynyl group having 2 to 30 carbon atoms, a substituted or unsubstituted alkylsilyl group having 3 to 30 carbon atoms, a substituted or unsubstituted arylsilyl group having 6 to 60 ring carbon atoms, a substituted or unsubstituted arylphosphoryl group having 6 to 60 ring carbon atoms, a hydroxy group, a substituted or unsubstituted alkoxy group having 1 to 30 carbon atoms, a substituted or unsubstituted aryloxy group having 6 to 30 ring carbon atoms, an amino group, a substituted or unsubstituted alkylamino group having 2 to 30 carbon atoms, a substituted or unsubstituted arylamino group having 6 to 60 ring carbon atoms, a thiol group, a substituted or unsubstituted alkylthio group having 1 to 30 carbon atoms, or a substituted or unsubstituted arylthio group having 6 to 30 ring carbon atoms; and

a plurality of R₄₂ are mutually the same or different.

In the formula (300), L₃ is bonded to a carbon atom at one of positions a, b, c and d shown in the formula (300) and three R₄₂ are bonded to respective ones of carbon atoms at remaining three of the positions a, b, c and d not bonded to L₃.

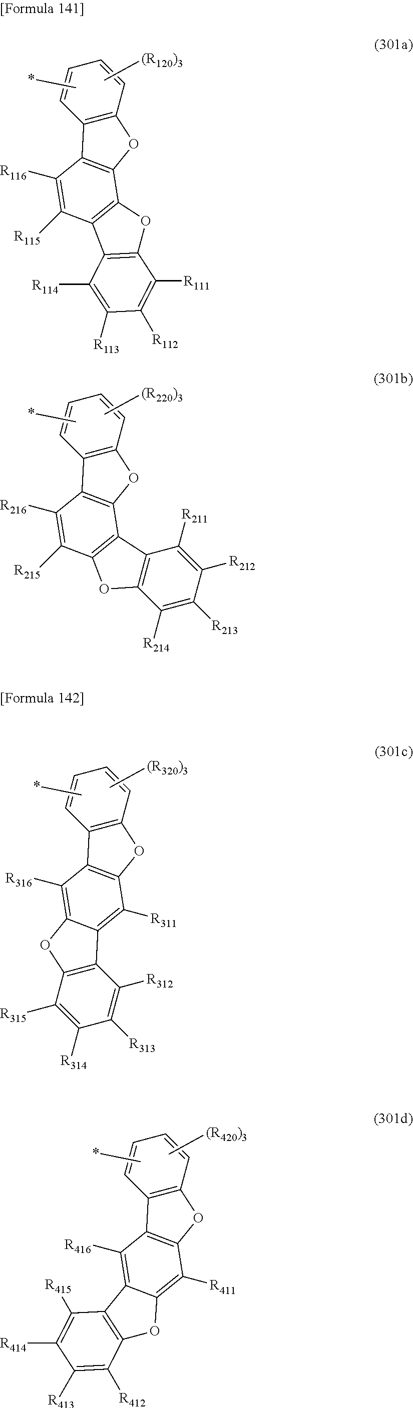

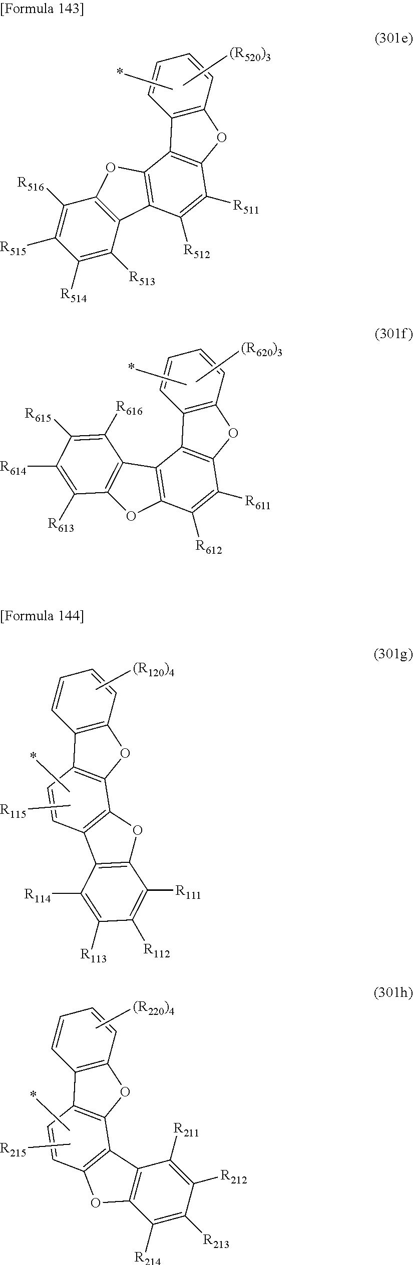

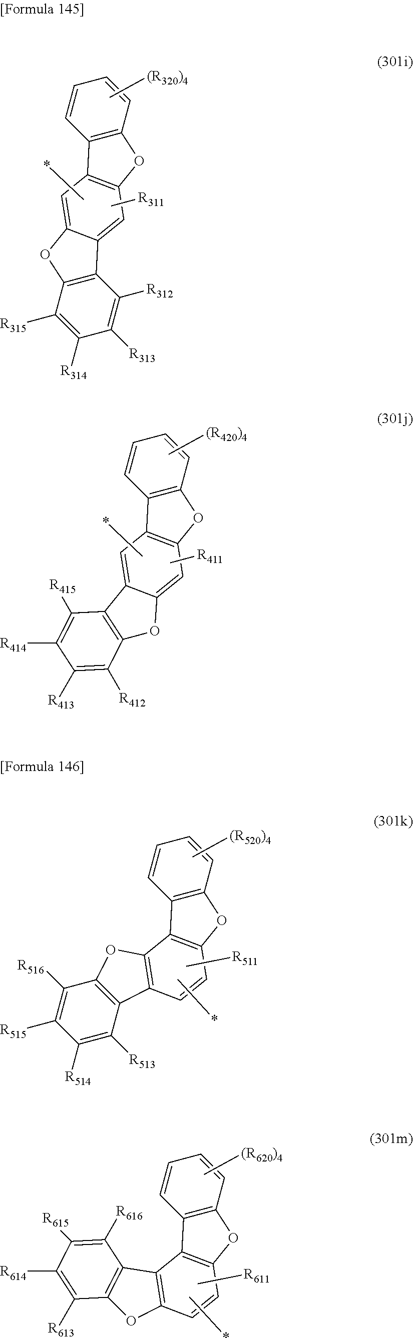

In the formulae (301a) to (301k) and (301m):

R₁₁₁ to R₁₁₆, R₂₁₁ to R₂₁₆, R₃₁₁ to R₃₁₆, R₄₁₁ to R₄₁₆, R₅₁₁ to R₅₁₆, R₆₁₁ to R₆₁₆, R₁₂₀, R₂₂₀, R₃₂₀, R₄₂₀, R₅₂₀ and R₆₂₀ are each independently a hydrogen atom or a substituent,

R₁₁₁ to R₁₁₆, R₂₁₁ to R₂₁₆, R₃₁₁ to R₃₁₆, R₄₁₁ to R₄₁₆, R₅₁₁ to R₅₁₆, R₆₁₁ to R₆₁₆, R₁₂₀, R₂₂₀, R₃₂₀, R₄₂₀, R₅₂₀ and R₆₂₀ as a substituent are each independently a halogen atom, a cyano group, a substituted or unsubstituted aryl group having 6 to 30 ring carbon atoms, a substituted or unsubstituted heterocyclic group having 5 to 30 ring atoms, a substituted or unsubstituted alkyl group having 1 to 30 carbon atoms, a substituted or unsubstituted cycloalkyl group having 3 to 30 ring carbon atoms, a substituted or unsubstituted alkyl halide group having 1 to 30 carbon atoms, a substituted or unsubstituted alkenyl group having 2 to 30 carbon atoms, a substituted or unsubstituted alkynyl group having 2 to 30 carbon atoms, a substituted or unsubstituted alkylsilyl group having 3 to 30 carbon atoms, a substituted or unsubstituted arylsilyl group having 6 to 60 ring carbon atoms, a substituted or unsubstituted arylphosphoryl group having 6 to 60 ring carbon atoms, a hydroxy group, a substituted or unsubstituted alkoxy group having 1 to 30 carbon atoms, a substituted or unsubstituted aryloxy group having 6 to 30 ring carbon atoms, an amino group, a substituted or unsubstituted alkylamino group having 2 to 30 carbon atoms, a substituted or unsubstituted arylamino group having 6 to 60 ring carbon atoms, a thiol group, a substituted or unsubstituted alkylthio group having 1 to 30 carbon atoms, or a substituted or unsubstituted arylthio group having 6 to 30 ring carbon atoms;

R₁₁₁ to R₁₁₄, R₂₁₁ to R₂₁₄, R₃₁₂ to R₃₁₅, R₄₁₂ to R₄₁₅, R₅₁₃ to R₅₁₆, R₆₁₃ to R₆₁₆, R₁₂₀, R₂₂₀, R₃₂₀, R₄₂₀, R₅₂₀ and R₆₂₀ are each not an unsubstituted triphenylenyl group; and

a plurality of R₁₂₀ are mutually the same or different, a plurality of R₂₂₀ are mutually the same or different, a plurality of R₃₂₀ are mutually the same or different, a plurality of R₄₂₀ are mutually the same or different, a plurality of R₅₂₀ are mutually the same or different, a plurality of R₆₂₀ are mutually the same or different, and * represents a bonding position to L₃.

In the compound of the third exemplary embodiment, it is preferable that L₃ is bonded to a carbon atom at the position b or c shown in the formula (300).

In the compound of the third exemplary embodiment, it is also preferable that L₃ is bonded to a carbon atom at the position a or d shown in the formula (300).

In the compound of the third exemplary embodiment, it is preferable that R₁₁₁ to R₁₁₄, R₂₁₁ to R₂₁₄, R₃₁₂ to R₃₁₅, R₄₁₂ to R₄₁₅, R₅₁₃ to R₅₁₆, R₆₁₃ to R₆₁₆, R₁₂₀, R₂₂₀, R₃₂₀, R₄₂₀, R₅₂₀ and R₆₂₀ are each not a substituted triphenylenyl group.

In the compound of the third exemplary embodiment, R₃₁ to R₃₈, R₄₂, R₁₁₁ to R₁₁₆, R₂₁₁ to R₂₁₆, R₃₁₁ to R₃₁₆, R₄₁₁ to R₄₁₆, R₅₁₁ to R₅₁₆, R₆₁₁ to R₆₁₆, R₁₂₀, R₂₂₀, R₃₂₀, R₄₂₀, R₅₂₀ and R₆₂₀ are preferably each independently a hydrogen atom or a substituted or unsubstituted aryl group having 6 to 30 ring carbon atoms, more preferably a hydrogen atom or a substituted or unsubstituted phenyl group, further preferably a hydrogen atom.

In the compound of the third exemplary embodiment, A₃ is preferably a group represented by a formula (310a), (310b), (310c), (310d), (310e) or (310f) below.

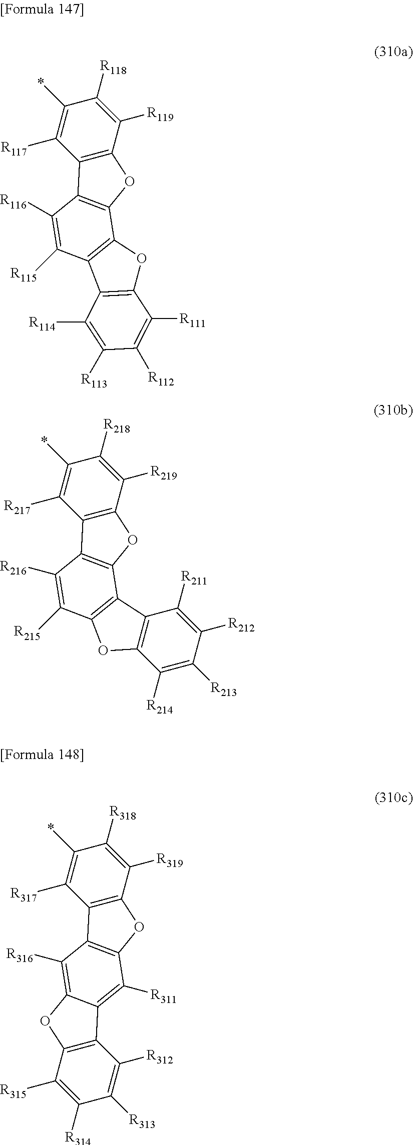

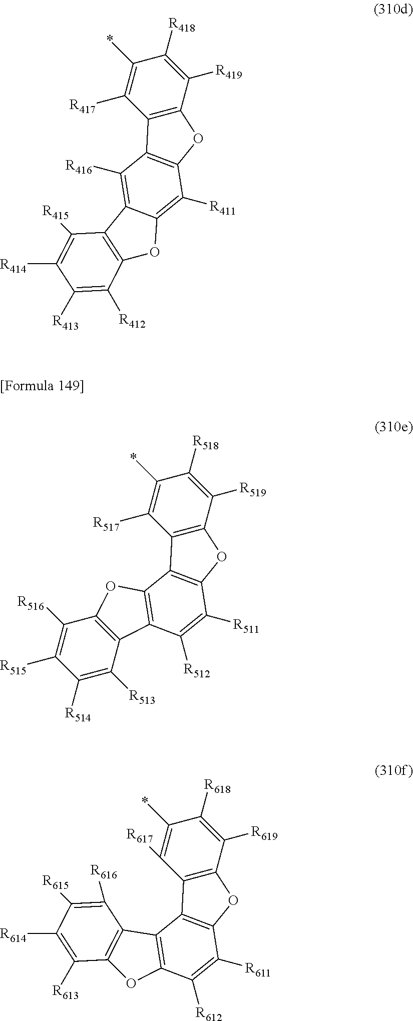

In the formula (310a), R₁₁₁ to R₁₁₉ each independently represent the same as R₁₁₅ in the formula (301a), and R₁₁₁ to R₁₁₄ and R₁₁₇ to R₁₁₉ are each not an unsubstituted triphenylenyl group;

in the formula (310b), R₂₁₁ to R₂₁₉ each independently represent the same as R₂₁₅ in the formula (301b), and R₂₁₁ to R₂₁₄ and R₂₁₇ to R₂₁₉ are each not an unsubstituted triphenylenyl group;

in the formula (310c), R₃₁₁ to R₃₁₉ each independently represent the same as R₃₁₁ in the formula (301c), and R₃₁₂ to R₃₁₅ and R₃₁₇ to R₃₁₉ are each not an unsubstituted triphenylenyl group;

in the formula (310d), R₄₁₁ to R₄₁₉ each independently represent the same as R₄₁₁ in the formula (301d), and R₄₁₂ to R₄₁₅ and R₄₁₇ to R₄₁₉ are each not an unsubstituted triphenylenyl group;

in the formula (310e), R₅₁₁ to R₅₁₉ each independently represent the same as R₅₁₁ in the formula (301e), and R₅₁₃ to R₅₁₆ and R₅₁₇ to R₅₁₉ are each not an unsubstituted triphenylenyl group;

in the formula (310f), R₆₁₁ to R₆₁₉ each independently represent the same as R₆₁₁ in the formula (301f), and R₆₁₃ to R₆₁₆ and R₆₁₇ to R₆₁₉ are each not an unsubstituted triphenylenyl group; and

* represents a bonding position to L₃.

In the formulae (310a) to (301f), it is preferable that R₁₁₁ to R₁₁₄, R₁₁₇ to R₁₁₉, R₂₁₁ to R₂₁₄, R₂₁₇ to R₂₁₉, R₃₁₂ to R₃₁₅, R₃₁₇ to R₃₁₉, R₄₁₂ to R₄₁₅, R₄₁₇ to R₄₁₉, R₅₁₃ to R₅₁₆, R₅₁₇ to R₅₁₉, R₆₁₃ to R₆₁₆ and R₆₁₇ to R₆₁₉ are each not a substituted triphenylenyl group.

In the formulae (310a) to (301f), R₁₁₁ to R₁₁₉, R₂₁₁ to R₂₁₉, R₃₁₁ to R₃₁₉, R₄₁₁ to R₄₁₉, R₅₁₁ to R₅₁₉ and R₆₁₁ to R₆₁₉ are preferably each independently a hydrogen atom or a substituted or unsubstituted aryl group having 6 to 30 ring carbon atoms, more preferably a hydrogen atom or a substituted or unsubstituted phenyl group, further preferably a hydrogen atom.

In the compound of the third exemplary embodiment, A₃ is also preferably a group represented by one of formulae (311a) to (314a), (311b) to (314b), (311c) to (314c), (311 d) to (315d), (311e) to (314e) and (311 f) to (314f) below.

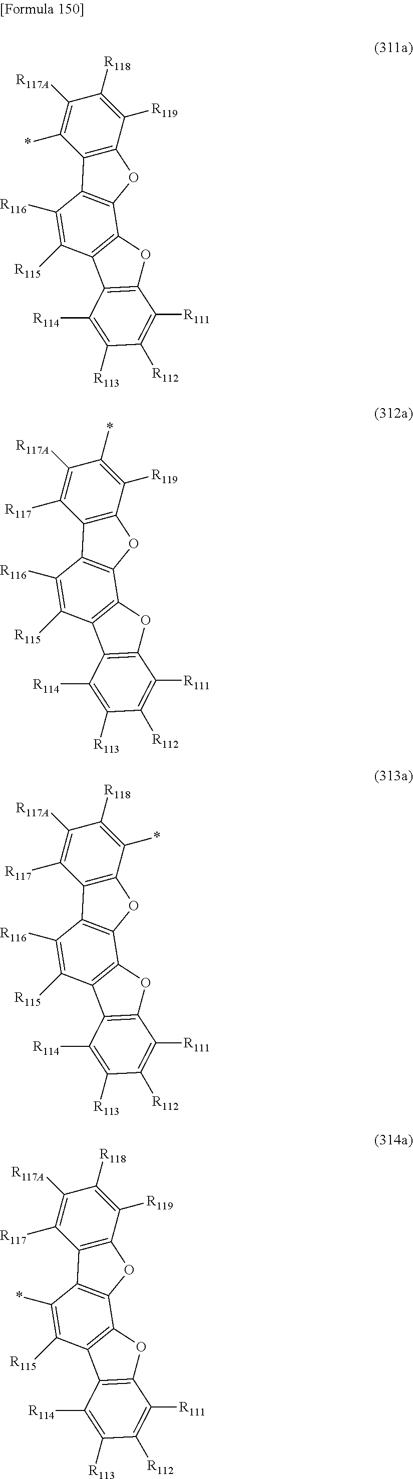

In the formulae (311a) to (314a): R₁₁₁ to R₁₁₉ and R_{117A} each independently represent the same as R₁₁₅ in the formula (301a); R₁₁₁ to R₁₁₄, R₁₁₇ to R₁₁₉ and R_{117A} are each not an unsubstituted triphenylenyl group; and * represents a bonding position to L₃.

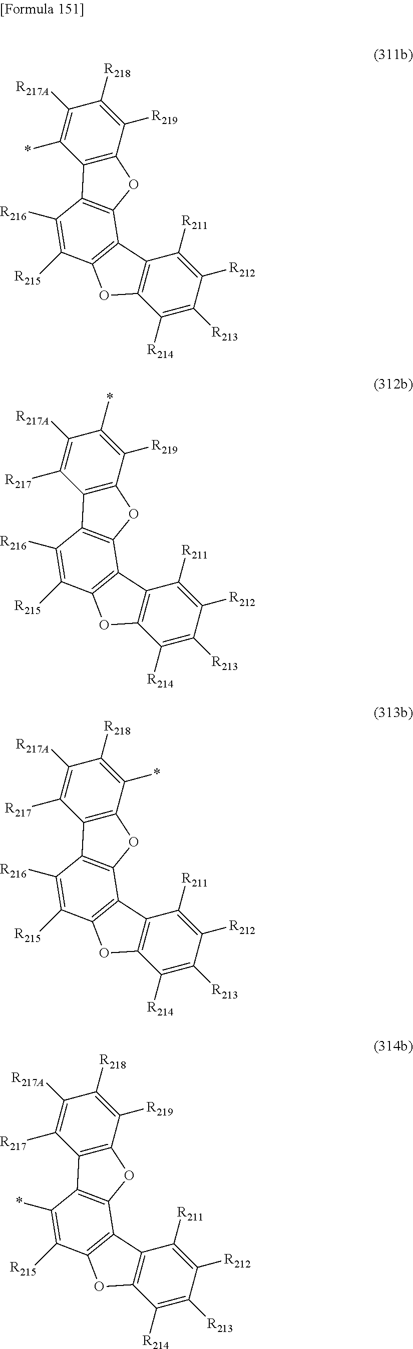

In the formulae (311b) to (314b): R₂₁₁ to R₂₁₉ and R_{217A} each independently represent the same as R₂₁₅ in the formula (301b); R₂₁₁ to R₂₁₄, R₂₁₇ to R₂₁₉ and R_{217A} are each not an unsubstituted triphenylenyl group; and * represents a bonding position to L₃.

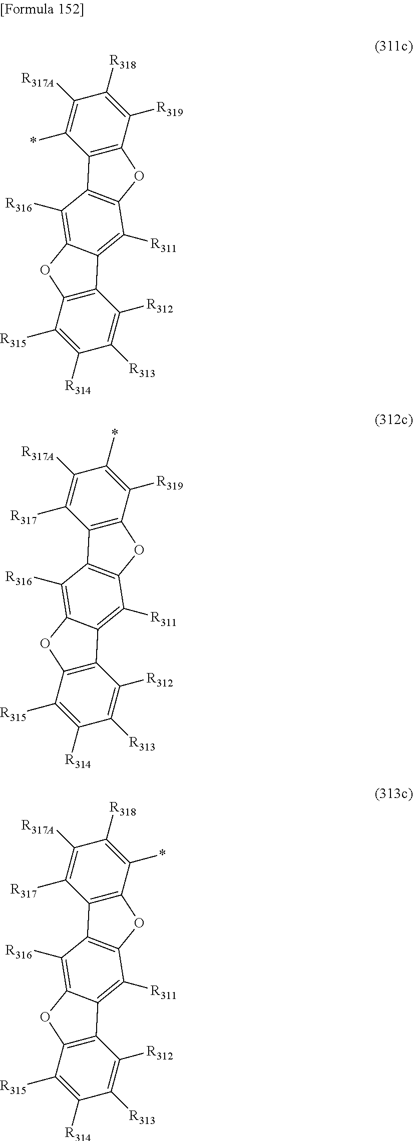

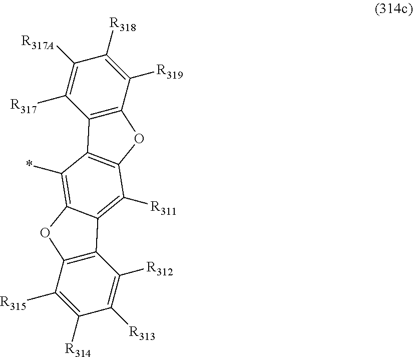

In the formulae (311c) to (314c): R₃₁₁ to R₃₁₉ and R_{317A} each independently represent the same as R₃₁₁ in the formula (301c); R₃₁₂ to R₃₁₅, R₃₁₇ to R₃₁₉ and R_{317A} are each not an unsubstituted triphenylenyl group; and * represents a bonding position to L₃.

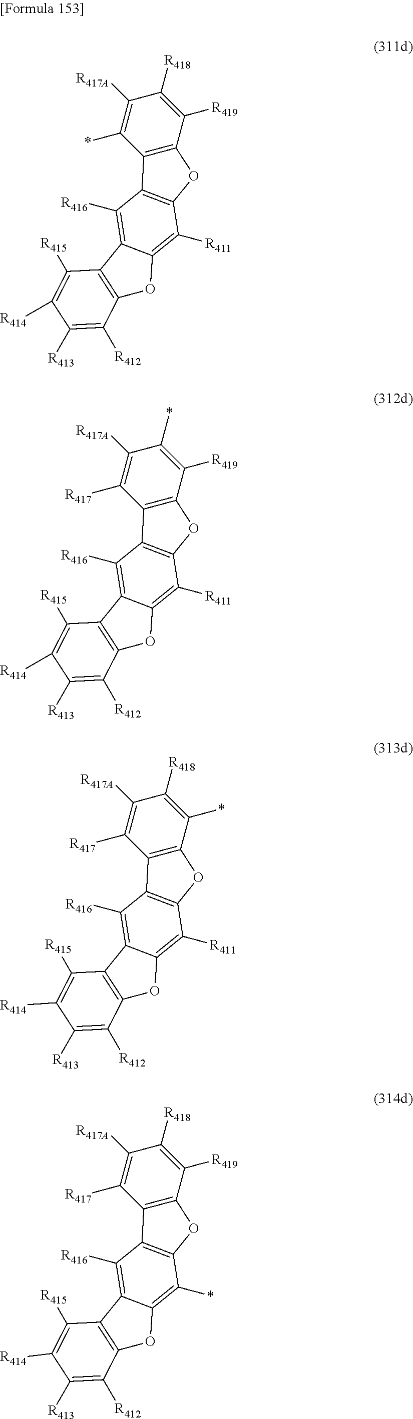

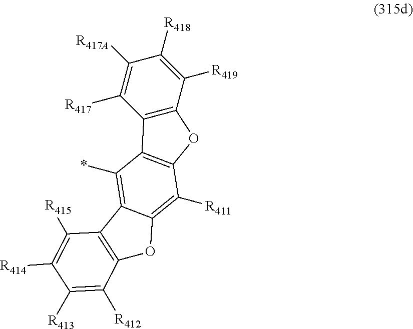

In the formulae (311 d) to (315d): R₄₁₁ to R₄₁₉ and R_{417A} each independently represent the same as R₄₁₁ in the formula (301d); R₄₁₂ to R₄₁₅, R₄₁₇ to R₄₁₉ and R_{417A} are each not an unsubstituted triphenylenyl group; and * represents a bonding position to L₃.

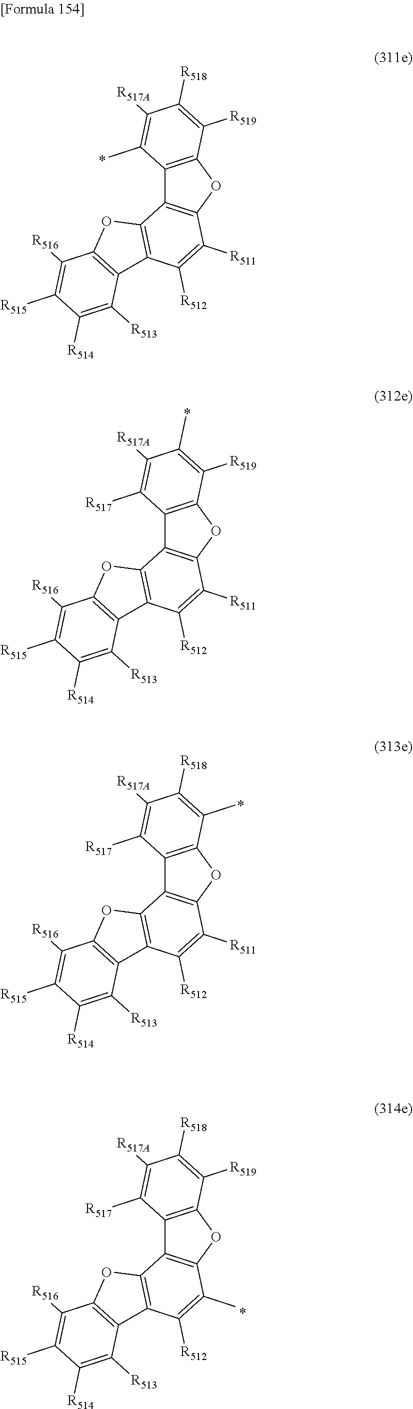

In the formulae (311e) to (314e): R₅₁₁ to R₅₁₉ and R_{517A} each independently represent the same as R₅₁₁ in the formula (301e); R₅₁₃ to R₅₁₆, R₅₁₇ to R₅₁₉ and R_{517A} are each not an unsubstituted triphenylenyl group; and * represents a bonding position to L₃.

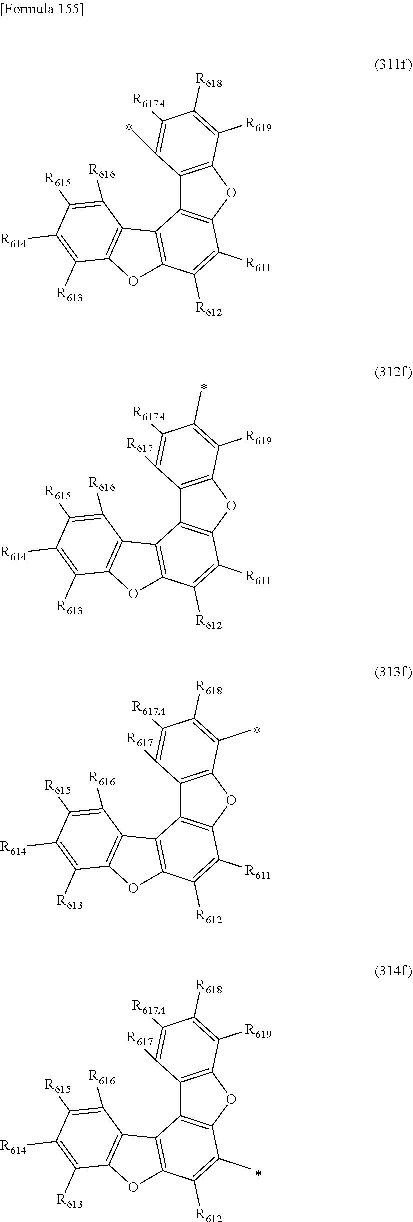

In the formulae (311f) to (314f): R₆₁₁ to R₆₁₉ and R_{617A} each independently represent the same as R₆₁₁ in the formula (301f); R₆₁₃ to R₆₁₆, R₆₁₇ to R₆₁₉ and R_{617A} are each not an unsubstituted triphenylenyl group; and * represents a bonding position to L₃.

In the formulae (311a) to (314a), (311b) to (314b), (311c) to (314c), (311d) to (315d), (311e) to (314e) and (311 f) to (314f), R₁₁₁ to R₁₁₉, R_{117A}, R₂₁₁ to R₂₁₉, R_{217A}, R₃₁₁ to R₃₁₉, R_{317A}, R₄₁₁ to R₄₁₉, R_{417A}, R₅₁₁ to R₅₁₉, R_{517A}, R₆₁₁ to R₆₁₉ and R_{617A} are preferably each independently a hydrogen atom or a substituted or unsubstituted aryl group having 6 to 30 ring carbon atoms, more preferably a hydrogen atom or a substituted or unsubstituted phenyl group, further preferably a hydrogen atom.

The compound of the third exemplary embodiment is preferably represented by a formula (301A), (302A), (303A), (304A), (305A) or (306A) below.

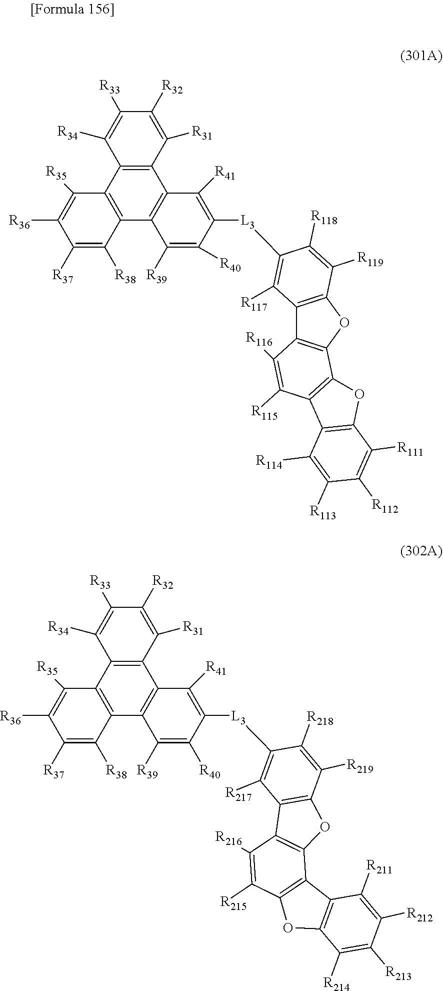

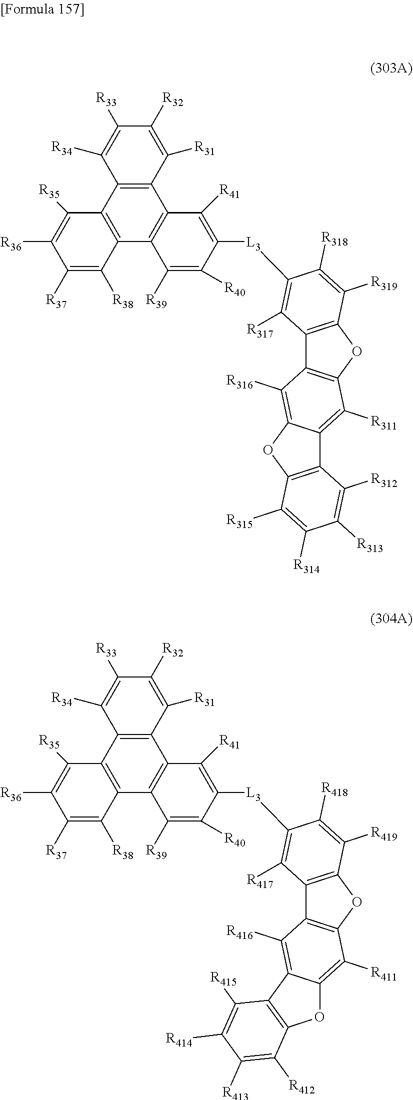

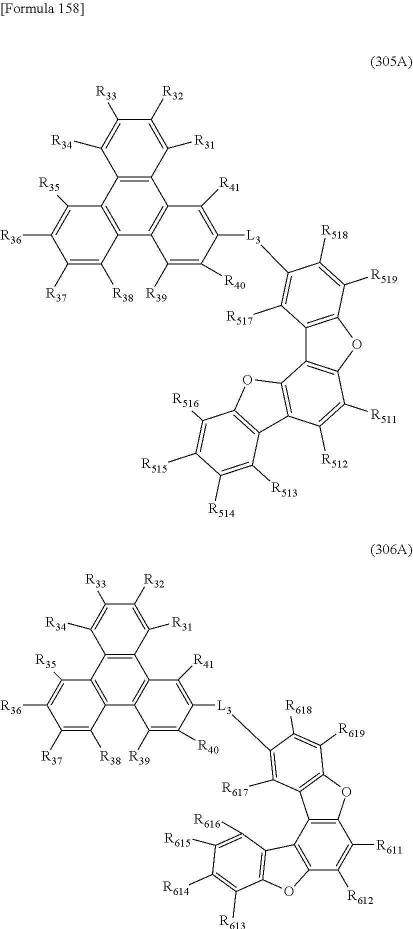

In the formula (301A), R₁₁₁ to R₁₁₉ each independently represent the same as R₁₁₅ in the formula (301a), and R₁₁₁ to R₁₁₄ and R₁₁₇ to R₁₁₉ are each not an unsubstituted triphenylenyl group;

in the formula (302A), R₂₁₁ to R₂₁₉ each independently represent the same as R₂₁₅ in the formula (301b), and R₂₁₁ to R₂₁₄ and R₂₁₇ to R₂₁₉ are each not an unsubstituted triphenylenyl group;

in the formula (303A), R₃₁₁ to R₃₁₉ each independently represent the same as R₃₁₁ in the formula (301c), and R₃₁₂ to R₃₁₅ and R₃₁₇ to R₃₁₉ are each not an unsubstituted triphenylenyl group;

in the formula (304A), R₄₁₁ to R₄₁₉ each independently represent the same as R₄₁₁ in the formula (301d), and R₄₁₂ to R₄₁₅ and R₄₁₇ to R₄₁₉ are each not an unsubstituted triphenylenyl group;

in the formula (305A), R₅₁₁ to R₅₁₉ each independently represent the same as R₅₁₁ in the formula (301e), and R₅₁₃ to R₅₁₆ and R₅₁₇ to R₅₁₉ are each not an unsubstituted triphenylenyl group;

in the formula (306A), R₆₁₁ to R₆₁₉ each independently represent the same as R₆₁₁ in the formula (301f), and R₆₁₃ to R₆₁₆ and R₆₁₇ to R₆₁₉ are each not an unsubstituted triphenylenyl group;

in the formulae (301A) to (306A), R₃₁ to R₄₁ each independently represent the same as R₄₂ in the formula (300); and

L₃ represents the same as L₃ in the formula (300).

In the formulae (301A) to (306A), R₃₁ to R₄₁, R₁₁₁ to R₁₁₉, R₂₁₁ to R₂₁₉, R₃₁₁ to R₃₁₉, R₄₁₁ to R₄₁₉, R₅₁₁ to R₅₁₉ and R₆₁₁ to R₆₁₉ are preferably each independently a hydrogen atom or a substituted or unsubstituted aryl group having 6 to 30 ring carbon atoms, more preferably a hydrogen atom or a substituted or unsubstituted phenyl group, further preferably a hydrogen atom.

The compound of the third exemplary embodiment is also preferably a compound represented by one of the formulae (307) to (331) in the first exemplary embodiment and falling under the formula (300). Specifically, the compound of the third exemplary embodiment is also preferably a compound represented by one of the formulae (307) to (331) and satisfying the following requirements.

In the formulae (307) to (310), R₁₁₁ to R₁₁₉ and R_{117A} each independently represent the same as R₁₁₅ in the formula (301a), and R₁₁₁ to R₁₁₄, R₁₁₇ to R₁₁₉ and R_{117A} are each not an unsubstituted triphenylenyl group.

In the formulae (311) to (314), R₂₁₁ to R₂₁₉ and R_{217A} each independently represent the same as R₂₁₅ in the formula (301b), and R₂₁₁ to R₂₁₄, R₂₁₇ to R₂₁₉ and R_{217A} are each not an unsubstituted triphenylenyl group.

In the formulae (315) to (318), R₃₁₁ to R₃₁₉ and R_{317A} each independently represent the same as R₃₁₁ in the formula (301c), and R₃₁₂ to R₃₁₅, R₃₁₇ to R₃₁₉ and R_{317A} are each not an unsubstituted triphenylenyl group.

In the formulae (319) to (323), R₄₁₁ to R₄₁₉ and R_{417A} each independently represent the same as R₄₁₁ in the formula (301d), and R₄₁₂ to R₄₁₅, R₄₁₇ to R₄₁₉ and R_{417A} are each not an unsubstituted triphenylenyl group.

In the formulae (324) to (327), R₅₁₁ to R₅₁₉ and R_{517A} each independently represent the same as R₅₁₁ in the formula (301e), and R₅₁₃ to R₅₁₆, R₅₁₇ to R₅₁₉ and R_{517A} are each not an unsubstituted triphenylenyl group.

In the formulae (328) to (331), R₆₁₁ to R₆₁₉ and R_{617A} each independently represent the same as R₆₁₁ in the formula (301f), and R₆₁₃ to R₆₁₆, R₆₁₇ to R₆₁₉ and R_{617A} are each not an unsubstituted triphenylenyl group.

In the formulae (307) to (331), R₃₁ to R₄₁ each independently represent the same as R₄₂ in the formula (300), and L₃ represents the same as L₃ in the formula (300).

When the compound of the third exemplary embodiment is a compound represented by one of the formulae (307) to (331) in the first exemplary embodiment, R₁₁₁ to R₁₁₉, R_{117A}, R₂₁₁ to R₂₁₉, R_{217A}, R₃₁₁ to R₃₁₉, R_{317A}, R₄₁₁ to R₄₁₉, R_{417A}, R₅₁₁ to R₅₁₉, R_{517A}, R₆₁₁ to R₆₁₉ and R_{617A} in the formulae (307) to (331) are preferably each independently a hydrogen atom or a substituted or unsubstituted aryl group having 6 to 30 ring carbon atoms, more preferably a hydrogen atom or a substituted or unsubstituted phenyl group, further preferably a hydrogen atom.

In the compound of the third exemplary embodiment, it is preferable that L₃ is a single bond or a linking group, and L₃ as a linking group is a substituted or unsubstituted phenylene group, a substituted or unsubstituted biphenylene group, a substituted or unsubstituted terphenylene group, or a substituted or unsubstituted naphthylene group.

In the compound of the third exemplary embodiment, L₃ as a linking group is also preferably a divalent group derived from a substituted or unsubstituted phenanthryl group, a divalent group derived from a substituted or unsubstituted triphenylenyl group, a divalent group derived from a substituted or unsubstituted fluorenyl group (preferably a 9,9′-dimethylfluorenyl group or a 9,9′-diphenylfluorenyl group), or a divalent group derived from a 9,9′-spirobifluorenyl group.

In the compound of the third exemplary embodiment, when L₃ as a linking group is a group represented by -L₃₁-L₃₂- or a group represented by -L₃₁-L₃₂-L₃₃-, L₃₁, L₃₂ and L₃₃ are preferably each independently a substituted or unsubstituted phenylene group or a substituted or unsubstituted naphthylene group.

In the compound of the third exemplary embodiment, a substituent, if present, for L₃ is preferably a substituted or unsubstituted aryl group having 6 to 30 ring carbon atoms, more preferably a substituted or unsubstituted phenyl group.

In the compound of the third exemplary embodiment, L₃ is also preferably a single bond.

In the compound of the third exemplary embodiment, L₃ is also preferably at least one group selected from the group consisting of groups represented by the formulae (L₃-1) to (L₃-18).

In the formulae (L₃-1) to (L₃-18), Q₁ to 014 are each independently a hydrogen atom or a substituent. Q₁ to Q₁₄ as a substituent are preferably each independently a hydrogen atom or a substituted or unsubstituted aryl group having 6 to 30 ring carbon atoms, more preferably a substituted or unsubstituted phenyl group, a substituted or unsubstituted phenanthryl group, a substituted or unsubstituted triphenylenyl group, a substituted or unsubstituted fluorenyl group (preferably a 9,9′-dimethylfluorenyl group or a 9,9′-diphenylfluorenyl group), or a 9,9′-spirobifluorenyl group, further preferably a substituted or unsubstituted phenyl group.

It should be noted that the compound of the third exemplary embodiment also corresponds to a compound with an exemplary arrangement of the compound M3 described in the first exemplary embodiment. Accordingly, specific examples of the compound of the third exemplary embodiment are also shown in the specific examples of the compound M3 described in the first exemplary embodiment.

### Organic EL Device

An organic EL device in an exemplary arrangement according to the third exemplary embodiment is an organic EL device in which the compound M3 in the organic EL device according to the first exemplary embodiment is replaced by the compound of the third exemplary embodiment.

Specifically, the organic EL device in an exemplary arrangement according to the third exemplary embodiment includes: an anode; a cathode; and an emitting layer provided between the anode and the cathode, in which the emitting layer contains the delayed fluorescent compound M2 and the compound of the third exemplary embodiment, and the singlet energy S₁(M2) of the compound M2 and a singlet energy S₁(M3′) of the compound of the third exemplary embodiment satisfy a relationship of a numerical formula (Numerical Formula 1′) below.

S₁(M3′)>S₁(M2) (Numerical Formula 1′)

The compound of the third exemplary embodiment can achieve higher performance of an organic EL device, for instance, extend the lifetime of the organic EL device.

Therefore, the organic EL device in an exemplary arrangement according to the third exemplary embodiment also has higher performance, for instance, emits light with a longer lifetime.

### Fourth Exemplary Embodiment

### Electronic Device

An electronic device according to a fourth exemplary embodiment is installed with any one of the organic EL devices according to the above exemplary embodiments. Examples of the electronic device include a display device and a light-emitting unit. Examples of the display device include a display component (e.g., an organic EL panel module), TV, mobile phone, tablet and personal computer. Examples of the light-emitting unit include an illuminator and a vehicle light.

### Fifth Exemplary Embodiment

### Organic-EL-Device Material

An organic-EL-device material of a fifth exemplary embodiment contains the compound of the third exemplary embodiment.

The organic-EL-device material of the fifth exemplary embodiment can achieve higher performance of an organic EL device, for instance, extend the lifetime of the organic EL device.

The organic-EL-device material of the fifth exemplary embodiment may further contain an additional compound. When the organic-EL-device material of the fifth exemplary embodiment further contains an additional compound, the additional compound may be solid or liquid.

### Modification of Embodiment(s)

The scope of the invention is not limited by the above-described exemplary embodiments but includes any modification and improvement as long as such modification and improvement are compatible with the invention.

For instance, the emitting layer is not limited to a single layer, but may be provided by laminating a plurality of emitting layers. When the organic EL device has the plurality of emitting layers, it is only required that at least one of the emitting layers satisfies the conditions described in the above exemplary embodiments. For instance, the rest of the emitting layers may be a fluorescent emitting layer or a phosphorescent emitting layer with use of emission caused by electron transfer from the triplet excited state directly to the ground state.

When the organic EL device includes a plurality of emitting layers, these emitting layers may be mutually adjacently provided, or may form a so-called tandem organic EL device, in which a plurality of emitting units are layered via an intermediate layer.

For instance, a blocking layer may be provided adjacent to at least one of a side of the emitting layer close to the anode or a side of the emitting layer close to the cathode. The blocking layer is preferably provided in contact with the emitting layer to block at least any of holes, electrons, excitons or combinations thereof.

For instance, when the blocking layer is provided in contact with the side of the emitting layer close to the cathode, the blocking layer permits transport of electrons, and blocks holes from reaching a layer provided closer to the cathode (e.g., the electron transporting layer) beyond the blocking layer. When the organic EL device includes the electron transporting layer, the blocking layer is preferably interposed between the emitting layer and the electron transporting layer.

When the blocking layer is provided in contact with the side of the emitting layer close to the anode, the blocking layer permits transport of holes and blocks electrons from reaching a layer provided closer to the anode (e.g., the hole transporting layer) beyond the blocking layer. When the organic EL device includes the hole transporting layer, the blocking layer is preferably interposed between the emitting layer and the hole transporting layer.

Alternatively, the blocking layer may be provided adjacent to the emitting layer so that the excitation energy does not leak out from the emitting layer toward neighboring layer(s). The blocking layer blocks excitons generated in the emitting layer from being transferred to a layer(s) (e.g., the electron transporting layer and the hole transporting layer) closer to the electrode(s) beyond the blocking layer.

The emitting layer is preferably bonded with the blocking layer.

Specific structure, shape and the like of the components in the invention may be designed in any manner as long as an object of the invention can be achieved.

### Other Explanations

Herein, numerical ranges represented by “x to y” represents a range whose lower limit is the value (x) recited before “to” and whose upper limit is the value (y) recited after “to.”

Herein, the phrase “Rx and Ry are mutually bonded to form a ring” means, for instance, that Rx and Ry include a carbon atom, a nitrogen atom, an oxygen atom, a sulfur atom or a silicon atom, the atom(s) contained in Rx (a carbon atom, a nitrogen atom, an oxygen atom, a sulfur atom or a silicon atom) and the atom(s) contained in Ry (a carbon atom, a nitrogen atom, an oxygen atom, a sulfur atom or a silicon atom) are bonded via a single bond(s), a double bond(s), a triple bond, and/or a divalent linking group(s) to form a ring having 5 or more ring atoms (specifically, a heterocycle or an aromatic hydrocarbon ring). x represents a number, a character or a combination of a number and a character. y represents a number, a character or a combination of a number and a character.

The divalent linking group is not limited. Examples of the divalent linking group include —O—, —CO—, —CO₂—, —S—, —SO—, —SO₂—, —NH—, —NRa-, and a group provided by a combination of two or more of these linking group.

Specific examples of the heterocyclic ring include a cyclic structure (heterocyclic ring) obtained by removing a bond from a “heteroaryl group Sub₂” exemplarily shown in the later-described “Description of Each Substituent in Formula.” The heterocyclic ring may have a substituent.

Specific examples of the heterocyclic ring include a cyclic structure (heterocyclic ring) obtained by removing a bond from an “aryl group Sub₁” exemplarily shown in the later-described “Description of Each Substituent in Formula.” The aromatic hydrocarbon ring may have a substituent.

Examples of Ra include a substituted or unsubstituted alkyl group Sub₃ having 1 to 30 carbon atoms, a substituted or unsubstituted aryl group Sub₁ having 6 to 40 ring carbon atoms, and a substituted or unsubstituted heteroaryl group Sub₂ having 5 to 30 ring atoms, which are exemplarily shown in the later-described “Description of Each Substituent in Formula.”

Rx and Ry are mutually bonded to form a ring, which means, for instance, that: an atom contained in Rx₁ and an atom contained in Ry₁ in a molecular structure represented by a formula (E₁) below form a ring (cyclic structure) E represented by a formula (E₂); an atom contained in Rx₁ and an atom contained in Ry₁ in a molecular structure represented by a formula (F1) below form a ring F represented by a formula (F2); an atom contained in Rx₁ and an atom contained in Ry₁ in a molecular structure represented by a formula (G1) below form a ring G represented by a formula (G2); an atom contained in Rx₁ and an atom contained in Ry₁ in a molecular structure represented by a formula (H1) below form a ring H represented by a formula (H2); and an atom contained in Rx₁ and an atom contained in Ry₁ in a molecular structure represented by a formula (I1) below form a ring I represented by a formula (I2).

In the formulae (E₁) to (I1), * each independently represent a bonding position to another atom in a molecule. The two * in the formulae (E₁), (F1), (G1), (H1) and (I1) correspond to two * in the formulae (E₂), (F2), (G2), (H2) and (I2), respectively.

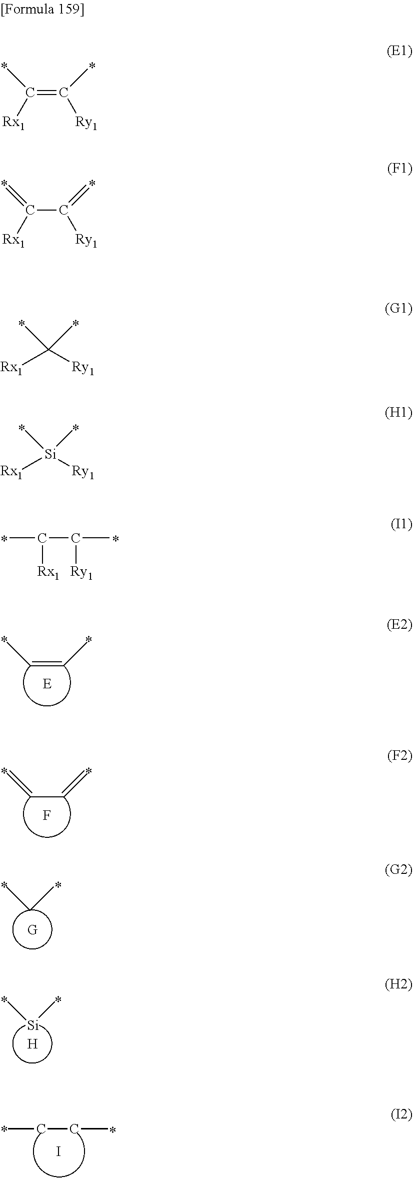

In the molecular structures represented by the formulae (E₂) to (I2), E to I each represent a cyclic structure (the ring having 5 or more ring atoms). In the formulae (E₂) to (I2), * each independently represent a bonding position to another atom in a molecule. The two * in the formula (E₂) correspond to two * in the formula (E₁). Similarly, two * in each of the formulae (F2) to (I2) correspond one-to-one to two * in in each of the formulae (F1) to (11).

For instance, in the formula (E₁), when Rx₁ and Ry₁ are mutually bonded to form the ring E in the formula (E₂) and the ring E is an unsubstituted benzene ring, the molecular structure represented by the formula (E₁) is a molecular structure represented by a formula (E₃) below. Herein, two * in the formula (E₃) correspond one-to-one to two * in each of the formulae (E₂) and (E₁).

For instance, in the formula (E₁), when Rx₁ and Ry₁ are mutually bonded to form the ring E in the formula (E₂) and the ring E is an unsubstituted pyrrole ring, the molecular structure represented by the formula (E₁) is a molecular structure represented by a formula (E₄) below. Herein, two * in the formula (E₄) correspond one-to-one to two * in each of the formulae (E₂) and (E₁). In the formulae (E₃) and (E₄), * each independently represent a bonding position to another atom in a molecule.

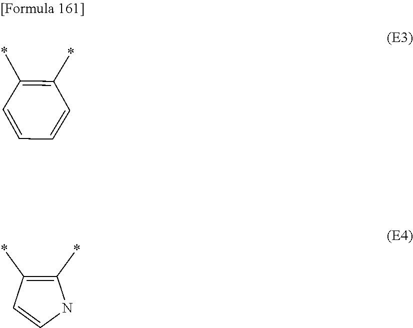 [SMILES: *c1ccccc1* ; *c1cc[nH]c1*]

Herein, the ring carbon atoms refer to the number of carbon atoms among atoms forming a ring of a compound (e.g., a monocyclic compound, fused-ring compound, crosslinking compound, carbon ring compound, and heterocyclic compound) in which the atoms are bonded to each other to form the ring. When the ring is substituted by a substituent(s), carbon atom(s) contained in the substituent(s) is not counted in the ring carbon atoms. Unless specifically described, the same applies to the “ring carbon atoms” described later. For instance, a benzene ring has 6 ring carbon atoms, a naphthalene ring has 10 ring carbon atoms, a pyridinyl group has 5 ring carbon atoms, and a furanyl group has 4 ring carbon atoms. When a benzene ring and/or a naphthalene ring is substituted by a substituent (e.g., an alkyl group), the number of carbon atoms of the alkyl group is not counted in the number of the ring carbon atoms. When a fluorene ring is substituted by a substituent (e.g., a fluorene ring) (i.e., a spirofluorene ring is included), the number of carbon atoms of the fluorene ring as the substituent is not counted in the number of the ring carbon atoms of the fluorene ring.

Herein, the ring atoms refer to the number of atoms forming a ring of a compound (e.g., a monocyclic compound, fused-ring compound, crosslinking compound, carbon ring compound, and heterocyclic compound) in which the atoms are bonded to each other to form the ring (e.g., monocyclic ring, fused ring, ring assembly). Atom(s) not forming a ring and atom(s) included in a substituent when the ring is substituted by the substituent are not counted in the number of the ring atoms. Unless specifically described, the same applies to the “ring atoms” described later. For instance, a pyridine ring has six ring atoms, a quinazoline ring has ten ring atoms, and a furan ring has five ring atoms. A hydrogen atom(s) and/or an atom(s) of a substituent which are bonded to carbon atoms of a pyridine ring and/or quinazoline ring are not counted in the ring atoms. When a fluorene ring is substituted by a substituent (e.g., a fluorene ring) (i.e., a spirofluorene ring is included), the number of atoms of the fluorene ring as the substituent is not counted in the number of the ring atoms of the fluorene ring.

### Description of Each Substituent in Formulae Herein

The aryl group (occasionally referred to as an aromatic hydrocarbon group) herein is exemplified by an aryl group Sub₁. The aryl group Sub₁ preferably has 6 to 40 ring carbon atoms, more preferably 6 to 30 ring carbon atoms, further preferably 6 to 20 ring carbon atoms, still further preferably 6 to 14 ring carbon atoms, still further more preferably 6 to 12 ring carbon atoms.

The aryl group Sub₁ herein is at least one group selected from the group consisting of a phenyl group, biphenyl group, terphenyl group, naphthyl group, anthryl group, phenanthryl group, fluorenyl group, pyrenyl group, chrysenyl group, fluoranthenyl group, benz[a]anthryl group, benzo[c]phenanthryl group, triphenylenyl group, benzo[k]fluoranthenyl group, benzo[g]chrysenyl group, benzo[b]triphenylenyl group, picenyl group, and perylenyl group.

Among the aryl group Sub₁, a phenyl group, biphenyl group, naphthyl group, phenanthryl group, terphenyl group and fluorenyl group are preferable. A carbon atom in a position 9 of each of 1-fluorenyl group, 2-fluorenyl group, 3-fluorenyl group and 4-fluorenyl group is preferably substituted by a substituted or unsubstituted alkyl group Sub₃ or a substituted or unsubstituted aryl group Sub₁ described later herein.

The heteroaryl group (occasionally referred to as a heterocyclic group, heteroaromatic ring group or aromatic heterocyclic group) herein is exemplified by a heterocyclic group Sub₂. The heterocyclic group Sub₂ is a group containing, as a hetero atom(s), at least one atom selected from the group consisting of nitrogen, sulfur, oxygen, silicon, selenium atom and germanium atom. The heterocyclic group Sub₂ preferably contains, as a hetero atom(s), at least one atom selected from the group consisting of nitrogen, sulfur and oxygen. The heterocyclic group Sub₂ preferably has 5 to 30 ring atoms, more preferably 5 to 20 ring atoms, further preferably 5 to 14 ring atoms.

The heterocyclic group Sub₂ herein are, for instance, at least one group selected from the group consisting of a pyridyl group, pyrimidinyl group, pyrazinyl group, pyridazinyl group, triazinyl group, quinolyl group, isoquinolinyl group, naphthyridinyl group, phthalazinyl group, quinoxalinyl group, quinazolinyl group, phenanthridinyl group, acridinyl group, phenanthrolinyl group, pyrrolyl group, imidazolyl group, pyrazolyl group, triazolyl group, tetrazolyl group, indolyl group, benzimidazolyl group, indazolyl group, imidazopyridinyl group, benzotriazolyl group, carbazolyl group, furyl group, thienyl group, oxazolyl group, thiazolyl group, isoxazolyl group, isothiazolyl group, oxadiazolyl group, thiadiazolyl group, benzofuranyl group, benzothienyl group, benzoxazolyl group, benzothiazolyl group, benzisoxazolyl group, benzisothiazolyl group, benzoxadiazolyl group, benzothiadiazolyl group, dibenzofuranyl group, dibenzothienyl group, piperidinyl group, pyrrolidinyl group, piperazinyl group, morpholyl group, phenazinyl group, phenothiazinyl group, and phenoxazinyl group.

Among the above heterocyclic group Sub₂, a 1-dibenzofuranyl group, 2-dibenzofuranyl group, 3-dibenzofuranyl group, 4-dibenzofuranyl group, 1-dibenzothienyl group, 2-dibenzothienyl group, 3-dibenzothienyl group, 4-dibenzothienyl group, 1-carbazolyl group, 2-carbazolyl group, 3-carbazolyl group, 4-carbazolyl group, and 9-carbazolyl group are further more preferable. A nitrogen atom in position 9 of 1-carbazolyl group, 2-carbazolyl group, 3-carbazolyl group and 4-carbazolyl group is preferably substituted by the substituted or unsubstituted aryl group Sub₁ or the substituted or unsubstituted heterocyclic group Sub₂ described herein.

Herein, the heterocyclic group Sub₂ may be a group derived from any one of partial structures represented by formulae (XY-1) to (XY-18) below.

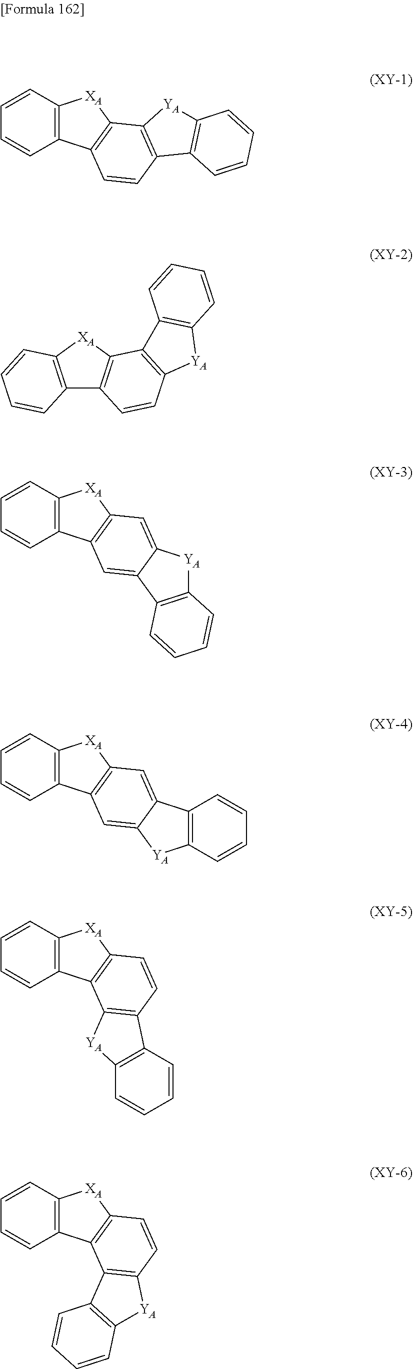

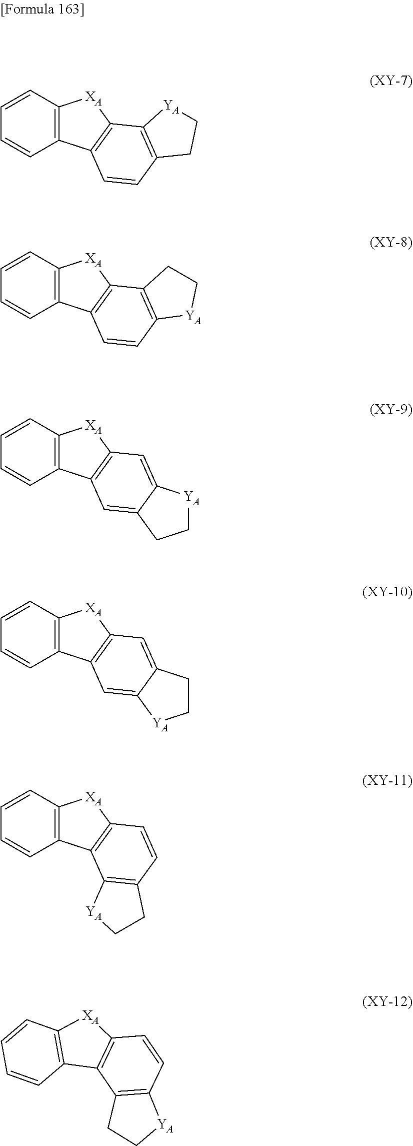

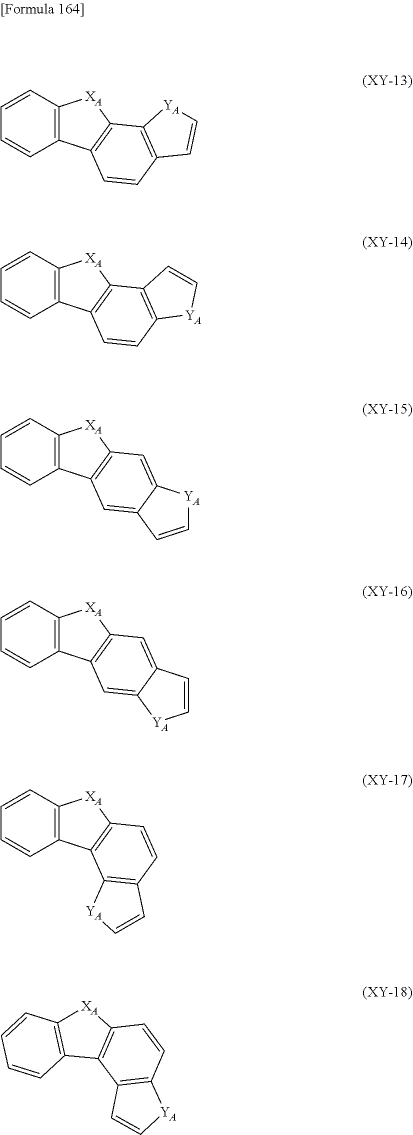

In the formulae (XY-1) to (XY-18), XA and YA each independently represent a hetero atom, and preferably represent an oxygen atom, sulfur atom, selenium atom, silicon atom or germanium atom. Each of the partial structures represented by the respective formulae (XY-1) to (XY-18) has a bond at any position to provide a heterocyclic group. The heterocyclic group may be substituted.

Herein, the heterocyclic group Sub₂ may be a group represented by one of formulae (XY-19) to (XY-22) below. Moreover, the position of the bond may be changed as needed.

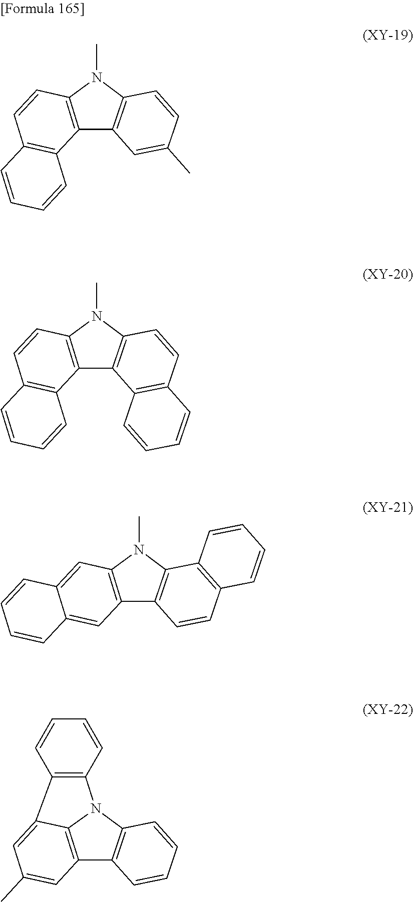

The alkyl group herein may be any one of a linear alkyl group, branched alkyl group and cyclic alkyl group.

The alkyl group herein is exemplified by an alkyl group Sub₃.

The linear alkyl group herein is exemplified by a linear alkyl group Sub₃₁.

The branched alkyl group herein is exemplified by a branched alkyl group Sub₃₂.

The cyclic alkyl group herein is exemplified by a cyclic alkyl group Sub₃₃ (also referred to as a cycloalkyl group Sub₃₃).

For instance, the alkyl group Sub₃ is at least one group selected from the group consisting of the linear alkyl group Sub₃₁, branched alkyl group Sub₃₂, and cyclic alkyl group Sub₃₃.

Herein, the linear alkyl group Sub₃₁ or branched alkyl group Sub₃₂ preferably has 1 to 30 carbon atoms, more preferably 1 to 20 carbon atoms, further preferably 1 to 10 carbon atoms, further more preferably 1 to 6 carbon atoms.

Herein, the cycloalkyl group Sub₃₃ preferably has 3 to 30 ring carbon atoms, more preferably 3 to 20 ring carbon atoms, further preferably 3 to 10 ring carbon atoms, still further preferably 5 to 8 ring carbon atoms.

The linear alkyl group Sub₃₁ or branched alkyl group Sub₃₂ herein is exemplified by at least one group selected from the group consisting of a methyl group, ethyl group, n-propyl group, isopropyl group, n-butyl group, s-butyl group, isobutyl group, t-butyl group, n-pentyl group, n-hexyl group, n-heptyl group, n-octyl group, n-nonyl group, n-decyl group, n-undecyl group, n-dodecyl group, n-tridecyl group, n-tetradecyl group, n-pentadecyl group, n-hexadecyl group, n-heptadecyl group, n-octadecyl group, neopentyl group, amyl group, isoamyl group, 1-methylpentyl group, 2-methylpentyl group, 1-pentylhexyl group, 1-butylpentyl group, 1-heptyloctyl group, and 3-methylpentyl group.

The linear alkyl group Sub₃₁ or branched alkyl group Sub₃₂ is further more preferably a methyl group, ethyl group, propyl group, isopropyl group, n-butyl group, s-butyl group, isobutyl group, t-butyl group, n-pentyl group, n-hexyl group, amyl group, isoamyl group and neopentyl group.

The cycloalkyl group Sub₃₃ herein is exemplified by at least one group selected from the group consisting of a cyclopropyl group, cyclobutyl group, cyclopentyl group, cyclohexyl group, 4-metylcyclohexyl group, adamantyl group and norbornyl group. Among the cycloalkyl group Sub₃₃, a cyclopentyl group and a cyclohexyl group are still further preferable.

Herein, an alkyl halide group is exemplified by an alkyl halide group Sub₄. The alkyl halide group Sub₄ is provided by substituting the alkyl group Sub₃ with at least one halogen atom, preferably at least one fluorine atom.

Herein, the alkyl halide group Sub₄ is exemplified by at least one group selected from the group consisting of a fluoromethyl group, difluoromethyl group, trifluoromethyl group, fluoroethyl group, trifluoromethylmethyl group, trifluoroethyl group, and pentafluoroethyl group.

Herein, a substituted silyl group is exemplified by a substituted silyl group Sub₅. The substituted silyl group Sub₅ is exemplified by at least one group selected from the group consisting of an alkylsilyl group Sub₅₁ and an arylsilyl group Sub₅₂.

Herein, the alkylsilyl group Sub₅₁ is exemplified by a trialkylsilyl group Sub₅₁₁ having the above-described alkyl group Suba.

The trialkylsilyl group Sub₅₁₁ is exemplified by at least one group selected from the group consisting of a trimethylsilyl group, triethylsilyl group, tri-n-butylsilyl group, tri-n-octylsilyl group, triisobutylsilyl group, dimethylethylsilyl group, dimethylisopropylsilyl group, dimethyl-n-propylsilyl group, dimethyl-n-butylsilyl group, dimethyl-t-butylsilyl group, diethylisopropylsilyl group, vinyl dimethylsilyl group, propyldimethylsilyl group, and triisopropylsilyl group. Three alkyl groups Sub₃ in the trialkylsilyl group Sub₅₁₁ may be mutually the same or different.

Herein, the arylsilyl group Sub₅₂ is exemplified by at least one group selected from the group consisting of a dialkylarylsilyl group Sub₅₂₁, alkyldiarylsilyl group Sub₅₂₂ and triarylsilyl group Sub₅₂₃.

The dialkylarylsilyl group Sub₅₂₁ is exemplified by a dialkylarylsilyl group including two alkyl groups Sub₃ and one aryl group Sub₁. The dialkylarylsilyl group Sub₅₂₁ preferably has 8 to 30 carbon atoms.

The alkyldiarylsilyl group Sub₅₂₂ is exemplified by an alkyldiarylsilyl group including one alkyl group Sub₃ and two aryl groups Sub₁. The alkyldiarylsilyl group Sub₅₂₂ preferably has 13 to 30 carbon atoms.

The triarylsilyl group Sub₅₂₃ is exemplified by a triarylsilyl group including three aryl groups Sub₁. The triarylsilyl group Sub₅₂₃ preferably has 18 to 30 carbon atoms.

Herein, a substituted or unsubstituted alkyl sulfonyl group is exemplified by an alkyl sulfonyl group Sub₆. The alkyl sulfonyl group Sub₆ is represented by —SO₂Rw. R_{w} in —SO₂R_{w} represents a substituted or unsubstituted alkyl group Sub₃ described above.

Herein, an aralkyl group (occasionally referred to as an arylalkyl group) is exemplified by an aralkyl group Sub₇. An aryl group in the aralkyl group Sub₇ includes, for instance, at least one of the above-described aryl group Sub₁ or the above-described heteroaryl group Sub₂.

The aralkyl group Sub₇ herein is preferably a group having the aryl group Sub₁ and is represented by —Z₃—Z₄. Z₃ is exemplified by an alkylene group corresponding to the above alkyl group Sub₃. Z₄ is exemplified by the above aryl group Sub₁.

In this aralkyl group Sub₇, an aryl moiety has 6 to 30 carbon atoms (preferably 6 to 20 carbon atoms, more preferably 6 to 12 carbon atoms) and an alkyl moiety has 1 to 30 carbon atoms (preferably 1 to 20 carbon atoms, more preferably 1 to 10 carbon atoms, further preferably 1 to 6 carbon atoms). The aralkyl group Sub₇ is exemplified by at least one group selected from the group consisting of a benzyl group, 2-phenylpropane-2-yl group, 1-phenylethyl group, 2-phenylethyl group, 1-phenylisopropyl group, 2-phenylisopropyl group, phenyl-t-butyl group, α-naphthylmethyl group, 1-α-naphthylethyl group, 2-α-naphthylethyl group, 1-α-naphthylisopropyl group, 2-α-naphthylisopropyl group, β-naphthylmethyl group, 1-β-naphthylethyl group, 2-β-naphthylethyl group, 1-β-naphthylisopropyl group, and 2-β-naphthylisopropyl group.

The alkoxy group herein is exemplified by an alkoxy group Sub₈. The alkoxy group Sub₈ is represented by −OZ₁. Z₁ is exemplified by the above alkyl group Sub₃. The alkoxy group Sub₈ preferably has 1 to 30 carbon atoms, more preferably 1 to 20 carbon atoms. The alkoxy group Sub₈ is exemplified by at least one group selected from the group consisting of a methoxy group, ethoxy group, propoxy group, butoxy group, pentyloxy group and hexyloxy group.

Herein, an alkoxy halide group is exemplified by an alkoxy halide group Sub₉. The alkoxy halide group Sub₉ is provided by substituting the alkoxy group Sub₈ with at least one halogen atom, preferably at least one fluorine atom.

Herein, an aryloxy group (occasionally referred to as an arylalkoxy group) is exemplified by an arylalkoxy group Sub₁₀. An aryl group in the arylalkoxy group Sub₁₀ includes at least one of the aryl group Sub₁ or the heteroaryl group Sub₂.

The arylalkoxy group Sub₁₀ herein is represented by —OZ₂. Z₂ is exemplified by the aryl group Sub₁ or the heteroaryl group Sub₂. The arylalkoxy group Sub₁₀ preferably has 6 to 30 ring carbon atoms, more preferably 6 to 20 ring carbon atoms. The arylalkoxy group Sub₁₀ is exemplified by a phenoxy group.

Herein, a substituted amino group is exemplified by a substituted amino group Sub₁₁. The substituted amino group Sub₁₁ is exemplified by at least one group selected from the group consisting of an arylamino group Sub₁₁₁ and an alkylamino group Sub₁₁₂.

The arylamino group Sub₁₁₁ is represented by —NHR_{V1} or —N(R_{V1})₂. R_{V1} is exemplified by the aryl group Sub₁. Two R_{V1} in —N(R_{V1})₂ are mutually the same or different.

The alkylamino group Sub₁₁₂ is represented by —NHR_{V2} or —N(R_{V2})₂. R_{V2} is exemplified by the alkyl group Sub₃. Two R_{V2} in —N(R_{V2})₂ are mutually the same or different.

Herein, the alkenyl group is exemplified by an alkenyl group Sub₁₂. The alkenyl group Sub₁₂, which is linear or branched, is exemplified by at least one group selected from the group consisting of a vinyl group, propenyl group, butenyl group, oleyl group, eicosapentaenyl group, docosahexaenyl group, styryl group, 2,2-diphenylvinyl group, 1,2,2-triphenylvinyl group, and 2-phenyl-2-propenyl group.

The alkynyl group herein is exemplified by an alkynyl group Sub₁₃. The alkynyl group Sub₁₃ may be linear or branched, and is exemplified by at least one group selected from the group consisting of an ethynyl group, a propynyl group and a 2-phenylethynyl group.

The alkylthio group herein is exemplified by an alkylthio group Sub₁₄.

The alkylthio group Sub₁₄ is represented by —SR_{V3}. R_{V3} is exemplified by the alkyl group Sub₃. The alkylthio group Sub₁₄ preferably has 1 to 30 carbon atoms, more preferably 1 to 20 carbon atoms.

The arylthio group herein is exemplified by an arylthio group Sub₁₅.

The arylthio group Sub₁₅ is represented by —SR_{V4}. R_{V4} is exemplified by the aryl group Sub₁. The arylthio group Sub₁₅ preferably has 6 to 30 ring carbon atoms, more preferably 6 to 20 ring carbon atoms.

Herein, examples of the halogen atom include a fluorine atom, a chlorine atom, a bromine atom and an iodine atom, among which a fluorine atom is preferable.

A substituted phosphino group herein is exemplified by a substituted phosphino group Sub₁₆. The substituted phosphino group Sub₁₆ is exemplified by a phenyl phosphanyl group.

An arylcarbonyl group herein is exemplified by an arylcarbonyl group Sub₁₇. The arylcarbonyl group Sub₁₇ is represented by —COY′. Y′ is exemplified by the aryl group Sub₁. Herein, the arylcarbonyl group Sub₁₇ is exemplified by at least one group selected from the group consisting of a phenyl carbonyl group, diphenyl carbonyl group, naphthyl carbonyl group, and triphenyl carbonyl group.

An acyl group herein is exemplified by an acyl group Sub₁₈. The acyl group Sub₁₈ is represented by —COR′. R′ is exemplified by the alkyl group Sub₃. The acyl group Sub₁₈ herein is exemplified by at least one group selected from the group consisting of an acetyl group and a propionyl group.

A substituted phosphoryl group herein is exemplified by a substituted phosphoryl group Sub₁₉. The substituted phosphoryl group Sub₁₉ is represented by a formula (P) below.

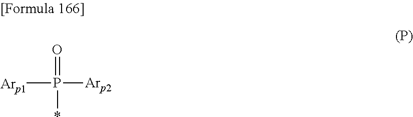

In the formula (P), Ar_{P1} and Ar_{P2} are any one substituent selected from the group consisting of the above alkyl group Sub₃ and the above aryl group Sub₁.

An ester group herein is exemplified by an ester group Sub₂₀. The ester group Sub₂₀ is exemplified by at least one group selected from the group consisting of an alkyl ester group and an aryl ester group.

An alkyl ester group herein is exemplified by an alkyl ester group Sub₂₀₁. The alkyl ester group Sub₂₀₁ is represented by —C(═O)OR^{E}. R^{E} is exemplified by a substituted or unsubstituted alkyl group Sub₃ described above.

An aryl ester group herein is exemplified by an aryl ester group Sub₂₀₂. The aryl ester group Sub₂₀₂ is represented by —C(═O)OR^{Ar}. R^{Ar} is exemplified by a substituted or unsubstituted aryl group Sub₁ described above.

A siloxanyl group herein is exemplified by a siloxanyl group Sub₂₁. The siloxanyl group Sub₂₁ is a silicon compound group through an ether bond. The siloxanyl group Sub₂₁ is exemplified by a trimethylsiloxanyl group.

A carbamoyl group herein is represented by —CONH₂.

A substituted carbamoyl group herein is exemplified by a carbamoyl group Sub₂₂. The carbamoyl group Sub₂₂ is represented by —CONH—Ar^{C} or —CONH—R^{C}. Ar^{C} is exemplified by at least one group selected from the group consisting of the above-described aryl group Sub₁ (preferably 6 to 10 ring carbon atoms) and the above-described heteroaryl group Sub₂ (preferably 5 to 14 ring atoms). Ar^{C} may be a group formed by bonding the aryl group Sub₁ and the heteroaryl group Sub₂.

R^{C} is exemplified by a substituted or unsubstituted alkyl group Sub₃ described above (preferably having 1 to 6 carbon atoms).

Herein, “carbon atoms forming a ring (ring carbon atoms)” mean carbon atoms forming a saturated ring, unsaturated ring, or aromatic ring. “Atoms forming a ring (ring atoms)” mean carbon atoms and hetero atoms forming a hetero ring including a saturated ring, unsaturated ring, or aromatic ring.

Herein, a hydrogen atom includes isotope having different numbers of neutrons, specifically, protium, deuterium and tritium.

In chemical formulae herein, it is assumed that a hydrogen atom (i.e. protium, deuterium and tritium) is bonded to each of bondable positions that are not annexed with signs “R” or the like or “D” representing a deuterium.

Hereinafter, an alkyl group Sub₃ means at least one group of a linear alkyl group Sub₃₁, a branched alkyl group Sub₃₂, or a cyclic alkyl group Sub₃₃ described in “Description of Each Substituent.”

Similarly, a substituted silyl group Sub₅ means at least one group of an alkylsilyl group Sub₅₁ or an arylsilyl group Sub₅₂.

Similarly, a substituted amino group Sub₁₁ means at least one group of an arylamino group Sub₁₁₁ or an alkylamino group Sub₁₁₂.

Herein, a substituent for a “substituted or unsubstituted” group is exemplified by a substituent R_{F1}. The substituent R_{F1} is at least one group selected from the group consisting of an aryl group Sub₁, heteroaryl group Sub₂, alkyl group Sub₃, alkyl halide group Sub₄, substituted silyl group Sub₅, alkylsulfonyl group Sub₆, aralkyl group Sub₇, alkoxy group Sub₈, alkoxy halide group Sub₉, arylalkoxy group Sub₁₀, substituted amino group Sub₁₁, alkenyl group Sub₁₂, alkynyl group Sub₁₃, alkylthio group Sub₁₄, arylthio group Sub₁₅, substituted phosphino group Sub₁₆, arylcarbonyl group Sub₁₇, acyl group Sub₁₈, substituted phosphoryl group Sub₁₉, ester group Sub₂₀, siloxanyl group Sub₂₁, carbamoyl group Sub₂₂, unsubstituted amino group, unsubstituted silyl group, halogen atom, cyano group, hydroxy group, nitro group, and carboxy group.

Herein, the substituent R_{F1} for a “substituted or unsubstituted” group may be a diaryl boron group (Ar_{B1}Ar_{B2}B—). Ar_{B1} and Ar_{B2} are exemplified by the above-described aryl group Sub₁. Ar_{B1} and Ar_{B2} in Ar_{B1}Ar_{B2}B— are the same or different.

Specific examples and preferable examples of the substituent R_{F1} are the same as those of the substituents described in “Description of Each Substituent” (e.g., an aryl group Sub₁, heteroaryl group Sub₂, alkyl group Sub₃, alkyl halide group Sub₄, substituted silyl group Sub₅, alkylsulfonyl group Sub₆, aralkyl group Sub₇, alkoxy group Sub₈, alkoxy halide group Sub₉, arylalkoxy group Sub₁₀, substituted amino group Sub₁₁, alkenyl group Sub₁₂, alkynyl group Sub₁₃, alkylthio group Sub₁₄, arylthio group Sub₁₅, substituted phosphino group Sub₁₆, arylcarbonyl group Sub₁₇, acyl group Sub₁₈, substituted phosphoryl group Sub₁₉, ester group Sub₂₀, siloxanyl group Sub₂₁, and carbamoyl group Sub₂₂).

The substituent R_{F1} for a “substituted or unsubstituted” group may be further substituted by at least one group (hereinafter, also referred to as a substituent R_{F2}) selected from the group consisting of an aryl group Sub₁, heteroaryl group Sub₂, alkyl group Sub₃, alkyl halide group Sub₄, substituted silyl group Sub₅, alkylsulfonyl group Sub₆, aralkyl group Sub₇, alkoxy group Sub₈, alkoxy halide group Sub₉, arylalkoxy group Sub₁₀, substituted amino group Sub₁₁, alkenyl group Sub₁₂, alkynyl group Sub₁₃, alkylthio group Sub₁₄, arylthio group Sub₁₅, substituted phosphino group Sub₁₆, arylcarbonyl group Sub₁₇, acyl group Sub₁₈, substituted phosphoryl group Sub₁₉, ester group Sub₂₀, siloxanyl group Sub₂₁, carbamoyl group Sub₂₂, unsubstituted amino group, unsubstituted silyl group, halogen atom, cyano group, hydroxy group, nitro group, and carboxy group. Moreover, a plurality of substituents R_{F2} may be bonded to each other to form a ring.

“Unsubstituted” for a “substituted or unsubstituted” group means that a group is not substituted by the above-described substituent R_{F1} but bonded with a hydrogen atom.

Herein, “XX to YY carbon atoms” in the description of “substituted or unsubstituted ZZ group having XX to YY carbon atoms” represent carbon atoms of an unsubstituted ZZ group and do not include carbon atoms of the substituent R_{F1} of the substituted ZZ group.

Herein, “XX to YY atoms” in the description of “substituted or unsubstituted ZZ group having XX to YY atoms” represent atoms of an unsubstituted ZZ group and do not include atoms of the substituent R_{F1} of the substituted ZZ group.

The same description as the above applies to “substituted or unsubstituted” in compounds or partial structures thereof described herein.

Herein, when the substituents are bonded to each other to form a ring, the ring is structured to be a saturated ring, an unsaturated ring, an aromatic hydrocarbon ring or a hetero ring.

Herein, examples of the aromatic hydrocarbon group in the linking group include a divalent or multivalent group obtained by eliminating one or more atoms from the above monovalent aryl group Sub₁.

Herein, examples of the heterocyclic group in the linking group include a divalent or multivalent group obtained by eliminating one or more atoms from the above monovalent heteroaryl group Sub₂.

### EXAMPLES

### Compounds

Structures of compounds M3 represented by the formula (3) and used for manufacturing organic EL devices are shown below.

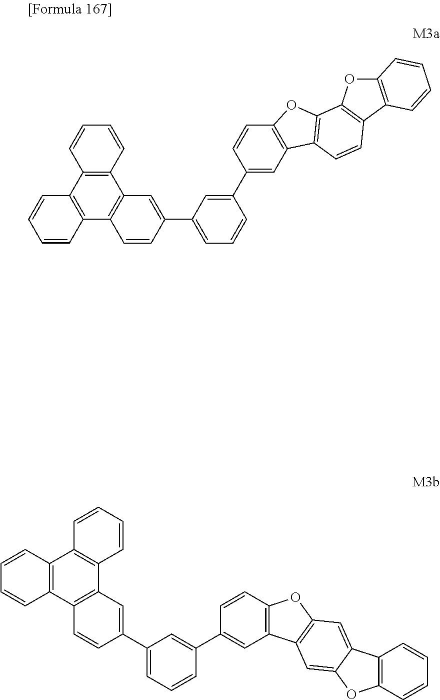 [SMILES: c1cc(-c2ccc3oc4c(ccc5c6ccccc6oc54)c3c2)cc(-c2ccc3c4ccccc4c4ccccc4c3c2)c1 ; c1cc(-c2ccc3oc4cc5c(cc4c3c2)oc2ccccc25)cc(-c2ccc3c4ccccc4c4ccccc4c3c2)c1]

Structures of compounds used for manufacturing organic EL devices in Comparatives are shown below.

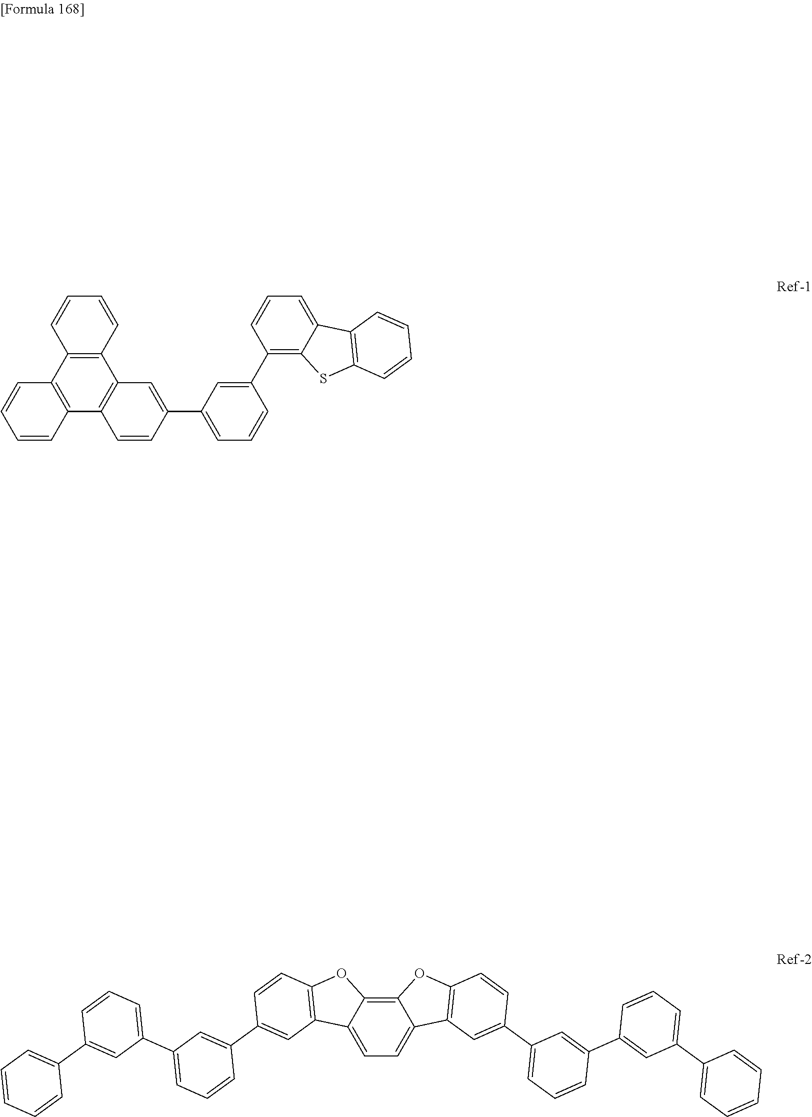 [SMILES: c1cc(-c2ccc3c4ccccc4c4ccccc4c3c2)cc(-c2cccc3c2sc2ccccc23)c1 ; c1ccc(-c2cccc(-c3cccc(-c4ccc5oc6c(ccc7c8cc(-c9cccc(-c%10cccc(-c%11ccccc%11)c%10)c9)ccc8oc76)c5c4)c3)c2)cc1]

Structures of other compounds used for manufacturing the organic EL devices in Examples and Comparatives are shown below.

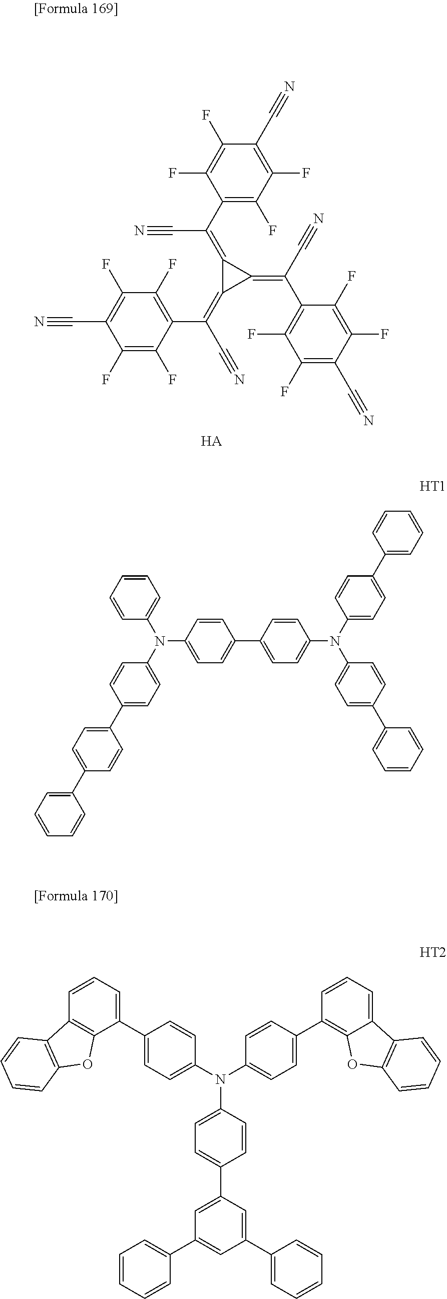 [SMILES: N#CC(=C1C(=C(C#N)c2c(F)c(F)c(C#N)c(F)c2F)C1=C(C#N)c1c(F)c(F)c(C#N)c(F)c1F)c1c(F)c(F)c(C#N)c(F)c1F ; "HA" ; c1ccc(-c2ccc(-c3ccc(N(c4ccccc4)c4ccc(-c5ccc(N(c6ccc(-c7ccccc7)cc6)c6ccc(-c7ccccc7)cc6)cc5)cc4)cc3)cc2)cc1 ; c1ccc(-c2cc(-c3ccccc3)cc(-c3ccc(N(c4ccc(-c5cccc6c5oc5ccccc56)cc4)c4ccc(-c5cccc6c5oc5ccccc56)cc4)cc3)c2)cc1]

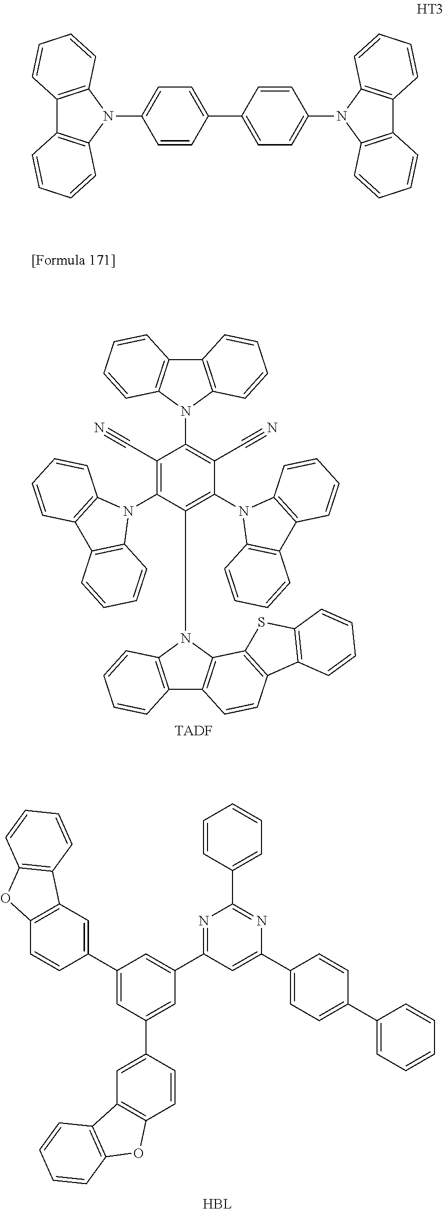

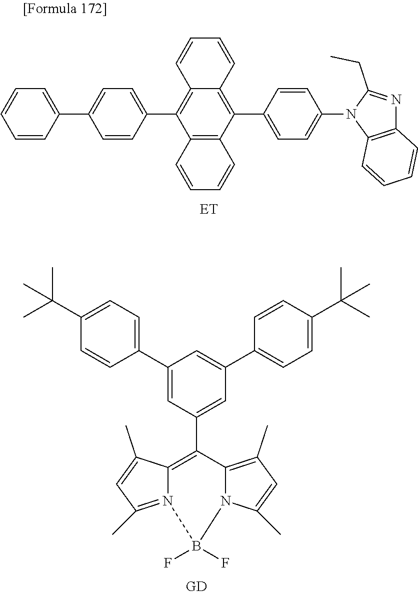

### Manufacture 1 of Organic EL Device

The organic EL devices were manufactured and evaluated as follows.

### Example 1

A glass substrate (size: 25 mm×75 mm×1.1 mm thick, manufactured by Geomatec Co., Ltd.) having an ITO transparent electrode (anode) was ultrasonic-cleaned in isopropyl alcohol for five minutes, and then UV-ozone-cleaned for one minute. The film thickness of ITO was 130 nm

After the glass substrate having the transparent electrode line was cleaned, the glass substrate was mounted on a substrate holder of a vacuum deposition apparatus. Firstly, a compound HT1 and a compound HA were co-deposited on a surface of the glass substrate where the transparent electrode line was provided in a manner to cover the transparent electrode, thereby forming a 10-nm-thick hole injecting layer. The concentrations of the compound HT1 and the compound HA in the hole injecting layer were 97 mass % and 3 mass %, respectively.

Next, the compound HT1 was vapor-deposited on the hole injecting layer to form a 110-nm-thick first hole transporting layer.

Next, a compound HT2 was vapor-deposited on the first hole transporting layer to form a 5-nm-thick second hole transporting layer.

Next, a compound HT3 was vapor-deposited on the second hole transporting layer to form a 5-nm-thick electron blocking layer.

Next, a compound M3a as the compound M3 and a compound TADF as the compound M2 were co-deposited on the electron blocking layer to form a 25-nm-thick emitting layer. The concentrations of the compound M3a and the compound TADF in the emitting layer were 50 mass % and 50 mass %, respectively.

Next, a compound HBL was vapor-deposited on the emitting layer to form a 5-nm-thick hole blocking layer.

Next, a compound ET was vapor-deposited on the hole blocking layer to form a 30-nm-thick electron transporting layer.

Next, lithium fluoride (LiF) was vapor-deposited on the electron transporting layer to form a 1-nm-thick electron injecting electrode (cathode).

Subsequently, metal aluminum (Al) was vapor-deposited on the electron injecting electrode to form an 80-nm-thick metal Al cathode.

A device arrangement of the organic EL device of Example 1 is roughly shown as follows.

ITO (130)/HT1:HA (10, 97%:3%)/HT1 (110)/HT2 (5)/HT3 (5)/M3a:TADF (25, 50%:50%)/HBL (5)/ET (30)/LiF (1)/A₁ (80)

The numerals in parentheses represent film thickness (unit: nm).

The numerals (97%:3%) represented by percentage in the same parentheses indicate a ratio (mass %) between the compound HT1 and the compound HA in the hole injecting layer. The numerals (50%:50%) represented by percentage in the same parentheses indicate a ratio (mass %) between the compound M3a and the compound TADF in the emitting layer.

### Comparative 1

The organic EL device of Comparative 1 was manufactured in the same manner as that of Example 1 except that the compound M3a in the emitting layer of Example 1 was replaced by the compound shown in Table 1.

### Comparative 2

The organic EL device of Comparative 2 was manufactured in the same manner as that of Example 1 except that the compound M3a in the emitting layer of Example 1 was replaced by the compound shown in Table 1.

### Evaluation of Organic EL Device

The organic EL devices manufactured in Example 1 and Comparatives 1 and 2 were evaluated as follows. Table 1 shows the results. Although the compounds Ref-1 and Ref-2 used in Comparatives 1 and 2 do not fall under the formula of the compound M3, the compounds Ref-1 and Ref-2 are shown in the same column as the compound M3a in Example 1 for convenience.

### Main Peak Wavelength (λₚ)

Voltage was applied on the organic EL devices such that a current density of the organic EL device was 10 mA/cm², where spectral radiance spectrum was measured by a spectroradiometer CS-2000 (manufactured by Konica Minolta, Inc.). The main peak wavelength λₚ (unit: nm) was calculated based on the obtained spectral-radiance spectra.

### Lifetime LT95

Voltage was applied on the resultant devices such that a current density was 50 mA/cm², where a time (unit: h) elapsed before a luminance intensity was reduced to 95% of the initial luminance intensity was measured using a spectroradiometer CS-200 (manufactured by Konica Minolta, Inc.).

Hereinafter, the time elapsed before the luminance intensity is reduced to 95% of the initial luminance intensity is referred to as “Lifetime LT95(h).”

TABLE 1

| | Compound M3 | | Compound M2 | | | Device Evaluation Results | |
|---|---|---|---|---|---|---|---|
| | | S₁ | | S₁ | λ | λ p | LT95 |
| | Type | [eV] | Type | [eV] | [nm] | [nm] | [h] |
| Example 1 | M3a | 3.67 | TADF | 2.61 | 506 | 555 | 13 |
| Comparative 1 | Ref-1 | 3.58 | TADF | 2.61 | 506 | 555 | 4 |
| Comparative 2 | Ref-2 | 3.64 | TADF | 2.61 | 506 | 555 | 9 |

The organic EL device of Example 1 had a longer lifetime than the organic EL devices of Comparatives 1 and 2.

### Manufacture 2 of Organic EL Device

The organic EL devices were manufactured and evaluated as follows.

### Example 2

A glass substrate (size: 25 mm×75 mm×1.1 mm thick, manufactured by Geomatec Co., Ltd.) having an ITO transparent electrode (anode) was ultrasonic-cleaned in isopropyl alcohol for five minutes, and then UV-ozone-cleaned for one minute. The film thickness of ITO was 130 nm

After the glass substrate having the transparent electrode line was cleaned, the glass substrate was mounted on a substrate holder of a vacuum deposition apparatus. Firstly, the compound HT1 and the compound HA were co-deposited on a surface of the glass substrate where the transparent electrode line was provided in a manner to cover the transparent electrode, thereby forming a 10-nm-thick hole injecting layer. The concentrations of the compound HT1 and the compound HA in the hole injecting layer were 97 mass % and 3 mass %, respectively.

Next, the compound HT1 was vapor-deposited on the hole injecting layer to form a 110-nm-thick first hole transporting layer.

Next, the compound HT2 was vapor-deposited on the first hole transporting layer to form a 5-nm-thick second hole transporting layer.

Next, the compound HT3 was vapor-deposited on the second hole transporting layer to form a 5-nm-thick electron blocking layer.

Next, the compound M3a as the compound M3, the compound TADF as the compound M2 and a compound GD as the compound M1 were co-deposited on the electron blocking layer to form a 25-nm-thick emitting layer. The concentrations of the compound M3a, the compound TADF and the compound GD as the compound M1 in the emitting layer were 49 mass %, 50 mass % and 1 mass %, respectively.

Next, the compound HBL was vapor-deposited on the emitting layer to form a 5-nm-thick hole blocking layer.

Next, the compound ET was vapor-deposited on the hole blocking layer to form a 30-nm-thick electron transporting layer.

Next, lithium fluoride (LiF) was vapor-deposited on the electron transporting layer to form a 1-nm-thick electron injecting electrode (cathode).

Subsequently, metal aluminum (Al) was vapor-deposited on the electron injecting electrode to form an 80-nm-thick metal Al cathode.

A device arrangement of the organic EL device of Example 2 is roughly shown as follows.

ITO (130)/HT1:HA (10, 97%:3%)/HT1 (110)/HT2 (5)/HT3 (5)/M3a:TADF:GD (25, 49%:50%:1%)/HBL (5)/ET (30)/LiF (1)/Al (80)

The numerals in parentheses represent film thickness (unit: nm).

The numerals (97%:3%) represented by percentage in the same parentheses indicate a ratio (mass %) between the compound HT1 and the compound HA in the hole injecting layer. The numerals (49%:50%:1%) represented by percentage in the same parentheses indicate a ratio (mass %) between the compound M3a, the compound TADF and the compound GD in the emitting layer.

### Example 3 and Comparatives 3 and 4

The organic EL devices of Example 3 and Comparatives 3 and 4 were manufactured in the same manner as that of Example 2 except that the compound M3a in the emitting layer of Example 2 was replaced by the compounds shown in Table 2.

### Evaluation of Organic EL Device

The organic EL devices manufactured in Examples 2 and 3 and Comparatives 3 and 4 were evaluated as follows. Table 2 shows the results. Although the compounds Ref-1 and Ref-2 used in Comparatives 3 and 4 do not fall under the formula of the compound M3, the compounds Ref-1 and Ref-2 are shown in the same column as the compound M3a in Example 2 for convenience.

### Main Peak Wavelength (λₚ)

The main peak wavelength λₚ (unit: nm) was calculated in the same manner as in Example 1.

External Quantum Efficiency EQE

Voltage was applied on the organic EL devices so that a current density was 10 mA/cm², where spectral radiance spectrum was measured by a spectroradiometer CS-2000 (manufactured by Konica Minolta, Inc.). The external quantum efficiency EQE (unit: %) was calculated based on the obtained spectral-radiance spectra, assuming that the spectra was provided under a Lambertian radiation.

Using a numerical formula (Numerical Formula 100) below, EQE (%) of each of Examples was calculated as “EQE (relative value: %)” relative to EQE (%) of Comparative 3 defined as 100.

EQE (relative value: %) of each Example=(EQE (%) of Example/EQE (%) of Comparative 3)×100 (Numerical Formula 100)

TABLE 2

| | | | | | | | | | Device Evaluation Results | |
|---|---|---|---|---|---|---|---|---|---|---|
| | Compound M3 | | Compound M2 | | | Compound M1 | | | | EQE (Relative |
| | | S₁ | | S₁ | λ | | S₁ | λ | λ p | Value: %) |
| | Type | [eV] | Type | [eV] | [nm] | Type | [eV] | [nm] | [nm] | @ 10 mA/cm² |
| Example 2 | M3a | 3.67 | TADF | 2.61 | 506 | GD | 2.39 | 516 | 522 | 115 |
| Example 3 | M3b | 3.57 | TADF | 2.61 | 506 | GD | 2.39 | 516 | 522 | 112 |
| Comparative 3 | Ref-1 | 3.58 | TADF | 2.61 | 506 | GD | 2.39 | 516 | 522 | 100 |
| Comparative 4 | Ref-2 | 3.64 | TADF | 2.61 | 506 | GD | 2.39 | 516 | 522 | 85 |

The organic EL devices of Examples 2 and 3 had a higher EQE than the organic EL device of Comparatives 3 and 4.

### Evaluation of Compounds

Values of physical properties of the compounds shown in Tables 1 and 2 were measured by the following method.

### Thermally Activated Delayed Fluorescence

### Delayed Fluorescence of Compound TADF

Thermally activated delayed fluorescence was checked by measuring transient PL using a device shown in FIG. 2. The compound TADF was dissolved in toluene to prepare a dilute solution with an absorbance of 0.05 or less at the excitation wavelength to eliminate the contribution of self-absorption. In order to prevent quenching due to oxygen, the sample solution was frozen and degassed and then sealed in a cell with a lid under an argon atmosphere to obtain an oxygen-free sample solution saturated with argon.

The fluorescence spectrum of the above sample solution was measured with a spectrofluorometer FP-8600 (manufactured by JASCO Corporation), and the fluorescence spectrum of a 9,10-diphenylanthracene ethanol solution was measured under the same conditions. Using the fluorescence area intensities of both spectra, the total fluorescence quantum yield was calculated by an equation (1) in Morris et al. J. Phys. Chem. 80 (1976) 969.

Prompt emission was observed immediately when the excited state was achieved by exciting the compound TADF with a pulse beam (i.e., a beam emitted from a pulse laser) having a wavelength to be absorbed by the compound TADF, and Delay emission was observed not immediately when the excited state was achieved but after the excited state was achieved. The delayed fluorescence in Examples means that an amount of Delay Emission is 5% or more with respect to an amount of Prompt Emission. Specifically, provided that the amount of Prompt emission is denoted by X_{P} and the amount of Delay emission is denoted by X_{D}, the delayed fluorescence means that a value of X_{D}/X_{P} is 0.05 or more.

An amount of Prompt emission, an amount of Delay emission and a ratio between the amounts thereof can be obtained according to the method as described in “Nature 492, 234-238, 2012” (Reference Document 1). The amount of Prompt emission and the amount of Delay emission may be calculated using a device different from one described in Reference Document 1 or one shown in FIG. 2.

It was confirmed that the amount of Delay Emission was 5% or more with respect to the amount of Prompt Emission in the compound TADF.

Specifically, the value of X_{D}/X_{P} was 0.05 or more in the compound TADF.

### Singlet Energy S₁

A singlet energy S₁ of each of the compounds M3a, the compound M3b, the compound TADF, the compound GD, and the comparative compounds Ref-1 and Ref-2 was measured according to the above-described solution method. Measurement results are shown in Tables 1 and 2.

### ΔST and Energy Gap at 77K

An energy gap T_{77K} of each of the compound TADF, the compound M3a and the compound M3b at 77K was measured according to the measurement method of the energy gap T_{77K} described in the above “Relationship between Triplet Energy and Energy Gap at 77K.”

It was confirmed from the measurement result of T_{77K} of the compound TADF that ΔST of the compound TADF was less than 0.01 eV.

T_{77K} of the compound M3a was 2.74 eV. Thus, ΔST of the compound M3a was 0.93 eV.

T_{77K} of the compound M3b was 2.69 eV. Thus, ΔST of the compound M3b was 0.88 eV.

### Main Peak Wavelength λ of Compounds

A main peak wavelength λ of each of the compounds TADF and GD was measured according to the following method.

A toluene solution of a measurement target compound at a concentration of 5 μmol/L was prepared and put in a quartz cell. An emission spectrum (ordinate axis: luminous intensity, abscissa axis: wavelength) of the thus-obtained sample was measured at a normal temperature (300K). In Examples, the emission spectrum was measured using a spectrophotometer manufactured by Hitachi, Ltd. (device name: F-7000). It should be noted that the machine for measuring the emission spectrum is not limited to the machine used herein. A peak wavelength of the emission spectrum exhibiting the maximum luminous intensity was defined as a main peak wavelength λ.

### Synthesis of Compound

Compounds M3a, M3b and M3c represented by the formula (3) were synthesized.

### Synthesis Example 1

### (1) Synthesis Example 1: Synthesis of Compound M3a

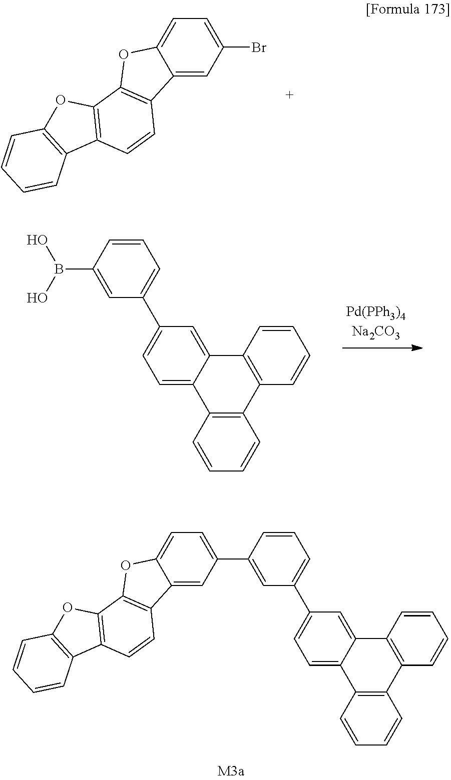 [SMILES: Brc1ccc2oc3c(ccc4c5ccccc5oc43)c2c1 ; OB(O)c1cccc(-c2ccc3c4ccccc4c4ccccc4c3c2)c1 ; c1cc(-c2ccc3oc4c(ccc5c6ccccc6oc54)c3c2)cc(-c2ccc3c4ccccc4c4ccccc4c3c2)c1]

Under nitrogen atmosphere, 1,2-dimethoxyethane (300 mL) and water (150 mL) were added into a mixture of 3-bromo-benzo[2,1-b:3,4-b′]bisbenzofuran (16.5 g, 49.0 mmol), (3-(triphenylene-2-yl)phenyl) boronic acid (17.1 g, 49.0 mmol), tetrakistriphenylphosphine palladium (2.83 g, 2.45 mmol) and sodium carbonate (15.6 g, 147 mmol) and stirred at 85 degrees C. for four hours. After the reaction, a solid was filtrated and recrystallized with toluene to obtain the compound M3a (17.5 g, a yield of 64%). The obtained compound was identified as the compound M3a by analysis according to LC-MS (Liquid chromatography mass spectrometry).

### Synthesis Example 2

### (2) Synthesis Example 2: Synthesis of Compound M3b

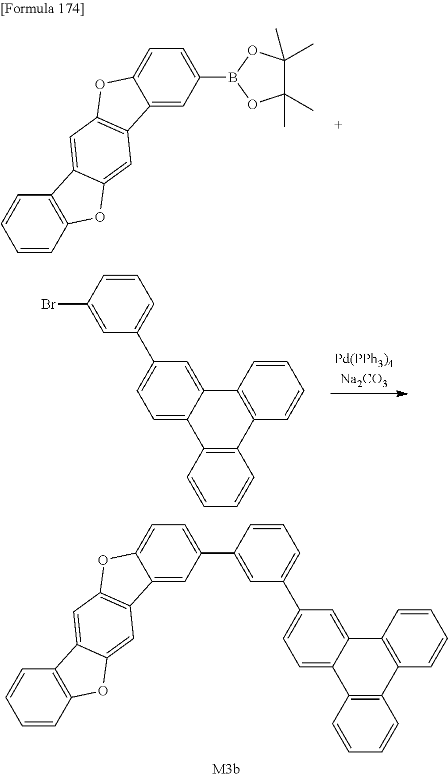

Under nitrogen atmosphere, 1,2-dimethoxyethane (120 mL) and water (30 mL) were added into a mixture of 2-(4,4,5,5-tetramethyl-1,3,2-dioxaborolane-2-yl)-benzo[1,2-b:4,5-b′]bisbenzofuran (3.07 g, 8.00 mmol), (3-(triphenylene-2-yl)phenyl) boronic acid (3.07 g, 8.00 mmol), tetrakistriphenylphosphine palladium (0.462 g, 0.400 mmol) and sodium carbonate (2.54 g, 24.0 mmol) and stirred at 85 degrees C. for five hours. After the reaction, a solid was filtrated and recrystallized with toluene to obtain the compound M3b (2.33 g, a yield of 52%). The obtained compound was identified as the compound M3b by analysis according to LC-MS (Liquid chromatography mass spectrometry).

### Synthesis Example 3

### (3) Synthesis Example 3: Synthesis of Compound M3c

### (3-1) Synthesis of Intermediate M3c-1

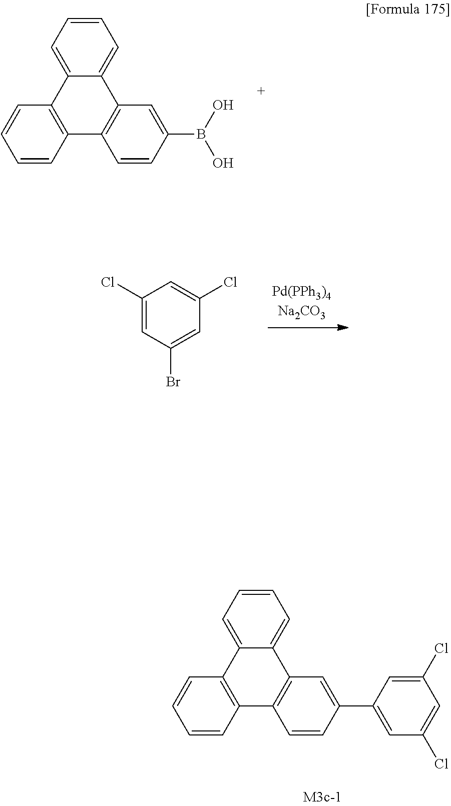 [SMILES: OB(O)c1ccc2c3ccccc3c3ccccc3c2c1 ; Clc1cc(Cl)cc(Br)c1 ; Clc1cc(Cl)cc(-c2ccc3c4ccccc4c4ccccc4c3c2)c1]

Under nitrogen atmosphere, 1,2-dimethoxyethane (130 mL) and water (65 mL) were added into a mixture of triphenylene-2-yl boronic acid (5.44 g, 20.0 mmol), 1-bromo-3,5-dichlorobenzene (4.52 g, 20.0 mmol), tetrakistriphenylphosphine palladium (1.16 g, 1.00 mmol) and sodium carbonate (6.36 g, 60.0 mmol) and stirred at 85 degrees C. for six hours. After the reaction, a solid was filtrated and recrystallized with toluene to obtain the intermediate M3c-1 (5.97 g, a yield of 80%).

### (3-2) Synthesis of Intermediate M3c-2

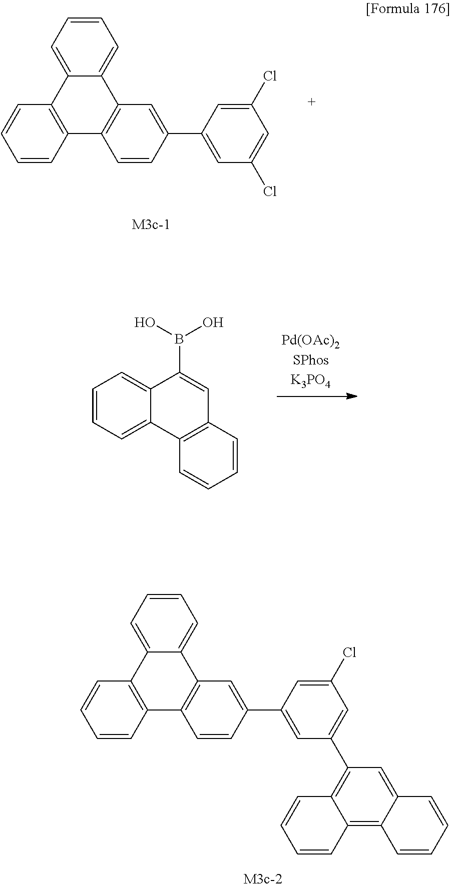 [SMILES: Clc1cc(Cl)cc(-c2ccc3c4ccccc4c4ccccc4c3c2)c1 ; OB(O)c1cc2ccccc2c2ccccc12 ; Clc1cc(-c2ccc3c4ccccc4c4ccccc4c3c2)cc(-c2cc3ccccc3c3ccccc23)c1]

Under nitrogen atmosphere, toluene (130 mL) was added into a mixture of the intermediate M3c-1 (5.23 g, 14.0 mmol), phenanthrene-9-yl boronic acid (3.11 g, 14.0 mmol), palladium acetate (0.157 g, 0.700 mmol), SPhos (0.575 g, 1.40 mmol) and potassium phosphate (8.92 g, 42.0 mmol) and stirred at 110 degrees C. for eight hours. After the reaction, a solid was filtrated and recrystallized with toluene to obtain the intermediate M3c-2 (3.24 g, a yield of 45%).

### (3-3) Synthesis of Compound M3c

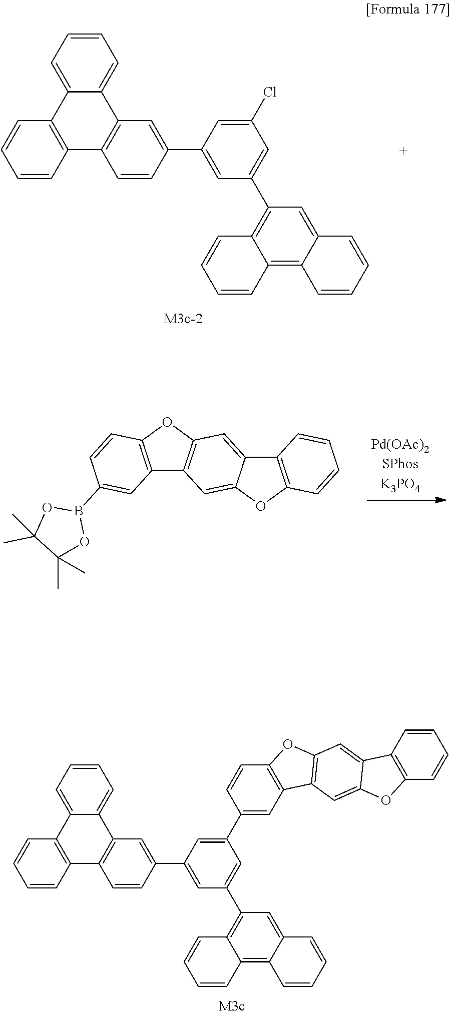 [SMILES: Clc1cc(-c2ccc3c4ccccc4c4ccccc4c3c2)cc(-c2cc3ccccc3c3ccccc23)c1 ; CC1(C)OB(c2ccc3oc4cc5c(cc4c3c2)oc2ccccc25)OC1(C)C ; c1ccc2c(c1)cc(-c1cc(-c3ccc4oc5cc6c(cc5c4c3)oc3ccccc36)cc(-c3ccc4c5ccccc5c5ccccc5c4c3)c1)c1ccccc12]

Under nitrogen atmosphere, toluene (130 mL) was added into a mixture of the intermediate M3c-2 (3.09 g, 6.00 mmol), 2-(4,4,5,5-tetramethyl-1,3,2-dioxaborolane-2-yl)-benzo[1,2-b:4,5-b′]bisbenzofuran (2.31 g, 6.00 mmol), palladium acetate (0.0674 g, 0.300 mmol), SPhos (0.246 g, 0.600 mmol) and potassium phosphate (3.82 g, 18.0 mmol) and stirred at 110 degrees C. for eight hours. After the reaction, a solid was filtrated and recrystallized with toluene to obtain the compound M3c (2.96 g, a yield of 67%). The obtained compound was identified as the compound M3c by analysis according to LC-MS (Liquid chromatography mass spectrometry).

### EXPLANATION OF CODE(S)

1 . . . organic EL device, 2 . . . substrate, 3 . . . anode, 4 . . . cathode, 5 . . . emitting layer, 6 . . . hole injecting layer, 7 . . . hole transporting layer, 8 . . . electron transporting layer, 9 . . . electron injecting layer

1. An organic electroluminescence device, comprising:
an anode;
a cathode; and
an emitting layer provided between the anode and the cathode, wherein:
the emitting layer comprises a delayed fluorescent compound M2 and a compound M3 represented by a formula (3) below, and
a singlet energy S₁(M2) of the compound M2 and a singlet energy S₁(M3) of the compound M3 satisfy a relationship of a numerical formula (Numerical Formula 1) below,
S₁(M3)>S₁(M2) (Numerical Formula 1), 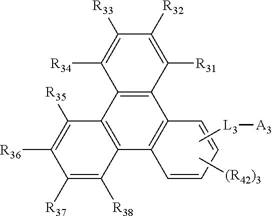
where, in the formula (3):
A₃ is a group represented by a formula (3a), (3b), (3c), (3d), (3e), or (3f) below;
L₃ is a single bond or a linking group, and L₃ as a linking group is a substituted or unsubstituted arylene group having 6 to 40 ring carbon atoms, a substituted or unsubstituted divalent heterocyclic group having 5 to 30 ring atoms, a group represented by -L₃₁-L₃₂-, or a group represented by -L₃₁-L₃₂-L₃₃-;
L₃₁, L₃₂, and L₃₃ are each independently a substituted or unsubstituted arylene group having 6 to 40 ring carbon atoms, or a substituted or unsubstituted divalent heterocyclic group having 5 to 30 ring atoms;
L₃₁, L₃₂, and L₃₃ are mutually the same or different;
R₃₁ to R₃₈ and R₄₂ are each independently a hydrogen atom or a substituent;
a substituent, if present, for L₃, and R₃₁ to R₃₈ and R₄₂ as a substituent are each independently a halogen atom, a cyano group, a substituted or unsubstituted aryl group having 6 to 30 ring carbon atoms, a substituted or unsubstituted heterocyclic group having 5 to 30 ring atoms, a substituted or unsubstituted alkyl group having 1 to 30 carbon atoms, a substituted or unsubstituted cycloalkyl group having 3 to 30 ring carbon atoms, a substituted or unsubstituted alkyl halide group having 1 to 30 carbon atoms, a substituted or unsubstituted alkenyl group having 2 to 30 carbon atoms, a substituted or unsubstituted alkynyl group having 2 to 30 carbon atoms, a substituted or unsubstituted alkylsilyl group having 3 to 30 carbon atoms, a substituted or unsubstituted arylsilyl group having 6 to 60 ring carbon atoms, a substituted or unsubstituted arylphosphoryl group having 6 to 60 ring carbon atoms, a hydroxy group, a substituted or unsubstituted alkoxy group having 1 to 30 carbon atoms, a substituted or unsubstituted aryloxy group having 6 to 30 ring carbon atoms, an amino group, a substituted or unsubstituted alkylamino group having 2 to 30 carbon atoms, a substituted or unsubstituted arylamino group having 6 to 60 ring carbon atoms, a thiol group, a substituted or unsubstituted alkylthio group having 1 to 30 carbon atoms, or a substituted or unsubstituted arylthio group having 6 to 30 ring carbon atoms; and
a plurality of R₄₂ are mutually the same or different, 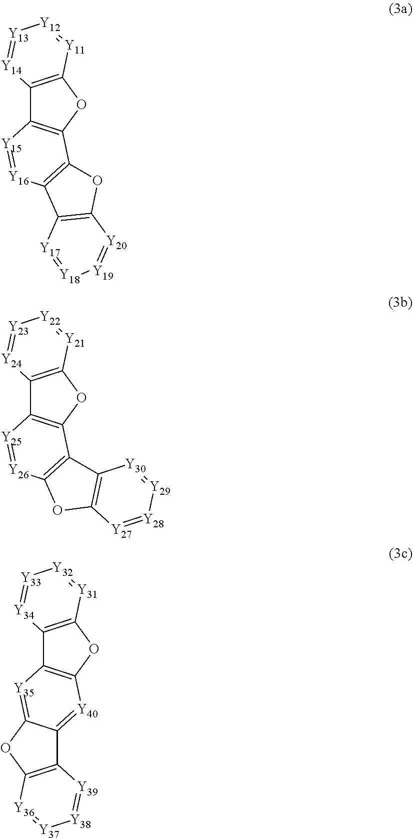 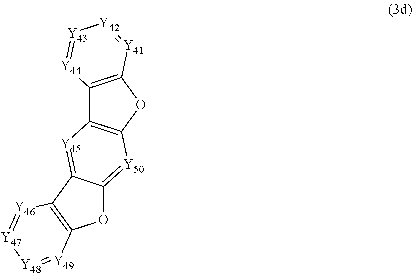 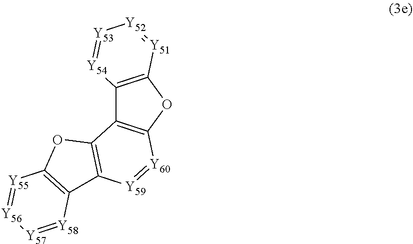 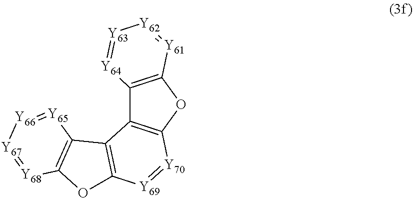
where, in the formula (3a):
Y₁₁ to Y₂₀ are each independently a nitrogen atom, CR₃₁₀, or a carbon atom bonded to L₃;
at least one of Y₁₁ to Y₂₀ is a carbon atom bonded to L₃;
in the formula (3b):
Y₂₁ to Y₃₀ are each independently a nitrogen atom, CR₃₁₀, or a carbon atom bonded to L₃;
at least one of Y₂₁ to Y₃₀ is a carbon atom bonded to L₃;
in the formula (3c):
Y₃₁ to Y₄₀ are each independently a nitrogen atom, CR₃₁₀, or a carbon atom bonded to L₃;
at least one of Y₃₁ to Y₄₀ is a carbon atom bonded to L₃;
in the formula (3d):
Y₄₁ to Y₅₀ are each independently a nitrogen atom, CR₃₁₀, or a carbon atom bonded to L₃;
at least one of Y₄₁ to Y₅₀ is a carbon atom bonded to L₃;
in the formula (3e):
Y₅₁ to Y₆₀ are each independently a nitrogen atom, CR₃₁₀, or a carbon atom bonded to L₃;
at least one of Y₅₁ to Y₆₀ is a carbon atom bonded to L₃;
in the formula (3f):
Y₆₁ to Y₇₀ are each independently a nitrogen atom, CR₃₁₀, or a carbon atom bonded to L₃;
at least one of Y₆₁ to Y₇₀ is a carbon atom bonded to L₃;
in the formulae (3a), (3b), (3c), (3d), (3e), and (3f):
R₃₁₀ is each independently a hydrogen atom or a substituent;
R₃₁₀ as a substituent is each independently a halogen atom, a cyano group, a substituted or unsubstituted aryl group having 6 to 30 ring carbon atoms, a substituted or unsubstituted heterocyclic group having 5 to 30 ring atoms, a substituted or unsubstituted alkyl group having 1 to 30 carbon atoms, a substituted or unsubstituted cycloalkyl group having 3 to 30 ring carbon atoms, a substituted or unsubstituted alkyl halide group having 1 to 30 carbon atoms, a substituted or unsubstituted alkenyl group having 2 to 30 carbon atoms, a substituted or unsubstituted alkynyl group having 2 to 30 carbon atoms, a substituted or unsubstituted alkylsilyl group having 3 to 30 carbon atoms, a substituted or unsubstituted arylsilyl group having 6 to 60 ring carbon atoms, a substituted or unsubstituted arylphosphoryl group having 6 to 60 ring carbon atoms, a hydroxy group, a substituted or unsubstituted alkoxy group having 1 to 30 carbon atoms, a substituted or unsubstituted aryloxy group having 6 to 30 ring carbon atoms, an amino group, a substituted or unsubstituted alkylamino group having 2 to 30 carbon atoms, a substituted or unsubstituted arylamino group having 6 to 60 ring carbon atoms, a thiol group, a substituted or unsubstituted alkylthio group having 1 to 30 carbon atoms, or a substituted or unsubstituted arylthio group having 6 to 30 ring carbon atoms; and
a plurality of R₃₁₀ are mutually the same or different.

2. The organic electroluminescence device according to claim 1, wherein:
the emitting layer further comprises a fluorescent compound M1, and
the singlet energy S₁(M2) of the compound M2 and a singlet energy S₁(M1) of the compound M1 satisfy a relationship of a numerical formula (Numerical Formula 2) below,
S₁(M2)>S₁(M1) (Numerical Formula 2).

3. The organic electroluminescence device according to claim 1, wherein:
R₃₁ to R_{3R}, R₁₂, and R₃₁₀ are each independently a hydrogen atom, or a substituted or unsubstituted aryl group having 6 to 30 ring carbon atoms.

4. The organic electroluminescence device according to claim 1, wherein:
R₃₁ to R₃₈, R₄₂, and R₃₁₀ are each independently a hydrogen atom, or a substituted or unsubstituted phenyl group.

5. The organic electroluminescence device according to claim 1, wherein;
R₃₁ to R₃₈, R₄₂, and R₃₁₀ are each a hydrogen atom.

6. The organic electroluminescence device according to claim 1, wherein:
A₃ is a group represented by a formula (30a), (30b), (30c), (30d), (30e), or (30f) below, 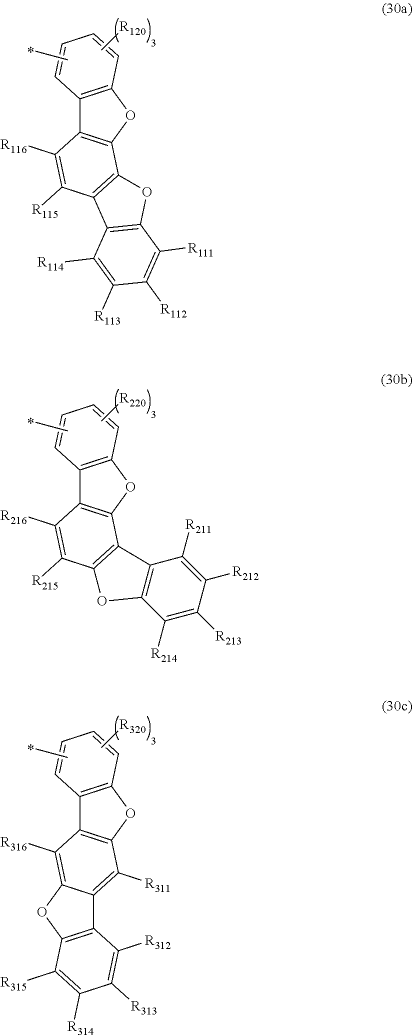 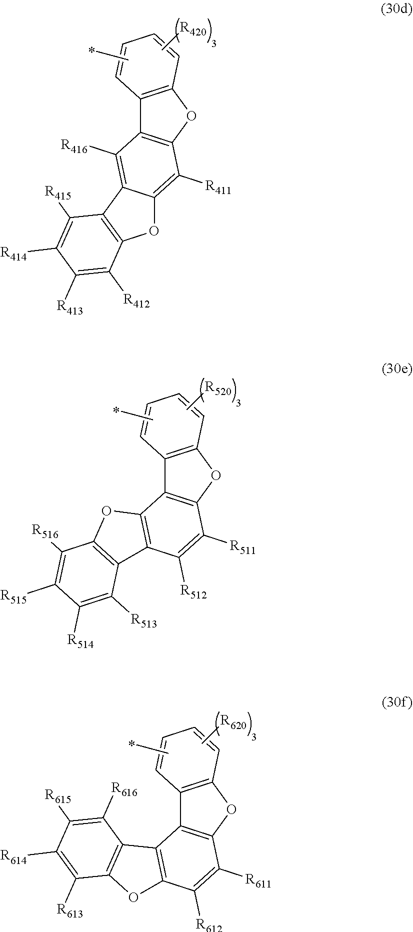
where, in the formulae (30a), (30b), (30c), (30d), (30e) and (30f):
R₁₁₁ to R₁₁₆, R₂₁₁ to R₂₁₆, R₃₁₁ to R₃₁₆, R₄₁₁ to R₄₁₆, R₅₁₁ to R₅₁₆, R₆₁₁ to R₆₁₆, R₁₂₀, R₂₂₀, R₃₂₀, R₄₂₀, R₅₂₀₀ and R₆₂₀ each independently represent the same as R₃₁₀ in the formulae (3a), (3b), (3c), (3d), (3e), and (3f);
a plurality of R₁₂₀ are mutually the same or different;
a plurality of R₂₂₀ are mutually the same or different;
a plurality of R₃₂₀ are mutually the same or different;
a plurality of R₄₂₀ are mutually the same or different;
a plurality of R₅₂₀ are mutually the same or different;
a plurality of R₆₂₀ are mutually the same or different; and
* represents a bonding position to L₃.

7. The organic electroluminescence device according to claim 6, wherein:
A₃ is a group represented by a formula (31a), (31b), (31c), (31d), (31e), or (31f) below, 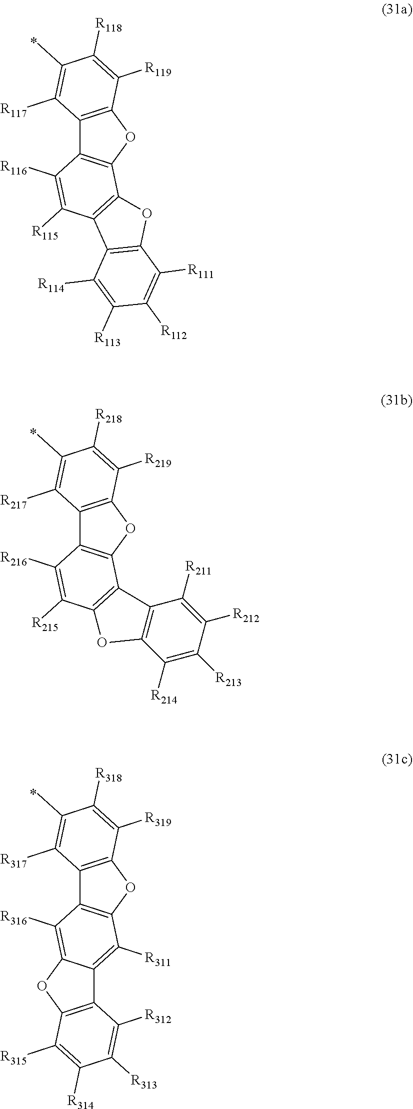 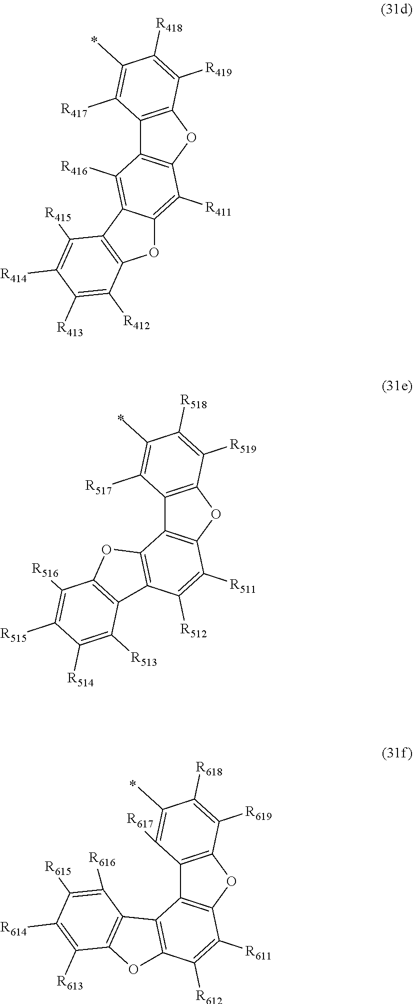
where, in the formulae (31a), (31b), (31c), (31d), (31e), and (31f):
R₁₁₁ to R₁₁₉, R₂₁₁ to R₂₁₉, R₃₁₁ to R₃₁₉, R₄₁₁ to R₄₁₉, R₅₁₁ to R₅₁₉, and R₆₁₁ to R₆₁₉ each independently represent the same as R₃₁₀ in the formulae (3a), (3b), (3c), (3d), (3e), and (3f); and
* represents a bonding position to L₃.

8. The organic electroluminescence device according to claim 6, wherein:
the compound M3 is represented by a formula (301), (302), (303), (304), (305), or (306) below, 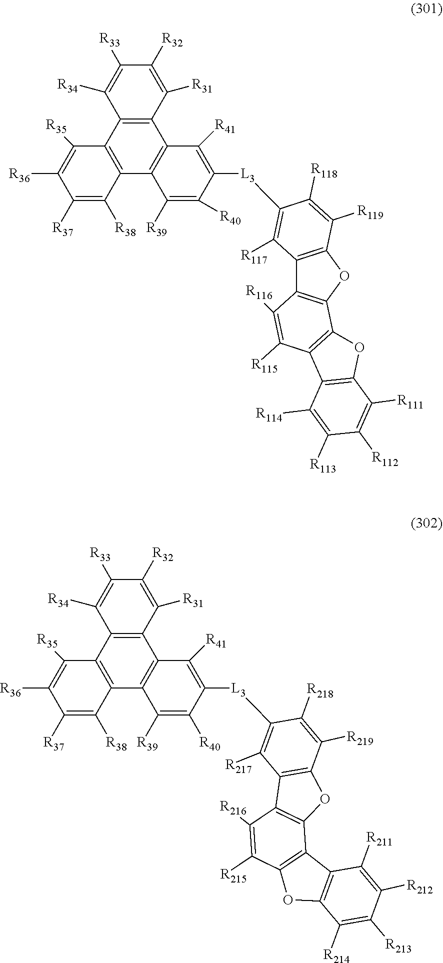 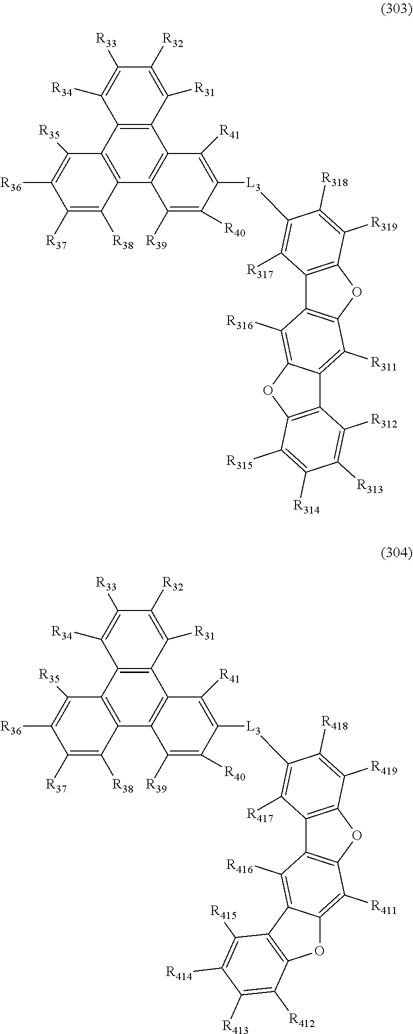 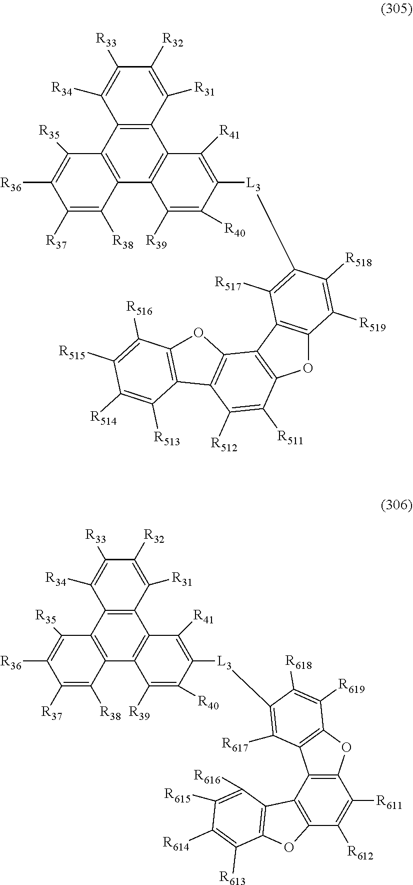
where, in the formulae (301), (302), (303), (304), (305), and (306):
R₁₁₁ to R₁₁₉, R₂₁₁ to R₂₁₉, R₃₁₁ to R₃₁₉, R₄₁₁ to R₄₁₉, R₅₁₁ to R₅₁₉, and R₆₁₁ to R₆₁₉ each independently represent the same as R₃₁₀ in the formulae (3a), (3b), (3c), (3d), (3e), and (3f);
R₃₁ to R₄₁ each represent the same as R₄₂ in the formula (3); and
L₃ represents the same as L₃ in the formula (3).

9. The organic electroluminescence device according to claim 6, wherein:
R₁₁₁ to R₁₂₀, R₂₁₁ to R₂₂₀, R₃₁₁ to R₃₂₀, R₄₁₁ to R₄₂₀, R₅₁₁ to R₅₂₀, R₆₁₁ to R₆₂₀, and R₃₁ to R₄₂ are each independently a hydrogen atom, or a substituted or unsubstituted aryl group having 6 to 30 ring carbon atoms.

10. The organic electroluminescence device according to claim 6, wherein:
R₁₁₁ to R₁₂₀, R₂₁₁ to R₂₂₀, R₃₁₁ to R₃₂₀, R₄₁₁ to R₄₂₀, R₅₁₁ to R₅₂₀, R₆₁₁ to R₆₂₀, and R₃₁ to R₄₂ are each independently a hydrogen atom, or a substituted or unsubstituted phenyl group.

11. The organic electroluminescence device according to claim 6, wherein:
R₁₁₁, to R₁₂₀, R₂₁₁ to R₂₂₀, R₃₁₁ to R₃₂₀, R₄₁₁ to R₄₂₀, R₅₁₁ to R₅₂₀, R₆₁₁ to R₆₂₀, and R₃₁ to R₄₂ are each a hydrogen atom.

12. The organic electroluminescence device according to claim 1, wherein:
L₃ is a single bond or a linking group, and L₃ as a linking group is a substituted or unsubstituted arylene group having 6 to 40 ring carbon atoms, a group represented by -L₃₁-L₃₂-, or a group represented by -L₃₁-L₃₂-L₃₃-, and
L₃₁, L₃₂, and L₃₃ are each independently a substituted or unsubstituted arylene group having 6 to 40 ring carbon atoms.

13. The organic electroluminescence device according to claim 1, wherein:
L₃ is a single bond or a linking group, and L₃ as a linking group is a substituted or unsubstituted phenylene group, a substituted or unsubstituted biphenylene group, a substituted or unsubstituted terphenylene group, or a substituted or unsubstituted naphthylene group.

14. The organic electroluminescence device according to claim 1, wherein:
a substituent, if present, for L₃ is a substituted or unsubstituted aryl group having 6 to 30 ring carbon atoms.

15. The organic electroluminescence device according to claim 1, wherein:
a substituent, if present, for L₃ is a substituted or unsubstituted phenyl group.

16. The organic electroluminescence device according to claim 1, wherein:
the emitting layer comprises no metal complex.

17. An electronic device, comprising the organic electroluminescence device according to claim 1.

18. A compound represented by a formula (300) below, 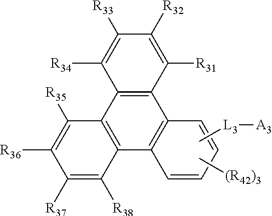
where, in the formula (300):
A₃ is a group represented by a formula (301a), (301b), (301c), (301d), (301e), (301f), (301g), (301h), (301i), (301j), (301k), or (301m) below;
L₃ is a single bond or a linking group, and L₃ as a linking group is a substituted or unsubstituted arylene group having 6 to 40 ring carbon atoms, a group represented by -L₃₁-L₃₂-, or a group represented by -L₃₁-L₃₂-L₃₃-;
L₃₁, L₃₂, and L₃₃ are each independently a substituted or unsubstituted arylene group having 6 to 40 ring carbon atoms;
L₃₁, L₃₂, and L₃₃ are mutually the same or different;
L₃, L₃₁, L₃₂, and L₃₃ are each not a substituted or unsubstituted anthracenylene group;
R₃₁ to R₃₈ and R₄₂ are each independently a hydrogen atom or a substituent;
a substituent, if present, for L₃, and R₃₁ to R₃₈, and R₄₂ as a substituent are each independently a halogen atom, a cyano group, a substituted or unsubstituted aryl group having 6 to 30 ring carbon atoms, a substituted or unsubstituted heterocyclic group having 5 to 30 ring atoms, a substituted or unsubstituted alkyl group having 1 to 30 carbon atoms, a substituted or unsubstituted cycloalkyl group having 3 to 30 ring carbon atoms, a substituted or unsubstituted alkyl halide group having 1 to 30 carbon atoms, a substituted or unsubstituted alkenyl group having 2 to 30 carbon atoms, a substituted or unsubstituted alkynyl group having 2 to 30 carbon atoms, a substituted or unsubstituted alkylsilyl group having 3 to 30 carbon atoms, a substituted or unsubstituted arylsilyl group having 6 to 60 ring carbon atoms, a substituted or unsubstituted arylphosphoryl group having 6 to 60 ring carbon atoms, a hydroxy group, a substituted or unsubstituted alkoxy group having 1 to 30 carbon atoms, a substituted or unsubstituted aryloxy group having 6 to 30 ring carbon atoms, an amino group, a substituted or unsubstituted alkylamino group having 2 to 30 carbon atoms, a substituted or unsubstituted arylamino group having 6 to 60 ring carbon atoms, a thiol group, a substituted or unsubstituted alkylthio group having 1 to 30 carbon atoms, or a substituted or unsubstituted arylthio group having 6 to 30 ring carbon atoms; and
a plurality of R₄₂ are mutually the same or different, 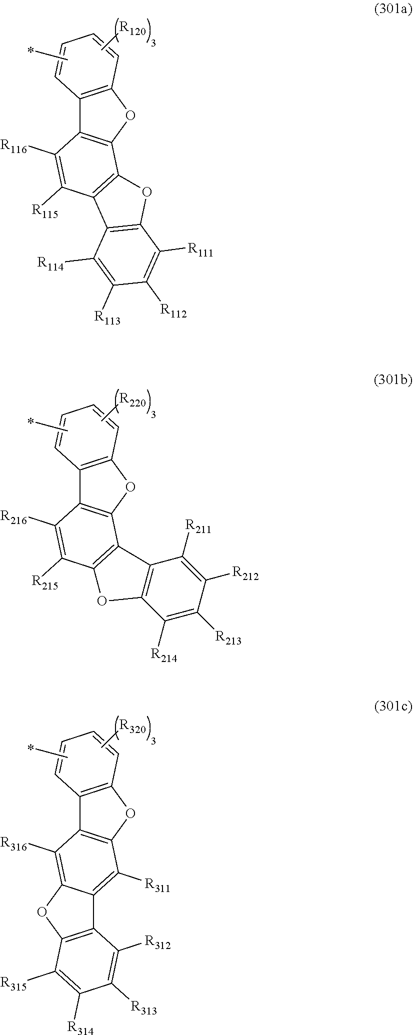 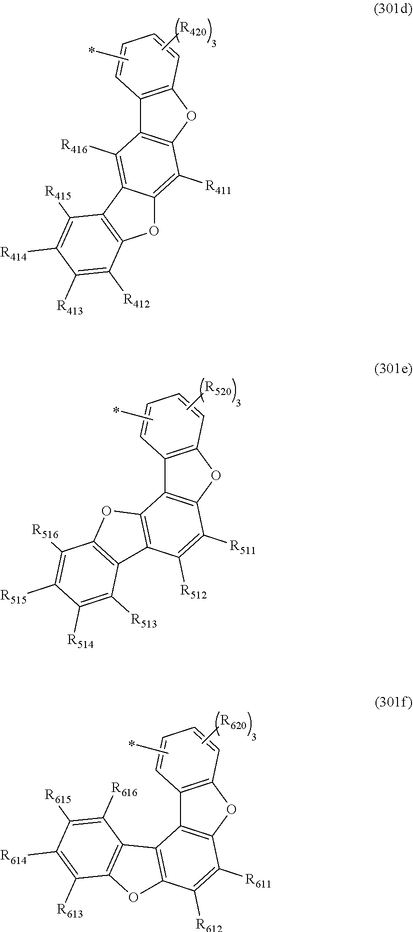 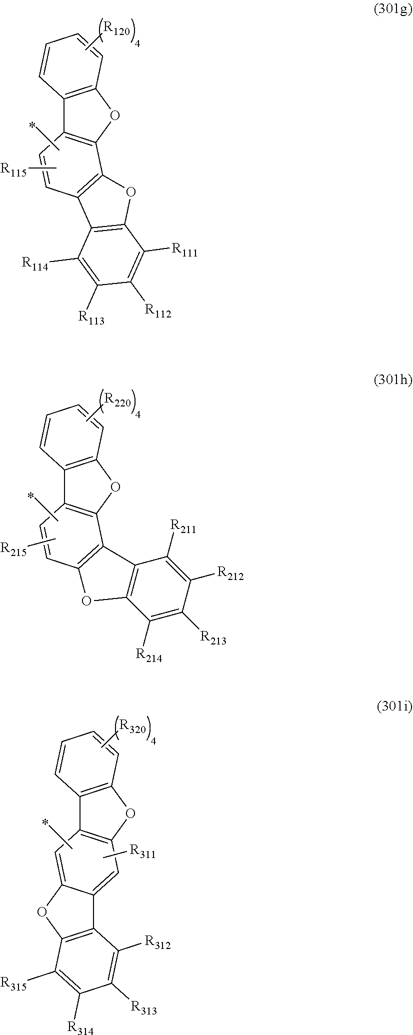 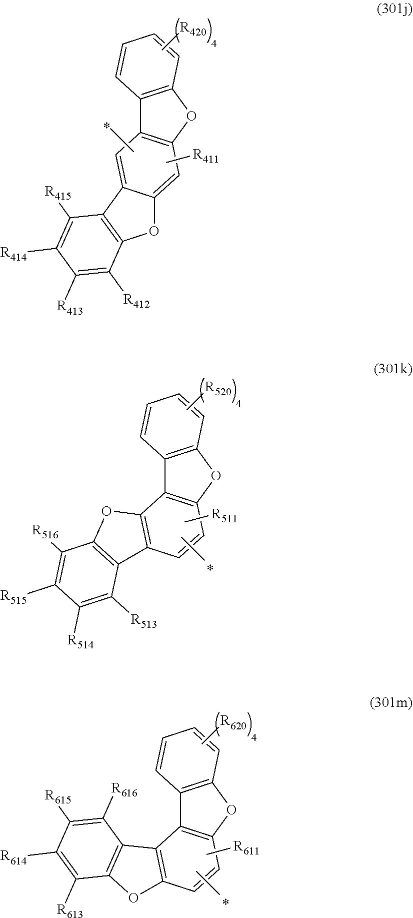
where, in the formulae (301a), (301b), (301c), (301d), (301e), (301f), (301g), (301h), (301i), (301j), (301k), and (301m):
R₁₁₁ to R₁₁₆, R₂₁₁ to R₂₁₆, R₃₁₁ to R₃₁₆, R₄₁₁ to R₄₁₆, R₅₁₁ to R₅₁₆, R₆₁₁ to R₆₁₆, R₁₂₀, R₂₂₀, R₃₂₀, R₄₂₀, R₅₂₀, and R₆₂₀ are each independently a hydrogen atom or a substituent;
R₁₁₁ to R₁₁₆, R₂₁₁ to R₂₁₆, R₃₁₁ to R₃₁₆, R₄₁₁ to R₄₁₆, R₅₁₁ to R₅₁₆, R₆₁₁ to R₆₁₆, R₁₂₀, R₂₂₀, R₃₂₀, R₄₂₀, R₅₂₀, and R₆₂₀ as a substituent are each independently a halogen atom, a cyano group, a substituted or unsubstituted aryl group having 6 to 30 ring carbon atoms, a substituted or unsubstituted heterocyclic group having 5 to 30 ring atoms, a substituted or unsubstituted alkyl group having 1 to 30 carbon atoms, a substituted or unsubstituted cycloalkyl group having 3 to 30 ring carbon atoms, a substituted or unsubstituted alkyl halide group having 1 to 30 carbon atoms, a substituted or unsubstituted alkenyl group having 2 to 30 carbon atoms, a substituted or unsubstituted alkynyl group having 2 to 30 carbon atoms, a substituted or unsubstituted alkylsilyl group having 3 to 30 carbon atoms, a substituted or unsubstituted arylsilyl group having 6 to 60 ring carbon atoms, a substituted or unsubstituted arylphosphoryl group having 6 to 60 ring carbon atoms, a hydroxy group, a substituted or unsubstituted alkoxy group having 1 to 30 carbon atoms, a substituted or unsubstituted aryloxy group having 6 to 30 ring carbon atoms, an amino group, a substituted or unsubstituted alkylamino group having 2 to 30 carbon atoms, a substituted or unsubstituted arylamino group having 6 to 60 ring carbon atoms, a thiol group, a substituted or unsubstituted alkylthio group having 1 to 30 carbon atoms, or a substituted or unsubstituted arylthio group having 6 to 30 ring carbon atoms;
R₁₁₁ to R₁₁₄, R₂₁₁ to R₂₁₄, R₃₁₂ to R₃₁₅, R₄₁₂ to R₄₁₅, R₅₁₃ to R₅₁₆, R₆₁₃ to R₆₁₆, R₁₂₀, R₂₂₀, R₃₂₀, R₄₂₀, R₅₂₀, and R₆₂₀ are each not an unsubstituted triphenylenyl group;
a plurality of R₁₂₀ are mutually the same or different;
a plurality of R₂₂₀ are mutually the same or different;
a plurality of R₃₂₀ are mutually the same or different;
a plurality of R₄₂₀ are mutually the same or different;
a plurality of R₅₂₀ are mutually the same or different;
a plurality of R₆₂₀ are mutually the same or different; and
* represents a bonding position to L₃.

19. The compound according to claim 18, wherein:
A₃ is a group represented by a formula (310a), (310b), (310c), (310d), (310e), or (310f) below, 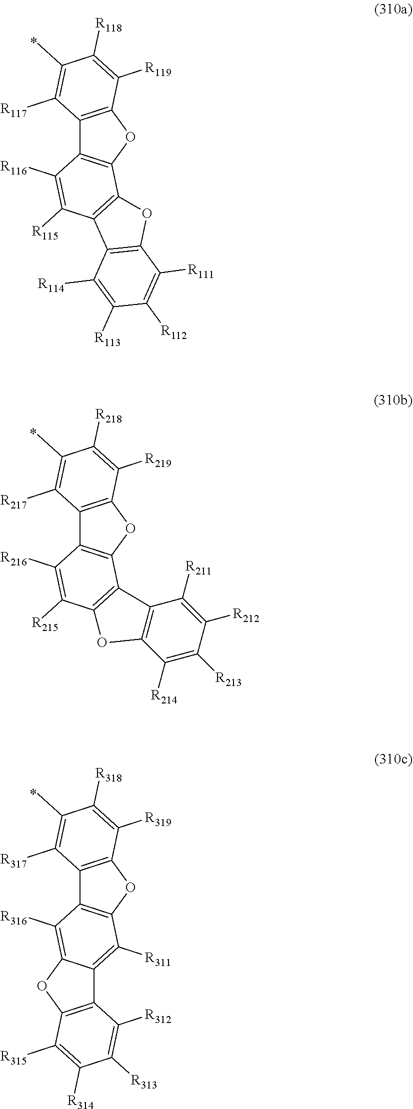 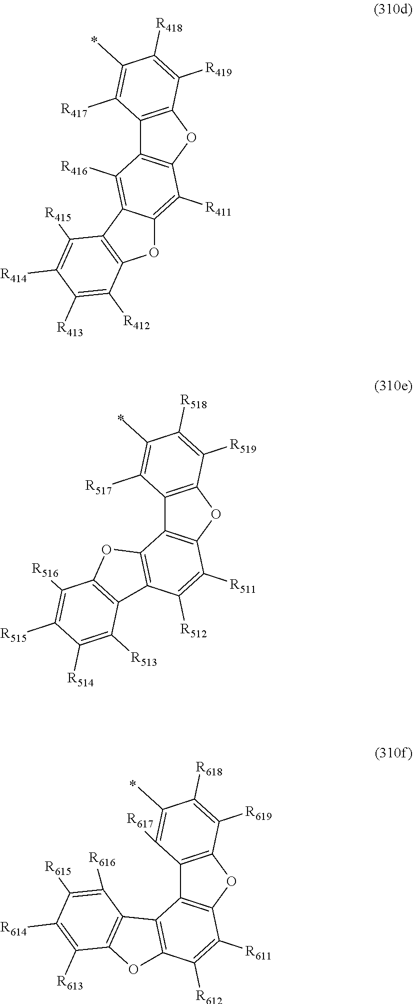
where, in the formula (310a): R₁₁₁ to R₁₁₉ each independently represent the same as R₁₁₅ in the formula (301a), and R₁₁₁ to R₁₁₄ and R₁₁₇ to R₁₁₉ are each not an unsubstituted triphenylenyl group;
in the formula (310b): R₂₁₁ to R₂₁₉ each independently represent the same as R₂₁₅ in the formula (301b), and R₂₁₁ to R₂₁₄ and R₂₁₇ to R₂₁₉ are each not an unsubstituted triphenylenyl group;
in the formula (310c): R₃₁₁ to R₃₁₉ each independently represent the same as R₃₁₁ in the formula (301c), and R₃₁₂ to R₃₁₅ and R₃₁₇ to R₃₁₉ are each not an unsubstituted triphenylenyl group;
in the formula (310d): R₄₁₁ to R₄₁₉ each independently represent the same as R₄₁₁ in the formula (301d), and R₄₁₂ to R₄₁₅ and R₄₁₇ to R₄₁₉ are each not an unsubstituted triphenylenyl group;
in the formula (310e): R₅₁₁ to R₅₁₉ each independently represent the same as R₅₁₁ in the formula (301e), and R₅₁₃ to R₅₁₆ and R₅₁₇ to R₅₁₉ are each not an unsubstituted triphenylenyl group;
in the formula (310f): R₆₁₁ to R₆₁₉ each independently represent the same as R₆₁₁ in the formula (301f), and R₆₁₃ to R₆₁₆ and R₆₁₇ to R₆₁₉ are each not an unsubstituted triphenylenyl group; and
* represents a bonding position to L₃.

20. The compound according to claim 18, wherein:
the compound is represented by a formula (301A), (302A), (303A), (304A), (305A), or (306A) below, 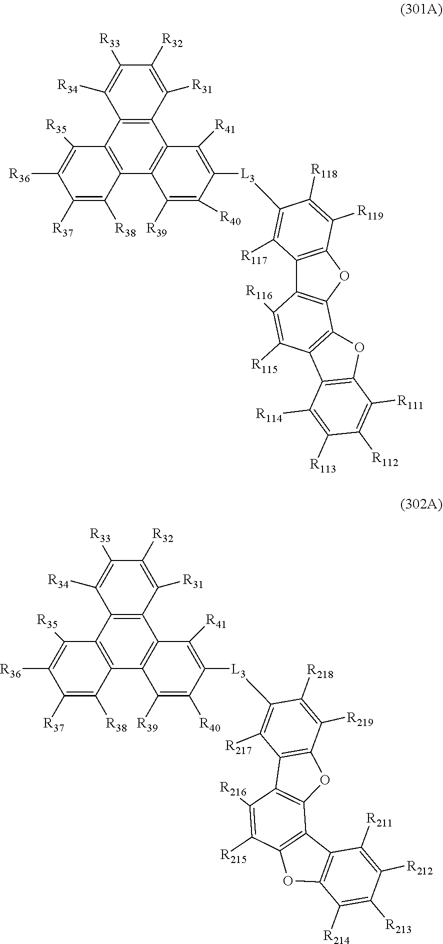 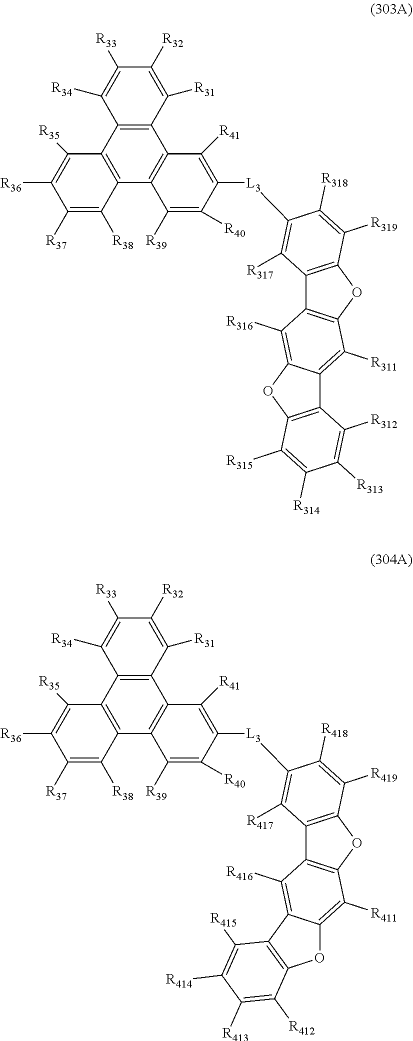 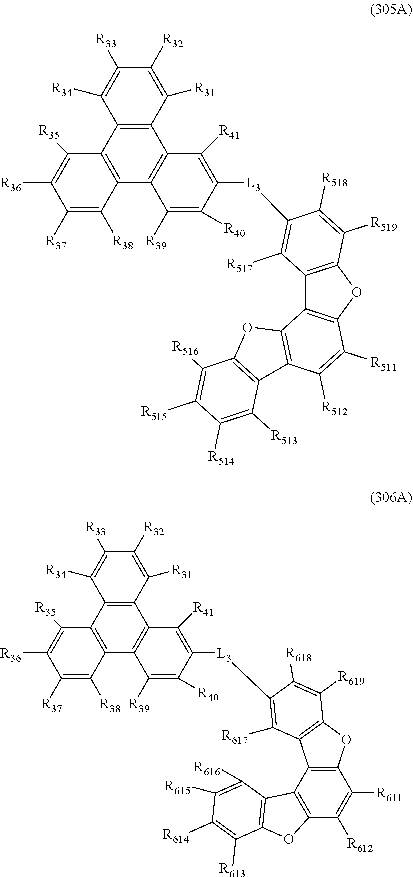
where, in the formula (301A): R₁₁₁ to R₁₁₉ each independently represent the same as R₁₁₅ in the formula (301a), and R₄₁₂ to R₄₁₄ and R₄₁₇ to R₄₁₉ are each not an unsubstituted triphenylenyl group;
in the formula (302A): R₂₁₁ to R₂₁₉ each independently represent the same as R₂₁₅ in the formula (301b), and R₂₁ to R₂₁₄ and R₂₁₇ to R₂₁₉ are each not an unsubstituted triphenylenyl group;
in the formula (303A): R₃₁₁ to R₃₁₉ each independently represent the same as R₃₁₁ in the formula (301c), and R₃₁₂ to R₃₁₅ and R₃₁₇ to R₃₁₉ are each not an unsubstituted triphenylenyl group;
in the formula (304A): R₄₁₁ to R₄₁₉ each independently represent the same as R₄₁₁ in the formula (301d), and R₄₁₂ to R₄₁₅ and R₄₁₇ to R₄₁₉ are each not an unsubstituted triphenylenyl group;
in the formula (305A): R₅₁₁ to R₅₁₉ each independently represent the same as R₄₂₁ in the formula (301e), and R₅₁₃ to R₅₁₆ and R₅₁₇ to R₅₁₉ are each not an unsubstituted triphenylenyl group;
in the formula (306A): R₆₁₁ to R₆₁₉ each independently represent the same as R₆₁₁ in the formula (301f), and R₆₁₃ to R₆₁₆ and R₆₁₇ to R₆₁₉ are each not an unsubstituted triphenylenyl group;
in the formulae (301A) to (306A):
R₃₁ to R₄₁ each independently represent the same as R₄₂ in the formula (300); and
L₃ represents the same as L₃ in the formula (300).

21. The compound according to claim 18, wherein:
R₃₁ to R₄₂, R₁₁₁ to R₁₂₀, R₂₁₁ to R₂₂₀, R₃₁₁ to R₃₂₀, R₄₁₁ to R₄₂₀, R₅₁₁ to R₅₂₀, and R₆₁₁ to R₆₂₀ are each independently a hydrogen atom, or a substituted or unsubstituted aryl group having 6 to 30 ring carbon atoms.

22. The compound according to claim 18, wherein:
R₃₁ to R₄₂, R₁₁₁ to R₁₂₀, R₂₁₁ to R₂₂₀, R₃₁₁ to R₃₂₀, R₄₁₁ to R₄₂₀, R₅₁₁ to R₅₂₀, and R₆₁₁ to R₆₂₀ are each independently a hydrogen atom, or a substituted or unsubstituted phenyl group.

23. The compound according to claim 18, wherein:
R₃₁ to R₄₂, R₁₁₁ to R₁₁₉, R₂₁₁ to R₂₁₉, R₃₁₁ to R₃₁₉, R₄₁₁ to R₄₁₉, R₅₁₁ to R₅₁₉, R₆₁₁ to R₆₁₉, R₁₂₀, R₂₂₀, R₃₂₀, R₄₂₀, R₅₂₀, and R₆₂₀ are each a hydrogen atom.

24. The compound according to claim 18, wherein:
L₃ is a single bond or a linking group, and L₃ as a linking group is a substituted or unsubstituted phenylene group, a substituted or unsubstituted biphenylene group, a substituted or unsubstituted terphenylene group, or a substituted or unsubstituted naphthylene group.

25. The compound according to claim 18, wherein:
a substituent, if present, for L₃ is a substituted or unsubstituted aryl group having 6 to 30 ring carbon atoms.

26. The compound according to claim 18, wherein:
a substituent, if present, for L₃ is a substituted or unsubstituted phenyl group.

27. An organic electroluminescence device, comprising:
an anode;
a cathode; and
an emitting layer provided between the anode and the cathode, wherein:
the emitting layer comprises a delayed fluorescent compound M2 and the compound according to claim 18.

28. An electronic device, comprising the organic electroluminescence device according to claim 27.
